(19) United States
(12) Patent Application Publication
(10) Pub. No.: US 2024/0158385 A1
(43) Pub. Date: May 16, 2024

(54) NOVEL COMPOUNDS

(71) Applicant: Cerevance, Inc., Boston, MA (US)

(72) Inventors: Roland Werner Burli, Cambridge (GB); Kevin Doyle, Cambridge (GB); Martin Teall, Cambridge (GB)

(21) Appl. No.: 18/275,862

(22) PCT Filed: Feb. 8, 2022
(86) PCT No.: PCT/GB2022/050324

§ 371 Date: Aug. 4, 2023

(30) Foreign Application Priority Data
Feb. 8, 2021 (GB) 2101734.8

(51) Int. Cl. C07D 413/14; C07D 405/14; C07D 409/14; C07D 417/14; C07D 471/04

(52) U.S. Cl. CPC C07D 413/14; C07D 405/14; C07D 409/14; C07D 417/14; C07D 471/04

(57) ABSTRACT

The present invention provides compounds of formula (I) and pharmaceutically acceptable salts, N-oxides, solvates and prodrugs thereof Formula (I) wherein R², R³, R⁴, X¹, X², X³ and X⁴ are as defined in the specification, processes for their preparation, pharmaceutical compositions containing them and their use in therapy, particularly for use in treating disorders associated with KCNK₁₃ activity.

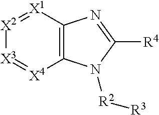

### FIELD OF THE INVENTION

The present invention relates to benzimidazoles and related compounds, processes for their preparation, pharmaceutical compositions containing them and their use in therapy, particularly for use in treating disorders associated with KCNK13 activity.

### BACKGROUND OF THE INVENTION

Inflammation & Neuroinflammation

Inflammation is part of the complex biological response of the body's tissue systems to harmful stimuli, such as invading pathogens or irritants and cellular damage. This is a generally protective response involving the cells of the immune system, blood vessels, and a diverse range of molecular mediators that function to eliminate the initial cause of irritation and cellular injury, clear out necrotic cells and tissues damaged from the original insult and initiate tissue repair. However, if inflammation becomes chronic or uncontrolled, then it can become causative or involved in the long-term progression of a range of diseases throughout the body, for example, arthritis, autoimmune disease, inflammatory bowel disorders, coeliac disease, hepatitis, asthma etc.

In the central nervous system (CNS) inflammation or neuroinflammation is a common underlying pathological feature of most neurological disorders and chronic neuroinflammation is evident in most if not all progressive neurodegenerative diseases such as Alzheimer's (AD) and Parkinson's disease (PD) (Heneka et al, 2014, Nat Rev Immunol, 14, 463-477), autoimmune disorders such as multiple sclerosis (Barclay & Shinohara, 2017, Brain Pathol, 27(2), 213-219) and can mediate ongoing damage to the CNS following brain injuries such as stroke (Jayaraj et al, 2019, J Neuroinflam, 16, 142-166) or traumatic brain injury (Simon et al, 2017, Nat Rev Neurol, 13(3), 171-191). Neuroinflammation has even been shown to be present and to play a role in psychiatric illnesses such as depression (Najjar et al, 2013, J Neuroinflammation, 10, 43-67; Wohleb et al, 2016, Nat Rev Neurosci, 17(8), 497-511) where overt tissue damage is less evident. The importance of neuroinflammation in disease is further underlined by findings that suggest that genes for immune receptors, such as TREM2 and CD33 are risk factors for, and afford selective vulnerability to a variety of neurodegenerative diseases including AD and PD (Jay et al, 2017, Mol Neurodegener, 12, 56-89). Many of these genes, including TREM2 and CD33, are exclusively expressed in brain microglia (MG) pointing to a key role of this cell type in neuroinflammation and pathogenic disease processes (Colonna & Butovsky, 2017, Annu Rev Immunol, 35, 441-468; Ransohoff, 2016, Science, 353, 777-783).

Microglia

Microglia (MG) are generally considered to be the brain's resident macrophages playing a central role in the development, homeostasis and ultimately diseases of the CNS. MG arise solely from yolk sac erythromyeloid precursors and interact with almost all CNS components during embryonic and postnatal development. Adult MG have a sentinel type role surveying their environment and interacting with essentially all CNS components and thus have a marked impact on normal brain functioning and maintenance of tissue integrity. In order to achieve this, MG have the ability to rapidly adapt to their environment, increasing their cell number and modifying their cellular function and activation states (of which they have a broad spectrum), mediating and responding to damage, infection and inflammation. Specifically, during these challenged environments MG change their morphology, from the ramified sentinel phenotype to more amoeboid, which is accompanied by higher levels of phagocytic activity; increased proliferation and a cascade of cellular biochemistry results in cytokine release and an orchestrated inflammatory response process to ultimately resolve the adverse event/challenge (Li & Barres, 2018, Nat Rev Immunol, 18, 225-242). This microglial activation is a salient feature of all neurodegenerative diseases and can alter disease processes and progression. Although microglial activation is an initially favourable response to environment, there is clear evidence that this becomes dysfunctional and ultimately plays a role in driving inflammation, cell damage and loss, progressing the neurodegenerative disease process. The biochemical processes involved are complex, but a number of pathways have been identified as being key to the disease processes and potential intervention points for therapeutic approaches; one such process is that involving the nod-like receptor family pyrin domain containing 3 (NLRP3) cascades (Heneka et al, 2018, Nat Revs Neurosci, 19, 610-621).

NLRP3

NLRP3 is a component of the innate immune system that functions as a pattern recognition receptor (PRR) that recognises pathogen-associated molecular patterns (PAMPs) and damage-associated molecular patterns (DAMPs) which are generated by endogenous stress and trigger downstream inflammatory pathways to eliminate microbial infection and repair damaged tissues (Kelley et al, 2019, Int J Mol Sci, 20, 3328-3352). The activation of the NLRP3 inflammasome requires a two-step process, comprising priming and then activation. Priming usually occurs through the stimulation of toll-like receptors (TLRs) (Toma et al, 2010, J Immunol, 184, 5287-5297; Qiao et al, 2012, FEBS Lett, 586, 1022-1026), which mediates upregulation of the nuclear factor-kappa B (NF-κB) pathway to increase the expression of NLRP3, caspase-1, and prointerleukin-1β (pro-IL-1β). The secondary step is then required to trigger the formation of the inflammasome complex comprising NLRP3 together with the adaptor ASC protein PYCARD and caspase-1. This activated NLRP3 inflammasome leads to activation of caspase-1 which in turn activates the inflammatory cytokine, IL-1β. The NLRP3 inflammasome appears to be activated by changes in intracellular potassium (K⁺), and K⁺ efflux in itself is capable of activating NLRP3, while high extracellular K⁺ blocks the activation of the NLRP3 inflammasome but not the other inflammasomes (Pétrilli et al, 2007, Cell Death Differ, 14, 1583-1589; Muñoz-Planillo et al, 2013, Immunity, 38, 1142-1153). Thus, a decrease of intracellular K⁺ has been considered to be the common trigger for NLRP3 inflammasome activation.

Genetic gain of function (GoF) mutations in the NLRP3 gene have been associated with a spectrum of dominantly inherited autoinflammatory diseases called cryopyrin-associated periodic syndrome (CAPS). These include familial cold autoinflammatory syndrome (FCAS), Muckle-Wells syndrome (MWS), chronic infantile neurological cutaneous and articular (CINCA) syndrome and neonatal onset multisystem inflammatory disease (NOMID). These diseases produce a diversity of immune-mediated organ changes and permanent central nervous system damage resulting in intellectual abnormalities (Izawa et al, 2012, DNA Research, 19(2), 143-152). In addition, exome sequencing data for genetic variation of NLRP3 in Parkinson's populations identified multiple single-nucleotide polymorphisms (SNPs) including rs7525979 that was associated with a significantly reduced risk of developing PD. Mechanistic studies indicated that the synonymous SNP, NLRP3 rs7525979, alters the efficiency of NLRP3 translation impacting NLRP3 protein stability and hence reducing NLRP3 inflammasome function (von Herrmann et al, 2018, NPJ Parkinsons Dis, 4, 2-10). Similarly, two functional single-nucleotide polymorphisms (SNPs) in the NLRP3 gene (rs2027432 and rs10754558) have been found to be associated with late-onset Alzheimer's disease in a Han Chinese population (Tan et al, 2013, Neuroimmunol, 265, 91-95).

NLRP3 Disease Association & Therapeutic Potential

These genetic observations have highlighted diseases caused, as with the genetic gain of function mutations, or involving NLRP3 dysfunction in the onset of and ongoing pathological processes. However, NLRP3 has been associated with a diverse range of diseases and conditions (Table 1) and is an important contributor to inflammatory diseases throughout the body (for general reviews, see Mangan et al, 2018, Nat Rev Drug Discov, 17, 588-606).

TABLE 1

| Disease type | Disease | Citations |
|---|---|---|
| Neurodegeneration | Alzheimer's disease | Heneka et al, 2013, Nature, 493, 674-678; |
| | | Dempsey et al, 2017, Brain Behav Immun, |
| | | 61, 306-316. |
| | Parkinson's disease | Gordon et al, 2018, Sci Transl Med, |
| | | 10(465), 1-25; |
| | | Haque et al, 2020, Mov Disord, 35(1), 20- |
| | | 33. |
| | Frontal temporal dementia/ | Ising et al, 2019, Nature, 575, 669-673; |
| | progressive supranuclear | Stancu et al, 2019, Acta Neuropathol, |
| | palsy (PSP)/tauopathies | 137(4), 599-617. |
| | Amyotrophic lateral sclerosis | Deora et al, 2020, Glia, 68(2), 407-421. |
| | (ALS)/motor neuron | |
| | disease (MND) | |
| | Traumatic brain injury | Irrera et al, 2017, Front Pharmacol, 8, |
| | | 459-469; |
| | | Ismael et al, 2018, J Neurotrauma, 35, |
| | | 1294-1303; |
| | | Kuwar et al, 2019, J Neuroinflam, 16, 81- |
| | | 91. |
| | Multiple sclerosis | Gris et al, 2010, J Immunol, 185, 974-981; |
| | | Coll et al, 2015, Nat Med, 21, 248-255. |
| | Stroke/ischemic insult | Yang et al, 2014, J Cereb Blood Flow |
| | | Metab, 34(4), 660-667; |
| | | Ward et al, 2019, Pharmacol Res, 142, |
| | | 237-250. |
| Psychiatric | Depression | Kaufmann et al, 2017, Brain Behav |
| | | Immun, 64, 367-383; |
| | | Su et al, 2017, Behav Brain Res, 322, 1-8. |
| | Stress/anxiety/PTSD | Lei et al, 2017, Brain Res, 1671, 43-54; |
| | | Wang et al, 2018, J Neuroinflammation, |
| | | 15(1), 21-35; |
| | | Dong et al, 2020, J Neuroinflammation, |
| | | 17, 205-221. |
| | Schizophrenia/bipolar | Giridharan et al, 2020, Cells, 9(3), 577- |
| | disorder | 591; |
| | | Ventura et al, 2020, Acta Neuropsychiatr, |
| | | 32(6), 321-327; |
| | | Kim et al, 2016, J Psychiatr Res, 72, 43- |
| | | 50. |
| Genetic | Cryopyrin-associated | Coll et al, 2015, Nat Med, 21, 248-255. |
| (GoF mutations) | periodic syndrome (CAPS)/ | |
| | Muckle-Wells syndrome | |
| | (MWS) | |
| Hearing Loss | Age and genetic related | Shi et al, 2017, Am J Transl Res, 9, 5611- |
| | hearing loss | 5618; |
| | | Nakanishi et al, 2020, Front Neurol, 11, |
| | | 141-148. |
| Ocular/retinal | Age related macular | Gao et al, 2015, Mediators Inflamm, 2015, |
| | degeneration | 690243; |
| | | Wooff et al, 2020, Sci Rep, 10(1), 2263- |
| | | 2283; |
| | | Puyang et al, 2016, Sci Reps, 6, 20998- |
| | | 21007. |
| | Diabetic retinopathy | Zhang et al, 2017, Cell Death Dis, 8, 2941; |
| | | Sui et al, 2020, Cell Death Dis, 1(10), 901- |
| | | 917. |
| Cardiovascular | Atherosclerosis | van der Heijden et al, 2017, Arterioscler |
| | | Thromb Vasc Biol, 37, 1457-1461; |
| | | Duewell et al, 2010, Nature, 464, 1357- |
| | | 1361. |
| | Myocardial infarction/ | Marchetti et al, 2014, J Cardiovasc |
| | ischemia | Pharmacol, 63, 316-322; |
| | | van Hout et al, 2017, Eur Heart J, 38, |
| | | 828-836; |
| | | Sandanger et al, 2013, Cardiovascular |
| | | Research, 99(1), 164-174. |
| Inflammatory/ | Rheumatoid arthritis/ | Guo, 2018, Clin Exp Immunol, 194(2), |
| autoimmune | Lupus | 231-243; |
| | | Vande Walle et al, 2014, Nature, 512, 69- |
| | | 73; |
| | | Fu et al, 2017, Arthritis Rheumatol, 69(8), |
| | | 1636-1646. |
| | Gout | Martinon et al, 2006, Nature, 440, 237- |
| | | 241. |
| | Asthma/respiratory | Ritter et al, 2014, Clin Exp Immunol, 178, |
| | inflammation | 212-223; |
| | | Kim et al, 2017, Am J Respir Crit Care |
| | | Med, 196(3), 283-297. |
| | Psoriasis/skin disease | Primiano et al, 2016, J Immunol, 197(6), |
| | | 2421-2433; |
| | | Wang et al, 2020, J Dermatol Sci, 98(3), |
| | | 146-151; |
| | | Zhu et al, 2020, J Invest Dermatol, |
| | | S0022-202X(20)32276-32284. |
| | Inflammatory bowel disease/ | Perera et al, 2018, Sci Reps, 8, 8618- |
| | colitis | 8633; |
| | | Zhen & Zhang, 2019, Front Immunol, 10, |
| | | 276-286. |
| Metabolic | NASH/NAFLD/fibrosis | Mridha et al, 2017, J Hepatol, 66, 1037- |
| | | 1046; |
| | | Wree et al, 2014, J Mol Med, 92, 1069- |
| | | 1082. |
| | Diabetes | Hu et al, 2015, Proc Natl Acad Sci USA, |
| | | 112, 11318-11323. |
| | Metabolic Syndrome | Cañadas-Lozano et al, 2020, Geroscience, |
| | | 42, 715-725; |
| | | Marín-Aguilar et al, 2020, J Gerontol A |
| | | Biol Sci Med Sci, 75(8), 1457-1464. |

Diseases of the brain, where neuroinflammation has been demonstrated to be a key driver of ongoing disease pathology, have seen considerable research focus. Many of these have identified microglial NLRP3 as being a key contributor to aberrant inflammatory processes and ongoing disease pathology (Table 1).

Genetic ablation of NLRP3 or pharmacological blockade of the inflammasome has been demonstrated to produce significant improvements in ongoing disease pathology in a range of preclinical models of neurodegenerative disease including Parkinson's (Gordon et al, 2018, Sci Transl Med, 10(465), 1-25; Haque et al, 2020, Mov Disord, 35(1), 20-33), Alzheimer's (Heneka et al, 2013, Nature, 493, 674-678; Dempsey et al, 2017, Brain Behav Immun, 61, 306-316), tauopathies such as Frontal Temporal Dementia (Ising et al, 2019, Nature, 575, 669-673), amyotrophic lateral sclerosis (ALS)/motor neuron disease (MND) (Debye et al, 2018, Brain Pathol, 28(1), 14-27; Gugliandolo et al, 2018, Inflammation, 41, 93-103; Deora et al, 2020, Glia, 68(2), 407-421), traumatic brain insults (Irrera et al, 2020, Int J Mol Sci, 21(17), 6204-6223; Wallisch et al, 2017, Neurocrit Care, 27(1), 44-50; O'Brien et al, 2020, J Neuroinflammation, 17(1), 104-116), multiple sclerosis (MS) (Barclay & Shinohara, 2017, Brain Pathol, 27, 213-219; Olcum et al, 2020, Adv Protein Chem Struct Biol, 119, 247-308) and stroke/ischaemic insults (Luo et al, 2019, Curr Neuropharmacol, 17(7), 582-589; Ward et al, 2019, Pharmacol Res, 142, 237-250) (for general reviews on neurodegeneration, see Heneka et al, 2018, Nat Revs Neurosci, 19, 610-621; Guan & Han, 2020, Front Integr Neurosci, 14, 37-46).

Interestingly, NLRP3 has also been shown to have an additional involvement in the inflammation associated with psychiatric diseases such as depression (Kaufmann et al, 2017, Brain Behav Immun, 64, 367-383; Su et al, 2017, Behav Brain Res, 322, 1-8), anxiety/stress disorders (Lei et al, 2017, Brain Res, 1671, 43-54; Wang et al, 2018, J Neuroinflammation, 15(1), 21-35), and schizophrenia and bipolar disorder (Giridharan et al, 2020, Cells, 9(3), 577-591; Ventura et al, 2020, Acta Neuropsychiatr, 32(6), 321-327; Kim et al, 2016, J Psychiatr Res, 72, 43-50).

Taken together these data suggest that modulating NLRP3 inflammasome-induced neuroinflammation would be of broad therapeutic benefit across a wide range of brain disorders.

Non brain disorders: NLRP3 is associated with a diverse range of diseases and conditions (Table 1) and is an important contributor to inflammatory diseases of the peripheral tissues and organs. These include retinal diseases such as age related macular degeneration and diabetic retinopathy (Gao et al, 2015, Mediators Inflamm, 2015, 690243; Lim et al, 2020, Int J Mol Sci, 21(3), 899-913), hearing loss (Nakanishi et al, 2020, Front Neurol, 11, 141-148; Shi et al, 2017, Am J Transl Res, 9, 5611-5618), cardiovascular diseases such as atherosclerosis (Grebe et al, 2018, Circ Res, 122, 1722-1740; Zhou et al, 2018, J Immunol Res, 2018, 5702103), inflammatory and autoimmune diseases such as psoriasis and asthma (Li et al, 2020, Biomed Pharmaco, 130, 110542-110554; Theofani et al, 2019, J Clin Med, 8, 1615-1643; Wang et al, 2020, J Dermatol Sci, 98(3), 146-151) and metabolic disorders and associated complications (Wan et al, 2016, Can J Gastroenterol Hepatol, 2016, 6489012-6489019; Ding et al, 2019, Biomolecules, 9(12), 850-865).

KCNK13 (THIK-1)

The central role of K⁺ flux in the activation of the conical NLRP3 activation has been well documented (see paragraph on NLRP3 above) and several channels have been suggested to be the mediators of this K⁺ current in microglia. One such channel is KCNK13 (K_{2P}13.1) or potassium two pore domain channel subfamily K member 13 gene which encodes for a two-pore forming domain potassium channel known as tandem pore domain halothane-inhibited K⁺ channel 1 or THIK-1. KCNK13 together with KCNK12 are members of the leak or background K⁺ channels (K_{2P}) first cloned by Rajan et al (2001, J Biol Chem, 276, 7302-7311). KCNK12 encodes a closely related channel THIK-2 which is silent as a homodimer but can heterodimerise with THIK-1 to form an active channel, albeit with reduced function vs THIK-1 homodimer (Blin et al, 2014, J Biol Chem, 289, 28202-28212). Electrophysiological studies show that THIK-1 displays an outward rectify current with a very small single-channel conductance (˜5 pS at +100 mV) and short open time duration (<0.5 ms) (Kang et al, 2014, Pflugers Arch, 466(7), 1289-1300). THIK-1 K⁺ channel conductance has been shown to play roles in modulating the biology of microglia and has a central role in mediating the proinflammatory response of microglia via the NLRP3 inflammasome (Madry et al, 2018, Neuron, 97, 299-312). Furthermore, blockade of THIK-1 conductance inhibits lipopolysaccharide (LPS)-induced production of proinflammatory IL-1β(Madry et al, 2018, Neuron, 97, 299-312). Our own data further confirm these findings demonstrating that inhibition of THIK-1 attenuates LPS- and K⁺-induced activation of caspase-1 and subsequent IL-1R production and release from isolated microglia (see example 3 below) and IL-1βrelease from LPS-treated rodent hippocampus. It can thus be concluded that selective inhibitors of THIK-1 reduce NLRP3 inflammasome mediated inflammation and thus have therapeutic utility in many of the NLRP3 related indications highlighted above and in Table 1.

There is a need for treatment of the above diseases and conditions and others described herein with compounds that are KCNK13 antagonists. The present invention provides antagonists of KCNK13.

### SUMMARY OF THE INVENTION

A first aspect of the present invention provides a compound of formula (I):

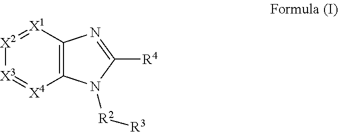

or a pharmaceutically acceptable salt, N-oxide, solvate or prodrug thereof, wherein:
each X¹, X², X³ and X⁴ is independently CH, CR¹ or N;
each —R¹ is independently halo, —CN, —R^{α}, —OH, —OR^{α}, —NH₂, —NHR^{α}, —N(R^{α})₂, —SR^{α}, —SOR^{α}, —SO₂R^{α}, —SO(NH)R^{α}, —SO₂NHR^{α}, —SO₂N(R^{α})₂, —NH—SOR^{α}, —NH—SO₂R^{α}, —NH—SO₂NHR^{α}, —NH—SO₂N(R^{α})₂, —NR^{α}—SOR^{α}, —NR^{α}—SO₂R^{α}, —NR^{α}—SO₂NH₂, —NR^{α}—SO₂NHR^{α}, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —C(R^{θ})₂—, —C(R^{θ})₂-C(R^{θ})₂—, —C(R^{θ})₂-O—, —C(R^{θ})₂-NR^{θ}—, —C(R^{θ})₂-CO—, or —C(R^{θ})₂-CONR^{θ}—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR^{δ}—SO₂R^{δ}, —NR^{δ}—SO₂NH₂, —NR₆—SO₂NHR^{δ}, —NR^{δ}—SO₂N(R^{δ})₂, —COR^{δ}, —COOR^{δ}, —OCOR^{δ}, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR^{δ}, —NH—CON(R^{δ})₂, or —NR^{δ}—CON(R^{δ})₂;
R⁴ is a 5-membered heteroaryl group with one or more heteroatoms N, O or S in the ring structure, wherein the heteroaryl group is optionally substituted with one, two, three or four substituents independently selected from halo, —CN, —Re, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR—, —SO₂N(R^{ε})₂, —NH—SO₂R^{ε}, —NH—SO₂NHR^{ε}, —NH—SO₂N(R^{ε})₂, or —NR^{ε}—SO₂R^{ε};
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and
each —R^{θ} is independently hydrogen or methyl;
   provided that the compound is not:

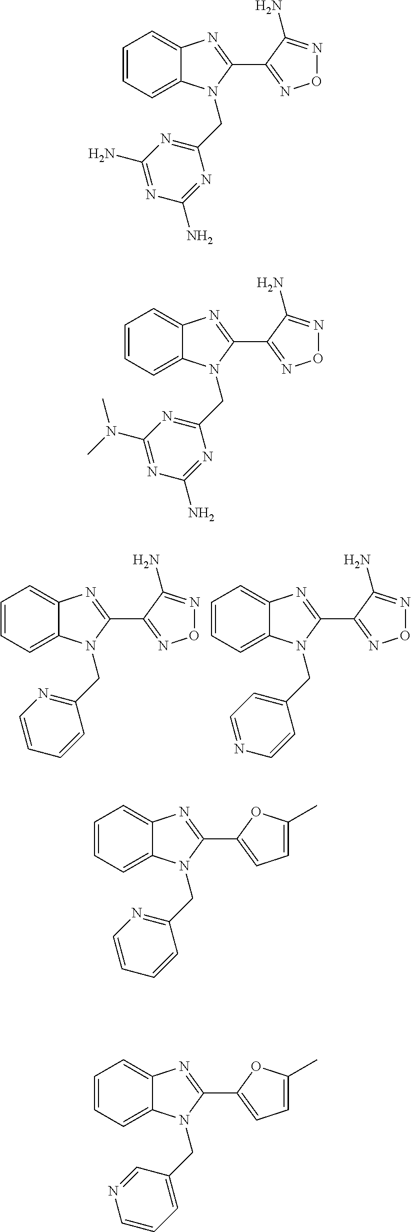

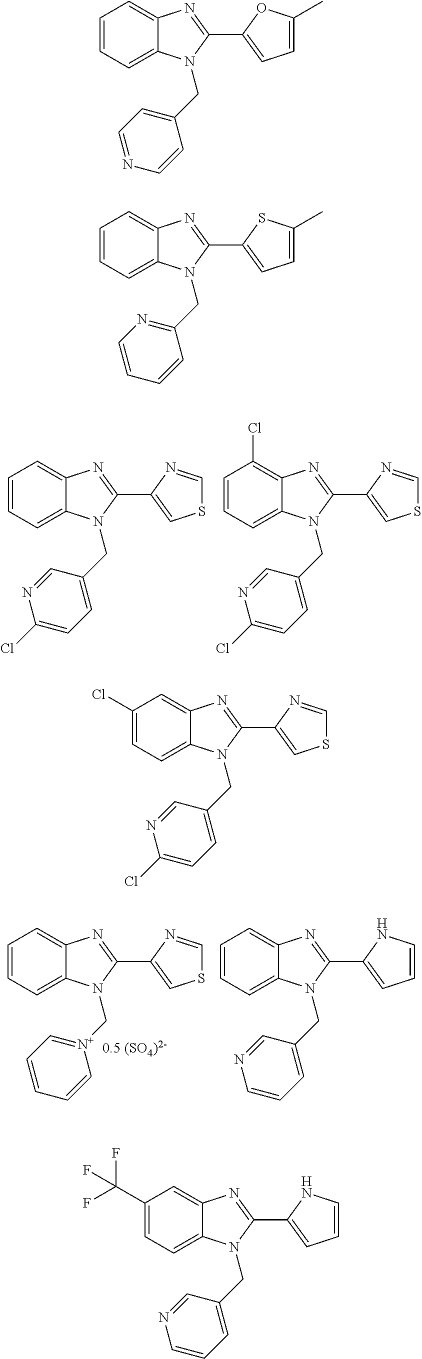

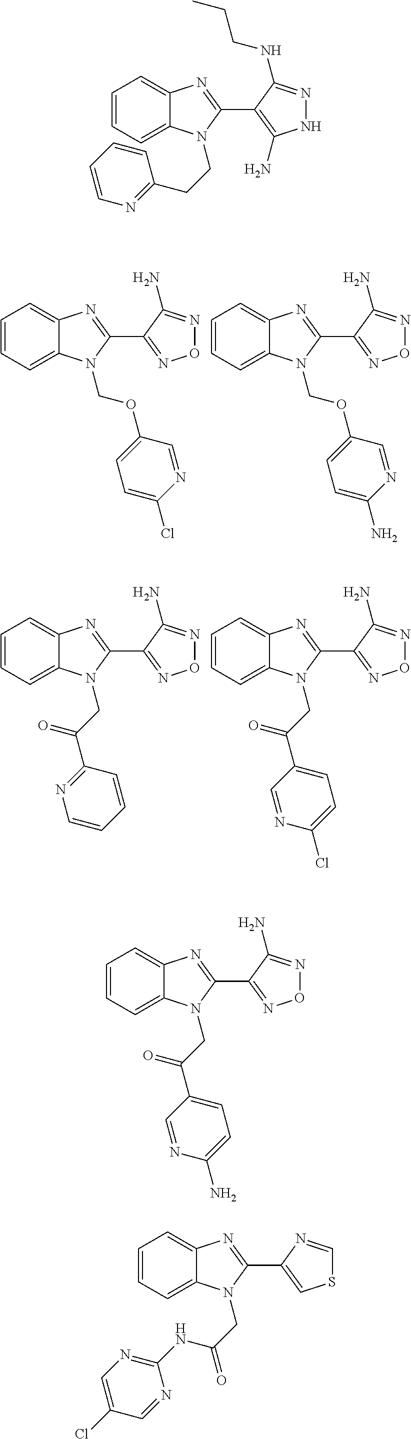

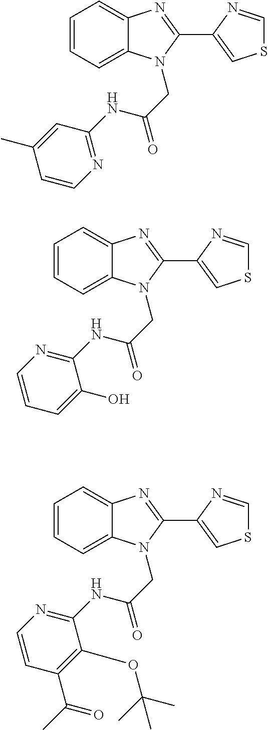

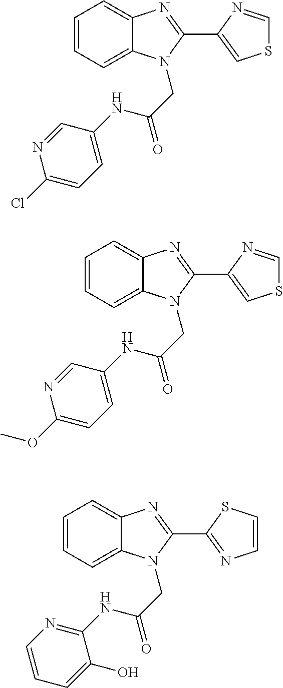 [SMILES: O=C(Cn1c(-c2cscn2)nc2ccccc21)Nc1ccc(Cl)nc1 ; COc1ccc(NC(=O)Cn2c(-c3cscn3)nc3ccccc32)cn1 ; O=C(Cn1c(-c2nccs2)nc2ccccc21)Nc1ncccc1O]

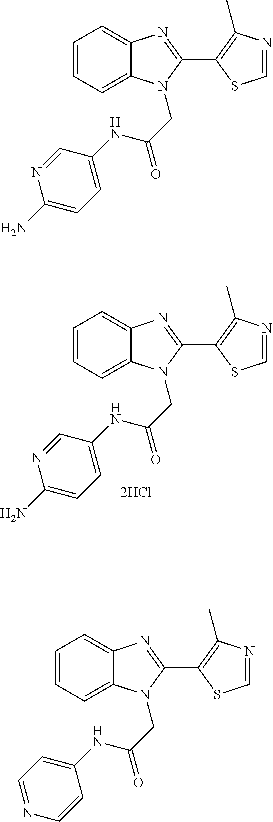 [SMILES: Cc1ncsc1-c1nc2ccccc2n1CC(=O)Nc1ccc(N)nc1 ; Cc1ncsc1-c1nc2ccccc2n1CC(=O)Nc1ccc(N)nc1 ; Cc1ncsc1-c1nc2ccccc2n1CC(=O)Nc1ccncc1]

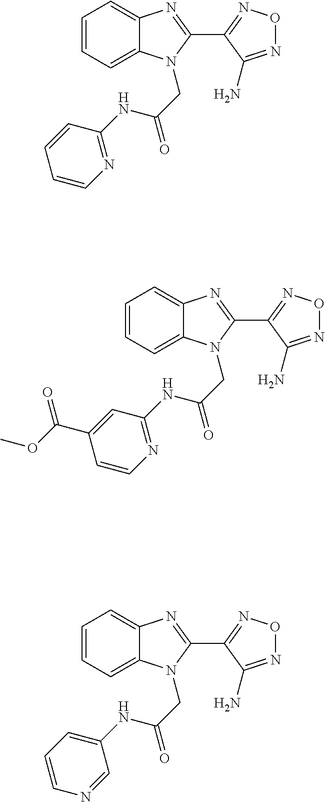 [SMILES: Nc1nonc1-c1nc2ccccc2n1CC(=O)Nc1ccccn1 ; COC(=O)c1ccnc(NC(=O)Cn2c(-c3nonc3N)nc3ccccc32)c1 ; Nc1nonc1-c1nc2ccccc2n1CC(=O)Nc1cccnc1]

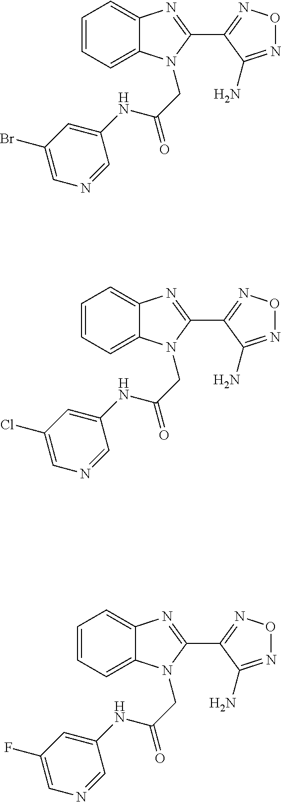 [SMILES: Nc1nonc1-c1nc2ccccc2n1CC(=O)Nc1cncc(Br)c1 ; Nc1nonc1-c1nc2ccccc2n1CC(=O)Nc1cncc(Cl)c1 ; Nc1nonc1-c1nc2ccccc2n1CC(=O)Nc1cncc(F)c1]

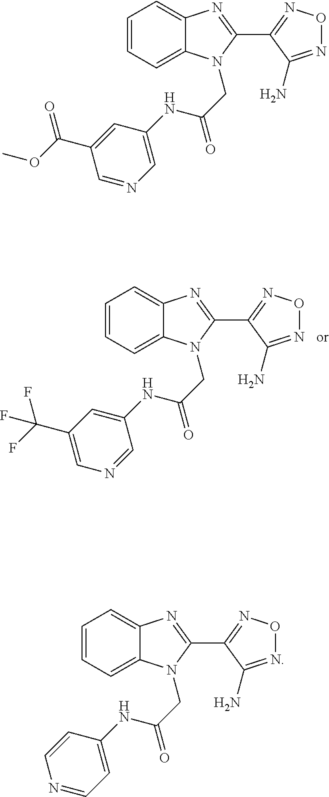 [SMILES: COC(=O)c1cncc(NC(=O)Cn2c(-c3nonc3N)nc3ccccc32)c1 ; Nc1nonc1-c1nc2ccccc2n1CC(=O)Nc1cncc(C(F)(F)F)c1 ; Nc1nonc1-c1nc2ccccc2n1CC(=O)Nc1ccncc1]

In the compound of the first aspect of the present invention, each X¹, X² X³ and X⁴ is independently CH, CR¹ or N.

In one embodiment, each X¹, X², X³ and X⁴ is independently CH or CR¹.

In another embodiment, one of X¹, X², X³ and X⁴ is N, and the remaining of X¹, X², X³ and X⁴ are independently CH or CR¹.

In a preferred embodiment, each X¹, X², X³ and X⁴ is independently CH or CR¹; or one of X¹, X², X³ and X⁴ is N, and the remaining of X¹, X², X³ and X⁴ are independently CH or CR¹.

In one embodiment, X¹ is N, and each X², X³ and X⁴ is independently CH or CR¹. In another embodiment, X² is N, and each X¹, X³ and X⁴ is independently CH or CR¹. In a preferred embodiment, X³ is N, and each X¹, X² and X⁴ is independently CH or CR¹. In another preferred embodiment, X⁴ is N, and each X¹, X² and X³ is independently CH or CR¹.

In the compound of the first aspect of the present invention, each —R¹ is independently halo, —CN, —R^{α}, —OH, —OR^{α}, —NH₂, —NHR^{α}, —N(R^{α})₂, —SR^{α}, —SOR^{α}, —SO₂R^{α}, —SO(NH)R^{α}, —SO₂NHR^{α}, —SO₂N(R^{α})₂, —NH—SOR^{α}, —NH—SO₂Ra, —NH—SO₂NHRα, —NH—SO₂N(Ra)₂, —NR^{α}—SOR^{α}, —NR^{α}—SO₂R^{α}, —NR^{α}—SO₂NH₂, —NR^{α}-SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic (e.g. with one, two, three or four heteroatoms N, O or S in the ring structure), or 5- or 6-membered heteroaryl (e.g. with one, two, three or four heteroatoms N, O or S in the ring structure) group, wherein the cycloalkyl, phenyl, heterocyclic or heteroaryl group is optionally substituted with one or two substituents independently selected from C₁-C₃ alkyl or —CO(C₁-C₃ alkyl); wherein each —R^{α} is independently C₁-C₃ alkyl, C₂-C₃ alkenyl, C₂-C₃ alkynyl or C₃-C₆ cycloalkyl, all optionally substituted with one or more (such as one, two, three, four or five) substituents independently selected from halo, —OH, —NH₂ or —SO₂CH₃.

In one embodiment, if —R¹ is a heterocyclic group with a nitrogen atom in the ring structure, said nitrogen atom may be substituted with C₁-C₃ alkyl, or —CO(C₁-C₃ alkyl).

The compound of the first aspect of the present invention comprises zero, one, two, three or four groups R¹. In one embodiment, the compound comprises zero, one, two or three groups R¹. In one embodiment, the compound comprises zero, one or two groups R¹. Preferably, the compound comprises one or two groups R¹, or the compound comprises one group R¹.

In one embodiment, each —R¹ is independently halo, —CN, —R^{α}, —OH, —OR^{α}, —NH₂, —NHRα, —N(R^{α})₂, —SR^{α}, —SOR^{α}, —SO₂R^{α}, —SO(NH)R^{α}, —SO₂NHRα, —SO₂N(R^{α})₂, —NH—SOR^{α}, —NH—SO₂R^{α}, —NR^{α}—SOR^{α}, —NR^{α}—SO₂R^{α}, —COR^{α}, —COOR^{α}, —OCOR^{α}, —CONH₂, —CONHR^{α}, —CON(R^{α})₂, C₃-C₆ cycloalkyl, phenyl, a 3- to 6-membered heterocyclic group with one, two, three or four heteroatoms N, O or S in the ring structure, or a 5- or 6-membered heteroaryl group with one, two, three or four heteroatoms N, O or S in the ring structure, wherein the cycloalkyl, phenyl, heterocyclic or heteroaryl group is optionally substituted with one or two substituents independently selected from C₁-C₃ alkyl or —CO(C₁-C₃ alkyl); wherein each —R^{α} is independently C₁-C₃ alkyl, C₂-C₃ alkenyl, C₂-C₃ alkynyl or C₃-C₆ cycloalkyl, all optionally substituted with one, two, three, four or five substituents independently selected from halo, —OH, —NH₂ or —SO₂CH₃.

In one embodiment, each —R¹ is independently halo, —CN, —R^{α}, —OH, OR^{α}, —NH₂, —NHRα, —N(R^{α})₂, —SR^{α}, —SOR^{α}, —SO₂R^{α}, —SO₂NHRα, —SO₂N(R^{α})₂, —NH—SO₂R^{α}, —NR^{α}—SO₂R^{α}, —COR^{α}, —COOR^{α}, —OCOR^{α}, —CONH₂, —CONHR^{α}, —CON(R^{α})₂, C₃-C₆ cycloalkyl, phenyl, a 3- to 6-membered heterocyclic group with one, two or three heteroatoms N, O or S in the ring structure, or a 5- or 6-membered heteroaryl group with one, two or three heteroatoms N, O or S in the ring structure, wherein the cycloalkyl, phenyl, heterocyclic or heteroaryl group is optionally substituted with one or two substituents independently selected from C₁-C₃ alkyl or —CO(C₁-C₃ alkyl); wherein each —R^{α} is independently C₁-C₃ alkyl, C₂-C₃ alkenyl, C₂-C₃ alkynyl or C₃-C₆ cycloalkyl, all optionally substituted with one, two, three, four or five halo.

In one embodiment, each —R¹ is independently halo, —CN, C₁-C₃ alkyl, C₁-C₃ haloalkyl, C₂-C₃ alkenyl, —OH, —O(C₁-C₃ alkyl), —O(C₁-C₃ haloalkyl), —NH₂, —NH(C₁-C₃ alkyl), —N(C₁-C₃ alkyl)₂, —S(C₁-C₃ alkyl), —SO(C₁-C₃ alkyl), —SO₂(C₁-C₃ alkyl), —NH—SO₂(C₁-C₃ alkyl), —CO(C₁-C₃ alkyl), —COO(C₁-C₃ alkyl), —OCO(C₁-C₃ alkyl), —CONH₂, —CONH(C₁-C₃ alkyl), —CON(C₁-C₃ alkyl)₂, C₃-C₆ cycloalkyl, or a 3- to 6-membered heterocyclic group with one or two heteroatoms N, O or S in the ring structure, wherein the cycloalkyl or heterocyclic group is optionally substituted with one or two substituents independently selected from C₁-C₃ alkyl or —CO(C₁-C₃ alkyl).

In one embodiment, each —R¹ is independently fluoro, chloro, bromo, iodo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SCH₃, —SOCH₃, —SO₂CH₃, —NH—SO₂CH₃, cyclopropyl, cyclobutyl, cyclopentyl, cyclohexyl, or a heterocyclic group selected from azetidinyl, oxetanyl, pyrrolidinyl, tetrahydrofuranyl, piperidinyl or tetrahydropyranyl, wherein the heterocyclic group is optionally substituted with C₁-C₃ alkyl or —CO(C₁-C₃ alkyl).

In one embodiment, each —R¹ is independently fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃.

In one embodiment, each —R¹ is independently fluoro, chloro, bromo, —CN, —CH₃, —CH₂CH₃, —CH═CH₂, —OH, —OCH₃, —OCH₂CH₃, —OCF₃, —NH₂, —NHCH₃, —N(CH₃)₂, —SO₂CH₃, or —NH—SO₂CH₃.

In one embodiment, each —R¹ is fluoro or chloro.

In one embodiment, each —R¹ is fluoro.

In one embodiment, the compound of the first aspect of the present invention comprises one or two groups R¹, and each R¹ is fluoro.

In the compound of the first aspect of the present invention, —R²— is —C(R^{θ})₂—, —C(R^{θ})₂—C(R^{θ})₂—, —C(R^{θ})₂—O—, —C(R^{θ})₂—NR^{θ}—, —C(R^{θ})₂—CO—, or —C(R^{θ})₂—CONR^{θ}—; wherein each —R^{θ} is independently hydrogen or methyl.

In one embodiment, —R²— is —CH₂—, —CH(CH₃)—, —C(CH₃)₂—, —CH₂—CH₂—, —CH(CH₃)—CH₂—, —C(CH₃)₂—CH₂—, —CH(CH₃)—CH(CH₃)—, —CH₂—O—, —CH(CH₃)—O—, —C(CH₃)₂—O—, —CH₂—NH—, —CH(CH₃)—NH—, —C(CH₃)₂—NH—, CH₂—N(CH₃)—, —CH(CH₃)—N(CH₃)—, —CH₂—CO—, —CH(CH₃)—CO—, —C(CH₃)₂—CO—, —CH₂—CO—NH—, —CH(CH₃)—CO—NH—, —C(CH₃)₂—CO—NH—, —CH₂—CO—N(CH₃)—, or —CH(CH₃)—CO—N(CH₃)—.

In one embodiment, —R²— is —CH₂—, —CH(CH₃)—, —CH₂—CH₂—, —CH(CH₃)—CH₂—, —CH(CH₃)—CH(CH₃)—, —CH₂—O—, —CH(CH₃)—O—, —CH₂—NH—, —CH(CH₃)—NH—, CH₂—N(CH₃)—, —CH(CH₃)—N(CH₃)—, —CH₂—CO—, —CH(CH₃)—CO—, —CH₂—CO—NH—, —CH(CH₃)—CO—NH—, —CH₂—CO—N(CH₃)—, or —CH(CH₃)—CO—N(CH₃)—.

In one embodiment, —R²— is —CH₂—, —CH(CH₃)—, —CH₂—CH₂—, —CH(CH₃)—CH₂—, —CH₂—O—, —CH(CH₃)—O—, —CH₂—NH—, —CH(CH₃)—NH—, CH₂—N(CH₃)—, —CH₂—CO—, —CH(CH₃)—CO—, —CH₂—CO—NH—, —CH(CH₃)—CO—NH—, or —CH₂—CO—N(CH₃)—.

In one embodiment, —R²— is —CH₂—, —CH(CH₃)—, —CH₂—CH₂—, —CH₂—O—, or —CH₂—CO—NH—.

In one embodiment, —R²— is —CH₂—, —CH(CH₃)—, —CH₂—CH₂—, or —CH₂—O—.

In one embodiment, —R²— is —CH₂— or —CH(CH₃)—.

In one embodiment, —R²— is —CH₂—.

For the avoidance of doubt, it is noted that, for example, —CH₂— includes-CHD- and -CD₂-.

In the compound of the first aspect of the present invention, —R³ is a 6-membered heteroaryl group with one or more (such as one, two, three or four) nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R⁶⁷, —SO(NR^{δ})R^{δ}, —SO₂NH₂, —SO₂NHR^{δ}, —SO₂N(R^{δ})₂, —NH—SOR^{δ}, —NH—SO₂R⁶⁷, —NH—SO₂NHR⁶⁷, —NH—SO₂N(R^{δ})₂, —NR^{δ}—SOR^{δ}, —NR—SO₂R, —NR^{δ}—SO₂NH₂, —NR^{δ}—SO₂NHR^{δ}, —NR^{δ}—SO₂N(R^{δ})₂, —COR^{δ}, —COOR^{δ}, —OCOR^{δ}, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR⁶, —NH—CON(R^{δ})₂, or —NR^{δ}—CON(R^{δ})₂; wherein each —R^{δ} is independently C₁-C₃ alkyl, C₂-C₃ alkenyl, C₂-C₃ alkynyl or C₃-C₆ cycloalkyl, all optionally substituted with one or more (such as one, two, three, four or five) substituents independently selected from halo, —OH, —NH₂ or —SO₂CH₃. In one embodiment, each —R⁶ is independently C₁-C₃ alkyl, C₂-C₃ alkenyl, C₂-C₃ alkynyl or C₃-C₆ cycloalkyl, all optionally substituted with one or more (such as one, two, three, four or five) substituents independently selected from halo or —SO₂CH₃.

In one embodiment, —R³ is a 6-membered heteroaryl group with one, two, three or four nitrogen atoms in the ring structure (such as pyridinyl, pyridazinyl, pyrimidinyl, pyrazinyl, triazinyl or tetrazinyl), wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH,-SRs, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NR^{δ}—SOR^{δ}, —NR^{δ}—SO₂R^{δ}, —COR^{δ}, —COOR^{δ}, —OCOR^{δ}, —CONH₂, —CONHR^{δ}, or —CON(R^{δ})₂; wherein each —R^{δ} is independently C₁-C₃ alkyl, C₂-C₃ alkenyl, C₂-C₃ alkynyl or C₃-C₆ cycloalkyl, all optionally substituted with one, two, three, four or five substituents independently selected from halo or —SO₂CH₃.

In one embodiment, —R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO₂NHR^{δ}, —SO₂N(R^{δ})₂, —NH—SO₂R^{δ}, —NR^{δ}—SO₂R^{α}, —COR^{δ}, —COOR^{δ}, —OCOR^{δ}, —CONH₂, —CONHR^{δ}, or —CON(R^{δ})₂; wherein each —R^{δ} is independently C₁-C₃ alkyl, C₂-C₃ alkenyl, C₂-C₃ alkynyl or C₃-C₆ cycloalkyl, all optionally substituted with one, two, three, four or five halo.

In one embodiment, —R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two, three or four substituents independently selected from halo, —CN, C₁-C₃ alkyl, C₁-C₃ haloalkyl, C₂-C₃ alkenyl, C₃-C₆ cycloalkyl, —OH, —O(C₁-C₃ alkyl), —O(C₁-C₃ haloalkyl), —NH₂, —NH(C₁-C₃ alkyl), —N(C₁-C₃ alkyl)₂, —SH, —S(C₁-C₃ alkyl), —SO(C₁-C₃ alkyl), —SO₂(C₁-C₃ alkyl), —SO₂NH(C₁-C₃ alkyl), —SO₂N(C₁-C₃ alkyl)₂, —NH—SO₂(C₁-C₃ alkyl), —CO(C₁-C₃ alkyl), —COO(C₁-C₃ alkyl), —OCO(C₁-C₃ alkyl), —CONH₂, —CONH(C₁-C₃ alkyl), or —CON(C₁-C₃ alkyl)₂.

In one embodiment, —R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two, three or four substituents independently selected from halo, —CN, C₁-C₃ alkyl, C₁-C₃ haloalkyl, C₂-C₃ alkenyl, C₃-C₆ cycloalkyl, —OH, —O(C₁-C₃ alkyl), —O(C₁-C₃ haloalkyl), —NH₂, —NH(C₁-C₃ alkyl), —N(C₁-C₃ alkyl)₂, —SH, —S(C₁-C₃ alkyl), —SO(C₁-C₃ alkyl), —SO₂(C₁-C₃ alkyl), —NH—SO₂(C₁-C₃ alkyl), —CO(C₁-C₃ alkyl), —COO(C₁-C₃ alkyl), —OCO(C₁-C₃ alkyl), —CONH₂, —CONH(C₁-C₃ alkyl), or —CON(C₁-C₃ alkyl)₂.

In one embodiment, —R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two, three or four substituents independently selected from fluoro, chloro, bromo, iodo, —CN, —CH₃, —CH₂CH₃, —CH═CH₂, —CF₃, —CHF₂, —OH, —OCH₃, —OCH₂CH₃, —OCF₃, —NH₂, —NHCH₃, —N(CH₃)₂, —SH, —SCH₃, —SOCH₃, —SO₂CH₃, —SO₂CH₂CH₃, —SO₂—NHCH₃, —NH—SO₂CH₃, cyclopropyl, cyclobutyl, cyclopentyl, or cyclohexyl.

In one embodiment, —R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two, three or four substituents independently selected from fluoro, chloro, bromo, iodo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SH, —SCH₃, —SOCH₃, —SO₂CH₃, —NH—SO₂CH₃, cyclopropyl, cyclobutyl, cyclopentyl, or cyclohexyl.

In one embodiment, —R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two or three substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃.

In one embodiment, —R³ is pyridinyl, pyridazinyl, pyrimidinyl or pyrazinyl, each of which is optionally substituted with one or two substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃.

In one embodiment, —R³ is pyridinyl, pyridazinyl, pyrimidinyl or pyrazinyl, each of which is optionally substituted with one or two substituents independently selected from fluoro or chloro.

In the compound of the first aspect of the present invention, —R⁴ is a 5-membered heteroaryl group with one or more (such as one, two, three or four) heteroatoms N, O or S in the ring structure, wherein the heteroaryl group is optionally substituted with one, two, three or four substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR^{ε}, —SO₂N(R^{ε})₂, —NH—SO₂R^{ε}, —NH—SO₂NHR^{ε}, —NH—SO₂N(R^{ε})₂, or —NR^{ε}—SO₂R^{ε}; wherein each —R^{ε} is independently C₁-C₃ alkyl, C₂-C₃ alkenyl, C₂-C₃ alkynyl or C₃-C₆ cycloalkyl, all optionally substituted with one or more (such as one, two, three, four or five) substituents independently selected from halo, —OH, —NH₂ or —SO₂CH₃.

In one embodiment, —R⁴ is a 5-membered heteroaryl group with one, two, three or four heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl or thiatriazolyl), wherein the heteroaryl group is optionally substituted with one, two, three or four substituents independently selected from halo, —CN, —Re, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR—, —SO₂N(R^{ε})₂, —NH—SO₂R^{ε}, or —NR^{ε}—SO₂R^{ε}; wherein each —R^{ε} is independently C₁-C₃ alkyl, C₂-C₃ alkenyl, C₂-C₃ alkynyl or C₃-C₆ cycloalkyl, all optionally substituted with one, two, three, four or five substituents independently selected from halo, —OH, —NH₂ or —SO₂CH₃.

In one embodiment, —R⁴ is a 5-membered heteroaryl group with one, two, three or four heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl or thiatriazolyl), wherein the heteroaryl group is optionally substituted with one, two, three or four substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NHR—, —SO₂N(R^{ε})₂, —NH—SO₂R^{ε}, or —NR^{ε}—SO₂R^{ε}; wherein each —R^{ε} is independently C₁-C₃ alkyl, C₂-C₃ alkenyl, C₂-C₃ alkynyl or C₃-C₆ cycloalkyl, all optionally substituted with one, two, three, four or five halo.

In one embodiment, —R⁴ is a 5-membered heteroaryl group with one, two or three heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl or thiadiazolyl), wherein the heteroaryl group is optionally substituted with one, two or three substituents independently selected from halo, —CN, C₁-C₃ alkyl, C₁-C₃ haloalkyl, C₂-C₃ alkenyl, C₃-C₆ cycloalkyl, —OH, —O(C₁-C₃ alkyl), —O(C₁-C₃ haloalkyl), —NH₂, —NH(C₁-C₃ alkyl), —N(C₁-C₃ alkyl)₂, —SH, —S(C₁-C₃ alkyl), —SO(C₁-C₃ alkyl), —SO₂(C₁-C₃ alkyl), or —NH—SO₂(C₁-C₃ alkyl).

In one embodiment, —R⁴ is a 5-membered heteroaryl group with one, two or three heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl or thiadiazolyl), wherein the heteroaryl group is optionally substituted with one, two or three substituents independently selected from fluoro, chloro, bromo, iodo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SH, —SCH₃, —SOCH₃, —SO₂CH₃, —NH—SO₂CH₃, cyclopropyl, cyclobutyl, cyclopentyl, or cyclohexyl.

In one embodiment, —R⁴ is a 5-membered heteroaryl group with one, two or three heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl or thiadiazolyl; in particular a 5-membered heteroaryl group with two or three heteroatoms N, O or S in the ring structure such as pyrazolyl, imidazolyl, oxazolyl, isoxazolyl, thiazolyl, isothiazolyl, triazolyl, oxadiazolyl or thiadiazolyl; more particularly a 5-membered heteroaryl group with three heteroatoms N, O or S in the ring structure such as triazolyl, oxadiazolyl or thiadiazolyl), wherein the heteroaryl group is optionally substituted with one, two or three (in particular one or two; more particularly one) substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃.

In one embodiment, —R⁴ is a 5-membered heteroaryl group with two or three heteroatoms N, O or S in the ring structure (such as pyrazolyl, imidazolyl, oxazolyl, isoxazolyl, thiazolyl, isothiazolyl, triazolyl, oxadiazolyl or thiadiazolyl; in particular a 5-membered heteroaryl group with three heteroatoms N, O or S in the ring structure such as triazolyl, oxadiazolyl or thiadiazolyl), wherein the heteroaryl group is optionally substituted with one or two (in particular one) substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃.

In one embodiment, —R⁴ is a 5-membered heteroaryl group with three heteroatoms N, O or S in the ring structure (such as triazolyl, oxadiazolyl or thiadiazolyl), wherein the heteroaryl group is optionally substituted with one substituent independently selected from fluoro, chloro, bromo, —CN, —CH₃, —CH₂CH₃, —CH═CH₂, —CF₃, —OH, —OCH₃, —OCH_{Z}CH₃, —OCF₃, —NH₂, —NHCH₃, —N(CH₃)₂, —SO₂CH₃, or —NH—SO₂CH₃.

In one embodiment, —R⁴ is oxadiazolyl optionally substituted with —CH₃ or —NH₂.

In one specific embodiment of the first aspect, the present invention provides a compound of formula (I), or a pharmaceutically acceptable salt, N-oxide, solvate or prodrug thereof, wherein:
each X¹, X², X³ and X⁴ is independently CH, CR¹ or N;
each —R¹ is independently halo, —CN, —R^{α}, —OH, OR^{α}, —NH₂, —NHRα, —N(R^{α})₂, —SR^{α}, —SOR^{α}, —SO₂R^{α}, —SO(NH)R^{α}, —SO₂NHRα, —SO₂N(R^{α})₂, —NH—SOR^{α}, —NH—SO₂R^{α}, —NH—SO₂NHRα, —NH—SO₂N(R^{α})₂, —NR^{α}—SOR^{α}, —NR^{α}—SO₂R^{α}, —NR^{α}—SO₂NH₂, —NR^{α}-SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —C(R⁶⁷)₂—, —C(R⁶⁷)₂-C(R⁶⁷)₂—, —C(R⁶⁷)₂-O—, —C(R⁶⁷)₂-NR⁶⁷—, or —C(Re)₂—CO—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR^{δ}—SO₂R^{δ}, —NR^{δ}—SO₂NH₂, —NR^{δ}—SO₂NHR^{δ}, —NR^{δ}—SO₂N(R^{δ})₂, —COR⁶⁷, —COOR^{δ}, —OCOR⁶⁷, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR^{δ}, —NH—CON(R^{δ})₂, or —NR^{δ}—CON(R^{δ})₂;
R⁴ is a 5-membered heteroaryl group with one or more heteroatoms N, O or S in the ring structure, wherein the heteroaryl group is optionally substituted with one, two, three or four substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR—, —SO₂N(R^{ε})₂, —NH—SO₂R^{ε}, —NH—SO₂NHR—, —NH—SO₂N(R^{ε})₂, or —NR^{ε}—SO₂R^{ε};
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃:
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and each —R^{θ} is independently hydrogen or methyl;
   provided that the compound is not:

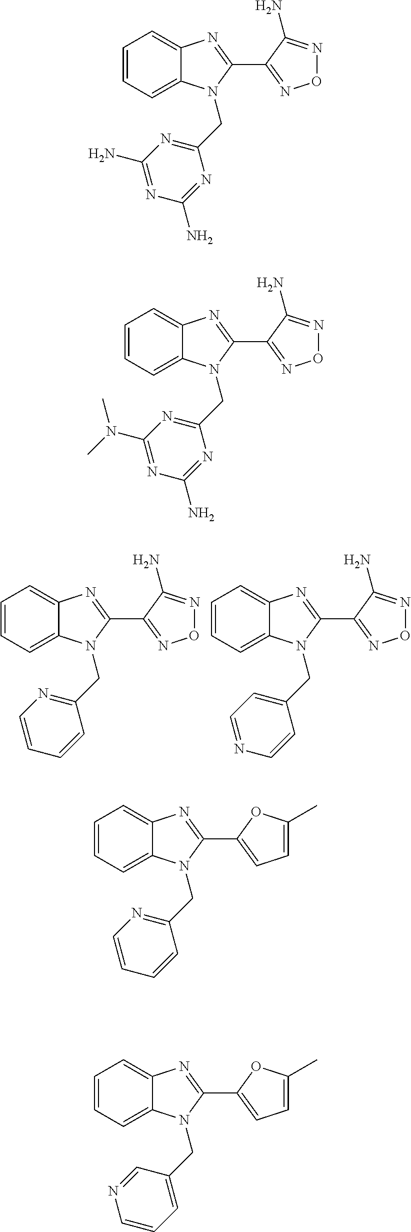

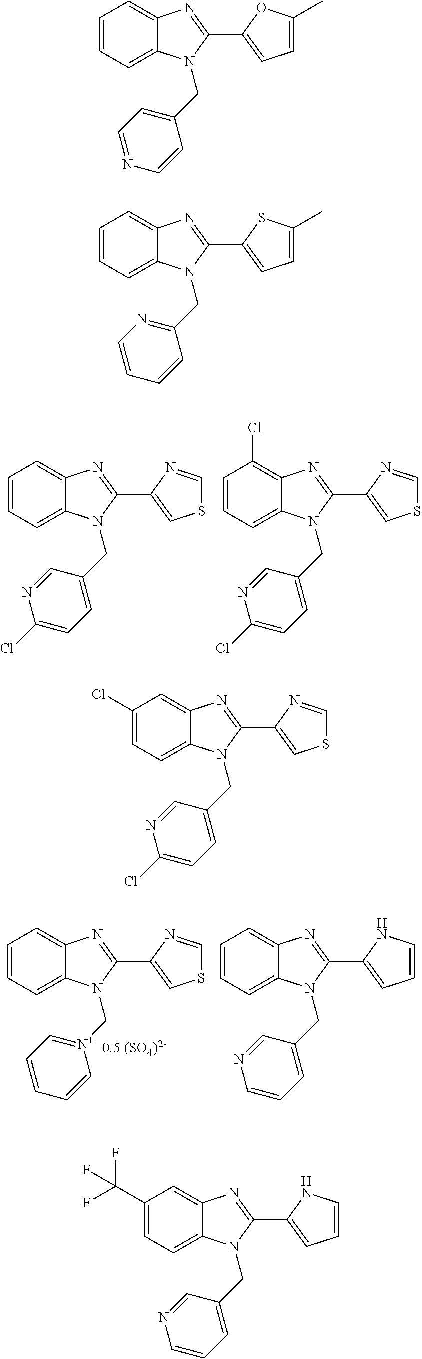

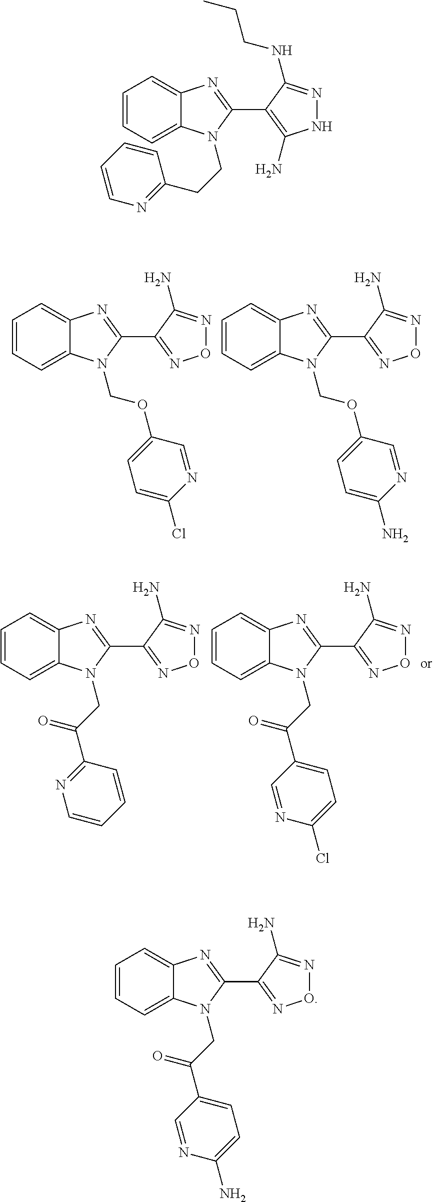

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH, CR¹ or N;
each —R¹ is independently halo, —CN, —R^{α}, —OH, OR^{α}, —NH₂, —NHRα, —N(R^{α})₂, —SR^{α}, —SOR^{α}, —SO₂R^{α}, —SO(NH)R^{α}, —SO₂NHRα, —SO₂N(R^{α})₂, —NH—SOR^{α}, —NH—SO₂R^{α}, —NH—SO₂NHRα, —NH—SO₂N(R^{α})₂, —NR^{α}—SOR^{α}, —NR^{α}—SO₂R^{α}, —NR^{α}-SO₂NH₂, —NR^{α}-SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —CH₂— or —CH(CH₃)—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR^{δ}—SO₂R^{δ}, —NR^{δ}—SO₂NH₂, —NR^{δ}—SO₂NHR^{δ}, —NR^{δ}—SO₂N(R^{δ})₂, —COR⁶⁷, —COOR^{δ}, —OCOR⁶⁷, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR⁶, —NH—CON(R^{δ})₂, or —NR^{δ}—CON(R^{δ})₂;
R⁴ is a 5-membered heteroaryl group with one or more heteroatoms N, O or S in the ring structure, wherein the heteroaryl group is optionally substituted with one, two, three or four substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR—, —SO₂N(R^{ε})₂, —NH—SO₂R^{ε}, —NH—SO₂NHR^{F}, —NH—SO₂N(R^{F})₂, or —NR^{ε}—SO₂R^{ε};
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
provided that the compound is not:

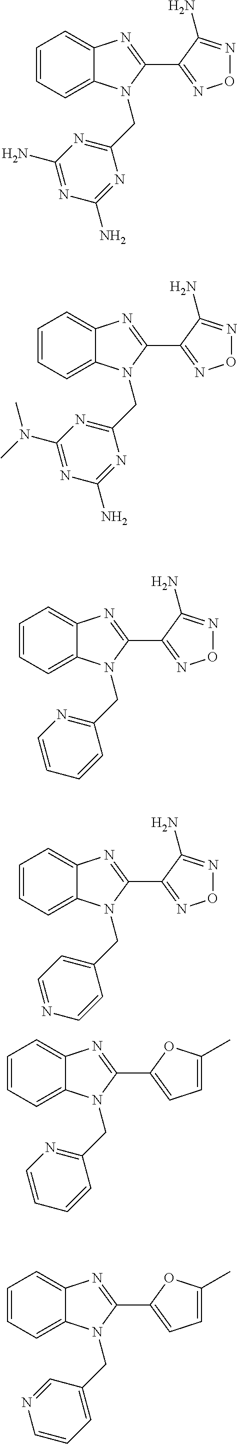

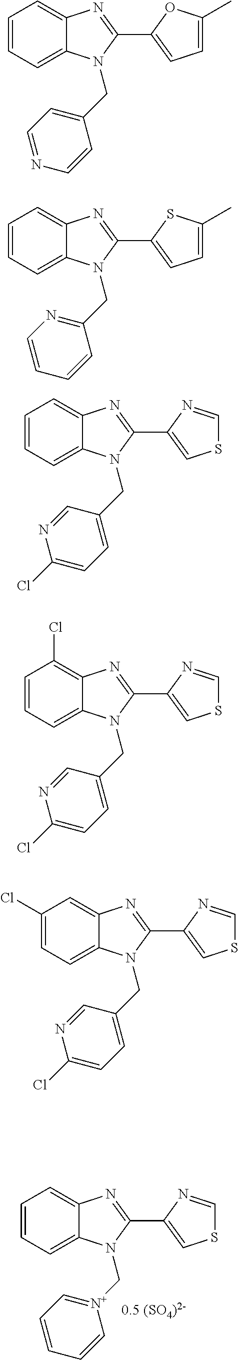

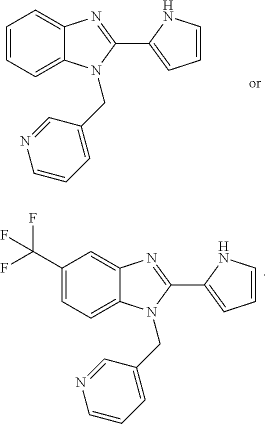

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH, CR¹ or N;
each —R¹ is independently halo, —CN, —R^{α}, —OH, OR^{α}, —NH₂, —NHRα, —N(R^{α})₂, —SR^{α}, —SOR^{α}, —SO₂R^{α}, —SO(NH)R^{α}, —SO₂NHRα, —SO₂N(R^{α})₂, —NH—SOR^{α}, —NH—SO₂R^{α}, —NH—SO₂NHRα, —NH—SO₂N(R^{α})₂, —NR^{α}—SOR^{α}, —NR^{α}—SO₂R^{α}, —NR^{α}—SO₂NH₂, —NR^{α}-SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —CH₂— or —CH(CH₃)—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR^{δ}—SO₂R^{δ}, —NR^{δ}—SO₂NH₂, —NR^{δ}—SO₂NHR^{δ}, —NR^{δ}—SO₂N(R^{δ})₂, —COR^{δ}, —COOR^{δ}, —OCOR⁶⁷, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR^{δ}, —NH—CON(R^{δ})₂, or —NR^{δ}—CON(R^{δ})₂;
R⁴ is a 5-membered heteroaryl group with three heteroatoms N, O or S in the ring structure (such as triazolyl, oxadiazolyl or thiadiazolyl), wherein the heteroaryl group is optionally substituted with one or two (in particular one) substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR—, —SO₂N(R^{ε})₂, —NH—SO₂R^{ε}, —NH—SO₂NHR—, —NH—SO₂N(R^{ε})₂, or —NR^{ε}—SO₂R^{ε} (in particular wherein the heteroaryl group is optionally substituted with one substituent independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃);
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃:
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
provided that the compound is not:

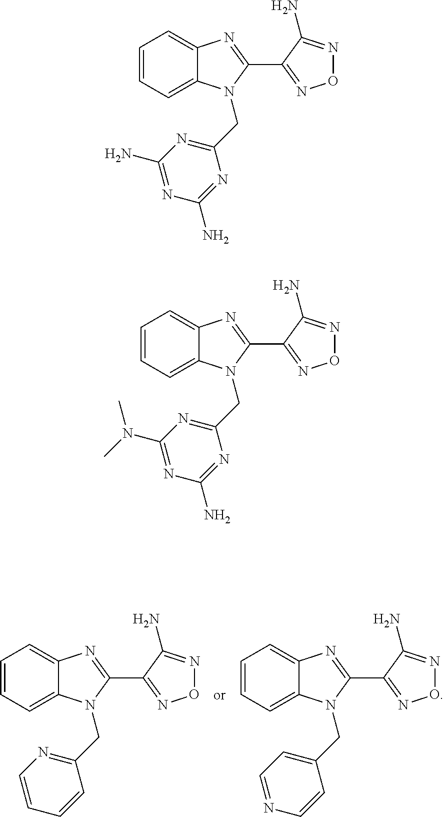 [SMILES: Nc1nc(N)nc(Cn2c(-c3nonc3N)nc3ccccc32)n1 ; CN(C)c1nc(N)nc(Cn2c(-c3nonc3N)nc3ccccc32)n1 ; Nc1nonc1-c1nc2ccccc2n1Cc1ccccn1 ; Nc1nonc1-c1nc2ccccc2n1Cc1ccncc1]

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH, CR¹ or N;
each —R¹ is independently halo, —CN, —R^{α}, —OH, OR^{α}, —NH₂, —NHRα, —N(R^{α})₂, —SR^{α}, —SOR^{α}, —SO₂R^{α}, —SO(NH)R^{α},-SO₂NHRα, —SO₂N(R^{α})₂, —NH—SOR^{α}, —NH—SO₂R^{α}, —NH—SO₂NHRα, —NH—SO₂N(R^{α})₂, —NR^{α}—SOR^{α}, —NR^{α}—SO₂R^{α}, —NR^{α}—SO₂NH₂, —NR^{α}—SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —CH₂— or —CH(CH₃)—;
R³ is pyridinyl, pyridazinyl, pyrimidinyl or pyrazinyl, each of which is optionally substituted with one, two, three or four substituents independently selected from halo, —CN, —R⁶⁷, —OH, —OR⁶⁷, —NH₂, —NHR^{δ}, —N(R^{δ})₂, —SH, —SR^{δ}, —SOR^{δ}, —SO₂R^{δ}, —SO(NH)R^{δ}, —SO(NR)R^{δ}—SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR—SO₂R, —NR^{δ}—SO₂NH₂, —NR^{δ}—SO₂NHR^{δ}, —NR^{δ}—SO₂N(R^{δ})₂, —COR^{δ}, —COOR^{δ}, —OCOR⁶⁷, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR^{δ}, —NH—CON(R^{δ})₂, or —NR^{δ}—CON(R^{δ})₂ (in particular —R³ is pyridinyl, pyridazinyl, pyrimidinyl or pyrazinyl, each of which is optionally substituted with one or two substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃);
R⁴ is a 5-membered heteroaryl group with three heteroatoms N, O or S in the ring structure (such as triazolyl, oxadiazolyl or thiadiazolyl), wherein the heteroaryl group is optionally substituted with one or two (in particular one) substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR^{F}, —SO₂N(R^{F})₂, —NH—SO₂R^{ε}, —NH—SO₂NHR^{F}, —NH—SO₂N(R^{F})₂, or —NR^{ε}—SO₂R^{ε} (in particular wherein the heteroaryl group is optionally substituted with one substituent independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃);
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
provided that the compound is not:

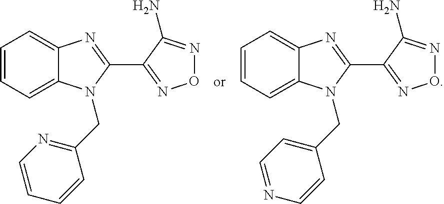 [SMILES: Nc1nonc1-c1nc2ccccc2n1Cc1ccccn1 ; Nc1nonc1-c1nc2ccccc2n1Cc1ccncc1]

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH, CR¹ or N; wherein the compound comprises one, two, three or four R¹ (in particular wherein the compound comprises one, two or three R¹, in particular wherein the compound comprises one or two R¹);
each —R¹ is independently halo, —CN, —R^{α}, —OH, OR^{α}, —NH₂, —NHRα, —N(R^{α})₂, —SR^{α}, —SOR^{α}, —SO₂R^{α}, —SO(NH)R^{α}, —SO₂NHRα, —SO₂N(R^{α})₂, —NH—SOR^{α}, —NH—SO₂R^{α}, —NH—SO₂NHRα, —NH—SO₂N(R^{α})₂, —NR^{α}—SOR^{α}, —NR^{α}—SO₂R^{α}, —NR^{α}—SO₂NH₂, —NR^{α}-SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —C(R^{θ})₂—, —C(R^{θ})₂—(R^{θ})₂—, —(R^{θ})₂—O—, —(R^{θ})₂—NR^{θ}—, —(R^{θ})₂—CO—, or —(R^{θ})₂-CONR^{θ}—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR—SO₂R^{δ}, —NR^{δ}—SO₂NH₂, —NR^{δ}—SO₂NHR^{δ}, —NR^{δ}—SO₂N(R^{δ})₂, —COR^{δ}, —COOR^{δ}, —OCOR⁶⁷, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR^{δ}, —NH—CON(R^{δ})₂, or —NR^{δ}—CON(R^{δ})₂;
R⁴ is a 5-membered heteroaryl group with one or more heteroatoms N, O or S in the ring structure, wherein the heteroaryl group is optionally substituted with one, two, three or four substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR—, —SO₂N(R^{ε})₂, —NH—SO₂R^{ε}, —NH—SO₂NHR^{F}, —NH—SO₂N(R^{F})₂, or —NR^{ε}—SO₂R^{ε};
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and
each —R^{θ} is independently hydrogen or methyl;
   provided that the compound is not:

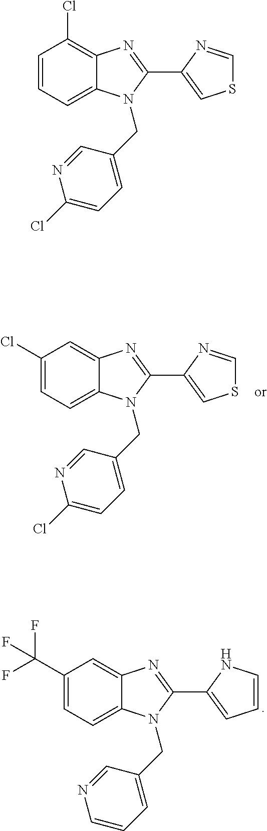 [SMILES: Clc1ccc(Cn2c(-c3cscn3)nc3c(Cl)cccc32)cn1 ; Clc1ccc2c(c1)nc(-c1cscn1)n2Cc1ccc(Cl)nc1 ; FC(F)(F)c1ccc2c(c1)nc(-c1ccc[nH]1)n2Cc1cccnc1]

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH, CR¹ or N; wherein the compound comprises one, two, three or four R¹ (in particular wherein the compound comprises one, two or three R¹, in particular wherein the compound comprises one or two R¹);
each —R¹ is independently halo, —CN, —R^{α}, —OH, OR^{α}, —NH₂, —NHRα, —N(R^{α})₂, —SR^{α}, —SOR^{α}, —SO₂R^{α}, —SO(NH)R^{α}, —SO₂NHRα, —SO₂N(R^{α})₂, —NH—SOR^{α}, —NH—SO₂R^{α}, —NH—SO₂NHRα, —NH—SO₂N(R^{α})₂, —NR^{α}—SOR^{α}, —NR^{α}—SO₂R^{α}, —NR^{α}—SO₂NH₂, —NR^{α}—SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —C(R^{θ})₂—, —C(R^{θ})₂-C(R^{θ})₂—, —C(R^{θ})₂-O—, —C(R^{θ})₂-NR^{θ}—, —C(R^{θ})₂-CO—, or —C(R^{θ})₂-CONR^{θ}—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR)R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR^{δ}—SO₂R^{δ}, —NR^{δ}—SO₂NH₂, —NR^{δ}—SO₂NHR^{δ}, —NR^{δ}—SO₂N(R^{δ})₂, —COR^{δ}, —COOR^{δ}, —OCOR⁶⁷, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR^{δ}, —NH—CON(R^{δ})₂, or —NR^{δ}—CON(R^{δ})₂;
R⁴ is a 5-membered heteroaryl group with one or more heteroatoms N, O or S in the ring structure, wherein the heteroaryl group is not pyrrolyl or thiazolyl (in particular wherein the heteroaryl group is furanyl, thiophenyl, pyrazolyl, imidazolyl, oxazolyl, isoxazolyl, isothiazolyl, triazolyl, oxadiazolyl, thiadiazolyl, tetrazolyl, oxatriazolyl or thiatriazolyl), and wherein the heteroaryl group is optionally substituted with one, two, three or four substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR^{F}, —SO₂N(R^{F})₂, —NH—SO₂R^{ε}, —NH—SO₂NHR^{F}, —NH—SO₂N(R^{F})₂, or —NR^{ε}—SO₂R^{ε};
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and each —R^{θ} is independently hydrogen or methyl.
   In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH, CR¹ or N; wherein the compound comprises one, two, three or four R¹ (in particular wherein the compound comprises one, two or three R¹, in particular wherein the compound comprises one or two R);
each —R¹ is independently halo, —CN, —R^{α}, —OH, OR^{α}, —NH₂, —NHRα, —N(R^{α})₂, —SR^{α}, —SOR^{α}, —SO₂R^{α}, —SO(NH)R^{α}, —SO₂NHRα, —SO₂N(R^{α})₂, —NH—SOR^{α}, —NH—SO₂R^{α}, —NH—SO₂NHRα, —NH—SO₂N(R^{α})₂, —NR^{α}—SOR^{α}, —NR^{α}—SO₂R^{α}, —NR^{α}—SO₂NH₂, —NR^{α}—SO₂NHRα, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —C(R^{θ})₂—, —C(R^{θ})₂-C(R^{θ})₂—, —C(R^{θ})₂-O—, —C(R^{θ})₂-NR^{θ}—, —C(R^{θ})₂-CO—, or —C(R^{θ})₂-CONR^{θ}—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR)R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR^{δ}—SOR^{δ}, —NR^{δ}—SO₂R^{δ}, —NR^{δ}—SO₂NH₂, —NR^{δ}—SO₂NHR^{δ}, —NR^{δ}—SO₂N(R^{δ})₂, —COR^{δ}, —COOR^{δ}, —OCOR⁶⁷, —NH—CHO, —NR^{δ}—CHO, —NH—COR^{δ}, —NR—COR^{δ}, —NH—COOR^{δ}, —NR^{δ}—COOR^{δ}, —CONH₂, —CONHR^{δ}, —CON(R^{δ})₂, —NH—CONHR^{δ}, —NR^{δ}—CONHR^{δ}, —NH—CON(R^{δ})₂, or —NR^{δ}—CON(R^{δ})₂;
R⁴ is a 5-membered heteroaryl group with three heteroatoms N, O or S in the ring structure (such as triazolyl, oxadiazolyl or thiadiazolyl), wherein the heteroaryl group is optionally substituted with one or two (in particular one) substituents independently selected from halo, —CN, —R^{ε}, —OH, —OR^{ε}, —NH₂, —NHR^{δ}, —N(R¹)₂, —SH, —SR^{ε}, —SOR^{ε}, —SO₂R^{ε}, —SO₂NH₂, —SO₂NHR^{F}, —SO₂N(R^{F})₂, —NH—SO₂R^{ε}, —NH—SO₂NHR^{F}, —NH—SO₂N(R^{F})₂, or —NR^{ε}—SO₂R^{ε} (in particular wherein the heteroaryl group is optionally substituted with one substituent independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃);
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{ε} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and
each —R^{θ} is independently hydrogen or methyl.

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH or CR¹; or one of X¹, X², X³ and X⁴ is N, and the remaining of X¹, X², X³ and X⁴ are independently CH or CR¹;
each —R¹ is independently halo, —CN, C₁-C₃ alkyl, C₁-C₃ haloalkyl, C₂-C₃ alkenyl, —OH, —O(C₁-C₃ alkyl), —O(C₁-C₃ haloalkyl), —NH₂, —NH(C₁-C₃ alkyl), —N(C₁-C₃ alkyl)₂, —S(C₁-C₃ alkyl), —SO(C₁-C₃ alkyl), —SO₂(C₁-C₃ alkyl), —NH—SO₂(C₁-C₃ alkyl), —CO(C₁-C₃ alkyl), —COO(C₁-C₃ alkyl), —OCO(C₁-C₃ alkyl), —CONH₂, —CONH(C₁-C₃ alkyl), —CON(C₁-C₃ alkyl)₂, C₃-C₆ cycloalkyl, or a 3- to 6-membered heterocyclic group with one or two heteroatoms N, O or S in the ring structure, wherein the cycloalkyl or heterocyclic group is optionally substituted with one or two substituents independently selected from C₁-C₃ alkyl or —CO(C₁-C₃ alkyl);
R²— is —CH₂—, —CH(CH₃)—, —CH₂—CH₂—, —CH₂—O—, or —CH₂—CO—NH—;
R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two, three or four substituents independently selected from halo, —CN, C₁-C₃ alkyl, C₁-C₃ haloalkyl, C₂-C₃ alkenyl, C₃-C₆ cycloalkyl, —OH, —O(C₁-C₃ alkyl), —O(C₁-C₃ haloalkyl), —NH₂, —NH(C₁-C₃ alkyl), —N(C₁-C₃ alkyl)₂, —SH, —S(C₁-C₃ alkyl), —SO(C₁-C₃ alkyl), —SO₂(C₁-C₃ alkyl), —NH—SO₂(C₁-C₃ alkyl), —CO(C₁-C₃ alkyl), —COO(C₁-C₃ alkyl), —OCO(C₁-C₃ alkyl), —CONH₂, —CONH(C₁-C₃ alkyl), or —CON(C₁-C₃ alkyl)₂; and
R⁴ is a 5-membered heteroaryl group with one, two or three heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl or thiadiazolyl), wherein the heteroaryl group is optionally substituted with one, two or three substituents independently selected from halo, —CN, C₁-C₃ alkyl, C₁-C₃ haloalkyl, C2-C₃ alkenyl, C₃-C₆ cycloalkyl, —OH, —O(C₁-C₃ alkyl), —O(C₁-C₃ haloalkyl), —NH₂, —NH(C₁-C₃ alkyl), —N(C₁-C₃ alkyl)₂, —SH, —S(C₁-C₃ alkyl), —SO(C₁-C₃ alkyl), —SO₂(C₁-C₃ alkyl), or —NH—SO₂(C₁-C₃ alkyl);
provided that the compound is not:

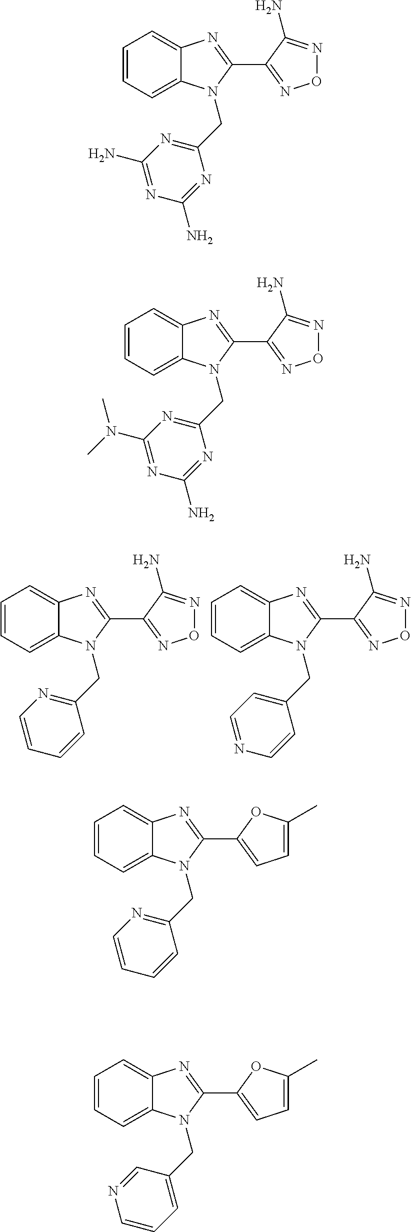

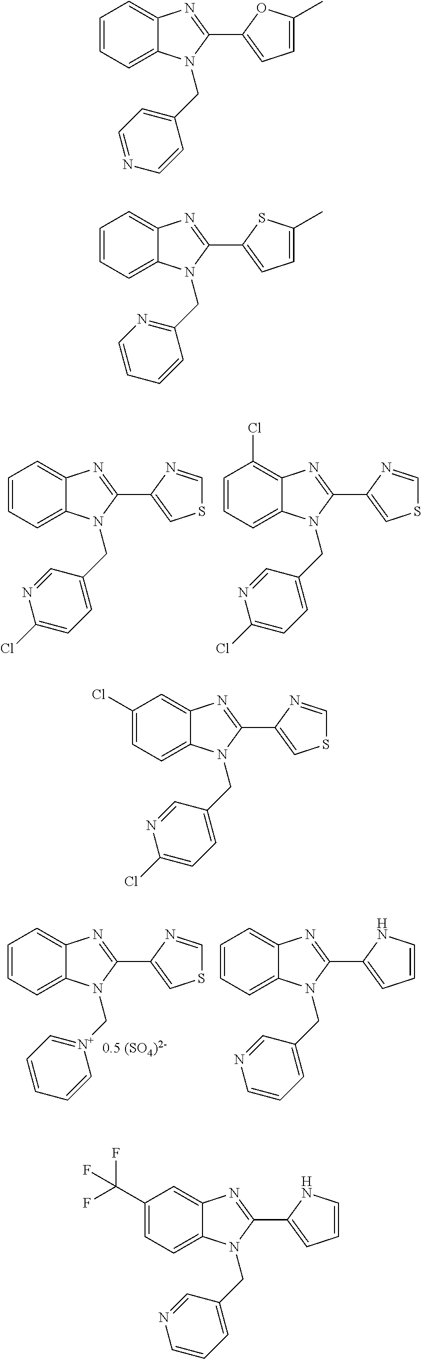

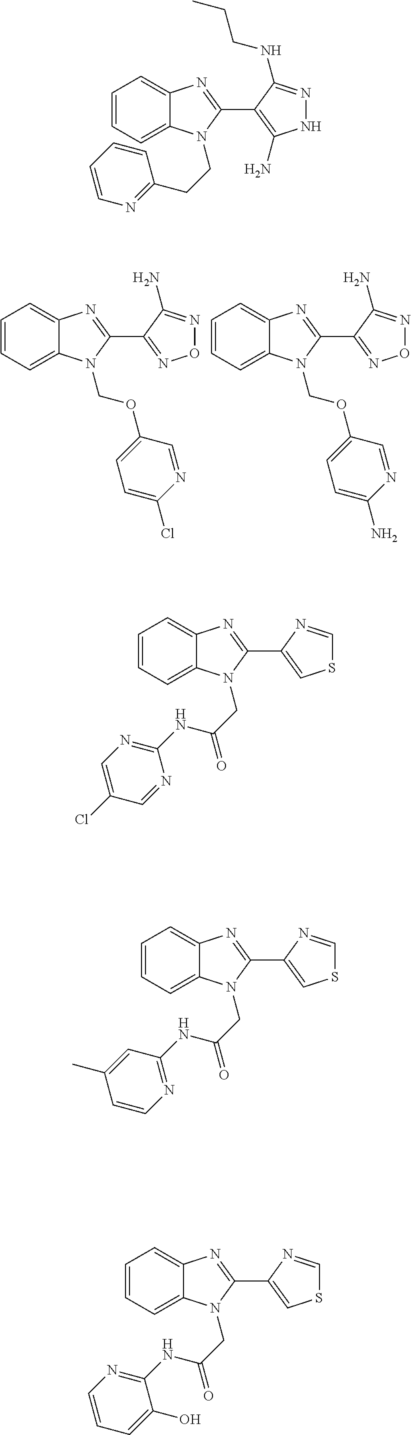

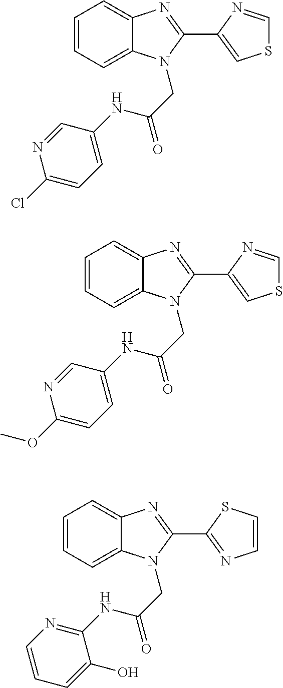 [SMILES: O=C(Cn1c(-c2cscn2)nc2ccccc21)Nc1ccc(Cl)nc1 ; COc1ccc(NC(=O)Cn2c(-c3cscn3)nc3ccccc32)cn1 ; O=C(Cn1c(-c2nccs2)nc2ccccc21)Nc1ncccc1O]

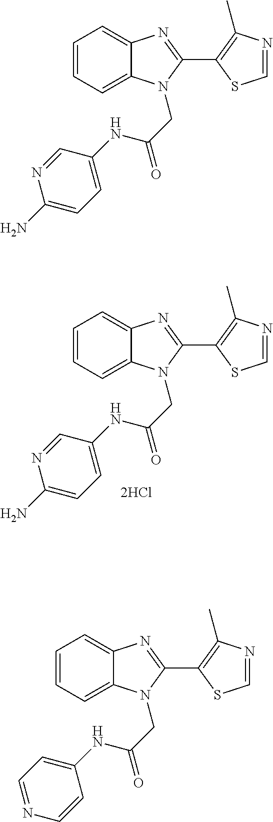 [SMILES: Cc1ncsc1-c1nc2ccccc2n1CC(=O)Nc1ccc(N)nc1 ; Cc1ncsc1-c1nc2ccccc2n1CC(=O)Nc1ccc(N)nc1 ; Cc1ncsc1-c1nc2ccccc2n1CC(=O)Nc1ccncc1]

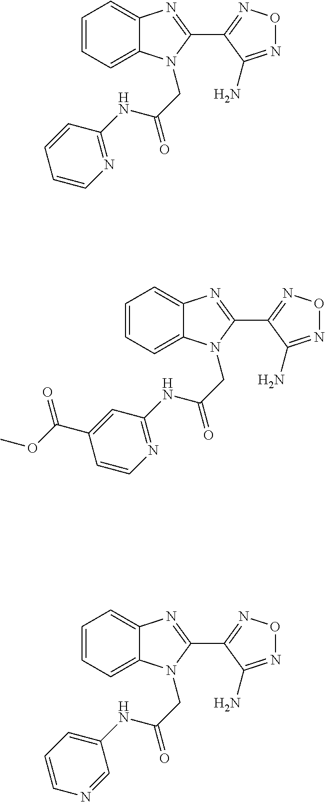 [SMILES: Nc1nonc1-c1nc2ccccc2n1CC(=O)Nc1ccccn1 ; COC(=O)c1ccnc(NC(=O)Cn2c(-c3nonc3N)nc3ccccc32)c1 ; Nc1nonc1-c1nc2ccccc2n1CC(=O)Nc1cccnc1]

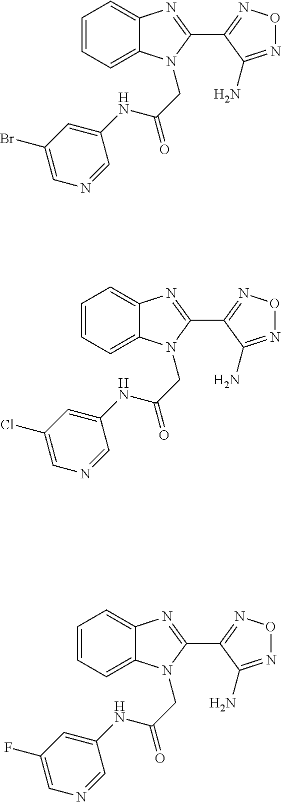 [SMILES: Nc1nonc1-c1nc2ccccc2n1CC(=O)Nc1cncc(Br)c1 ; Nc1nonc1-c1nc2ccccc2n1CC(=O)Nc1cncc(Cl)c1 ; Nc1nonc1-c1nc2ccccc2n1CC(=O)Nc1cncc(F)c1]

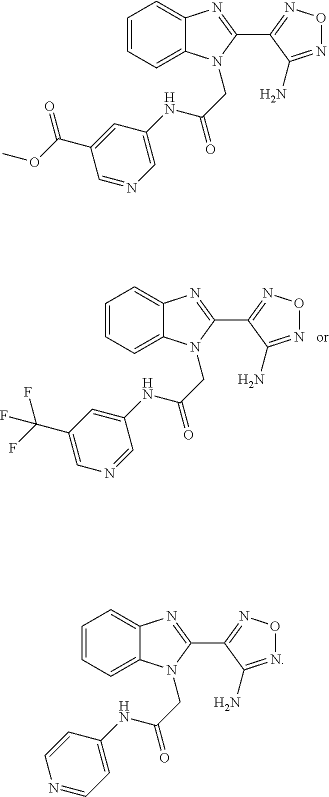 [SMILES: COC(=O)c1cncc(NC(=O)Cn2c(-c3nonc3N)nc3ccccc32)c1 ; Nc1nonc1-c1nc2ccccc2n1CC(=O)Nc1cncc(C(F)(F)F)c1 ; Nc1nonc1-c1nc2ccccc2n1CC(=O)Nc1ccncc1]

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH or CR¹; or one of X¹, X², X³ and X⁴ is N, and the remaining of X¹. X². X³ and X⁴ are independently CH or CR¹;
each —R¹ is independently fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃;
R²— is —CH₂— or —CH(CH₃)—;
R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two or three substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃; and
R⁴ is a 5-membered heteroaryl group with one, two or three heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl or thiadiazolyl; in particular a 5-membered heteroaryl group with two or three heteroatoms N, O or S in the ring structure such as pyrazolyl, imidazolyl, oxazolyl, isoxazolyl, thiazolyl, isothiazolyl, triazolyl, oxadiazolyl or thiadiazolyl; more particularly a 5-membered heteroaryl group with three heteroatoms N, O or S in the ring structure such as triazolyl, oxadiazolyl or thiadiazolyl), wherein the heteroaryl group is optionally substituted with one, two or three (in particular one or two; more particularly one) substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃;
provided that the compound is not:

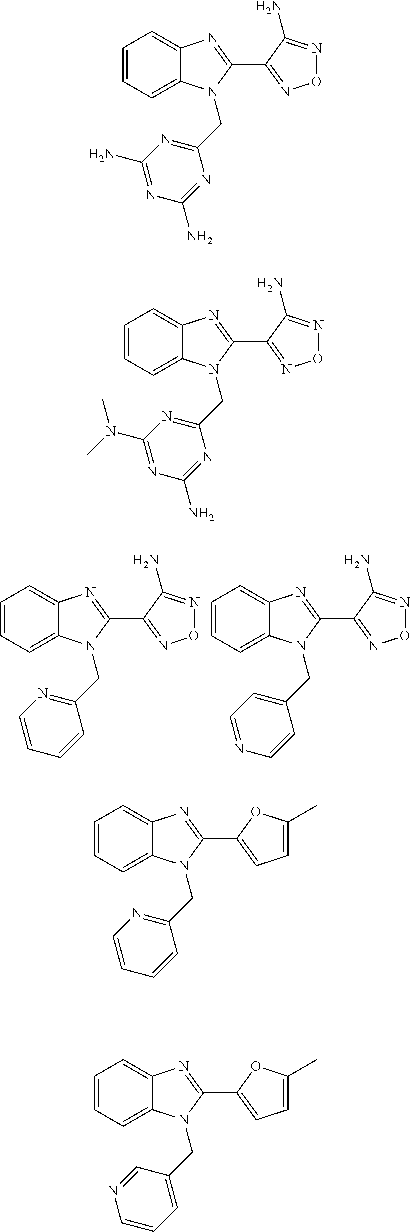

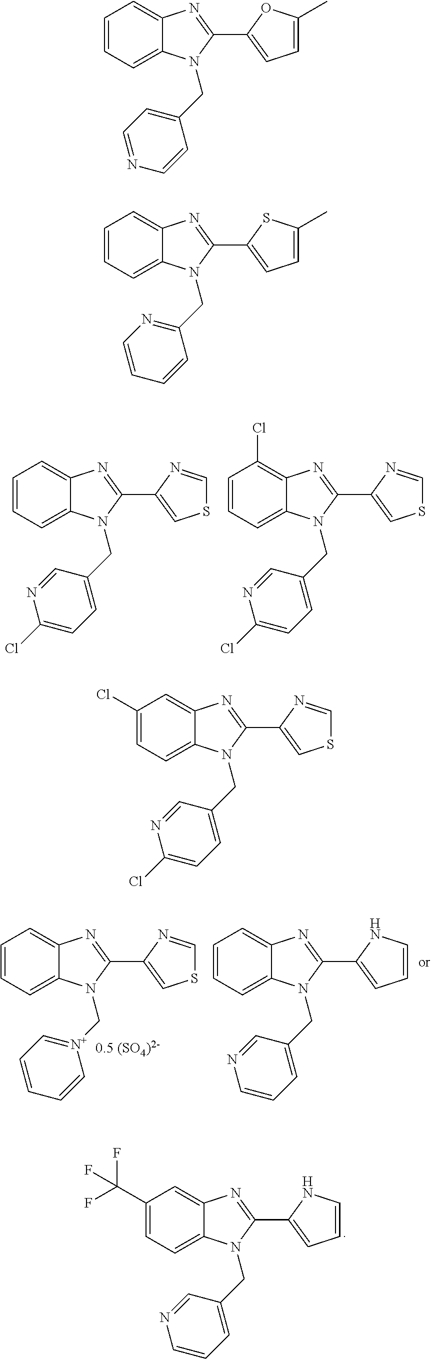

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH or CR¹; or one of X¹, X², X³ and X⁴ is N, and the remaining of X¹, X², X³ and X⁴ are independently CH or CR¹;
each —R¹ is independently fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃;
R²— is —CH₂— or —CH(CH₃)—;
R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two or three substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃; and
R⁴ is a 5-membered heteroaryl group with three heteroatoms N, O or S in the ring structure (such as triazolyl, oxadiazolyl or thiadiazolyl), wherein the heteroaryl group is optionally substituted with one or two (in particular one) substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃;
provided that the compound is not:

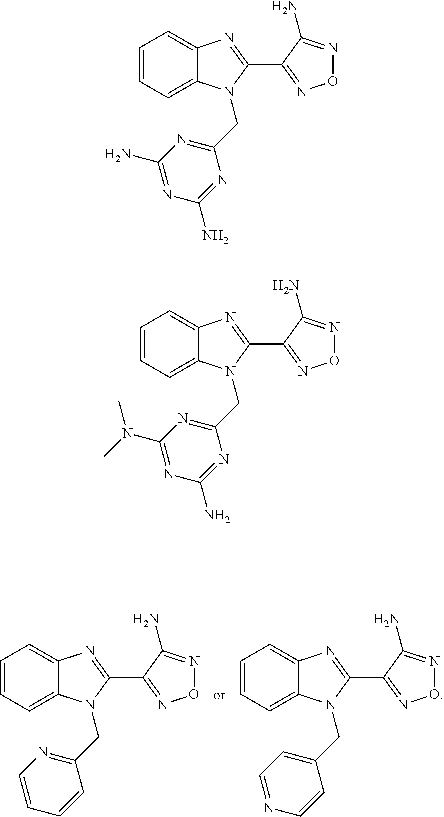 [SMILES: Nc1nc(N)nc(Cn2c(-c3nonc3N)nc3ccccc32)n1 ; CN(C)c1nc(N)nc(Cn2c(-c3nonc3N)nc3ccccc32)n1 ; Nc1nonc1-c1nc2ccccc2n1Cc1ccccn1 ; Nc1nonc1-c1nc2ccccc2n1Cc1ccncc1]

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH or CR¹; or one of X¹, X², X³ and X⁴ is N, and the remaining of X¹, X², X³ and X⁴ are independently CH or CR¹; wherein the compound comprises one or two R¹;
each —R¹ is independently fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃;
R²— is —CH₂— or —CH(CH₃)—;
R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two or three substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃; and
R⁴ is a 5-membered heteroaryl group with two or three heteroatoms N, O or S in the ring structure (such as pyrazolyl, imidazolyl, oxazolyl, isoxazolyl, thiazolyl, isothiazolyl, triazolyl, oxadiazolyl or thiadiazolyl; in particular a 5-membered heteroaryl group with three heteroatoms N, O or S in the ring structure such as triazolyl, oxadiazolyl or thiadiazolyl), wherein the heteroaryl group is optionally substituted with one or two (in particular one) substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃;
provided that the compound is not:

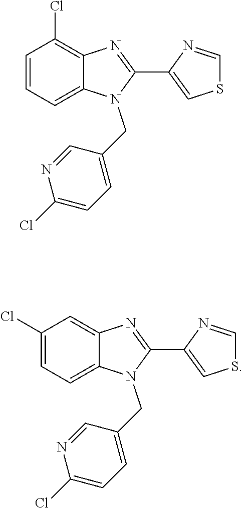 [SMILES: Clc1ccc(Cn2c(-c3cscn3)nc3c(Cl)cccc32)cn1 ; Clc1ccc2c(c1)nc(-c1cscn1)n2Cc1ccc(Cl)nc1]

In another specific embodiment of the first aspect, the present invention provides a compound of formula (I), or a pharmaceutically acceptable salt, N-oxide, solvate or prodrug thereof, wherein:
each X¹, X², X³ and X⁴ is independently CH or CR¹; or one of X¹, X², X³ and X⁴ is N, and the remaining of X¹, X², X³ and X⁴ are independently CH or CR¹; wherein the compound comprises one or two R¹;
each —R¹ is independently fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃;
R²— is —CH₂— or —CH(CH₃)—;
R³ is a 6-membered heteroaryl group with one, two or three nitrogen atoms in the ring structure (such as pyridinyl, pyridazinyl, pyrimidinyl, pyrazinyl or triazinyl), wherein the heteroaryl group is optionally substituted with one, two or three substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃; and
R⁴ is a 5-membered heteroaryl group selected from pyrazolyl, imidazolyl, oxazolyl, isoxazolyl, isothiazolyl, triazolyl, oxadiazolyl or thiadiazolyl (in particular selected from triazolyl, oxadiazolyl or thiadiazolyl), wherein the heteroaryl group is optionally substituted with one or two (in particular one) substituents independently selected from fluoro, chloro, bromo, —CN, —CH₃, —CH₂CH₃, —CH═CH₂, —CF₃, —OH, —OCH₃, —OCH₂CH₃, —OCF₃, —NH₂, —NHCH₃, —N(CH₃)₂, —SO₂CH₃, or —NH—SO₂CH₃.

In another specific embodiment of the first aspect, the present invention provides a compound of formula (I), or a pharmaceutically acceptable salt, solvate or prodrug thereof, wherein:
each X¹, X², X³ and X⁴ is independently CH or CR¹; or one of X¹, X², X³ and X⁴ is N, and the remaining of X¹, X², X³ and X⁴ are independently CH or CR¹; wherein the compound comprises one or two R¹;
each —R¹ is fluoro or chloro;
R²— is —CH₂—;
R³ is pyridinyl, pyridazinyl, pyrimidinyl or pyrazinyl, each of which is optionally substituted with one or two substituents independently selected from fluoro or chloro; and
R⁴ is oxadiazolyl optionally substituted with —CH₃ or —NH₂.

A second aspect of the present invention provides a compound selected from:
5-[[6,7-difluoro-2-(4-methyl-1,2,5-oxadiazol-3-yl)benzimidazol-1-yl]methyl]pyrimidine-2-carbonitrile;
5-[[2-(4-amino-1,2,5-oxadiazol-3-yl)-6,7-difluoro-benzimidazol-1-yl]methyl]pyrimidine-2-carbonitrile;
4-[7-fluoro-1-(pyrimidin-5-ylmethyl)benzimidazol-2-yl]-1,2,5-oxadiazol-3-amine;
4-[6-fluoro-1-(pyrimidin-5-ylmethyl)benzimidazol-2-yl]-1,2,5-oxadiazol-3-amine;
4-[5-fluoro-1-(pyrimidin-5-ylmethyl)benzimidazol-2-yl]-1,2,5-oxadiazol-3-amine;
4-[7-fluoro-1-(pyridazin-3-ylmethyl)benzimidazol-2-yl]-1,2,5-oxadiazol-3-amine;
3-[6,7-difluoro-1-(pyrimidin-5-ylmethyl)benzimidazol-2-yl]-4-methyl-1,2,5-oxadiazole;
5-[[2-(4-amino-1,2,5-oxadiazol-3-yl)-4-fluoro-benzimidazol-1-yl]methyl]pyridine-2-carbonitrile;
3-[4-fluoro-1-(pyrimidin-5-ylmethyl)benzimidazol-2-yl]-4-methyl-1,2,5-oxadiazole;
4-[7-fluoro-1-[(6-methoxypyridin-3-yl)methyl]benzimidazol-2-yl]-1,2,5-oxadiazol-3-amine;
5-[[2-(4-amino-1,2,5-oxadiazol-3-yl)imidazo[4,5-b]pyridin-3-yl]methyl]pyrimidine-2-carbonitrile;
4-[6-fluoro-3-(pyrimidin-5-ylmethyl)imidazo[4,5-b]pyridin-2-yl]-1,2,5-oxadiazol-3-amine;
3-methyl-4-[3-(pyrimidin-5-ylmethyl)imidazo[4,5-b]pyridin-2-yl]-1,2,5-oxadiazole;
4-[3-[(6-methoxypyridin-3-yl)methyl]imidazo[4,5-b]pyridin-2-yl]-1,2,5-oxadiazol-3-amine;
4-[3-(pyrimidin-5-ylmethyl)imidazo[4,5-b]pyridin-2-yl]-1,2,5-oxadiazol-3-amine;
3-[3-[(6-methoxypyridin-3-yl)methyl]imidazo[4,5-b]pyridin-2-yl]-4-methyl-1,2,5-oxadiazole;
3-methyl-4-[3-[[6-(trifluoromethyl)pyridin-3-yl]methyl]imidazo[4,5-b]pyridin-2-yl]-1,2,5-oxadiazole;
6-[[2-(4-methyl-1,2,5-oxadiazol-3-yl)imidazo[4,5-b]pyridin-3-yl]methyl]pyridazine-3-carbonitrile;
4-[3-[[6-(trifluoromethyl)pyridin-3-yl]methyl]imidazo[4,5-b]pyridin-2-yl]-1,2,5-oxadiazol-3-amine;
3-methyl-4-[3-(pyridazin-3-ylmethyl)imidazo[4,5-b]pyridin-2-yl]-1,2,5-oxadiazole;
4-[1-(pyrimidin-5-ylmethyl)benzimidazol-2-yl]-1,2,5-oxadiazol-3-amine;
4-[4,7-difluoro-1-(pyridin-3-ylmethyl)benzimidazol-2-yl]-1,2,5-oxadiazol-3-amine;
4-[3-[(6-chloropyridin-3-yl)methyl]imidazo[4,5-b]pyridin-2-yl]-1,2,5-oxadiazol-3-amine;
3-[3-[(6-chloropyridin-3-yl)methyl]imidazo[4,5-b]pyridin-2-yl]-4-methyl-1,2,5-oxadiazole;
5-[[2-(4-amino-1,2,5-oxadiazol-3-yl)-7-fluoro-benzimidazol-1-yl]methyl]pyrimidine-2-carbonitrile;
5-[[2-(4-amino-1,2,5-oxadiazol-3-yl)-4-fluoro-benzimidazol-1-yl]methyl]pyrimidine-2-carbonitrile;
3-[5,7-difluoro-1-(pyridin-3-ylmethyl)benzimidazol-2-yl]-4-methyl-1,2,5-oxadiazole;
5-[[2-(4-amino-1,2,5-oxadiazol-3-yl)-4,7-difluoro-benzimidazol-1-yl]methyl]pyrimidine-2-carbonitrile;
4-[3-[(6-chloropyridin-3-yl)methyl]-6-fluoro-imidazo[4,5-b]pyridin-2-yl]-1,2,5-oxadiazol-3-amine;
3-[1-[dideuterio(pyridin-3-yl)methyl]-4-fluoro-benzimidazol-2-yl]-4-methyl-1,2,5-oxadiazole;
3-[1-[dideuterio(pyridin-3-yl)methyl]-7-fluoro-benzimidazol-2-yl]-4-methyl-1,2,5-oxadiazole;
4-[7-fluoro-1-(pyrazin-2-ylmethyl)benzimidazol-2-yl]-1,2,5-oxadiazol-3-amine;
4-[5-bromo-1-(pyridin-3-ylmethyl)benzimidazol-2-yl]-1,2,5-oxadiazol-3-amine;
4-[5-(dimethylamino)-1-(pyridin-3-ylmethyl)benzimidazol-2-yl]-1,2,5-oxadiazol-3-amine;
5-[[2-(4-amino-1,2,5-oxadiazol-3-yl)-7-fluoro-benzimidazol-1-yl]methyl]pyrazine-2-carbonitrile;
5-[[2-(4-amino-1,2,5-oxadiazol-3-yl)-4-fluoro-benzimidazol-1-yl]methyl]pyrazine-2-carbonitrile;
5-[[2-(4-methyl-1,2,5-oxadiazol-3-yl)benzimidazol-1-yl]methyl]pyridine-2-carbonitrile;
3-[1-[(6-chloropyridin-3-yl)methyl]-7-fluoro-benzimidazol-2-yl]-4-methyl-1,2,5-oxadiazole;
3-[1-[(6-chloropyridin-3-yl)methyl]-4-fluoro-benzimidazol-2-yl]-4-methyl-1,2,5-oxadiazole;
5-[[4-fluoro-2-(4-methyl-1,2,5-oxadiazol-3-yl)benzimidazol-1-yl]methyl]pyridine-2-carbonitrile;
5-[[7-fluoro-2-(4-methyl-1,2,5-oxadiazol-3-yl)benzimidazol-1-yl]methyl]pyrazine-2-carbonitrile;
5-[[4-fluoro-2-(4-methyl-1,2,5-oxadiazol-3-yl)benzimidazol-1-yl]methyl]pyrazine-2-carbonitrile;
3-[7-fluoro-1-[(6-methoxypyridin-3-yl)methyl]benzimidazol-2-yl]-4-methyl-1,2,5-oxadiazole;
5-[[7-fluoro-2-(4-methyl-1,2,5-oxadiazol-3-yl)benzimidazol-1-yl]methyl]pyrimidine-2-carbonitrile;
5-[[4-fluoro-2-(4-methyl-1,2,5-oxadiazol-3-yl)benzimidazol-1-yl]methyl]pyrimidine-2-carbonitrile;
6-[[7-fluoro-2-(4-methyl-1,2,5-oxadiazol-3-yl)benzimidazol-1-yl]methyl]pyridazine-3-carbonitrile;
6-[[4-fluoro-2-(4-methyl-1,2,5-oxadiazol-3-yl)benzimidazol-1-yl]methyl]pyridazine-3-carbonitrile;
3-[1-[(6-ethoxypyridin-3-yl)methyl]-4-fluoro-benzimidazol-2-yl]-4-methyl-1,2,5-oxadiazole;
3-[7-fluoro-1-(pyrimidin-5-ylmethyl)benzimidazol-2-yl]-4-methyl-1,2,5-oxadiazole;
5-[[2-(4-methyl-1,2,5-oxadiazol-3-yl)imidazo[4,5-b]pyridin-3-yl]methyl]pyridine-2-carbonitrile;
5-[[2-(4-amino-1,2,5-oxadiazol-3-yl)imidazo[4,5-b]pyridin-3-yl]methyl]pyridine-2-carbonitrile;
5-[[7-fluoro-2-(4-methyl-1,2,5-oxadiazol-3-yl)benzimidazol-1-yl]methyl]pyridine-2-carbonitrile;
3-[1-[(6-methoxypyridin-3-yl)methyl]benzimidazol-2-yl]-4-methyl-1,2,5-oxadiazole;
5-[[2-(4-methyl-1,2,5-oxadiazol-3-yl)benzimidazol-1-yl]methyl]pyridin-2-ol;
5-[[6-fluoro-2-(4-methyl-1,2,5-oxadiazol-3-yl)benzimidazol-1-yl]methyl]pyridine-2-carbonitrile;
3-[6,7-difluoro-1-(pyridin-3-ylmethyl)benzimidazol-2-yl]-4-methyl-1,2,5-oxadiazole;
3-methyl-4-[1-(pyrimidin-5-ylmethyl)benzimidazol-2-yl]-1,2,5-oxadiazole;
3-methyl-4-[1-[(6-(methylsulfonyl)pyridin-3-yl)methyl]benzimidazol-2-yl]-1,2,5-oxadiazole;
4-[3-(pyridin-3-ylmethyl)imidazo[4,5-b]pyridin-2-yl]-1,2,5-oxadiazol-3-amine;
3-[1-[(6-chloropyridin-3-yl)methyl]benzimidazol-2-yl]-4-methyl-1,2,5-oxadiazole;
5-[[4-chloro-2-(4-methyl-1,2,5-oxadiazol-3-yl)benzimidazol-1-yl]methyl]pyridine-2-carbonitrile;
5-[[7-chloro-2-(4-methyl-1,2,5-oxadiazol-3-yl)benzimidazol-1-yl]methyl]pyridine-2-carbonitrile;
3-methyl-4-[1-[(6-methylpyridin-3-yl)methyl]benzimidazol-2-yl]-1,2,5-oxadiazole;
3-methyl-4-[1-[(2-methylpyrimidin-5-yl)methyl]benzimidazol-2-yl]-1,2,5-oxadiazole;
3-[4,7-difluoro-1-(pyridin-3-ylmethyl)benzimidazol-2-yl]-4-methyl-1,2,5-oxadiazole;
3-[1-[(2-methoxypyridin-4-yl)methyl]benzimidazol-2-yl]-4-methyl-1,2,5-oxadiazole;
3-[[2-(4-methyl-1,2,5-oxadiazol-3-yl)benzimidazol-1-yl]methyl]pyridine-2-carbonitrile;
5-[7-fluoro-1-(pyridin-3-ylmethyl)benzimidazol-2-yl]-1,2,3-thiadiazole;
3-methyl-4-[1-[(3-methylpyridin-2-yl)methyl]benzimidazol-2-yl]-1,2,5-oxadiazole;
3-[7-ethoxy-1-(pyridin-4-ylmethyl)benzimidazol-2-yl]-4-methyl-1,2,5-oxadiazole;
2-(4-methyl-1,2,5-oxadiazol-3-yl)-3-(pyridin-3-ylmethyl)benzimidazol-4-amine;
N-methyl-5-[[2-(4-methyl-1,2,5-oxadiazol-3-yl)benzimidazol-1-yl]methyl]pyridin-2-amine;
3-methyl-4-[1-[(2-methylpyridin-4-yl)methyl]benzimidazol-2-yl]-1,2,5-oxadiazole;
3-[1-[(4,6-dimethylpyridin-2-yl)methyl]benzimidazol-2-yl]-4-methyl-1,2,5-oxadiazole;
3-methyl-4-[1-[(1-oxidopyridin-1-ium-3-yl)methyl]benzimidazol-2-yl]-1,2,5-oxadiazole;
3-methyl-4-[1-[(1-oxidopyridin-1-ium-4-yl)methyl]benzimidazol-2-yl]-1,2,5-oxadiazole;
3-methyl-4-[1-[(6-methylpyridin-2-yl)methyl]benzimidazol-2-yl]-1,2,5-oxadiazole;
3-methyl-4-[1-(pyridin-2-ylmethyl)benzimidazol-2-yl]-1,2,5-oxadiazole;
5-[[6-fluoro-2-(4-methyl-1,2,5-thiadiazol-3-yl)benzimidazol-1-yl]methyl]pyridine-2-carbonitrile;
N-methyl-2-(4-methyl-1,2,5-oxadiazol-3-yl)-3-(pyridin-3-ylmethyl)benzimidazol-4-amine;
3-[1-[(3-fluoropyridin-2-yl)methyl]benzimidazol-2-yl]-4-methyl-1,2,5-oxadiazole;
5-[[4,7-difluoro-2-(4-methyl-1,2,5-oxadiazol-3-yl)benzimidazol-1-yl]methyl]pyrimidine-2-carbonitrile;
3-[4,7-difluoro-1-(pyrimidin-5-ylmethyl)benzimidazol-2-yl]-4-methyl-1,2,5-oxadiazole;
rac-4-[1-[1-(pyridin-3-yl)ethyl]benzimidazol-2-yl]-1,2,5-oxadiazol-3-amine;
4-[7-fluoro-1-(pyridin-3-ylmethyl)benzimidazol-2-yl]-1,2,5-oxadiazol-3-amine;
4-[4-fluoro-1-(pyridin-3-ylmethyl)benzimidazol-2-yl]-1,2,5-oxadiazol-3-amine;
4-(1-((6-bromopyridin-3-yl)methyl)benzimidazol-2-yl)-1,2,5-oxadiazol-3-amine;
3-[1-(pyridin-3-ylmethyl)benzimidazol-2-yl]-4-vinyl-1,2,5-thiadiazole;
4-[1-(pyridin-3-ylmethyl)benzimidazol-2-yl]-1,2,5-oxadiazol-3-amine;
4-[6-fluoro-1-(pyridin-4-ylmethyl)benzimidazol-2-yl]-1,2,5-oxadiazol-3-amine;
4-[5-fluoro-1-(pyridin-4-ylmethyl)benzimidazol-2-yl]-1,2,5-oxadiazol-3-amine;
4-[1-[(2-methoxypyridin-4-yl)methyl]benzimidazol-2-yl]-1,2,5-oxadiazol-3-amine;
4-[1-[[2-(trifluoromethyl)pyridin-4-yl]methyl]benzimidazol-2-yl]-1,2,5-oxadiazol-3-amine;
4-[1-[[5-(trifluoromethyl)pyridin-3-yl]methyl]benzimidazol-2-yl]-1,2,5-oxadiazol-3-amine;
N-methyl-4-[1-(pyridin-3-ylmethyl)benzimidazol-2-yl]-1,2,5-thiadiazol-3-amine;
4-[1-[[6-(trifluoromethyl)pyridin-3-yl]methyl]benzimidazol-2-yl]-1,2,5-oxadiazol-3-amine;
3-methyl-4-[1-(pyridin-4-ylmethyl)benzimidazol-2-yl]-1,2,5-oxadiazole;
4-[1-(pyridin-3-ylmethyl)benzimidazol-2-yl]-1,2,5-thiadiazole-3-carbonitrile; 2-(3-methylthiophen-2-yl)-1-(pyridin-3-ylmethyl)benzimidazole;
3-methyl-4-[1-(pyridin-3-ylmethyl)benzimidazol-2-yl]-1,2,5-oxadiazole;
5-methyl-4-[1-(pyridin-3-ylmethyl)benzimidazol-2-yl]-1,2,3-thiadiazole;
5-methyl-4-[1-(pyridin-3-ylmethyl)benzimidazol-2-yl]oxazole;
4-methyl-3-[1-(pyridin-3-ylmethyl)benzimidazol-2-yl]isoxazole;
3-ethyl-4-[1-(pyridin-3-ylmethyl)benzimidazol-2-yl]-1,2,5-thiadiazole;
4-[1-(pyrimidin-4-ylmethyl)benzimidazol-2-yl]-1,2,5-oxadiazol-3-amine;
4-[1-(pyridazin-4-ylmethyl)benzimidazol-2-yl]-1,2,5-oxadiazol-3-amine;
3-fluoro-4-[1-(pyridin-3-ylmethyl)benzimidazol-2-yl]-1,2,5-thiadiazole;
4-[1-[[2-(trifluoromethyl)pyridin-3-yl]methyl]benzimidazol-2-yl]-1,2,5-oxadiazol-3-amine;
3-methyl-4-[3-(pyridin-3-ylmethyl)imidazo[4,5-c]pyridin-2-yl]-1,2,5-oxadiazole;
4,5-dimethyl-3-[1-(pyridin-3-ylmethyl)benzimidazol-2-yl]isoxazole;
3-methyl-4-[1-(pyridin-3-ylmethyl)benzimidazol-2-yl]isoxazole;
2-(1,4-dimethylpyrazol-3-yl)-1-(pyridin-3-ylmethyl)benzimidazole;
2-(1-methylpyrazol-5-yl)-1-(pyridin-3-ylmethyl)benzimidazole;
3-ethyl-4-[1-(pyridin-3-ylmethyl)benzimidazol-2-yl]-1,2,5-oxadiazole;
2-(furan-2-yl)-1-(pyridin-4-ylmethyl)benzimidazole;
4-[6-fluoro-1-(pyridin-3-ylmethyl)benzimidazol-2-yl]-1,2,5-oxadiazol-3-amine;
4-[5-fluoro-1-(pyridin-3-ylmethyl)benzimidazol-2-yl]-1,2,5-oxadiazol-3-amine;
4-[7-fluoro-1-(pyridin-4-ylmethyl)benzimidazol-2-yl]-1,2,5-oxadiazol-3-amine;
4-[4-fluoro-1-(pyridin-4-ylmethyl)benzimidazol-2-yl]-1,2,5-oxadiazol-3-amine;
3-[7-fluoro-1-(pyridin-3-ylmethyl)benzimidazol-2-yl]-4-methyl-1,2,5-oxadiazole;
3-[4-fluoro-1-(pyridin-3-ylmethyl)benzimidazol-2-yl]-4-methyl-1,2,5-oxadiazole;
3-[4-fluoro-1-(pyridin-3-ylmethyl)benzimidazol-2-yl]-4-methyl-1,2,5-thiadiazole;
3-[7-fluoro-1-(pyridin-3-ylmethyl)benzimidazol-2-yl]-4-methyl-1,2,5-thiadiazole;
3-bromo-4-(1-(pyridin-3-ylmethyl)benzimidazol-2-yl)-1,2,5-thiadiazole;
3-methyl-4-[1-(pyridin-3-ylmethyl)benzimidazol-2-yl]-1,2,5-thiadiazole;
4-[7-fluoro-1-(pyridin-3-ylmethyl)benzimidazol-2-yl]-5-methyl-1,2,3-thiadiazole;
4-[4-fluoro-1-(pyridin-3-ylmethyl)benzimidazol-2-yl]-5-methyl-1,2,3-thiadiazole;
4-methyl-5-[1-(pyridin-3-ylmethyl)benzimidazol-2-yl]isoxazole;
4-[4-fluoro-1-(pyrimidin-5-ylmethyl)benzimidazol-2-yl]-1,2,5-oxadiazol-3-amine;
4-(7-fluoro-1-(pyridazin-4-ylmethyl)-benzimidazol-2-yl)-1,2,5-oxadiazol-3-amine;
4-(4-fluoro-1-(pyridazin-4-ylmethyl)-benzimidazol-2-yl)-1,2,5-oxadiazol-3-amine;
4-(7-fluoro-1-(pyrimidin-4-ylmethyl)-benzimidazol-2-yl)-1,2,5-oxadiazol-3-amine;
4-(4-fluoro-1-(pyrimidin-4-ylmethyl)-benzimidazol-2-yl)-1,2,5-oxadiazol-3-amine;
4-(5,7-difluoro-1-(pyridin-4-ylmethyl)-benzimidazol-2-yl)-1,2,5-oxadiazol-3-amine;
3-(7-fluoro-1-((6-(methylsulfonyl)pyridin-3-yl)methyl)-benzimidazol-2-yl)-4-methyl-1,2,5-oxadiazole;
3-(4-fluoro-1-((6-(methylsulfonyl)pyridin-3-yl)methyl)-benzimidazol-2-yl)-4-methyl-1,2,5-oxadiazole;
3-(7-fluoro-1-(pyridazin-3-ylmethyl)-benzimidazol-2-yl)-4-methyl-1,2,5-thiadiazole;
3-(4-fluoro-1-(pyridazin-3-ylmethyl)-benzimidazol-2-yl)-4-methyl-1,2,5-thiadiazole;
4-(5,7-difluoro-1-(pyridazin-3-ylmethyl)-benzimidazol-2-yl)-1,2,5-oxadiazol-3-amine;
4-(4,6-difluoro-1-(pyridazin-3-ylmethyl)-benzimidazol-2-yl)-1,2,5-oxadiazol-3-amine;
4-(7-fluoro-1-(pyridazin-3-ylmethyl)-benzimidazol-2-yl)-N-methyl-1,2,5-thiadiazol-3-amine;
4-(4-fluoro-1-(pyridazin-3-ylmethyl)-benzimidazol-2-yl)-N-methyl-1,2,5-thiadiazol-3-amine;
4-(6,7-difluoro-1-(pyridin-4-ylmethyl)-benzimidazol-2-yl)-1,2,5-oxadiazol-3-amine;
3-(5,7-difluoro-1-(pyridazin-3-ylmethyl)-benzimidazol-2-yl)-4-methyl-1,2,5-oxadiazole;
3-(4,6-difluoro-1-(pyridazin-3-ylmethyl)-benzimidazol-2-yl)-4-methyl-1,2,5-oxadiazole;
4-(1-((6-chloropyridazin-3-yl)methyl)-7-fluoro-benzimidazol-2-yl)-1,2,5-oxadiazol-3-amine;
4-(1-((6-chloropyridazin-3-yl)methyl)-4-fluoro-benzimidazol-2-yl)-1,2,5-oxadiazol-3-amine;
6-((2-(4-amino-1,2,5-oxadiazol-3-yl)-7-fluoro-benzimidazol-1-yl)methyl)pyridazin-3-ol;
4-(1-((6-deuteriopyridazin-3-yl)methyl)-7-fluoro-benzimidazol-2-yl)-1,2,5-oxadiazol-3-amine;
4-(7-fluoro-1-((6-methoxypyridazin-3-yl)methyl)-benzimidazol-2-yl)-1,2,5-oxadiazol-3-amine;
4-(4-fluoro-1-((6-methoxypyridazin-3-yl)methyl)-benzimidazol-2-yl)-1,2,5-oxadiazol-3-amine;
4-(4,7-difluoro-1-(pyridazin-3-ylmethyl)-benzimidazol-2-yl)-1,2,5-oxadiazol-3-amine;
4-(4,7-difluoro-1-(pyridin-4-ylmethyl)-benzimidazol-2-yl)-1,2,5-oxadiazol-3-amine;
4-(7-fluoro-1-(pyridazin-3-ylmethyl)-benzimidazol-2-yl)-1,2,5-thiadiazol-3-amine;
4-(4-fluoro-1-(pyridazin-3-ylmethyl)-benzimidazol-2-yl)-1,2,5-thiadiazol-3-amine;
4-(7-fluoro-1-(pyridazin-3-ylmethyl)-benzimidazol-2-yl)isoxazol-3-amine;
4-(l-((6-chloropyridin-3-yl)methyl)-6-fluoro-1H-imidazo[4,5-b]pyridin-2-yl)-1,2,5-oxadiazol-3-amine;
4-(6-fluoro-1-(pyrimidin-5-ylmethyl)-1H-imidazo[4,5-b]pyridin-2-yl)-1,2,5-oxadiazol-3-amine;
(S)-4-(7-fluoro-1-(1-(pyridin-3-yl)ethyl)-benzimidazol-2-yl)-1,2,5-oxadiazol-3-amine;
4-(6,7-difluoro-1-(pyridazin-3-ylmethyl)-benzimidazol-2-yl)-1,2,5-oxadiazol-3-amine;
4-(4,5-difluoro-1-(pyridazin-3-ylmethyl)-benzimidazol-2-yl)-1,2,5-oxadiazol-3-amine;
N-methyl-5-((2-(4-methyl-1,2,5-oxadiazol-3-yl)-benzimidazol-1-yl)methyl)pyridine-2-sulfonamide;
5-((2-(4-methyl-1,2,5-oxadiazol-3-yl)-3H-imidazo[4,5-b]pyridin-3-yl)methyl)pyrimidine-2-carbonitrile;
4-(7-fluoro-1-((6-(trifluoromethyl)pyridazin-3-yl)methyl)-benzimidazol-2-yl)-1,2,5-oxadiazol-3-amine;
4-(4-fluoro-1-((6-(trifluoromethyl)pyridazin-3-yl)methyl)-benzimidazol-2-yl)-1,2,5-oxadiazol-3-amine;
4-(7-fluoro-1-((6-methylpyridazin-3-yl)methyl)-benzimidazol-2-yl)-1,2,5-oxadiazol-3-amine;
4-(4-fluoro-1-((6-methylpyridazin-3-yl)methyl)-benzimidazol-2-yl)-1,2,5-oxadiazol-3-amine;
3-(6,7-difluoro-1-(pyridazin-3-ylmethyl)-benzimidazol-2-yl)-4-methyl-1,2,5-oxadiazole;
3-(4,5-difluoro-1-(pyridazin-3-ylmethyl)-benzimidazol-2-yl)-4-methyl-1,2,5-oxadiazole;
4-(1-((6-(difluoromethyl)pyridazin-3-yl)methyl)-7-fluoro-benzimidazol-2-yl)-1,2,5-oxadiazol-3-amine;
4-(1-((6-(difluoromethyl)pyridazin-3-yl)methyl)-4-fluoro-benzimidazol-2-yl)-1,2,5-oxadiazol-3-amine;
6-((2-(4-amino-1,2,5-oxadiazol-3-yl)-7-fluoro-benzimidazol-1-yl)methyl)pyridazine-3-carbonitrile;
6-((2-(4-amino-1,2,5-oxadiazol-3-yl)-4-fluoro-benzimidazol-1-yl)methyl)pyridazine-3-carbonitrile;
4-(7-fluoro-1-((6-(trifluoromethoxy)pyridin-3-yl)methyl)-benzimidazol-2-yl)-1,2,5-oxadiazol-3-amine;
6-((2-(4-amino-1,2,5-oxadiazol-3-yl)-3H-imidazo[4,5-b]pyridin-3-yl)methyl)pyridazine-3-carbonitrile;
4-(7-fluoro-1-(pyridazin-3-ylmethyl)-benzimidazol-2-yl)-N-methyl-1,2,5-oxadiazol-3-amine;
4-(4-fluoro-1-(pyridazin-3-ylmethyl)-benzimidazol-2-yl)-1,2,5-oxadiazol-3-amine;
3-(1-((6-(ethylsulfonyl)pyridin-3-yl)methyl)-benzimidazol-2-yl)-4-methyl-1,2,5-oxadiazole;
3-methyl-4-(1-((6-(methylthio)pyridin-3-yl)methyl)-benzimidazol-2-yl)-1,2,5-oxadiazole;
3-(7-fluoro-1-(pyridazin-3-ylmethyl)-benzimidazol-2-yl)-4-methylisoxazole;
4-(7-fluoro-1-((2-methoxypyridin-4-yl)methyl)-benzimidazol-2-yl)-1,2,5-oxadiazol-3-amine;
4-(4-fluoro-1-((2-methoxypyridin-4-yl)methyl)-benzimidazol-2-yl)-1,2,5-oxadiazol-3-amine;
3-(4,7-difluoro-1-(pyridazin-3-ylmethyl)-benzimidazol-2-yl)-4-methyl-1,2,5-oxadiazole;
3-(7-fluoro-1-(pyridazin-3-ylmethyl)-benzimidazol-2-yl)isoxazol-4-amine;
4-(7-fluoro-1-(pyridazin-3-ylmethyl)-1H-imidazo[4,5-c]pyridin-2-yl)-1,2,5-oxadiazol-3-amine;
4-(7-fluoro-3-(pyridazin-3-ylmethyl)-3H-imidazo[4,5-c]pyridin-2-yl)-1,2,5-oxadiazol-3-amine;
   or an enantiomer of any of the foregoing;
   or a pharmaceutically acceptable salt, solvate or prodrug of any of the foregoing.

A third aspect of the present invention provides a process for the preparation of a compound of formula (I) or a pharmaceutically acceptable salt, N-oxide, solvate or prodrug thereof, according to the first aspect of the present invention, wherein the process comprises:
(A) reacting a compound of formula (V)

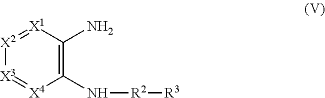

or a salt thereof, with a compound of formula (VI), R⁴—CO₂H (VI), or a salt thereof, or a compound of formula (VIII), R⁴—CHO (VIII), or a salt thereof, or a compound of formula (IX), Cl—C(NOH)—R⁴ (IX), or a salt thereof, wherein R², R³, R⁴, X¹, X², X³ and X⁴ are as defined in the first aspect of the present invention; or
(B) reacting a compound of formula (XII)

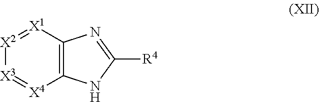

or a salt thereof, with a compound of formula (XIII), Z—R²—R³ (XIII), or a salt thereof, wherein R², R³, R⁴, X¹, X², X³ and X⁴ are as defined in the first aspect of the present invention, and Z is a leaving group;
and optionally thereafter carrying out one or more of the following procedures:
   converting a compound of formula (I) into another compound of formula (I);
   removing any protecting groups;
   forming a pharmaceutically acceptable salt or N-oxide.

In one embodiment of the process of the present invention, a compound of formula (V)

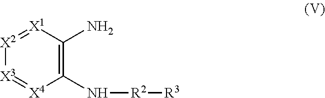

or a salt thereof, is reacted with a compound of formula (VI), R⁴—CO₂H (VI), or a salt thereof, or a compound of formula (VIII), R⁴—CHO (VIII), or a salt thereof, or a compound of formula (IX), Cl—C(NOH)—R⁴ (IX), or a salt thereof, wherein R², R³, R⁴, X¹, X², X³ and X⁴ are as defined in the first aspect of the present invention. This process is depicted schematically in scheme 1.

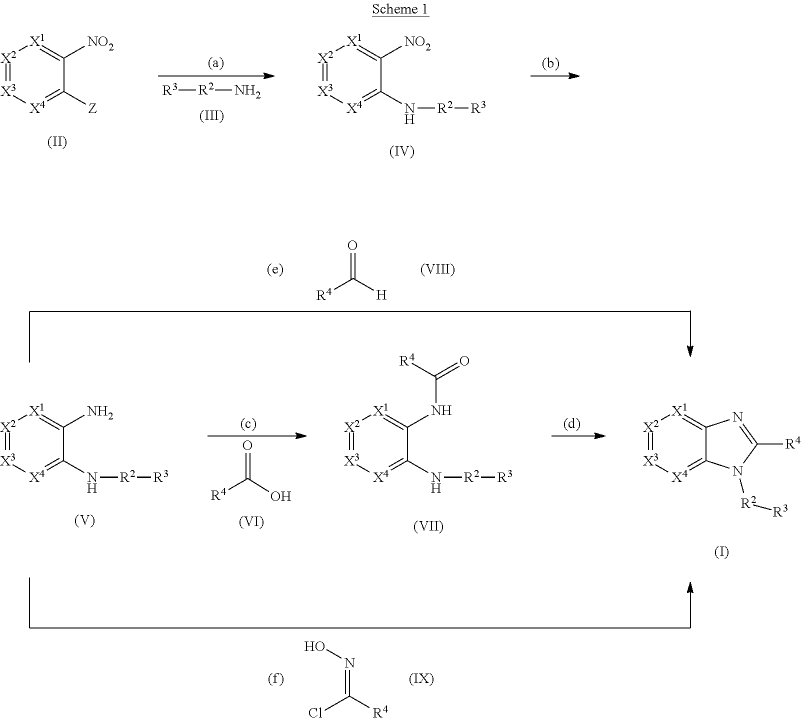

In step (c), a compound of formula (V) or a salt thereof, is reacted with a compound of formula (VI) or a salt thereof, to provide a compound of formula (VII) or a salt thereof. The reaction is typically carried out in the presence of a coupling agent such as T3P, HATU or DCC, optionally in the presence of HOBt, typically in the presence of a base, such as TEA or DIPEA, typically in a solvent such as DMF or DCM. The reaction is typically carried out at a temperature of about 5-25° C. for about 1-12 hours.

In step (d), a compound of formula (VII) or a salt thereof, is cyclised to provide a compound of formula (I) or a salt thereof, typically by heating in the presence of an acid, such as AcOH, typically at a temperature of about 90-115° C. for about 0.5-10 hours.

The conversion achieved by steps (c) and (d), can alternatively be achieved by reacting a compound of formula (V) or a salt thereof, with a compound of formula (VIII) or a salt thereof, as shown in step (e). The reaction is typically carried out in the presence of Na₂S₂O₅, typically in a solvent such as EtOH, typically at a temperature of about 50-75° C. for about 10-16 hours.

Alternatively still, the conversion achieved by steps (c) and (d), can be achieved by reacting a compound of formula (V) or a salt thereof, with a compound of formula (IX) or a salt thereof, as shown in step (f), typically by heating in a solvent, such as EtOH, typically at a temperature of about 80-90° C. for about 1-24 hours.

A compound of formula (V) or a salt thereof, can be prepared in a two-step process as shown in steps (a) and (b). In step (a), a compound of formula (II) or a salt thereof, is reacted with a compound of formula (III) or a salt thereof, to provide a compound of formula (IV) or a salt thereof, wherein Z is a leaving group such as fluoro, chloro, bromo, iodo, tosylate, mesylate, or triflate. The reaction is typically carried out in the presence of a base, such as TEA or DIPEA, typically in a solvent such as MeCN or n-BuOH. The reaction is typically carried out at a temperature of about 20-110° C. for about 0.5-8 hours.

In step (b), a compound of formula (IV) or a salt thereof, is reduced to a compound of formula (V) or a salt thereof. The reduction can be carried out using a reducing agent such as Na₂S₂O₄ or Fe and NH₄Cl, in a solvent such as ethanol and water, typically at a temperature of about 80-110° C. for about 0.1-2 hours.

In another embodiment of the process of the present invention, a compound of formula (XII)

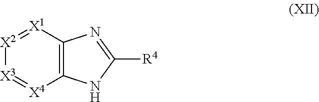

or a salt thereof, is reacted with a compound of formula (XIII), Z—R²—R³ (XIII), or a salt thereof, wherein R², R³, R⁴, X¹, X², X³ and X⁴ are as defined in the first aspect of the present invention, and Z is a leaving group. This process is depicted schematically in scheme 2.

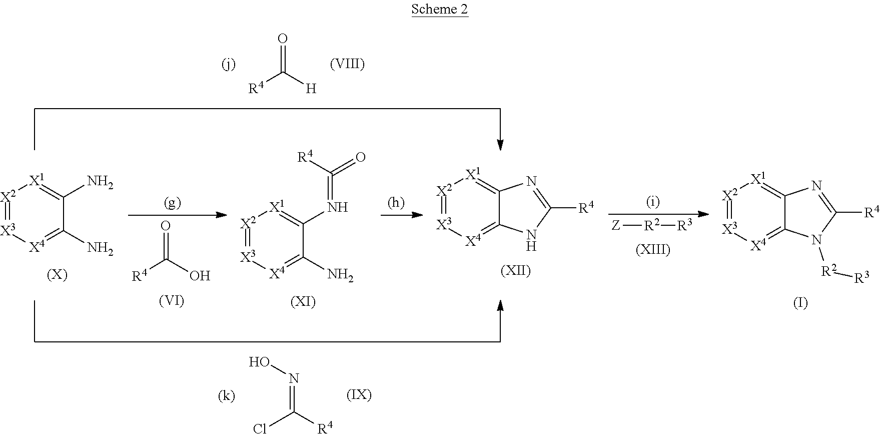

In step (i), a compound of formula (XII) or a salt thereof, is reacted with a compound of formula (XIII) or a salt thereof, to provide a compound of formula (I) or a salt thereof, wherein Z is a leaving group such as fluoro, chloro, bromo, iodo, tosylate, mesylate, or triflate. The reaction is typically carried out in the presence of a base such as K₂CO₃ or Cs₂CO₃, optionally in the presence of KI. The reaction is typically carried out in a solvent such as DMF or DMSO, typically at a temperature of about 20-120° C. for about 1-16 hours.

A compound of formula (XII) or a salt thereof, can be prepared in a two-step process as shown in steps (g) and (h). In step (g), a compound of formula (X) or a salt thereof, is reacted with a compound of formula (VI) or a salt thereof, to provide a compound of formula (XI) or a salt thereof. The reaction is typically carried out in the presence of a coupling agent such as T3P, HATU or DCC, optionally in the presence of HOBt, typically in the presence of a base, such as TEA or DIPEA, typically in a solvent such as DMF or DCM. The reaction is typically carried out at a temperature of about 5-25° C. for about 1-12 hours.

In step (h), a compound of formula (XI) or a salt thereof, is cyclised to provide a compound of formula (XII) or a salt thereof, typically by heating in the presence of an acid, such as AcOH, typically at a temperature of about 90-115° C. for about 0.5-10 hours.

The conversion achieved by steps (g) and (h), can alternatively be achieved by reacting a compound of formula (X) or a salt thereof, with a compound of formula (VIII) or a salt thereof, as shown in step (j). The reaction is typically carried out in the presence of Na₂S₂O₅, typically in a solvent such as EtOH, typically at a temperature of about 50-75° C. for about 10-16 hours.

Alternatively still, the conversion achieved by steps (g) and (h), can be achieved by reacting a compound of formula (X) or a salt thereof, with a compound of formula (IX) or a salt thereof, as shown in step (k), typically by heating in a solvent, such as EtOH, typically at a temperature of about 80-90° C. for about 1-24 hours.

In the reactions of the steps (a) to (k) depicted in schemes 1 and 2, R², R³, R⁴, X¹, X², X³ and X⁴ are as defined in the first aspect of the present invention, and Z is a leaving group.

Where a salt is used in any of the steps (a) to (k), this is typically a hydrochloride or hydrobromide salt.

It will be appreciated by those skilled in the art that in the processes of the present invention certain functional groups such as phenol, hydroxy or amino groups in the reagents may need to be protected by protecting groups. Thus, the preparation of the compounds, salts, N-oxides, solvates and prodrugs of the present invention may involve, at an appropriate stage, the introduction and/or removal of one or more protecting groups.

The protection and deprotection of functional groups are described, for example, in ‘Protective Groups in Organic Chemistry’, edited by J. W. F. McOmie, Plenum Press (1973); ‘Greene's Protective Groups in Organic Synthesis’, 4th edition, T. W. Greene and P. G. M. Wuts, Wiley-Interscience (2007); and ‘Protecting Groups’, 3rd edition, P. J. Kocienski, Thieme (2005).

The compounds of formula (I) may be converted into a pharmaceutically acceptable salt thereof, preferably an acid addition salt such as a formate, hemi-formate, hydrochloride, hydrobromide, benzenesulfonate (besylate), saccharin (e.g. monosaccharin), trifluoroacetate, sulfate, nitrate, phosphate, acetate, fumarate, semi-fumarate, maleate, tartrate, lactate, citrate, pyruvate, succinate, valerate, propanoate, butanoate, malonate, oxalate, 1-hydroxy-2-naphthoate (xinafoate), methanesulfonate or p-toluenesulfonate salt. In one embodiment of the invention, the compounds of formula (I) are in the form of a hydrochloride, formate, hemi-formate, or fumarate salt.

A salt of a compound of formula (I) may also be formed between a protic acid functionality of a compound of formula (I) and a suitable cation. Suitable cations include, but are not limited to lithium, sodium, potassium, magnesium, calcium and ammonium. In one embodiment of the invention, the salt is a mono- or di-sodium salt or a mono- or di-potassium salt.

Compounds of formula (I) and their salts and N-oxides may be in the form of hydrates or solvates which form another embodiment of the present invention. Such solvates may be formed with common organic solvents, including but not limited to, alcoholic solvents e.g. methanol, ethanol or isopropanol.

In one embodiment of the present invention, therapeutically inactive prodrugs are provided. Prodrugs are compounds which, when administered to a subject such as a human, are converted in whole or in part to a compound of formula (I). Generally, the prodrugs are pharmacologically inert chemical derivatives that can be converted in vivo to the active drug molecules to exert a therapeutic effect. Any of the compounds of formula (I) can be administered as a prodrug to increase the activity, bioavailability, or stability of the compound of formula (I) or to otherwise alter the properties of the compound of formula (I). Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts, N-oxides and solvates of such prodrugs as described above.

Where the compounds, salts, N-oxides, solvates and prodrugs of the present invention are capable of existing in stereoisomeric forms, it will be understood that the invention encompasses the use of all geometric and optical isomers (including atropisomers) and mixtures thereof. The use of tautomers and mixtures thereof also forms an embodiment of the present invention. The compounds, salts, N-oxides, solvates and prodrugs of the present invention may contain at least one chiral centre. The compounds, salts, N-oxides, solvates and prodrugs may therefore exist in at least two isomeric forms. The present invention encompasses racemic mixtures of the compounds, salts, N-oxides, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight. Enantiomerically pure isomers are particularly desired.

The compounds, salts, N-oxides, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I. Therefore, the term “hydrogen”, for example, encompasses ¹H, ²H (D) and ³H (T). Similarly, carbon atoms are to be understood to include ¹¹C, ¹²C, ¹³C and ¹⁴C, nitrogen atoms are to be understood to include ¹³N, ¹⁴N and ¹⁵N, oxygen atoms are to be understood to include ¹⁵O, ¹⁶O, ¹⁷O and ¹⁸O, fluorine atoms are to be understood to include ¹⁸F and ¹⁹F, and iodine atoms are to be understood to include ¹²³I, ¹²⁴I, ¹²⁵I, ¹²⁷I and ¹³¹I.

In one embodiment, the compounds, salts, N-oxides, solvates and prodrugs of the present invention may be isotopically labelled. As used herein, an “isotopically labelled” compound is one in which the abundance of a particular nuclide at a particular atomic position within the molecule is increased above the level at which it occurs in nature. Any of the compounds, salts, N-oxides, solvates and prodrugs of the present invention can be isotopically labelled, for example, any of examples 1 to 188.

In one embodiment, the compounds, salts, N-oxides, solvates and prodrugs of the present invention may bear one or more radiolabels. Such radiolabels may be introduced by using radiolabel-containing reagents in the synthesis of the compounds, salts, N-oxides, solvates or prodrugs, or may be introduced by coupling the compounds, salts, N-oxides, solvates or prodrugs to chelating moieties capable of binding to a radioactive metal atom. Such radiolabelled versions of compounds, salts, N-oxides, solvates and prodrugs may be used, for example, in diagnostic imaging studies.

In one embodiment, the compounds, salts, N-oxides, solvates and prodrugs of the present invention may be tritiated, i.e. they contain one or more 3H (T) atoms. Any of the compounds, salts, N-oxides, solvates and prodrugs of the present invention can be tritiated, for example, any of examples 1 to 188.

The compounds, salts, N-oxides, solvates and prodrugs of the present invention may be amorphous or in a polymorphic form or a mixture of any of these, each of which is an embodiment of the present invention.

The compounds, salts, N-oxides, solvates and prodrugs of the present invention have activity as pharmaceuticals and may be used in treating or preventing a disease, disorder or condition associated with KCNK13 activity.

Therefore, a fourth aspect of the present invention provides a compound of formula (I) or a pharmaceutically acceptable salt, N-oxide, solvate or prodrug thereof, according to the first aspect of the present invention, for use in therapy, in particular for use in treating or preventing a neurodegenerative disease, a psychiatric disease, a genetic disease, hearing loss, an ocular or retinal disease, a cardiovascular disease, an inflammatory disease, an autoimmune disease, or a metabolic disease.

The fourth aspect of the present invention also provides a compound of formula (I) or a pharmaceutically acceptable salt, N-oxide, solvate or prodrug thereof, according to the first aspect of the present invention, for use in treating or preventing Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes.

A fifth aspect of the present invention provides a use of a compound of formula (I) or a pharmaceutically acceptable salt, N-oxide, solvate or prodrug thereof, according to the first aspect of the present invention, for the manufacture of a medicament for treating or preventing a neurodegenerative disease, a psychiatric disease, a genetic disease, hearing loss, an ocular or retinal disease, a cardiovascular disease, an inflammatory disease, an autoimmune disease, or a metabolic disease.

The fifth aspect of the present invention also provides a use of a compound of formula (I) or a pharmaceutically acceptable salt, N-oxide, solvate or prodrug thereof, according to the first aspect of the present invention, for the manufacture of a medicament for treating or preventing Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes.

A sixth aspect of the present invention provides a method of treating or preventing a neurodegenerative disease, a psychiatric disease, a genetic disease, hearing loss, an ocular or retinal disease, a cardiovascular disease, an inflammatory disease, an autoimmune disease, or a metabolic disease; the method comprising administering a therapeutically or prophylactically effective amount of a compound of formula (I) or a pharmaceutically acceptable salt, N-oxide, solvate or prodrug thereof, according to the first aspect of the present invention, to a patient in need thereof.

The sixth aspect of the present invention also provides a method of treating or preventing Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes; the method comprising administering a therapeutically or prophylactically effective amount of a compound of formula (I) or a pharmaceutically acceptable salt, N-oxide, solvate or prodrug thereof, according to the first aspect of the present invention, to a patient in need thereof.

Unless stated otherwise, in any of the fourth, fifth or sixth aspects of the invention, the subject or patient may be any human or other animal. Typically, the subject or patient is a mammal, more typically a human or a domesticated mammal such as a cow, pig, lamb, sheep, goat, horse, cat, dog, rabbit, mouse etc. Most typically, the subject is a human.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

The terms “treat”, “treatment” and “treating” include improvement of the conditions described herein. The terms “treat”, “treatment” and “treating” include all processes providing slowing, interrupting, arresting, controlling, or stopping of the state or progression of the conditions described herein, but does not necessarily indicate a total elimination of all symptoms or a cure of the condition. The terms “treat”, “treatment” and “treating” are intended to include therapeutic as well as prophylactic treatment of such conditions.

For the above-mentioned therapeutic uses the dosage administered will, of course, vary with the compound employed, the mode of administration, the treatment desired and the disorder indicated. For example, the daily dosage of a compound of the invention (that is, a compound of formula (I) or a pharmaceutically acceptable salt, N-oxide, solvate or prodrug thereof), if inhaled, may be in the range from 0.05 micrograms per kilogram body weight (μg/kg) to 1 milligram per kilogram body weight (mg/kg). Alternatively, if the compound is administered orally or parenterally, then the daily dosage of the compound of the invention may be in the range from 0.01 micrograms per kilogram body weight (μg/kg) to 500 milligrams per kilogram body weight (mg/kg). The desired dosage may be presented at an appropriate interval such as once every other day, once a day, twice a day, three times a day or four times a day.

The compounds of formula (I) and pharmaceutically acceptable salts, N-oxides, solvates and prodrugs thereof may be used on their own, but will generally be administered in the form of a pharmaceutical composition in which the active ingredient is in association with a pharmaceutically acceptable adjuvant, diluent or carrier.

Therefore, a seventh aspect of the present invention provides a pharmaceutical composition comprising a compound of formula (I) or a pharmaceutically acceptable salt, N-oxide, solvate or prodrug thereof, according to the first aspect of the present invention, in association with a pharmaceutically acceptable adjuvant, diluent or carrier, and optionally one or more other therapeutic agents.

The invention still further provides a process for the preparation of a pharmaceutical composition of the invention which comprises mixing a compound of formula (I) or a pharmaceutically acceptable salt, N-oxide, solvate or prodrug thereof, according to the first aspect of the present invention, with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, dipotassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally, ocularly, topically or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intradermal, intravenous, intramuscular, intra-articular, intrasynovial, intrasternal, intrathecal, intralesional, intracranial, intratracheal, intraperitoneal, intraarticular, and epidural injection or infusion techniques. The term topical as used herein includes transdermal, mucosal, sublingual and topical ocular administration.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to, capsules, tablets, caplets, troches, lozenges, powders, granules, and aqueous suspensions, solutions and dispersions. These dosage forms are prepared according to techniques well-known in the art of pharmaceutical formulation. In the case of tablets for oral use, carriers which are commonly used include lactose, sodium and calcium carbonate, sodium and calcium phosphate, and corn starch. Lubricating agents, such as magnesium stearate, stearic acid or tale, are also typically added. If desired, the tablets may be coated with a material, such as glyceryl monostearate or glyceryl distearate, to delay absorption in the gastrointestinal tract. Tablets may also be effervescent and/or dissolving tablets. For oral administration in capsule form, useful diluents include lactose and dried corn starch. When aqueous suspensions are administered orally, the active ingredient may be combined with emulsifying and suspending agents. If desired, certain sweetening and/or flavouring and/or colouring agents and/or preservatives may be added to any oral dosage form.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

For ocular administration, the compounds, salts, N-oxides, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels, and ocular inserts. Alternatively, the compounds, salts, N-oxides, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, N-oxides, solvates or prodrugs of the invention will generally be provided in the form of ointments, cataplasms (poultices), pastes, powders, dressings, creams, plasters or patches.

Depending on the mode of administration, the pharmaceutical composition will preferably comprise from 0.05 to 99% by weight, more preferably from 0.05 to 80% by weight, still more preferably from 0.10 to 70% by weight, and even more preferably from 0.10 to 50% by weight of active ingredient, all percentages by weight being based on total composition.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered with another therapeutic agent or agents for the treatment of one or more of the conditions previously indicated. The compound of the invention or the pharmaceutical composition or formulation comprising the compound of the invention may be administered simultaneously with, separately from or sequentially to the one or more other therapeutic agents. The compound of the invention and the one or more other therapeutic agents may be comprised in the same pharmaceutical composition or formulation, or in separate pharmaceutical compositions or formulations, i.e. in the form of a kit. The one or more other therapeutic agents may, for example, be an antibody designed to clear forms of tau, alpha synuclein, or fragments of amyloid.

Typically, the mode of administration selected is that most appropriate to the disorder, disease or condition to be treated or prevented. Where one or more further active agents are administered, the mode of administration may be the same as or different to the mode of administration of the compound or pharmaceutical composition of the invention.

Such combination products employ the compounds of this invention within the dosage range described herein and the other pharmaceutically active agent(s) within approved dosage ranges.

### Definitions

An “alkyl” group may be linear (i.e. straight-chained) or branched. Examples of alkyl groups include methyl, ethyl, n-propyl, iso-propyl, n-butyl, iso-butyl, sec-butyl, tert-butyl, n-pentyl, 2-methyl-1-butyl, 3-methyl-1-butyl, 3-methyl-2-butyl, and 2,2-dimethyl-1-propyl groups. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” group is an unsaturated alkyl group having one or more carbon-carbon double bonds. Examples of alkenyl groups include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” group is an unsaturated alkyl group having one or more carbon-carbon triple bonds. Examples of alkynyl groups include ethynyl, propargyl, but-1-ynyl and but-2-ynyl groups. Typically an alkynyl group is a C₂-C₁₂ alkynyl group. More typically an alkynyl group is a C₂-C₆ alkynyl group. An “alkynylene” group is similarly defined as a divalent alkynyl group.

A “cycloalkyl” group is a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl group may be monocyclic, bicyclic (e.g. bridged, fused or spiro), or polycyclic.

A “cycloalkenyl” group is a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl group may be monocyclic, bicyclic (e.g. bridged, fused or spiro), or polycyclic.

An “aryl” group is an aromatic hydrocarbyl ring. The term “aryl” includes monocyclic aromatic hydrocarbons (such as phenyl) and polycyclic fused-ring aromatic hydrocarbons (such as naphthyl, anthracenyl and phenanthrenyl). Unless stated otherwise, the term “aryl” does not include “heteroaryl”.

A “heterocyclic” group is a non-aromatic cyclic group which includes one or more carbon atoms and one or more (such as one, two, three or four) heteroatoms, e.g. N, O or S, in the ring structure. A heterocyclic group may be monocyclic, bicyclic (e.g. bridged, fused or spiro), or polycyclic. Typically, a heterocyclic group is a 4- to 14-membered heterocyclic group, which means it contains from 4 to 14 ring atoms. Heterocyclic groups include unsaturated heterocyclic groups (such as azetinyl, tetrahydropyridinyl, and 2-oxo-1H-pyridinyl) and saturated heterocyclic groups. Examples of saturated monocyclic heterocyclic groups are azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups. Examples of saturated bicyclic heterocyclic groups are quinuclidinyl, 8-azabicyclo[3.2.1]octanyl, 2-azaspiro[3.3]heptanyl, 6-azaspiro[2.5]octanyl and hexahydro-1H-pyrrolizinyl groups.

A “heteroaryl” group is an aromatic cyclic group which includes one or more carbon atoms and one or more (such as one, two, three or four) heteroatoms, e.g. N, O or S, in the ring structure. Typically, a heteroaryl group is a 5- to 14-membered heteroaryl group, which means it contains from 5 to 14 ring atoms. The term “heteroaryl” includes monocyclic aromatic heterocycles (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyridazinyl, pyrimidinyl, pyrazinyl, triazinyl and tetrazinyl) and polycyclic fused-ring aromatic heterocycles (such as indolyl, benzofuranyl, benzothiophenyl, benzoxazolyl, benzisoxazolyl, benzothiazolyl, benzoisothiazolyl, benzimidazole, 1H-imidazo[4,5-b]pyridine, 1H-imidazo[4,5-c]pyridine, 3H-imidazo[4,5-b]pyridine, 3H-imidazo[4,5-c]pyridine, quinolinyl, isoquinolinyl, quinoxalinyl, quinazolinyl, phthalazinyl and cinnolinyl). Examples of heteroaryl groups include the following:

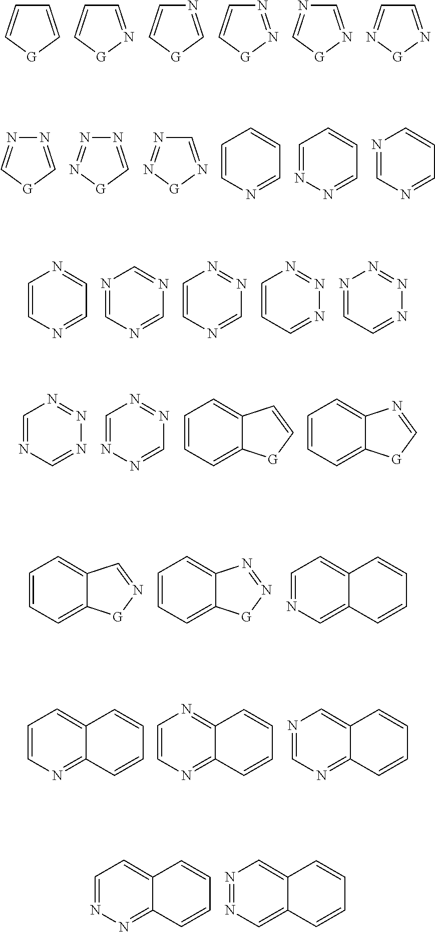

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

The term “halo” includes fluoro, chloro, bromo and iodo. In one embodiment, halo is fluoro.

Unless stated otherwise, where a group is prefixed by the term “halo”, such as a haloalkyl or halomethyl group, it is to be understood that the group in question is substituted with one or more (such as one, two, three, four or five) halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more (such as one, two, three, four or five) of the specific halo groups. For example, the term “fluoromethyl” refers to a methyl group substituted with one, two or three fluoro groups, and the term “fluoroethyl” refers to an ethyl group substituted with one, two, three, four or five fluoro groups.

Unless stated otherwise, any reference to an element is to be considered a reference to all isotopes of that element. Thus, for example, unless stated otherwise, any reference to hydrogen is considered to encompass all isotopes of hydrogen including ¹H, ²H (D) and 3H (T). Therefore, for the avoidance of doubt, it is noted that, for example, the terms “alkyl” and “methyl” include, for example, trideuteriomethyl.

Unless stated otherwise, any reference to a compound or group is to be considered a reference to all tautomers of that compound or group.

When any chemical group or moiety is described as substituted, it will be appreciated that the number and nature of substituents will be selected so as to avoid sterically undesirable combinations.

Further, it will be appreciated that the invention does not encompass any unstable ring or other structures or any O—O or S—S bonds.

### EXAMPLES

The present invention will now be further explained by reference to the following illustrative examples, in which the starting materials and reagents used are available from commercial suppliers or prepared via literature procedures or procedures similar to the ones described in this application. ‘Room temperature’, as used in the present specification, means a temperature in the range from about 18° C. to about 25° C.

Purity was assessed by HPLC.

For the purposes of the present invention, for all of the experimental details described below, where there are reaction conditions described, such as reagents, solvents and temperatures, above and/or below an arrow in a graphical representation, it is to be understood that these reaction conditions, in particular solvents and temperatures, are not essential to the reaction being carried out and may be varied.

### Abbreviations

AcOH acetic acid
DavePhos 2-dicyclohexylphosphino-2′-(N,N-dimethylamino)biphenyl
DIPEA N,N-diisopropylethylamine
EtOAc ethyl acetate
FA formic acid
IPA isopropyl alcohol
MeOH methanol
min minutes
NBS N-bromosuccinimide
NMP N-methyl-2-pyrrolidone
Pd(dba)₂ bis(dibenzylideneacetone)palladium(o)
Pd₂(dba)₃ Tris(dibenzylideneacetone)dipalladium(o)
quant. quantitative
RT room temperature
T₃P propylphosphonic anhydride
TEA triethylamine
Xantphos 4,5-bis(diphenylphosphino)-9,9-dimethylxanthene

### 1. Synthetic Examples

### Example 1: 5-[[6,7-difluoro-2-(4-methyl-1,2,5-oxadiazol-3-yl)benzimidazol-1-yl]methyl]pyrimidine-2-carbonitrile

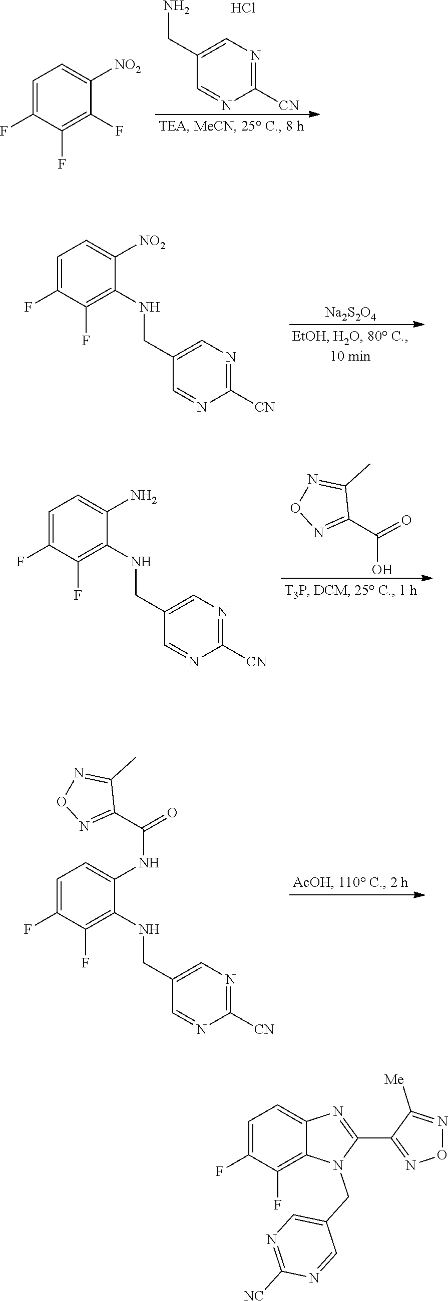

Step 1: A mixture of 1,2,3-trifluoro-4-nitro-benzene (210 mg, 1.19 mmol), 5-(aminomethyl)pyrimidine-2-carbonitrile hydrochloride (prepared as described for CN111393415) (206.36 mg, 1.21 mmol) and TEA (240.00 mg, 2.37 mmol) in MeCN (1.5 mL) was stirred at 25° C. for 8 hours. The mixture was concentrated to dryness and the residue was purified by flash chromatography to give 5-[(2,3-difluoro-6-nitro-anilino)methyl]pyrimidine-2-carbonitrile (170 mg, 0.584 mmol, 49.2% yield) as yellow oil.

MS ES⁺: 292.1

Step 2: A solution of Na₂S₂O₄ (508.19 mg, 2.92 mmol) in H₂O (2 mL) was added into the mixture of 5-[(2,3-difluoro-6-nitro-anilino)methyl]pyrimidine-2-carbonitrile (170 mg, 0.584 mmol) in EtOH (2 mL). The mixture was stirred at 80° C. for 10 min. The mixture was concentrated to remove most of the EtOH. Then the mixture was extracted with ethyl acetate (8 mL×3). The combined organic layers were dried with Na₂SO₄ and filtered. The filtrate was concentrated to afford 5-[(6-amino-2,3-difluoro-anilino)methyl]pyrimidine-2-carbonitrile (80 mg, 0.306 mmol, 52.5% yield) as yellow solid which was used for the next step without further purification.

MS ES⁺: 262.1

Step 3: To a mixture of 5-[(6-amino-2,3-difluoro-anilino)methyl]pyrimidine-2-carbonitrile (60 mg, 0.230 mmol) and 4-methyl-1,2,5-oxadiazole-3-carboxylic acid (Intermediate 2) (29.42 mg, 0.230 mmol) in DCM (1 mL) was added TEA (69.72 mg, 0.689 mmol) in one portion at ° C. Then TP (292.32 mg, 0.459 mmol, 50% purity in ethyl acetate) was added dropwise into the mixture at ° C. The mixture was stirred at 25° C. for 1 hour and then extracted with ethyl acetate (5 mL×3). The combined organic layers were dried with Na₂SO₄ and filtered. The filtrate was concentrated to afford N-[2-[(2-cyanopyrimidin-5-yl)methylamino]-3,4-difluoro-phenyl]-4-methyl-1,2,5-oxadiazole-3-carboxamide (80 mg, crude) as yellow solid which was used for the next step without further purification.

MS ES⁺: 372.1

Step 4: A mixture of N-[2-[(2-cyanopyrimidin-5-yl)methylamino]-3,4-difluoro-phenyl]-4-methyl-1,2,5-oxadiazole-3-carboxamide (80 mg, crude) in AcOH (2 mL) was stirred at 110° C. for 2 hours. The mixture was cooled down to RT and quenched by sat. NaHCO₃ (aq.) (8 mL). The mixture was extracted with ethyl acetate (5 mL×3). The combined organic layers were evaporated to dryness to obtained crude product which was purified by prep. HPLC (column: Phenomenex Gemini-NX C₁₈ 75*30 mm*3 μm, Mobile Phase A: water (0.05% NH₃·H₂O+10 mM NH₄HCO₃), Mobile Phase B: MeCN, Flow rate: 25 mL/min, gradient condition from 35% B to 75%). The pure fractions were collected and the volatiles were removed under vacuum. The residue was partitioned between acetonitrile (2 mL) and water (10 mL). The solution was lyophilized to dryness to give 5-[[6,7-difluoro-2-(4-methyl-1,2,5-oxadiazol-3-yl)benzimidazol-1-yl]methyl]pyrimidine-2-carbonitrile (12.6 mg, 0.036 mmol, 16.5% yield, 99.8% purity) as a white powder.

MS ES⁺: 354.3

¹H NMR (400 MHz, DMSO-d₆) 9.06 (s, 2H), 7.83 (d, J=8.6 Hz, 1H), 7.53 (d, J=11.4 Hz, 1H), 6.15 (s, 2H), 2.84 (s, 3H).

### Example 2: 5-[[2-(4-amino-1,2,5-oxadiazol-3-yl)-6,7-difluoro-benzimidazol-1-yl]methyl]pyrimidine-2-carbonitrile

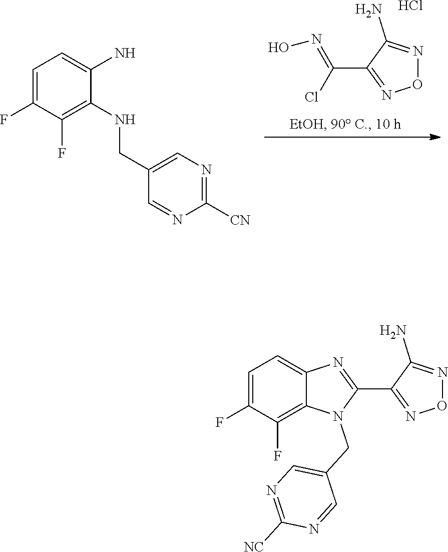 [SMILES: Cl ; N#Cc1ncc(CNc2c(N)ccc(F)c2F)cn1 ; Nc1nonc1/C(Cl)=N/O ; N#Cc1ncc(Cn2c(-c3nonc3N)nc3ccc(F)c(F)c32)cn1]

5-[(6-amino-2,3-difluoro-anilino)methyl]pyrimidine-2-carbonitrile (50 mg, 191.40 μmol, 1 eq) and (3Z)-4-amino-N-hydroxy-1,2,5-oxadiazole-3-carboximidoyl chloride hydrochloride (38.09 mg, 0.191 mmol) were added into EtOH (2 mL) in one portion at 25° C. The mixture was stirred at 90° C. for 10 hours and then concentrated to afford crude product which was purified by prep. HPLC (Column: Phenomenex Luna C₁₈ 75*30 mm*3 μm; Mobile phase A: water (0.225% FA), Mobile phase B: MeCN, Flow rate: 25 mL/min, gradient condition from 27% B to 65%). The pure fractions were collected and the volatiles were removed under vacuum. The residue was partitioned between acetonitrile (2 mL) and water (10 mL). The solution was lyophilized to dryness to give 5-[[2-(4-amino-1,2,5-oxadiazol-3-yl)-6,7-difluoro-benzimidazol-1-yl]methyl]pyrimidine-2-carbonitrile (2.59 mg, 0.007 mmol, 3.8% yield, 99.0% purity) as an off-white powder.

MS ES⁺: 355.3

¹H NMR (400 MHz, DMSO-d₆) 8.99 (s, 2H), 7.74 (dd, J=3.6, 8.8 Hz, 1H), 7.46 (ddd, J=7.6, 8.8, 11.4 Hz, 1H), 6.93 (s, 2H), 6.12 (s, 2H).

### Example 3: 4-[7-fluoro-1-(pyrimidin-5-ylmethyl)benzimidazol-2-yl]-1,2,5-oxadiazol-3-amine

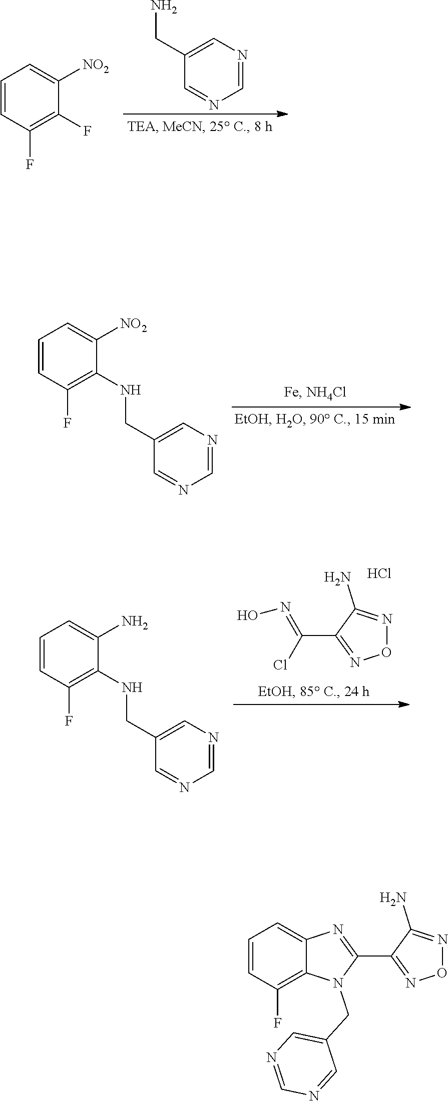

Step 1: A mixture of 1,2-difluoro-3-nitro-benzene (200 mg, 1.26 mmol), pyrimidin-5-ylmethanamine (137.19 mg, 1.26 mmol) and TEA (254.42 mg, 2.51 mmol) in MeCN (3 mL) was stirred at 25° C. for 8 hours. The reaction mixture was cooled down to RT and poured into H₂O (5 mL). Then the mixture was extracted with ethyl acetate (5 mL×3).

The combined organic phases were washed with brine (5 mL), dried over anhydrous Na₂SO₄ and filtered. The filtrate was concentrated in vacuum to give the crude product which was purified by silica gel column chromatography (petroleum ether: ethyl acetate=1:1) to afford 2-fluoro-6-nitro-N-(pyrimidin-5-ylmethyl)aniline (200 mg, 0.804 mmol, 64.0% yield, 99.8% purity) as a yellow solid.

MS ES⁺: 249.2

¹H NMR (400 MHz, DMSO-d₆) 9.07 (s, 1H), 8.77 (s, 2H), 8.15 (t, J=5.69 Hz, 1H), 7.89 (dt, J=8.66, 1.42 Hz, 1H), 7.43 (ddd, J=14.26, 7.94, 1.19 Hz, 1H), 6.75 (td, J=8.29, 4.82 Hz, 1H), 4.72 (dd, J=6.63, 4-38 Hz, 2H).

Step 2: A mixture of 2-fluoro-6-nitro-N-(pyrimidin-5-ylmethyl)aniline (200 mg, 0.804 mmol), NH₄Cl (215.51 mg, 4.03 mmol) and Fe (224.99 mg, 4.03 mmol) in EtOH (2 mL) and H₂O (2 mL) was stirred at 90° C. for 15 min. The reaction mixture cooled down to RT and poured into H₂O (5 mL). The mixture was extracted with ethyl acetate (5 mL×3). The combined organic phases were washed with brine (5 mL), dried over anhydrous Na₂SO₄ and filtered. The filtrate was concentrated in vacuum to give 3-fluoro-N²-(pyrimidin-5-ylmethyl)benzene-1,2-diamine (100 mg, 0.458 mmol, 56.9% yield) as a yellow liquid which was used for the next step without purification.

MS ES⁺: 219.2

Step 3: A mixture of 3-fluoro-N2-(pyrimidin-5-ylmethyl)benzene-1,2-diamine (100 mg, 0.458 mmol) and (3Z)-4-amino-N-hydroxy-1,2,5-oxadiazole-3-carboximidoyl chloride hydrochloride (91.19 mg, 0.458 mmol) in EtOH (2 mL) was stirred at 85° C. for 24 hours. The resulting mixture was cooled to RT. Then the mixture was dissolved in DMF (3 mL) and filtered to remove the insoluble. The filter liquor was concentrated in vacuo. The residue was further purified by prep. HPLC (Column: Phenomenex Luna C₁₈ 75*30 mm*3 μm, Mobile Phase A: water (0.05% NH₃·H₂O), Mobile Phase B: acetonitrile, Flow rate: 35 mL/min, gradient condition from 15% B to 55%). The pure fractions were collected, and the volatiles were removed under vacuum. The residue was partitioned between acetonitrile (2 mL) and water (10 mL). The mixture was lyophilized to dryness to give 4-[7-fluoro-1-(pyrimidin-5-ylmethyl)benzimidazol-2-yl]-1,2,5-oxadiazol-3-amine (1.13 mg, 0.004 mmol, 0.8% yield, 97.8% purity) as a white powder.

MS ES⁺: 312.2

¹H NMR (400 MHz, DMSO-d₆) 9.13 (s, 1H), 8.70 (s, 2H), 7.73 (d, J=8.00 Hz, 1H), 7.36 (td, J=70.97, 4.94 Hz, 1H), 7.26 (dd, J=11.63, 8.13 Hz, 1H), 6.96 (s, 2H), 6.04 (s, 2 H).

### Example 4: 4-[6-fluoro-1-(pyrimidin-5-ylmethyl)benzimidazol-2-yl]-1,2,5-oxadiazol-3-amine

Example 5: 4-[5-fluoro-1-(pyrimidin-5-ylmethyl)benzimidazol-2-yl]-1,2,5-oxadiazol-3-amine

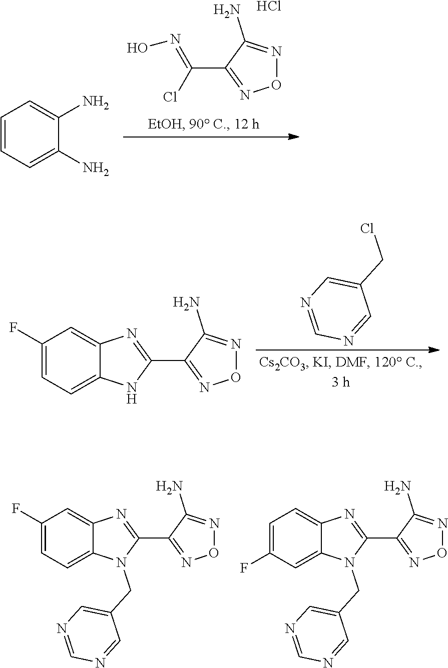

Step 1: A mixture of 4-fluorobenzene-1,2-diamine (5 g, 39.64 mmol) and (3Z)-4-amino-N-hydroxy-1,2,5-oxadiazole-3-carboximidoyl chloride hydrochloride (7.89 g, 39.64 mmol) in EtOH (100 mL) was stirred at 90° C. for 12 hours. The mixture was cooled down to RT with off-white precipitation formed. The precipitation was collected to give 4-(5-fluoro-1H-benzimidazol-2-yl)-1,2,5-oxadiazol-3-amine (7-5 g, 32.51 mmol, 82.0% yield, 95% purity) as an off-white solid which was used for the next step directly.

¹H NMR (400 MHz, DMSO-d₆) 13.82 (br s, 1H), 8.03-7.72 (m, 0.5H), 7.60 (br d, J=8.8 Hz, 1H), 7.37-7.33 (m, 0.5H), 7.29-7.02 (m, 1H), 6.93-6.67 (m, 2H).

Step 2: A solution of 4-(5-fluoro-1H-benzimidazol-2-yl)-1,2,5-oxadiazol-3-amine (200 mg, 0.913 mmol), 5-(chloromethyl)pyrimidine (211.16 mg, 1.64 mmol), Cs₂CO₃ (891.94 mg, 2.74 mmol) and KI (151.48 mg, 0.913 mmol) in DMF (3 mL) was stirred at 120° C. for 3 hours. The reaction mixture was cooled down to RT and filtered to remove the salt. The filtrate was purified by prep. HPLC (Column: Xtimate C₁₈ 100*30 mm*10 μm, Mobile Phase A: water (0.225% FA), Mobile Phase B: acetonitrile, Flow rate: 25 mL/min, gradient condition from 43% B to 63%). The pure fractions were collected and the volatiles were removed under vacuum. The residue was partitioned between acetonitrile (2 mL) and water (10 mL). The solution was lyophilized to dryness to give the title compounds as yellow solid. The title compounds were separated by SFC (separation condition: DAICEL CHIRALPAK AD (250 mm*30 mm, 10 μm); Mobile phase: A: Supercritical CO₂, B: 0.1% NH₃·H₂O EtOH, A:B=75:25 at 60 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fractions were collected and the volatiles were removed under vacuum. The residue was partitioned between acetonitrile (2 mL) and water (10 mL). The mixture was lyophilized to dryness to give Peak 1 as a white solid and Peak 2 as a white solid. Peak 2 was further purified by SFC (separation condition: DAICEL CHIRALCEL OD (250 mm*30 mm, 10 μm); Mobile phase: A: Supercritical CO₂, B: 0.1% NH₃·H₂O EtOH, A:B=75:25 at 60 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm). The pure fractions were collected and the volatiles were removed under vacuum. The residue was partitioned between acetonitrile (2 mL) and water (10 mL). The mixture was lyophilized to dryness to give Peak 2 as a white solid.

### Example 4 (Peak 1)

4-[6-Fluoro-1-(pyrimidin-5-ylmethyl)benzimidazol-2-yl]-1,2,5-oxadiazol-3-amine (53.67 mg, 0.171 mmol, 18.7% yield, 99.1% purity) was obtained as a white solid.

MS ES⁺: 312.2

¹H NMR (400 MHz, DMSO-d₆) 9.11 (s, 1H), 8.69 (s, 2H), 7.91 (dd, J=4.9, 8.9 Hz, 1H), 7.83 (dd, J=2.4, 9.3 Hz, 1H), 7.27 (dt, J=2.4, 9.3 Hz, 1H), 6.95 (s, 2H), 5.96 (s, 2H).

### Example 5 (Peak 2)

4-[5-Fluoro-1-(pyrimidin-5-ylmethyl)benzimidazol-2-yl]-1,2,5-oxadiazol-3-amine (32.48 mg, 0.104 mmol, 11.4% yield, 99.8% purity) was obtained as a white solid.

MS ES⁺: 312.2

¹H NMR (400 MHz, DMSO-d₆) 9.11 (s, 1H), 8.70 (s, 2H), 7.89 (dd, J=4.6, 9.0 Hz, 1H), 7.71 (dd, J=2.4, 9.4 Hz, 1H), 7.34 (dt, J=2.4, 9.3 Hz, 1H), 6.96 (s, 2H), 6.01 (s, 2H).

### Example 6: 4-[7-fluoro-1-(pyridazin-3-ylmethyl)benzimidazol-2-yl]-1,2,5-oxadiazol-3-amine

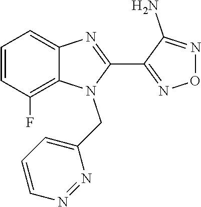 [SMILES: Nc1nonc1-c1nc2cccc(F)c2n1Cc1cccnn1]

Prepared as described for Example 3 using 1,2-difluoro-3-nitro-benzene (291.56 mg, 1.83 mmol) and pyridazin-3-ylmethanamine hydrochloride (200 mg, 1.83 mmol) to give 4-[7-fluoro-1-(pyridazin-3-ylmethyl)benzimidazol-2-yl]-1,2,5-oxadiazol-3-amine (6.54 mg, 0.020 mmol, 1.1% yield, 97.2% purity) as a grey solid.

MS ES⁺: 312.3

¹H NMR (400 MHz, DMSO-d₆) 9.16-9.11 (m, 1H), 7.79-7.69 (m, 3H), 7.38-7.32 (m, 1H), 7.26-7.19 (m, 1H), 7.03-6.96 (m, 2H), 6.30 (s, 2H).

### Example 7: 3-[6,7-difluoro-1-(pyrimidin-5-ylmethyl)benzimidazol-2-yl]-4-methyl-1,2,5-oxadiazole

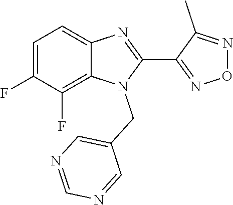 [SMILES: Cc1nonc1-c1nc2ccc(F)c(F)c2n1Cc1cncnc1]

Prepared as described for Example 1 using 1,2,3-trifluoro-4-nitro-benzene (300 mg, 1.69 mmol) and pyrimidin-5-ylmethanamine (184.88 mg, 1.69 mmol) to give 3-[6,7-difluoro-1-(pyrimidin-5-ylmethyl)benzimidazol-2-yl]-4-methyl-1,2,5-oxadiazole (2.13 mg, 0.006 mmol, 0.4% yield, 95.5% purity) as an off-white solid.

MS ES⁺: 329.2

¹H NMR (400 MHz, DMSO-d₆) 9.15 (s, 1H), 8.74 (s, 2H), 7.75 (d, J=9.2 Hz, 1H), 7.45 (d, J=11.2 Hz, 1H), 5.99 (s, 2H), 2.77 (s, 3H).

### Example 8: 5-[[2-(4-amino-1,2,5-oxadiazol-3-yl)-4-fluoro-benzimidazol-1-yl]methyl]pyridine-2-carbonitrile

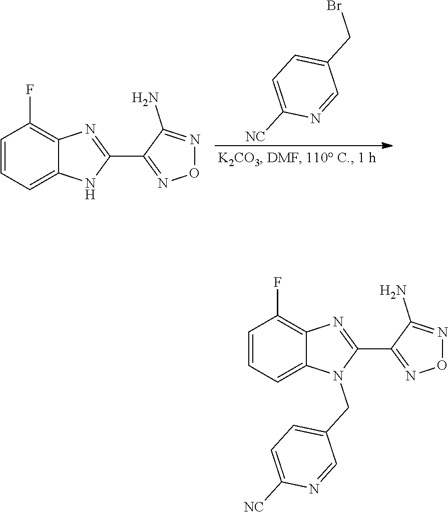 [SMILES: Nc1nonc1-c1nc2c(F)cccc2[nH]1 ; N#Cc1ccc(CBr)cn1 ; N#Cc1ccc(Cn2c(-c3nonc3N)nc3c(F)cccc32)cn1]

A mixture of 5-(bromomethyl)pyridine-2-carbonitrile (prepared as described for WO2007/28083) (62.93 mg, 0.319 mmol), 4-(4-fluoro-1H-benzimidazol-2-yl)-1,2,5-oxadiazol-3-amine (70 mg, 0.319 mmol) and K₂CO₃ (88.28 mg, 0.639 mmol) in DMF (1 mL) was stirred at 110° C. for 1 hour. The mixture was concentrated to afford the crude product which was purified by prep. HPLC (Column: Phenomenex Luna C₁₈ 75*30 mm*3 μm, Mobile Phase A: water (10 mM NH₄HCO₃)—acetonitrile, Mobile Phase B: acetonitrile, Flow rate: 25 mL/min, gradient condition from 30% B to 70%). The pure fractions were collected and the volatiles were removed under vacuum. The residue was partitioned between acetonitrile (20 mL) and water (100 mL). The solution was lyophilized to dryness to give 5-[[2-(4-amino-1,2,5-oxadiazol-3-yl)-4-fluoro-benzimidazol-1-yl]methyl]pyridine-2-carbonitrile (6.21 mg, 0.019 mmol, 5.8% yield, 100% purity) as a white powder.

MS ES⁺: 335.9

¹H NMR (400 MHz, DMSO-d₆) 8.74 (d, J=1.6 Hz, 1H), 7.95 (d, J=8.0 Hz, 1H), 7.71 (dd, J=2.4, 8.0 Hz, 1H), 7.62 (d, J=8.0 Hz, 1H), 7.46-7.40 (m, 1H), 7.25 (dd, J=8.0, 10.8 Hz, 1H), 6.94 (s, 2H), 6.10 (s, 2H).

### Example 9: 3-[4-fluoro-1-(pyrimidin-5-ylmethyl)benzimidazol-2-yl]-4-methyl-1,2,5-oxadiazole

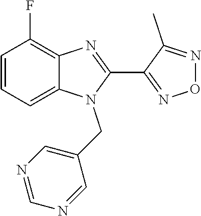 [SMILES: Cc1nonc1-c1nc2c(F)cccc2n1Cc1cncnc1]

Prepared as described for Example 1 used pyrimidin-5-ylmethanamine (200 mg, 1.83 mmol) and 1,3-difluoro-2-nitro-benzene (291.56 mg, 1.83 mmol) to give 3-[4-fluoro-1-(pyrimidin-5-ylmethyl)benzimidazol-2-yl]-4-methyl-1,2,5-oxadiazole (7.09 mg, 0.022 mmol, 3.6% yield, 97.1% purity) as an off-white solid.

MS ES⁺: 311.2

¹H NMR (400 MHz, DMSO-d₆) 9.12 (s, 1H), 8.72 (s, 2H), 7.65 (d, J=8.3 Hz, 1H), 7.43 (dt, J=4.9, 8.2 Hz, 1H), 7.23 (dd, J=7.9, 10.9 Hz, 1H), 5.98 (s, 2H), 2.79 (s, 3H).

### Example 10: 4-[7-fluoro-1-[(6-methoxypyridin-3-yl)methyl]benzimidazol-2-yl]-1,2,5-oxadiazol-3-amine

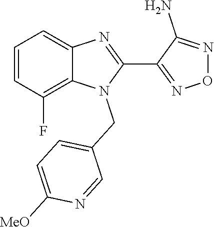 [SMILES: COc1ccc(Cn2c(-c3nonc3N)nc3cccc(F)c32)cn1]

Prepared as described for Example 3 using 1,2-difluoro-3-nitro-benzene (300 mg, 1.89 mmol) and (6-methoxypyridin-3-yl)methanamine (260.54 mg, 1.89 mmol) to give 4-[7-fluoro-1-[(6-methoxypyridin-3-yl)methyl]benzimidazol-2-yl]-1,2,5-oxadiazol-3-amine (18.12 mg, 0.053 mmol, 2.8% yield, 98.7% purity) as a white solid.

MS ES⁺: 341.3

¹H NMR (400 MHz, DMSO-d₆) 8.10-8.01 (m, 1H), 7.75-7.68 (m, 1H), 7.53-7.48 (m, 1H), 7.39-7.31 (m, 1H), 7.30-7.23 (m, 1H), 7.03-6.96 (m, 2H), 6.80-6.73 (m, 1H), 5.94 (s, 2H), 3.79 (s, 3H).

### Example 11: 5-[[2-(4-amino-1,2,5-oxadiazol-3-yl)imidazo[4,5-b]pyridin-3-yl]methyl]pyrimidine-2-carbonitrile

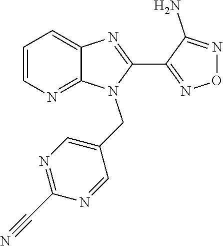 [SMILES: N#Cc1ncc(Cn2c(-c3nonc3N)nc3cccnc32)cn1]

Prepared as described for Example 3 using 2-fluoro-3-nitro-pyridine (100 mg, 0.704 mmol) and 5-(aminomethyl)pyrimidine-2-carbonitrile (145.73 mg, 0.704 mmol) to give 5-[[2-(4-amino-1,2,5-oxadiazol-3-yl)imidazo[4,5-b]pyridin-3-yl]methyl]pyrimidine-2-carbonitrile (2.92 mg, 0.009 mmol, 1.3% yield, 97.7% purity) as an off-white powder.

MS ES⁺: 320.1

¹H NMR (400 MHz, DMSO-d₆) 9.00 (s, 2H), 8.56 (dd, J=1.6, 4.8 Hz, 1H), 8.34 (dd, J=1.4, 8.0 Hz, 1H), 7.50 (dd, J=4.6, 8.0 Hz, 1H), 6.96 (s, 2H), 6.04 (s, 2H).

### Example 12: 4-[6-fluoro-3-(pyrimidin-5-ylmethyl)imidazo[4,5-b]pyridin-2-yl]-1,2,5-oxadiazol-3-amine

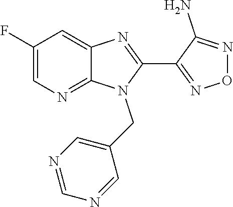 [SMILES: Nc1nonc1-c1nc2cc(F)cnc2n1Cc1cncnc1]

Prepared as described for Example 3 using 2,5-difluoro-3-nitro-pyridine (292.94 mg, 1.83 mmol) and pyrimidin-5-ylmethanamine (200 mg, 1.83 mmol) to give 4-[6-fluoro-3-(pyrimidin-5-ylmethyl)imidazo[4,5-b]pyridin-2-yl]-1,2,5-oxadiazol-3-amine (16.07 mg, 0.049 mmol, 2.7% yield, 95.6% purity) as an off-white solid.

MS ES⁺: 313.2

¹H NMR (400 MHz, DMSO-d₆) 9.11 (s, 1H), 8.79 (s, 2H), 8.62 (d, J=2.4 Hz, 1H), 8.31 (d, J=9.2 Hz, 1H), 6.94 (s, 2H), 5.94 (s, 2H).

### Example 13: 3-methyl-4-[3-(pyrimidin-5-ylmethyl)imidazo[4,5-b]pyridin-2-yl]-1,2,5-oxadiazole

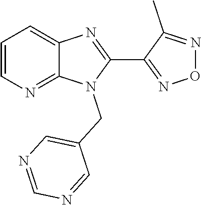 [SMILES: Cc1nonc1-c1nc2cccnc2n1Cc1cncnc1]

Prepared as described for Example 1 using 2-fluoro-3-nitro-pyridine (260.40 mg, 1.83 mmol) and pyrimidin-5-ylmethanamine (200 mg, 1.83 mmol) to give 3-methyl-4-[3-(pyrimidin-5-ylmethyl)imidazo[4,5-b]pyridin-2-yl]-1,2,5-oxadiazole (7.49 mg, 0.025 mmol, 1.4% yield, 98.5% purity) as a white solid.

MS ES⁺: 294.2

¹H NMR (400 MHz, DMSO-d₆) 9.11 (s, 1H), 8.81 (s, 2H), 8.57 (d, J=4.8 Hz, 1H), 8.37 (d, J=8.0 Hz, 1H), 7.49 (d, J=8.0 Hz, 1H), 5.93 (s, 2H), 2.78 (s, 3H).

### Example 14: 4-[3-[(6-methoxypyridin-3-yl)methyl]imidazo[4,5-b]pyridin-2-yl]-1,2,5-oxadiazol-3-amine

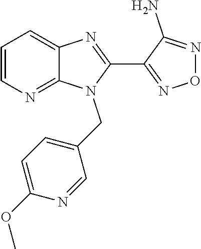 [SMILES: COc1ccc(Cn2c(-c3nonc3N)nc3cccnc32)cn1]

Prepared as described for Example 3 using 2-fluoro-3-nitro-pyridine (205.68 mg, 1.45 mmol) and (6-methoxypyridin-3-yl)methanamine (200 mg, 1.45 mmol) to give 4-[3-[(6-methoxypyridin-3-yl)methyl]imidazo[4,5-b]pyridin-2-yl]-1,2,5-oxadiazol-3-amine (7.26 mg, 0.022 mmol, 1.5% yield, 98.0% purity) as an off-white powder.

MS ES⁺: 324.3

¹H NMR (400 MHz, DMSO-d₆) 8.59 (dd, J=1.6, 4.8 Hz, 1H), 8.32 (dd, J=1.6, 8.0 Hz, 1H), 8.20 (d, J=2.4 Hz, 1H), 7.66-7.62 (m, 1H), 7.50 (dd, J=4.8, 8.0 Hz, 1H), 6.98 (s, 2H), 6.75 (d, J=8.8 Hz, 1H), 5.87 (s, 2H), 3.79 (s, 3H).

### Example 15: 4-[3-(pyrimidin-5-ylmethyl)imidazo[4,5-b]pyridin-2-yl]-1,2,5-oxadiazol-3-amine

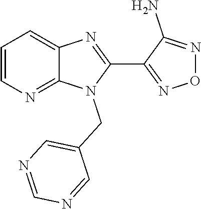 [SMILES: Nc1nonc1-c1nc2cccnc2n1Cc1cncnc1]

Prepared as described for Example 3 using 2-fluoro-3-nitro-pyridine (260.40 mg, 1.83 mmol) and pyrimidin-5-ylmethanamine (200 mg, 1.83 mmol) to give 4-[3-(pyrimidin-5-ylmethyl)imidazo[4,5-b]pyridin-2-yl]-1,2,5-oxadiazol-3-amine (1.3 mg, 0.004 mmol, 0.2% yield, 95.0% purity) as a yellow powder.

MS ES⁺: 295.1

¹H NMR (400 MHz, MeOH-d₄) 9.06 (s, 1H), 8.88 (s, 2H), 8.58 (d, J=4.8 Hz, 1H), 8.27 (d, J=8.0 Hz, 1H), 7.48 (d, J=8.0 Hz, 1H), 6.07 (s, 2H).

### Example 16: 3-[3-[(6-methoxypyridin-3-yl)methyl]imidazo[4,5-b]pyridin-2-yl]-4-methyl-1,2,5-oxadiazole

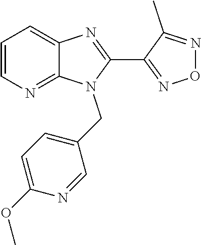 [SMILES: COc1ccc(Cn2c(-c3nonc3C)nc3cccnc32)cn1]

Prepared as described for Example 1 using 2-fluoro-3-nitro-pyridine (205.68 mg, 1.45 mmol) and (6-methoxypyridin-3-yl)methanamine (200 mg, 1.45 mmol) to give 3-[3-[(6-methoxypyridin-3-yl)methyl]imidazo[4,5-b]pyridin-2-yl]-4-methyl-1,2,5-oxadiazole (9.92 mg, 0.031 mmol, 13.9% yield, 99.6% purity) as a white powder.

MS ES⁺: 323.1

¹H NMR (400 MHz, DMSO-d₆) 8.58 (dd, J=1.6, 4.8 Hz, 1H), 8.34 (dd, J=1.6, 8.0 Hz, 1H), 8.20 (d, J=2.0 Hz, 1H), 7.66 (dd, J=2.4, 8.4 Hz, 1H), 7.48 (dd, J=4.8, 8.0 Hz, 1H), 6.75 (d, J=8.8 Hz, 1H), 5.83 (s, 2H), 3.78 (s, 3H), 2.77 (s, 3H).

### Example 17: 3-methyl-4-[3-[[6-(trifluoromethyl)pyridin-3-yl]methyl]imidazo[4,5-b]pyridin-2-yl]-1,2,5-oxadiazole

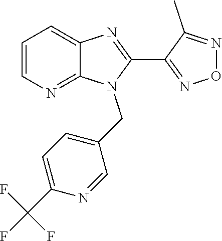 [SMILES: Cc1nonc1-c1nc2cccnc2n1Cc1ccc(C(F)(F)F)nc1]

Prepared as described for Example 1 using 2-fluoro-3-nitro-pyridine (500 mg, 3.52 mmol) and [6-(trifluoromethyl)pyridin-3-yl]methanamine (619.82 mg, 3.52 mmol) to give 3-methyl-4-[3-[[6-(trifluoromethyl)pyridin-3-yl]methyl]imidazo[4,5-b]pyridin-2-yl]-1,2,5-oxadiazole (61.74 mg, 0.167 mmol, 4.7% yield, 97.7% purity) as a black-brown solid.

MS ES⁺: 361.2

¹H NMR (400 MHz, DMSO-d₆) 8.84-8.78 (m, 1H), 8.61-8.54 (m, 1H), 8.43-8.36 (m, 1H), 7.93-7.87 (m, 1H), 7.87-7.82 (m, 1H), 7.54-7.48 (m, 1H), 6.05-6.00 (m, 2H), 2.81-2.78 (m, 3H).

### Example 18: 6-[[2-(4-methyl-1,2,5-oxadiazol-3-yl)imidazo[4,5-b]pyridin-3-yl]methyl]pyridazine-3-carbonitrile

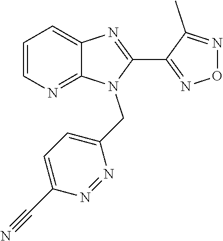 [SMILES: Cc1nonc1-c1nc2cccnc2n1Cc1ccc(C#N)nn1]

Prepared as described for Example 1 using 6-(aminomethyl)pyridazine-3-carbonitrile hydrochloride (80 mg, 0.386 mmol) and 2-fluoro-3-nitro-pyridine (54.90 mg, 0.386 mmol) to give 6-[[2-(4-methyl-1,2,5-oxadiazol-3-yl)imidazo[4,5-b]pyridin-3-yl]methyl]pyridazine-3-carbonitrile (1.30 mg, 0.004 mmol, 1.0% yield, 97.3% purity) as a grey solid.

MS ES⁺: 319.3

¹H NMR (400 MHz, DMSO-d₆) 8.53-8.49 (m, 1H), 8.42-8.37 (m, 1H), 8.37-8.32 (m, 1H), 8.09-8.05 (m, 1H), 7.52-7.46 (m, 1H), 6.31 (s, 2H), 2.80 (s, 3H).

### Example 19: 4-[3-[[6-(trifluoromethyl)pyridin-3-yl]methyl]imidazo[4,5-b]pyridin-2-yl]-1,2,5-oxadiazol-3-amine

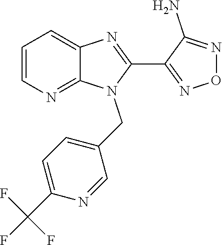 [SMILES: Nc1nonc1-c1nc2cccnc2n1Cc1ccc(C(F)(F)F)nc1]

Prepared as described for Example 3 using 2-fluoro-3-nitro-pyridine (300 mg, 2.11 mmol) and [6-(trifluoromethyl)pyridin-3-yl]methanamine (371.89 mg, 2.11 mmol) to give 4-[3-[[6-(trifluoromethyl)pyridin-3-yl]methyl]imidazo[4,5-b]pyridin-2-yl]-1,2,5-oxadiazol-3-amine (18.28 mg, 0.049 mmol, 4.4% yield, 96.5% purity) as a pink solid.

MS ES⁺: 362.3

¹H NMR (400 MHz, DMSO-d₆) 8.70 (s, 1H), 8.49 (dd, J=1.4, 4.7 Hz, 1H), 8.24 (dd, J=1.5, 8.1 Hz, 1H), 7.86-7.70 (m, 2H), 7.42 (dd, J=4.8, 8.1 Hz, 1H), 6.72 (s, 2H), 5.99 (s, 2H).

### Example 20: 3-methyl-4-[3-(pyridazin-3-ylmethyl)imidazo[4,5-b]pyridin-2-yl]-1,2,5-oxadiazole

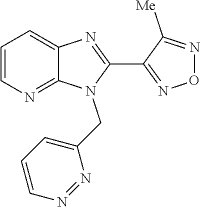 [SMILES: Cc1nonc1-c1nc2cccnc2n1Cc1cccnn1]

Prepared as described for Example 1 using 2-fluoro-3-nitro-pyridine (1.04 g, 7.33 mmol) and pyridazin-3-ylmethanamine (800 mg, 7.33 mmol) to give 3-methyl-4-[3-(pyridazin-3-ylmethyl)imidazo[4,5-b]pyridin-2-yl]-1,2,5-oxadiazole (4.59 mg, 0.016 mmol, 0.2% yield, 99.9% purity) as a brown solid.

MS ES⁺: 294.3

¹H NMR (400 MHz, DMSO-d₆) 9.12-9.09 (m, 1H), 8.52-8.50 (m, 1H), 8.40-8.37 (m, 1H), 7.74-7.66 (m, 2H), 7.50-7.46 (m, 1H), 6.20 (s, 2H), 2.80 (s, 3H).

### Example 21: 4-[1-(pyrimidin-5-ylmethyl)benzimidazol-2-yl]-1,2,5-oxadiazol-3-amine

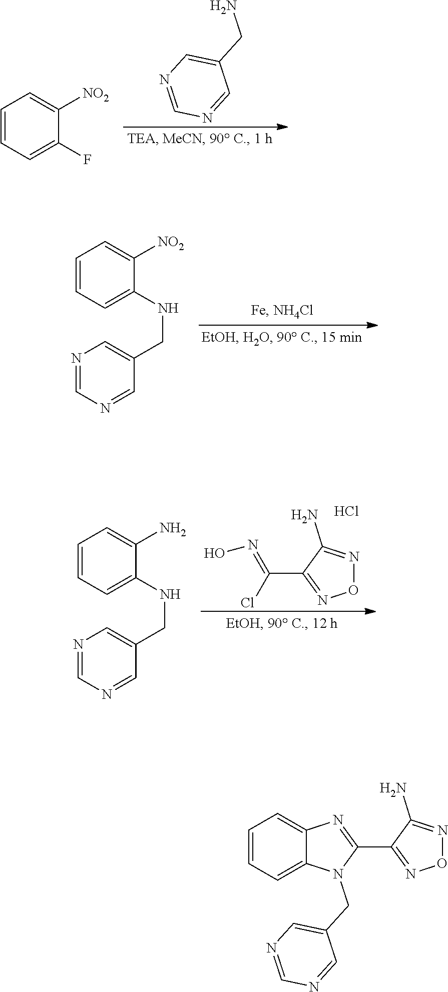

Step 1: A solution of 1-fluoro-2-nitro-benzene (300 mg, 2.13 mmol), pyrimidin-5-ylmethanamine (232.02 mg, 2.13 mmol) and TEA (1.08 g, 10.63 mmol) in MeCN (3 mL) was stirred at 90° C. for 1 hour. The reaction mixture was diluted with H₂O (50 mL) and extracted with ethyl acetate (30 mL×3). The combined organic layers were washed with brine (30 mL), dried over Na₂SO₄, filtered and concentrated under reduced pressure to give a residue which was purified by column chromatography (SiO₂, petroleum ether: ethyl acetate=1:0 to 1:1) to give 2-nitro-N-(pyrimidin-5-ylmethyl)aniline (280 mg, 1.18 mmol, 55.5% yield, 97% purity) as a yellow solid.

MS ES⁺: 231.1

¹H NMR (400 MHz, DMSO-d₆) 9.08 (s, 1H), 8.83 (s, 2H), 8.73-8.64 (m, 1H), 8.11-8.07 (m, 1H), 7.52-7.43 (m, 1H), 6.97 (d, J=8.1 Hz, 1H), 6.74-6.67 (m, 1H), 4.70 (d, J=6.4 Hz, 2H).

Step 2: A mixture of 2-nitro-N-(pyrimidin-5-ylmethyl)aniline (280 mg, 1.22 mmol), NH₄Cl (325.28 mg, 6.08 mmol) and Fe (339.60 mg, 6.08 mmol) in EtOH (3 mL) and H₂O (3 mL) was stirred at 90° C. for 15 min. The reaction mixture was diluted with H₂O (50 mL) and extracted with ethyl acetate (30 mL×3). The combined organic layers were washed with brine (30 mL), dried over Na₂SO₄, filtered and concentrated under reduced pressure to give N¹-(pyrimidin-5-ylmethyl)benzene-1,2-diamine (174 mg, 0.869 mmol, 71.5% yield) as a yellow solid which was used for the next step directly.

MS ES⁺: 201.2

Step 3: A solution of N¹-(pyrimidin-5-ylmethyl)benzene-1,2-diamine (174 mg, 0.869 mmol) and (3Z)-4-amino-N-hydroxy-1,2,5-oxadiazole-3-carboximidoyl chloride hydrochloride (172.92 mg, 0.869 mmol) in EtOH (1.5 mL) was stirred at 90° C. for 12 hours. The reaction mixture was filtered and the filtrate was concentrated under reduced pressure to give a residue. The residue was purified by prep. HPLC (column: Phenomenex Gemini-NX C₁₈ 75*30 mm*3 μm; mobile phase A: water (0.05% NH₃·H₂O+10 mM NH₄HCO₃), mobile phase B: MeCN; Flow rate: 25 mL/min, gradient condition from 15% B to 55%). The pure fractions were collected and the volatiles were removed under vacuum. The residue was partitioned between acetonitrile (2 mL) and water (10 mL). The solution was lyophilized to dryness to give a residue which was further purified by prep. HPLC (column: Phenomenex Luna C₁₈ 75*30 mm*3 μm; mobile phase A: water, mobile phase B: MeCN; Flow rate: 25 mL/min, gradient condition from 18% B to 48%). The pure fractions were collected and the volatiles were removed under vacuum. The residue was partitioned between acetonitrile (2 mL) and water (10 mL). The solution was lyophilized to dryness to give 4-[1-(pyrimidin-5-ylmethyl)benzimidazol-2-yl]-1,2,5-oxadiazol-3-amine (7.82 mg, 0.025 mmol, 2.9% yield, 95.5% purity) as a white solid.

MS ES⁺: 294.3

¹H NMR (400 MHz, DMSO-d₆) 9.10 (s, 1H), 8.69 (s, 2H), 7.92-7.81 (m, 2H), 7.48-7.37 (m, 2H), 6.99 (s, 2H), 6.01 (s, 2H).

### Example 22: 4-[4,7-difluoro-1-(pyridin-3-ylmethyl)benzimidazol-2-yl]-1,2,5-oxadiazol-3-amine

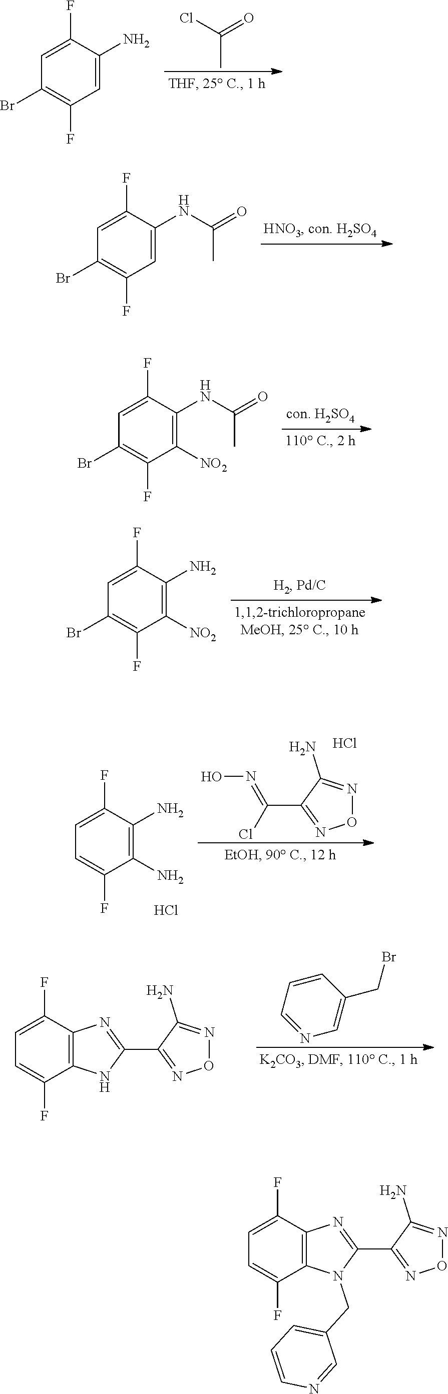

Step 1: To a mixture of 4-bromo-2,5-difluoro-aniline (10 g, 48.08 mmol) in THF (50 mL) was added acetyl chloride (3.77 g, 48.08 mmol) dropwise at 25° C. The mixture was stirred at 25° C. for 1 hour. Then the mixture was concentrated to dryness under reduced pressure to afford N-(4-bromo-2,5-difluorophenyl)acetamide (7 g, 28.00 mmol, 58.2% yield) as a grey solid which was used for the next step without further purification.

¹H NMR (400 MHz, DMSO-d₆) 10.02 (s, 1H), 8.08 (dd, J=6.8, 10.8 Hz, 1H), 7.75 (dd, J=6.5, 10.3 Hz, 1H), 2.11 (s, 3H).

Step 2: To a solution of N-(4-bromo-2,5-difluorophenyl)acetamide (2 g, 8.00 mmol) in concentrated H₂SO₄ (8 mL) was added dropwise HNO₃ (1.73 g, 18.67 mmol, 68% purity) at ° C. After addition was complete, the mixture was allowed to warm slowly to 25° C. and stirred for 3 hours. The mixture was poured into ice water (50 mL) slowly and stirred for 30 min, then the aqueous phase was extracted with ethyl acetate (100 mL×3). The combined organic phases were washed with brine (50 mL×3), dried with anhydrous Na₂SO₄, filtered and concentrated in vacuum to give N-(4-bromo-3,6-difluoro-2-nitrophenyl)acetamide (2.2 g, 7.46 mmol, 93.2% yield) as a yellow solid which was used for the next step without further purification.

¹H NMR (400 MHz, DMSO-d₆) 10.47 (s, 1H), 8.27 (dd, J=6.1, 9.4 Hz, 1H), 2.06 (s, 3H).

Step 3: N-(4-bromo-3,6-difluoro-2-nitrophenyl)acetamide (800 mg, 2.71 mmol) was added into conc. H₂SO₄ (5 mL) in portions. Then the mixture was stirred at 110° C. for 2 hours. The mixture was poured into ice water (10 mL) and adjusted pH=8 with NaOH (aq.) (50 mL, 2M in water). Then the mixture was extracted with ethyl acetate (50 mL×3). The combined organic phases were washed with brine (10 mL×3), dried with anhydrous Na₂SO₄, filtered and concentrated in vacuum to afford 4-bromo-3,6-difluoro-2-nitro-aniline (600 mg, 1.90 mmol, 69.9% yield, 80% purity) as a brown oil which was used for the next step without further purification. 10 ¹H NMR (400 MHz, DMSO-d₆) 7.78 (dd, J=6.1, 10.9 Hz, 1H), 7.07 (br s, 2H).

Step 4: To a solution of 4-bromo-3,6-difluoro-2-nitro-aniline (300 mg, 1.19 mmol) in MeOH (3 mL) was added 1,1,2-trichloropropane (262.23 mg, 1.78 mmol) and wet Pd/C (0.4 g, 10% purity in water) under Ar. The suspension was degassed under vacuum and purged with H2 (30 psi) several times. The mixture was stirred at 25° C. for 10 hours. The mixture was filtered and the filtrate was concentrated to afford 3,6-difluorobenzene-1,2-diamine hydrochloride (200 mg, 1.11 mmol, 93.4% yield, crude purity) as a brown solid which was used in the next step without further purification.

MS ES⁺: 145.0

Step 5: A mixture of 3,6-difluorobenzene-1,2-diamine hydrochloride (200 mg, 1.11 mmol) and (3Z)-4-amino-N-hydroxy-1,2,5-oxadiazole-3-carboximidoyl chloride hydrochloride (220.39 mg, 1.11 mmol) in EtOH (3 mL) was stirred at 90° C. for 12 hours. The mixture was concentrated to afford the crude product which was purified by prep. HPLC (Column: Welch Xtimate 75*40 mm*3 μm, Mobile Phase A: water (0.225% FA), Mobile Phase B: acetonitrile, Flow rate: 25 mL/min, gradient condition from 25% B to 55%). The pure fractions were collected and the volatiles were removed under vacuum. The residue was partitioned between acetonitrile (2 mL) and water (10 mL). The solution was lyophilized to dryness to give 4-(4,7-difluoro-benzimidazol-2-yl)-1,2,5-oxadiazol-3-amine (110 mg, 0.446 mmol, 40.3% yield, 96.2% purity) as a brown solid.

MS ES⁺: 237.8

Step 6: To a mixture of 4-(4,7-difluoro-benzimidazol-2-yl)-1,2,5-oxadiazol-3-amine (30 mg, 0.126 mmol) and 3-(bromomethyl)pyridine (21.76 mg, 0.126 mmol) in DMF (1 mL) was added K₂CO₃ (34.97 mg, 0.253 mmol) in one portion at 25° C. The mixture was stirred at 110° C. for 1 hour. Then the mixture was filtered and the filtrate was concentrated to afford the crude product which was purified by prep. HPLC (Column: Phenomenex Gemini-NX C₁₈ 75*30 mm*3 μm, Mobile Phase A: water (0.05% NH₃·H₂O+10 mM NH₄HCO₃), Mobile Phase B: acetonitrile, Flow rate: 25 mL/min, gradient condition from 18% B to 58%). The pure fractions were collected and the volatiles were removed under vacuum. The residue was partitioned between acetonitrile (2 mL) and water (10 mL). The solution was lyophilized to dryness to give 4-[4,7-difluoro-1-(pyridin-3-ylmethyl)benzimidazol-2-yl]-1,2,5-oxadiazol-3-amine (4.75 mg, 0.014 mmol, 11.3% yield, 99.0% purity) as an off-white powder.

MS ES⁺: 329.1

¹H NMR (400 MHz, DMSO-d₆) 8.54-8.46 (m, 2H), 7.57-7.50 (m, 1H), 7.34 (dd, J=4.8, 7.8 Hz, 1H), 7.30-7.17 (m, 2H), 6.92 (s, 2H), 6.02 (s, 2H).

### Example 23: 4-[3-[(6-chloropyridin-3-yl)methyl]imidazo[4,5-b]pyridin-2-yl]-1,2,5-oxadiazol-3-amine

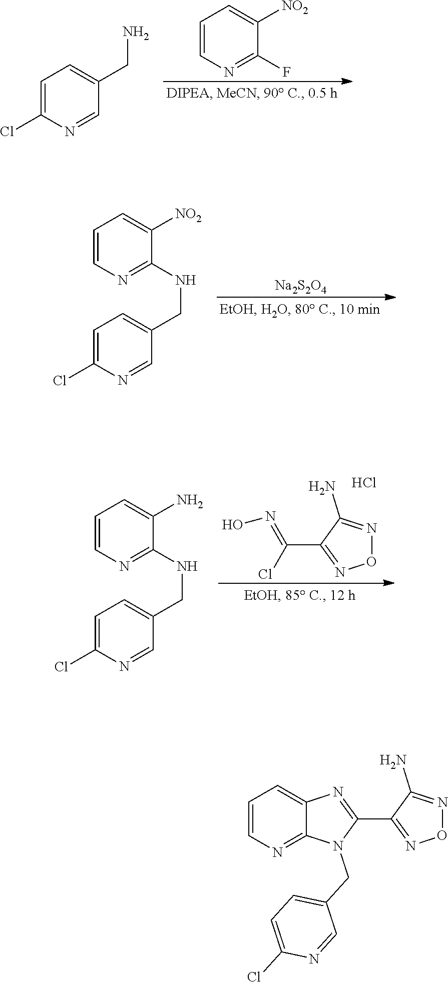

Step 1: A mixture of (6-chloropyridin-3-yl)methanamine (200 mg, 1.40 mmol), DIPEA (362.57 mg, 2.81 mmol) and 2-fluoro-3-nitro-pyridine (199.30 mg, 1.40 mmol) in MeCN (2 mL) was heated to 90° C. and stirred for 0.5 hour. The resulting mixture was cooled to RT and concentrated to dryness by vacuum. The residue was purified by flash silica gel chromatography (ISCO®; 4 g SepaFlash® Silica Flash Column, eluent of petroleum ether: ethyl acetate=3:1 @ 35 mL/min) to give N-[(6-chloropyridin-3-yl)methyl]-3-nitro-pyridin-2-amine (350 mg, 1.14 mmol, 81.1% yield, 86% purity) as a yellow solid which was used for the next step directly.

MS ES⁺: 264.8

Step 2: A solution of Na₂S₂O₄ (1.09 g, 6.23 mmol) in H₂O (3 mL) was added into the mixture of N-[(6-chloropyridin-3-yl)methyl]-3-nitro-pyridin-2-amine (330 mg, 1.25 mmol) in EtOH (6 mL) at 80° C. The mixture was stirred at 80° C. for 10 min. Then the mixture was cooled down to RT. The mixture was poured into water (10 mL) and extracted with ethyl acetate (10 mL×3). The combined organic layers were washed with brine (10 mL), dried over Na₂SO₄ and filtered. The filtrate was evaporated to dryness to give N²-[(6-chloropyridin-3-yl)methyl]pyridine-2,3-diamine (240 mg, 0.990 mmol, 79.4% yield, 96.8% purity) as a yellow solid which was used for the next step without purification.

MS ES⁺: 235.1

Step 3: A mixture of N²-[(6-chloropyridin-3-yl)methyl]pyridine-2,3-diamine (70 mg, 0.298 mmol) and (3Z)-4-amino-N-hydroxy-1,2,5-oxadiazole-3-carboximidoyl chloride hydrochloride (59.35 mg, 0.298 mmol) in EtOH (2 mL) was stirred at 85° C. for 12 hours. The resulting product was cooled to RT and filtered. The filtrate was concentrated in vacuo. The crude product was purified by prep. HPLC (Column: Phenomenex Luna C₁₈ 100*30 mm*3 μm, Mobile Phase A: water (0.225% FA), Mobile Phase B: acetonitrile, Flow rate: 25 mL/min, gradient condition from 40% B to 70%). The pure fractions were collected and the volatiles were removed under vacuum. The residue was partitioned between acetonitrile (2 mL) and water (10 mL). The solution was lyophilized to dryness to give 4-[3-[(6-chloropyridin-3-yl)methyl]imidazo[4,5-b]pyridin-2-yl]-1,2,5-oxadiazol-3-amine (1.07 mg, 0.003 mmol, 1.0% yield, 95.3% purity) as a yellow solid.

MS ES⁺: 328.2

¹H NMR (400 MHz, DMSO-d₆) 8.58 (dd, J=1.6, 4.8 Hz, 1H), 8.44 (d, J=2.4 Hz, 1H), 8.34 (dd, J=1.6, 8.0 Hz, 1H), 7.71 (dd, J=2.4, 8.4 Hz, 1H), 7.50 (dd, J=4.8, 8.0 Hz, 1H), 7.45 (d, J=8.4 Hz, 1H), 6.98 (s, 2H), 5.94 (s, 2H).

### Example 24: 3-[3-[(6-chloropyridin-3-yl)methyl]imidazo[4,5-b]pyridin-2-yl]-4-methyl-1,2,5-oxadiazole

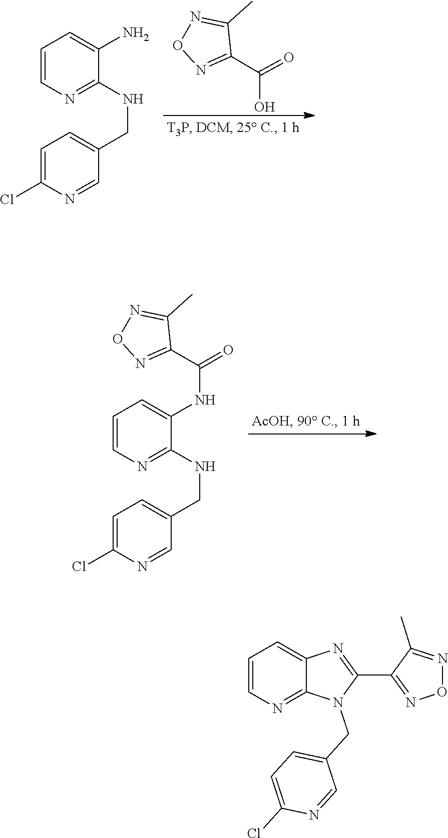

Step 1: To a mixture of N²-[(6-chloropyridin-3-yl)methyl]pyridine-2,3-diamine (80 mg, 0.341 mmol), 4-methyl-1,2,5-oxadiazole-3-carboxylic acid (Intermediate 2) (87.32 mg, 0.682 mmol) and TEA (68.99 mg, 0.682 mmol) in DCM (2 mL) was added T₃P (325.39 mg, 0.511 mmol, 50% in ethyl acetate) in portions at ° C. The mixture was stirred at 25° C. for 1 hour. Then the mixture was poured into water (5 mL) and extracted with DCM (5 mL×3). The combined organic layers were dried over Na₂SO₄, filtered and concentrated to afford N-[2-[(6-chloropyridin-3-yl)methylamino]pyridin-3-yl]-4-methyl-1,2,5-oxadiazole-3-carboxamide (100 mg, 0.290 mmol, 85.1% yield) as black solid which was used for the next step directly.

Step 2: A mixture of N-[2-[(6-chloropyridin-3-yl)methylamino]pyridin-3-yl]-4-methyl-1,2,5-oxadiazole-3-carboxamide (100 mg, 0.290 mmol) in AcOH (3 mL) was stirred at 90° C. for 1 hour. The mixture was cooled down to RT and adjusted pH=9 with sat. NaHCO₃ (aq.) (10 mL). Then the mixture was extracted with ethyl acetate (10 mL×3). The combined organic layers were dried with anhydrous Na₂SO₄ and concentrated to afford the crude product which was purified by prep. HPLC (Column: Phenomenex Gemini-NX C₁₈ 75*30 mm*3 μm, Mobile Phase A: water (0.04% NH₃·H₂O+10 mM NH₄HCO₃), Mobile Phase B: acetonitrile, Flow rate: 25 mL/min, gradient condition from 22% B to 72%). The pure fractions were collected and the volatiles were removed under vacuum. The residue was partitioned between acetonitrile (2 mL) and water (10 mL). The solution was lyophilized to dryness to give 3-[3-[(6-chloropyridin-3-yl)methyl]imidazo[4,5-b]pyridin-2-yl]-4-methyl-1,2,5-oxadiazole (21.55 mg, 0.064 mmol, 21.9% yield, 96.5% purity) as a yellow solid.

MS ES⁺: 327.2

¹H NMR (400 MHz, DMSO-d₆) 8.57 (dd, J=1.6, 4.8 Hz, 1H), 8.44 (d, J=2.0 Hz, 1H), 8.37 (dd, J=1.6, 8.0 Hz, 1H), 7.74 (dd, J=2.8, 8.4 Hz, 1H), 7.54-7.42 (m, 2H), 5.91 (s, 2H), 2.78 (s, 3H).

### Example 25: 5-[[2-(4-amino-1,2,5-oxadiazol-3-yl)-7-fluoro-benzimidazol-1-yl]methyl]pyrimidine-2-carbonitrile

### Example 26: 5-[[2-(4-amino-1,2,5-oxadiazol-3-yl)-4-fluoro-benzimidazol-1-yl]methyl]pyrimidine-2-carbonitrile

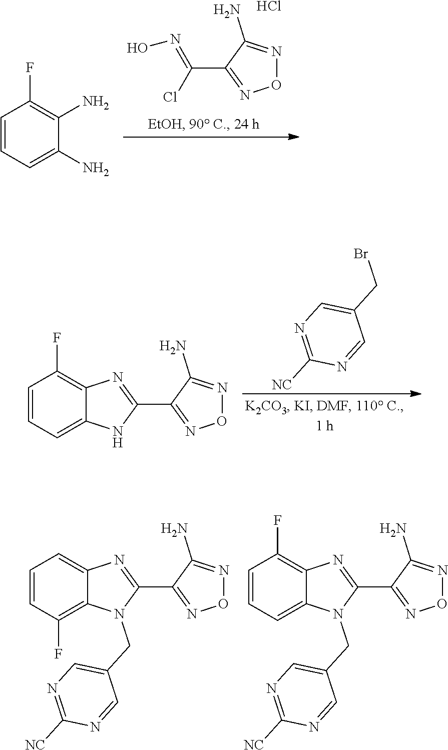

Step 1: A solution of 3-fluorobenzene-1,2-diamine (2 g, 15.86 mmol) and (3Z)-4-amino-N-hydroxy-1,2,5-oxadiazole-3-carboximidoyl chloride hydrochloride (3.16 g, 15.86 mmol) in EtOH (40 mL) was stirred at 85° C. for 24 hours. The reaction mixture was cooled down to RT and evaporated to dryness. The residue was dissolved in ethyl acetate (200 mL) and adjusted to pH=8-9 by sat. NaHCO₃ (aq.). The mixture was extracted with ethyl acetate (100 mL×3). The combined organic layers were dried over Na₂SO₄ and filtered. The filtrate was evaporated to dryness. The residue was purified by silica gel chromatography eluted with petroleum ether: ethyl acetate (1:1) to give 4-(4-fluoro-1H-benzimidazol-2-yl)-1,2,5-oxadiazol-3-amine (1.6 g, 7.08 mmol, 44.7% yield, 97% purity) as a yellow solid.

MS ES⁺: 220.0

Step 2: To a solution of 4-(4-fluoro-1H-benzimidazol-2-yl)-1,2,5-oxadiazol-3-amine (100 mg, 0.456 mmol), 5-(bromomethyl)pyrimidine-2-carbonitrile (prepared as described for US2018/079742) (90.35 mg, 0.456 mmol) and DMF (2 mL) was added K₂CO₃ (126.11 mg, 0.913 mmol) and KI (15.15 mg, 0.091 mmol). The mixture was stirred at 110° C. for 1 hour. The reaction mixture was concentrated under reduced pressure to give a residue which was purified by prep. HPLC (Column: Phenomenex Luna C₁₈ 75*30 mm*3 μm, Mobile Phase A: water (0.225% FA), Mobile Phase B: acetonitrile, Flow rate: 25 mL/min, gradient condition from 32% B to 62%). The pure fractions were collected and the volatiles were removed under vacuum. The residue was partitioned between acetonitrile (2 mL) and water (10 mL). The solution was lyophilized to dryness to give Peak 1 (5-[[2-(4-amino-1,2,5-oxadiazol-3-yl)-4-fluoro-benzimidazol-1-yl]methyl]pyrimidine-2-carbonitrile) (5.98 mg, 0.018 mmol, 3.9% yield, 99.1% purity) as a yellow solid and Peak 2 (5-[[2-(4-amino-1,2,5-oxadiazol-3-yl)-7-fluoro-benzimidazol-1-yl]methyl]pyrimidine-2-carbonitrile) (12.41 mg, 0.037 mmol, 8.1% yield, 99.8% purity) as a yellow solid.

### Example 25 (Peak 2): 5-[[2-(4-amino-1,2,5-oxadiazol-3-yl)-7-fluoro-benzimidazol-1-yl]methyl]pyrimidine-2-carbonitrile

MS ES⁺: 337.3

¹H NMR (400 MHz, DMSO-d₆) 8.99-8.89 (m, 2H), 7.77-7.69 (m, 1H), 7.40-7.32 (m, 1H), 7.29-7.22 (m, 1H), 6.98-6.90 (m, 2H), 6.12 (s, 2H).

### Example 26 (Peak 1): 5-[[2-(4-amino-1,2,5-oxadiazol-3-yl)-4-fluoro-benzimidazol-1-yl]methyl]pyrimidine-2-carbonitrile

MS ES⁺: 337.3

¹H NMR (400 MHz, DMSO-d₆) 8.91 (s, 2H), 7.68-7.61 (m, 1H), 7.46-7.40 (m, 1H), 7.28-7.21 (m, 1H), 6.92 (s, 2H), 6.10 (s, 2H).

### Example 27: 3-[5,7-difluoro-1-(pyridin-3-ylmethyl)benzimidazol-2-yl]-4-methyl-1,2,5-oxadiazole

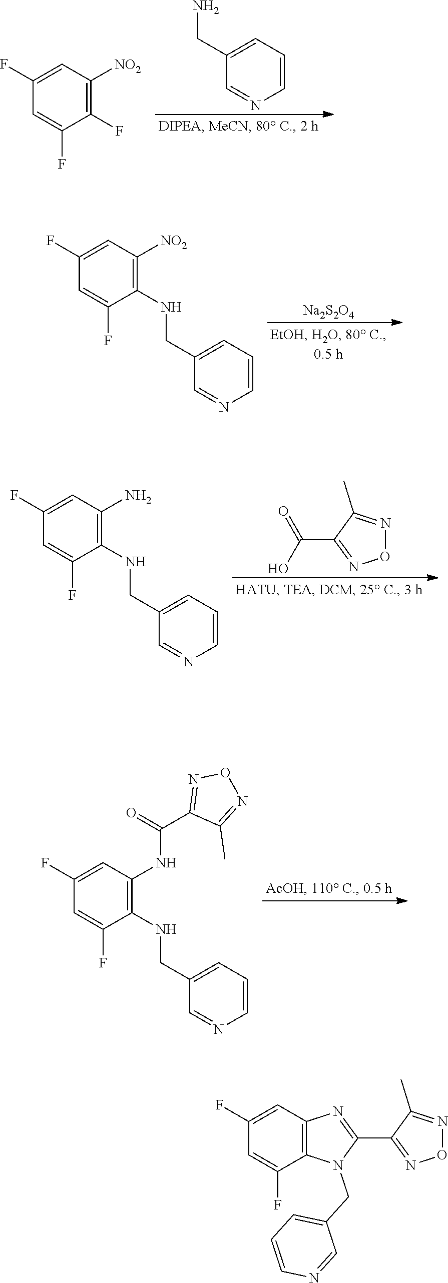

Step 1: A solution of 1,2,5-trifluoro-3-nitro-benzene (1 g, 5.65 mmol), pyridin-3-ylmethanamine (610.69 mg, 5.65 mmol) and DIPEA (1.46 g, 11.29 mmol) in MeCN (10 mL) was stirred at 80° C. for 2 hours. Then the mixture was cooled down to RT and extracted with ethyl acetate (20 mL×2). The combined organic layers were dried over Na₂SO₄ and filtered. The filtrate was evaporated to dryness which was purified by silica gel chromatography (column height: 250 mm, diameter: 100 mm, 100-200 mesh silica gel, petroleum ether: ethyl acetate=1:1) to afford 2,4-difluoro-6-nitro-N-(pyridin-3-ylmethyl)aniline (1.19 g, 4.49 mmol, 79.5% yield) as a yellow solid.

MS ES⁺: 266.1

Step 2: A mixture of 2,4-difluoro-6-nitro-N-(pyridin-3-ylmethyl)aniline (1 g, 3.77 mmol) in EtOH (20 mL) was heated to 80° C. and stirred for 10 min. Then a solution of sodium hydrosulfite (656.49 mg, 3.77 mmol) in water (20 mL) was added into the mixture and stirred for 0.5 hour until the resulting mixture turned from yellow to colourless. Then the mixture was cooled down to RT and extracted with DCM (20 mL×2). The combined organic layers were washed with brine (20 mL×2), dried over Na₂SO₄ and filtered. The filtrate was evaporated to dryness to give 3,5-difluoro-N²(pyridin-3-ylmethyl)benzene-1,2-diamine (500 mg, 2.13 mmol, 56.4% yield) as an off-white solid which was used for the next step without further purification.

Step 3: To a solution of 3,5-difluoro-N²-(pyridin-3-ylmethyl)benzene-1,2-diamine (200 mg, 0.850 mmol), TEA (258.10 mg, 2.55 mmol) and 4-methyl-1,2,5-oxadiazole-3-carboxylic acid (Intermediate 2) (108.90 mg, 0.850 mmol) in DCM (2 mL) was added HATU (646.56 mg, 1.70 mmol). The reaction mixture was stirred at 25° C. for 3 hours. Then the mixture was extracted with DCM (10 mL ×3). The combined organic layers were washed with brine (20 mL), dried over Na₂SO₄ and filtered. The filtrate was evaporated to dryness to give N-(3,5-difluoro-2-((pyridin-3-ylmethyl)amino)phenyl)-4-methyl-1,2,5-oxadiazole-3-carboxamide (250 mg, 0.724 mmol, 85.2% yield) as a brown solid which was used for the next step directly.

Step 4: A solution of N-(3,5-difluoro-2-((pyridin-3-ylmethyl)amino)phenyl)-4-methyl-1,2,5-oxadiazole-3-carboxamide (250 mg, 0.724 mmol) in AcOH (10 mL) was stirred at 110° C. for 0.5 hour. The reaction mixture was concentrated in vacuum to give the crude product which was further purified by prep. HPLC (Column: Welch Xtimate 75*40 mm*3 μm, Mobile Phase A: water (0.225% FA), Mobile Phase B: acetonitrile, Flow rate: 25 mL/min, gradient condition from 40% B to 70%). The pure fractions were collected and the volatiles were removed under vacuum. The residue was partitioned between acetonitrile (2 mL) and water (10 mL). The solution was lyophilized to dryness to give 3-[5,7-difluoro-1-(pyridin-3-ylmethyl)benzimidazol-2-yl]-4-methyl-1,2,5-oxadiazole (38.01 mg, 0.113 mmol, 15.5% yield, 96.9% purity) as a white solid.

MS ES⁺: 328.2

¹H NMR (400 MHz, DMSO-d₆) 8.52-8.45 (m, 2H), 7.67-7.62 (m, 1H), 7.57-7.51 (m, 1H), 7.40-7.32 (m, 2H), 5.96 (s, 2H), 2.76 (s, 3H).

### Example 28: 5-[[2-(4-amino-1,2,5-oxadiazol-3-yl)-4,7-difluoro-benzimidazol-1-yl]methyl]pyrimidine-2-carbonitrile

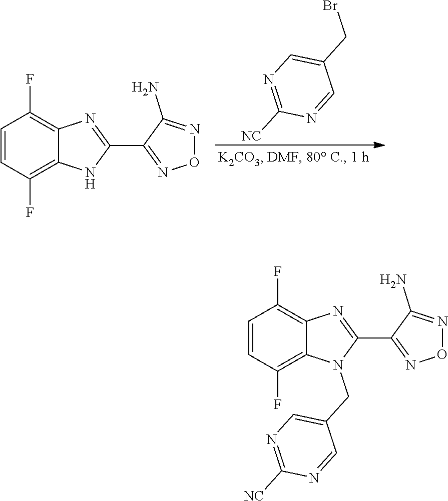 [SMILES: Nc1nonc1-c1nc2c(F)ccc(F)c2[nH]1 ; N#Cc1ncc(CBr)cn1 ; N#Cc1ncc(Cn2c(-c3nonc3N)nc3c(F)ccc(F)c32)cn1]

Prepared as described for Example 22 using 4-(4,7-difluoro-1H-benzimidazol-2-yl)-1,2,5-oxadiazol-3-amine (10 mg, 0.042 mmol) and 5-(bromomethyl)pyrimidine-2-carbonitrile (12.65 mg, 0.042 mmol, 66% purity) to give 5-[[2-(4-amino-1,2,5-oxadiazol-3-yl)-4,7-difluoro-benzimidazol-1-yl]methyl]pyrimidine-2-carbonitrile (4.81 mg, 0.014 mmol, 32.1% yield, 99.8% purity) as a white powder.

MS ES⁺: 355.3

¹H NMR (400 MHz, DMSO-d₆) 8.98 (s, 2H), 7.32-7.18 (m, 2H), 6.90 (s, 2H), 6.11 (s, 2H).

### Example 29: 4-[3-[(6-chloropyridin-3-yl)methyl]-6-fluoro-imidazo[4,5-b]pyridin-2-yl]-1,2,5-oxadiazol-3-amine

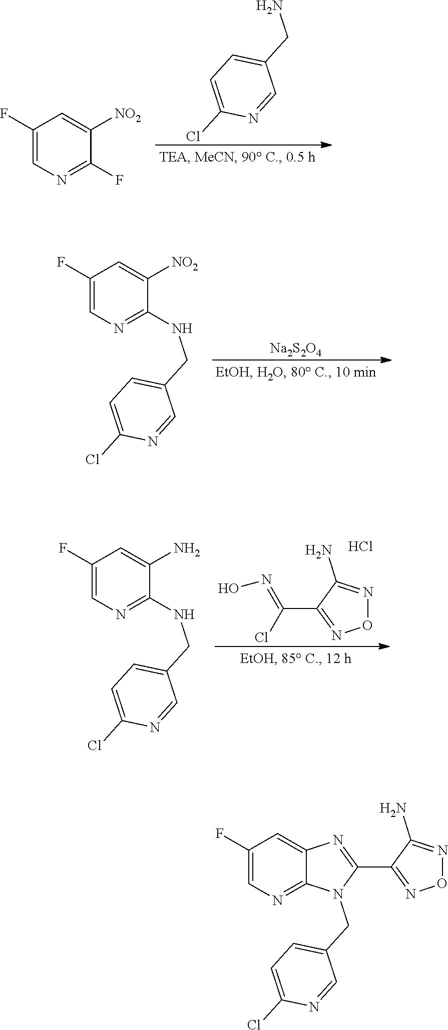

Prepared as described for Example 23 used 2,5-difluoro-3-nitro-pyridine (100 mg, 0.625 mmol) and (6-chloropyridin-3-yl)methanamine (89.07 mg, 0.625 mmol) to give 4-[3-[(6-chloropyridin-3-yl)methyl]-6-fluoro-imidazo[4,5-b]pyridin-2-yl]-1,2,5-oxadiazol-3-amine (1.03 mg, 0.003 mmol, 1.4% yield, 94.0% purity) as a brown solid.

MS ES⁺: 346.2

¹H NMR (400 MHz, DMSO-d₆) 8.66-8.57 (m, 1H), 8.43 (d, J=2.4 Hz, 1H), 8.31 (d, J=9.6 Hz, 1H), 7.72 (d, J=8.4 Hz, 1H), 7.45 (d, J=8.4 Hz, 1H), 6.95 (s, 2H), 5.92 (s, 2H).

### Example 30: 3-[1-[dideuterio(pyridin-3-yl)methyl]-4-fluoro-benzimidazol-2-yl]-4-methyl-1,2,5-oxadiazole

### Example 31: 3-[1-[dideuterio(pyridin-3-yl)methyl]-7-fluoro-benzimidazol-2-yl]-4-methyl-1,2,5-oxadiazole

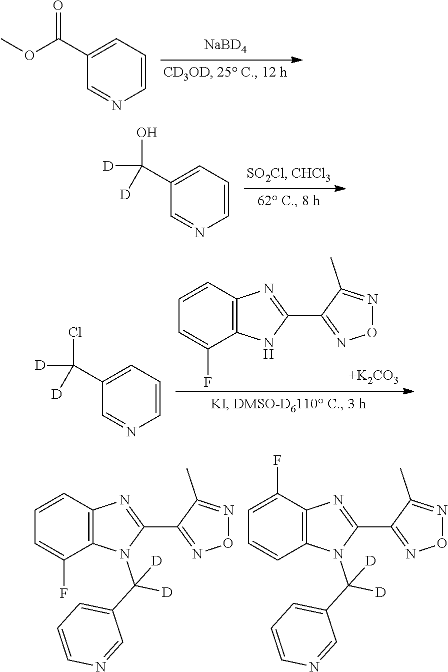

Step 1: Sodium tetradeuterioborate(II) (551.75 mg, 14.58 mmol) was added to a solution of methyl pyridine-3-carboxylate (1 g, 7.29 mmol) in methanol-d₄ (10 mL) at ° C. in portions. Then the mixture was stirred at 25° C. for 12 hours. The mixture was evaporated to dryness. The residue was extracted with ethyl acetate (10 mL×3) and the combined organic layers were washed with brine, dried over Na₂SO₄ and filtered. The filtrate was evaporated to dryness to give dideuterio(pyridin-3-yl)methanol (550 mg, 4.95 mmol, 67.9% yield) as colourless oil which was used for the next step directly.

Step 2: To a solution of dideuterio(pyridin-3-yl)methanol (30 mg, 0.270 mmol) and CHCl₃ (1 mL) was added SOCl₂ (128.46 mg, 1.08 mmol) at ° C. The mixture was stirred at 62° C. for 8 hours. The reaction mixture was concentrated under reduced pressure to give 3-[chloro(dideuterio)methyl]pyridine (45 mg) as a white solid which was used for the next step without further purification.

MS ES⁺: 130.0

Step 3: A mixture of 3-(7-fluoro-benzimidazol-2-yl)-4-methyl-1,2,5-oxadiazole (Intermediate 1) (60 mg, 0.275 mmol), K₂CO₃ (76.01 mg, 0.550 mmol) and deuterium oxide (1 mL) was stirred at 25° C. for 1 hour. The resulting mixture was concentrated to afford a residue which was partitioned between acetonitrile (10 mL) and deuterium oxide (20 mL). The solution was lyophilized to dryness to give crude potassium 7-fluoro-2-(4-methyl-1,2,5-oxadiazol-3-yl)benzo[d]imidazol-1-ide (136 mg) as a yellow solid. To a solution of 3-[chloro(dideuterio)methyl]pyridine (45 mg, 0.271 mmol), KI (9.00 mg, 0.054 mmol) and trideuterio(trideuteriomethylsulfinyl)methane (1 mL) was added crude potassium 7-fluoro-2-(4-methyl-1,2,5-oxadiazol-3-yl)benzo[d]imidazol-1-ide (136 mg). The mixture was stirred at 110° C. for 3 hours. The reaction mixture was filtered and the filtrated was purified by prep. HPLC (Column: Phenomenex Luna C₁₈ 75*30 mm*3 μm, Mobile Phase A: water (0.225% FA), Mobile Phase B: acetonitrile, Flow rate: 25 mL/min, gradient condition from 11% B to 41%) to give Peak 1 (3-[1-[dideuterio(pyridin-3-yl)methyl]-7-fluoro-benzimidazol-2-yl]-4-methyl-1,2,5-oxadiazole) (13.14 mg, 0.042 mmol, 15.6% yield, 100% purity) as an off-white solid and Peak 2 (3-[1-[dideuterio(pyridin-3-yl)methyl]-4-fluoro-benzimidazol-2-yl]-4-methyl-1,2,5-oxadiazole) (19.52 mg, 0.062 mmol, 23.0% yield, 99.3% purity) as a white solid.

### Example 30 (Peak 2): 3-[1-[dideuterio(pyridin-3-yl)methyl]-4-fluoro-benzimidazol-2-yl]-4-methyl-1,2,5-oxadiazole

MS ES⁺: 312.3

¹H NMR (400 MHz, DMSO-d₆) 8.52-8.43 (m, 2H), 7.58-7.49 (m, 2H), 7.44-7.36 (m, 1H), 7.35-7.29 (m, 1H), 7.24-7.15 (m, 1H), 2.75 (s, 3H).

### Example 31 (Peak 1): 3-[1-[dideuterio(pyridin-3-yl)methyl]-7-fluoro-benzimidazol-2-yl]-4-methyl-1,2,5-oxadiazole

MS ES⁺: 312.3

¹H NMR (400 MHz, DMSO-d₆) 8.51-8.38 (m, 2H), 7.75-7.68 (m, 1H), 7.55-7.49 (m, 1H), 7.38-7.30 (m, 2H), 7.26-7.18 (m, 1H), 2.75 (s, 3H).

### Example 32: 4-[7-fluoro-1-(pyrazin-2-ylmethyl)benzimidazol-2-yl]-1,2,5-oxadiazol-3-amine

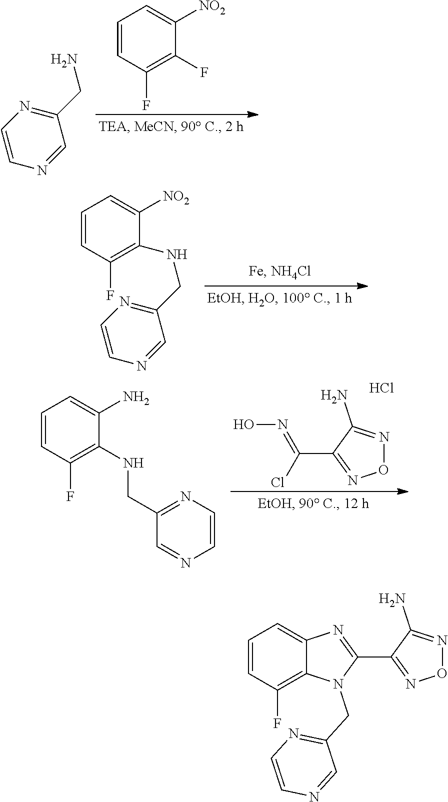

Prepared as described for Example 21 using pyrazin-2-ylmethanamine (500 mg, 4.58 mmol) and 1,2-difluoro-3-nitro-benzene (728.91 mg, 4.58 mmol) to give 4-[7-fluoro-1-(pyrazin-2-ylmethyl)benzimidazol-2-yl]-1,2,5-oxadiazol-3-amine (32.35 mg, 0.103 mmol, 2.2% yield, 99.4% purity) as an off-white solid.

MS ES⁺: 312.2

¹H NMR (400 MHz, DMSO-d₆) 8.80 (d, J=1.1 Hz, 1H), 8.55 (d, J=2.6 Hz, 1H), 8.44 (dd, J=1.6, 2.4 Hz, 1H), 7.71 (d, J=8.1 Hz, 1H), 7.34 (dt, J=40.9, 8.1 Hz, 1H), 7.22 (dd, J=8.0, 11.8 Hz, 1H), 7.03-6.93 (m, 2H), 6.20 (s, 2H).

### Example 33: 4-[5-bromo-1-(pyridin-3-ylmethyl)benzimidazol-2-yl]-1,2,5-oxadiazol-3-amine

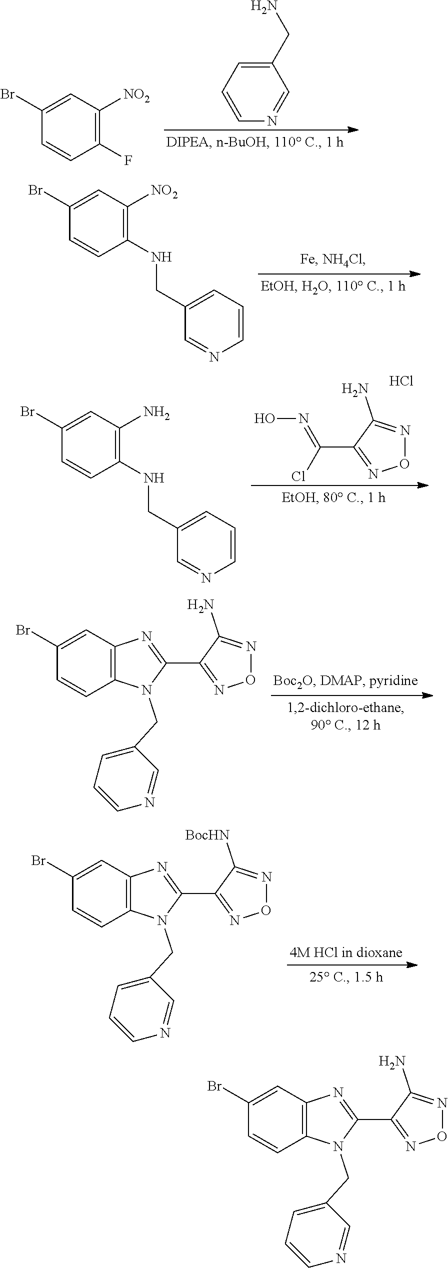

Step 1: A solution of 4-bromo-1-fluoro-2-nitro-benzene (20 g, 90.91 mmol), pyridin-3-ylmethanamine (9.83 g, 90.91 mmol) and DIPEA (35.25 g, 272.73 mmol) in n-BuOH (100 mL) was stirred at 110° C. for 1 hour. The resulting mixture was cooled down to RT with yellow precipitation formed. The precipitation was filtered and washed with EtOH (50 mL) to give 4-bromo-2-nitro-N-(pyridin-3-ylmethyl)aniline (25 g, 77.08 mmol, 84.8% yield, 95% purity) as a yellow solid which was used for the next step directly.

MS ES⁺: 310.1

¹H NMR (400 MHz, DMSO-d₆) 8.77 (t, J=6.2 Hz, 1H), 8.60 (d, J=1.6 Hz, 1H), 8.46 (dd, J=1.4, 4.7 Hz, 1H), 8.22-8.14 (m, 1H), 7.75 (br d, J=7.9 Hz, 1H), 7.58 (dd, J=2.0, 9.3 Hz, 1H), 7.35 (dd, J=40.8, 7.9 Hz, 1H), 6.91 (d, J=9.3 Hz, 1H), 4.68 (d, J=6.3 Hz, 2H).

Step 2: A mixture of 4-bromo-2-nitro-N-(pyridin-3-ylmethyl)aniline (5 g, 16.23 mmol), NH₄Cl (4.34 g, 81.13 mmol) and Fe powder (2.72 g, 48.68 mmol) in EtOH (50 mL) and water (50 mL) was stirred at 110° C. for 1 hour. The mixture was cooled down to RT and filtered. The filtrate was evaporated to remove most of the EtOH and the mixture was extracted with DCM (100 mL×3). The combined organic layers were washed with brine (100 mL), dried over Na₂SO₄ and filtered. The filtrated was evaporated to dryness to obtain 4-bromo-N¹-(pyridin-3-ylmethyl)benzene-1,2-diamine (4.3 g, 14.69 mmol, 90.5% yield, 95% purity) as an off-white solid which was used for the next step directly.

MS ES⁺: 280.0

Step 3: A solution of 4-bromo-N¹-(pyridin-3-ylmethyl)benzene-1,2-diamine (3.3 g, 11.86 mmol) and (Z)-4-amino-N-hydroxy-1,2,5-oxadiazole-3-carbimidoyl chloride hydrochloride (1.74 g, 10.68 mmol) in EtOH (100 mL) was stirred at 80° C. for 1 hour. The resulting product was added to water (50 mL) and brown precipitation formed. The precipitation was collected to give 4-[5-bromo-1-(pyridin-3-ylmethyl)benzimidazol-2-yl]-1,2,5-oxadiazol-3-amine (2.5 g, 6.74 mmol, 56.8% yield) as a brown solid which was used in the next step without further purification.

MS ES⁺: 370.9

¹H NMR (400 MHz, DMSO-d₆) 8.52 (s, 1H), 8.47 (d, J=4.0 Hz, 1H), 8.10 (d, J=1.4 Hz, 1H), 7.78 (d, J=8.8 Hz, 1H), 7.57 (dd, J=1.5, 8.8 Hz, 1H), 7.50 (br d, J=8.0 Hz, 1H), 7.31 (dd, J=40.9, 7.8 Hz, 1H), 6.97 (s, 2H), 5.99 (s, 2H).

Step 4: A solution of 4-[5-bromo-1-(pyridin-3-ylmethyl)benzimidazol-2-yl]-1,2,5-oxadiazol-3-amine (3.2 g, 8.62 mmol) in 1,2-dichloro-ethane (30 mL) and pyridine (25-91 g, 327.59 mmol) was treated with DMAP (1.16 g, 9.48 mmol) and Boc₂O (2.82 g, 12.93 mmol). The solution was heated to 90° C. and stirred for 12 hours. The mixture was cooled to RT and concentrated in reduced pressure. The residue was poured into ice water (w/w=1/1) (20 mL) and stirred for 2 min. The aqueous phase was extracted with ethyl acetate (20 mL×3). The combined organic phases were washed with brine (20 mL×3), dried with anhydrous Na₂SO₄, filtered and concentrated in vacuum. The residue was purified by silica gel chromatography (petroleum ether: ethyl acetate=1:0 to 0:1) to afford tert-butyl N-[4-[5-bromo-1-(pyridin-3-ylmethyl)benzimidazol-2-yl]-1,2,5-oxadiazol-3-yl]carbamate (1.9 g, 4.03 mmol, 46.8% yield) as a white solid.

¹H NMR (400 MHz, DMSO-d₆) 10.16 (s, 1H), 8.55 (d, J=1.9 Hz, 1H), 8.48 (dd, J=1.6, 4.7 Hz, 1H), 8.14 (d, J=1.6 Hz, 1H), 7.76 (d, J=8.8 Hz, 1H), 7.61-7.54 (m, 2H), 7.34-7.28 (m, 1H), 6.97 (s, 1H), 6.02-5.98 (m, 1H), 1.47 (s, 9H).

Step 5: A solution of tert-butyl N-[4-[5-bromo-1-(pyridin-3-ylmethyl)benzimidazol-2-yl]-1,2,5-oxadiazol-3-yl]carbamate (50 mg, 0.106 mmol) in 4M HCl/dioxane (5 mL) was stirred at 25° C. for 1.5 hours. The resulting mixture was concentrated under vacuum to give 4-[5-bromo-1-(pyridin-3-ylmethyl)benzimidazol-2-yl]-1,2,5-oxadiazol-3-amine (35.44 mg, 0.091 mmol, 86.1% yield, 95.7% purity) as a white solid.

MS ES⁺: 373.1

¹H NMR (400 MHz, DMSO-d₆) 8.68-8.83 (m, 2H) 8.11-8.15 (m, 1H) 8.04 (s, 1H) 7.79-7.84 (m, 1H) 7.70-7.79 (m, 1H) 7.57-7.65 (m, 1H) 6.98 (s, 2H) 6.10 (s, 2H).

### Example 34: 4-[5-(dimethylamino)-1-(pyridin-3-ylmethyl)benzimidazol-2-yl]-1,2,5-oxadiazol-3-amine

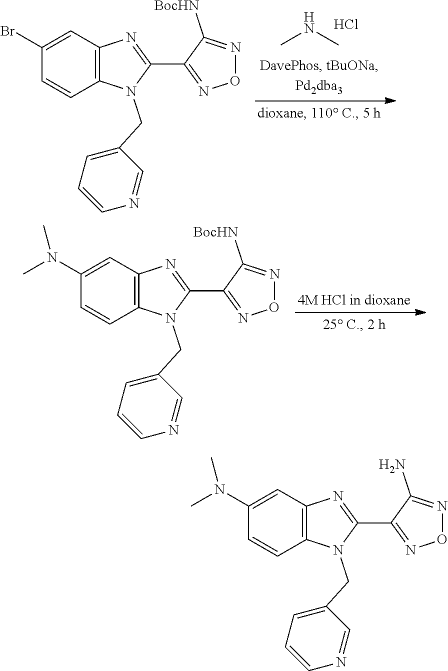

Step 1: To a solution of tert-butyl N-[4-[5-bromo-1-(pyridin-3-ylmethyl)benzimidazol-2-yl]-1,2,5-oxadiazol-3-yl]carbamate (50 mg, 0.106 mmol), N-methylmethanamine hydrochloride (8.65 mg, 0.106 mmol) and tBuONa (30.59 mg, 0.318 mmol) in dioxane (2 mL) was added Pd₂(dba)₃ (19.43 mg, 0.021 mmol) and DavePhos (8.35 mg, 0.021 mmol) under N₂ at 25° C. The mixture was heated to 110° C. and stirred for 5 hours under N₂. The mixture was cooled to room temperature and extracted with DCM (10 mL×3). The combined organics were dried over Na₂SO₄, filtered and dried in vacuo. The residue was purified by preparative TLC to give tert-butyl N-[4-[5-(dimethylamino)-1-(pyridin-3-ylmethyl)benzimidazol-2-yl]-1,2,5-oxadiazol-3-yl]carbamate (46 mg, 0.105 mmol, 99% yield) as a yellow solid.

MS ES⁺: 436

Step 2: A solution of tert-butyl N-[4-[5-(dimethylamino)-1-(pyridin-3-ylmethyl)benzimidazol-2-yl]-1,2,5-oxadiazol-3-yl]carbamate (46 mg, 0.105 mmol) in 4M HCl in dioxane (25 mL) was stirred at 25° C. for 2 hours. The reaction mixture was concentrated under vacuum to dryness. The residue was purified by prep. HPLC (Column: Welch Xtimate 75*40 mm*3 μm, Mobile Phase A: water (0.225% FA), Mobile Phase B: acetonitrile, Flow rate: 25 mL/min, gradient condition from 15% B to 45%).

The pure fractions were collected and the volatiles were removed under vacuum. The residue was partitioned between acetonitrile (2 mL) and water (10 mL). The solution was lyophilized to dryness to give 4-[5-(dimethylamino)-1-(pyridin-3-ylmethyl)benzimidazol-2-yl]-1,2,5-oxadiazol-3-amine (1.0 mg, 0.003 mmol, 2.7% yield, 98.3% purity) as a yellow powder.

MS ES⁺: 336.0

¹H NMR (400 MHz, DMSO-d₆) 8.53-8.43 (m, 2H), 7.63-7.56 (m, 1H), 7.53-7.45 (m, 1H), 7.37-7.27 (m, 1H), 7.10-6.96 (m, 4H), 5.93 (s, 2H), 2.93 (s, 6H).

### Example 35: 5-[[2-(4-amino-1,2,5-oxadiazol-3-yl)-7-fluoro-benzimidazol-1-yl]methyl]pyrazine-2-carbonitrile

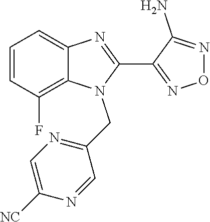 [SMILES: N#Cc1cnc(Cn2c(-c3nonc3N)nc3cccc(F)c32)cn1]

Prepared as described for Example 26 using 4-(4-fluoro-1H-benzimidazol-2-yl)-1,2,5-oxadiazol-3-amine (80 mg, 0.365 mmol) and 5-(chloromethyl)pyrazine-2-carbonitrile (56.05 mg, 0.365 mmol) to give 5-[[2-(4-amino-1,2,5-oxadiazol-3-yl)-7-fluoro-benzimidazol-1-yl]methyl]pyrazine-2-carbonitrile (5.09 mg, 0.014 mmol, 3.8% yield, 91.3% purity) as a yellow solid.

MS ES⁺: 337.3

¹H NMR (400 MHz, DMSO-d₆) 9.11-9.07 (m, 1H), 9.03-8.98 (m, 1H), 7.74-7.71 (m, 1H), 7.39-7.32 (m, 1H), 7.28-7.21 (m, 1H), 7.00-6.93 (m, 2H), 6.34-6.30 (m, 2H).

### Example 36: 5-[[2-(4-amino-1,2,5-oxadiazol-3-yl)-4-fluoro-benzimidazol-1-yl]methyl]pyrazine-2-carbonitrile

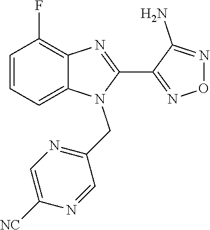 [SMILES: N#Cc1cnc(Cn2c(-c3nonc3N)nc3c(F)cccc32)cn1]

Prepared as described for Example 26 using 4-(4-fluoro-1H-benzimidazol-2-yl)-1,2,5-oxadiazol-3-amine (80 mg, 0.365 mmol) and 5-(chloromethyl)pyrazine-2-carbonitrile (56.05 mg, 0.365 mmol) to give 5-[[2-(4-amino-1,2,5-oxadiazol-3-yl)-4-fluoro-benzimidazol-1-yl]methyl]pyrazine-2-carbonitrile (8.43 mg, 0.024 mmol, 6.7% yield, 97.4% purity) as a yellow solid.

MS ES⁺: 337.3

¹H NMR (400 MHz, DMSO-d₆) 9.11-9.05 (m, 1H), 9.02-8.95 (m, 1H), 7.67-7.59 (m, 1H), 7.46-7.37 (m, 1H), 7.27-7.19 (m, 1H), 6.98-6.89 (m, 2H), 6.32-6.27 (m, 2H).

### Example 37: 5-[[2-(4-methyl-1,2,5-oxadiazol-3-yl)benzimidazol-1-yl]methyl]pyridine-2-carbonitrile

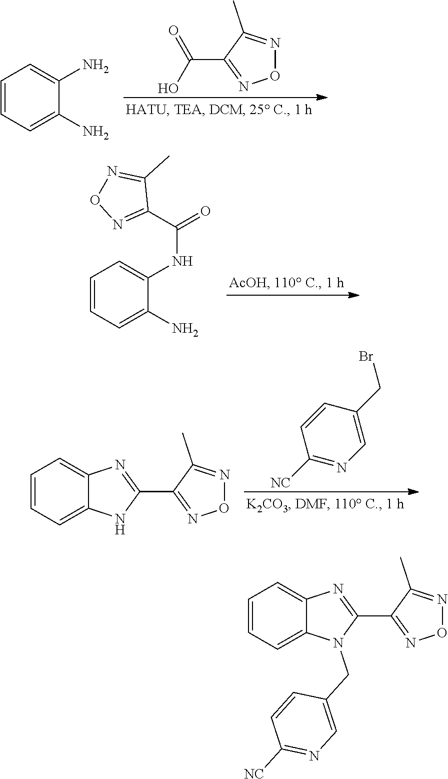

Step 1: A solution of benzene-1,2-diamine (2 g, 18.49 mmol), 4-methyl-1,2,5-oxadiazole-3-carboxylic acid (Intermediate 2) (2.37 g, 18.49 mmol), HATU (8.44 g, 22.19 mmol) and TEA (5.61 g, 55.48 mmol) in DCM (50 mL) was stirred at 25° C. for 1 hour. The mixture was extracted with DCM (100 mL×3). The combined organic layers were washed with brine (50 mL), dried over Na₂SO₄ and filtered. The filtrate was evaporated to dryness to give N-(2-aminophenyl)-4-methyl-1,2,5-oxadiazole-3-carboxamide (4.0 g, 18.33 mmol, 99.1% yield) as a yellow sticky oil which was used for the next step directly.

Step 2: A solution of N-(2-aminophenyl)-4-methyl-1,2,5-oxadiazole-3-carboxamide (4 g, 18.33 mmol) in AcOH (100 mL) was stirred at 110° C. for 1 hour. The mixture was evaporated to dryness. The residue was extracted with DCM (100 mL×3), washed with sat. NaHCO₃ (aq.) (500 mL), dried over Na₂SO₄ and filtered. The filtrate was evaporated to dryness. The residue was purified by silica gel chromatography (column height: 250 mm, diameter: 100 mm, 100-200 mesh silica gel, petroleum ether: ethyl acetate=5:1) to afford 3-(1H-benzimidazol-2-yl)-4-methyl-1,2,5-oxadiazole (2.0 g, 9.99 mmol, 54.5% yield) as a white solid. Then some 3-(1H-benzimidazol-2-yl)-4-methyl-1,2,5-oxadiazole (50 mg, 0.250 mmol) was further purified by prep. HPLC (Column: Welch Xtimate 75*40 mm*3 μm, Mobile Phase A: water (0.225% FA), Mobile Phase B: acetonitrile, Flow rate: 25 mL/min, gradient condition from 45% B to 75%). The pure fractions were collected and the volatiles were removed under vacuum. The residue was partitioned between acetonitrile (2 mL) and water (10 mL). The solution was lyophilized to dryness to give 3-(1H-benzimidazol-2-yl)-4-methyl-1,2,5-oxadiazole (30 mg, 0.149 mmol, 59.8% yield, 99.7% purity) as a white solid.

MS ES⁺: 201.3

¹H NMR (400 MHz, DMSO-d₆) 7.76-7.63 (m, 2H), 7.37-7.29 (m, 2H), 2.78 (s, 3H).

Step 3: To a solution of 3-(1H-benzimidazol-2-yl)-4-methyl-1,2,5-oxadiazole (80 mg, 0.400 mmol) and 5-(bromomethyl)pyridine-2-carbonitrile (78.74 mg, 0.400 mmol) in DMF (1 mL) was added K₂CO₃ (110.46 mg, 0.799 mmol). The reaction mixture was stirred at 110° C. for 1 hour. The reaction was filtered and the filtrate was purified by prep. HPLC (Column: Welch Xtimate 75*40 mm*3 μm, Mobile Phase A: water (0.225% FA)), Mobile Phase B: acetonitrile, Flow rate: 25 mL/min, gradient condition from 50% B to 80%). The pure fractions were collected and the volatiles were removed under vacuum. The residue was partitioned between acetonitrile (2 mL) and water (10 mL).

The solution was lyophilized to dryness to give 5-[[2-(4-methyl-1,2,5-oxadiazol-3-yl)benzimidazol-1-yl]methyl]pyridine-2-carbonitrile (48.90 mg, 0.153 mmol, 38.3% yield, 99.1% purity) as an off-white solid.

MS ES⁺: 317.3

¹H NMR (400 MHz, DMSO-d₆) 8.72 (d, J=1.6 Hz, 1H), 7.95 (d, J=8.0 Hz, 1H), 7.93-7.88 (m, 1H), 7.76-7.67 (m, 2H), 7.46-7.36 (m, 2H), 6.05 (s, 2H), 2.79 (s, 3H).

### Example 38: 3-[i-[(6-chloropyridin-3-yl)methyl]-7-fluoro-benzimidazol-2-yl]-4-methyl-1,2,5-oxadiazole

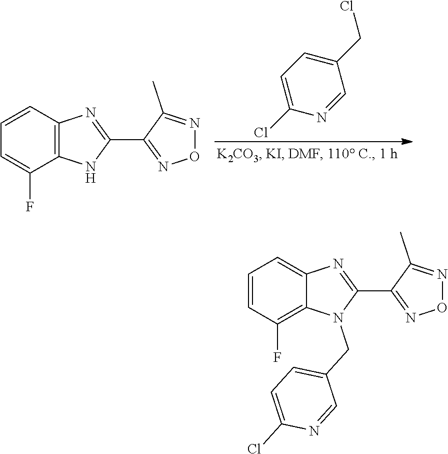 [SMILES: Cc1nonc1-c1nc2cccc(F)c2[nH]1 ; ClCc1ccc(Cl)nc1 ; Cc1nonc1-c1nc2cccc(F)c2n1Cc1ccc(Cl)nc1]

To a mixture of 2-chloro-5-(chloromethyl)pyridine (74.26 mg, 0.458 mmol) and 3-(7-fluoro-benzimidazol-2-yl)-4-methyl-1,2,5-oxadiazole (Intermediate 1) (100 mg, 0.458 mmol) in DMF (2 mL) were added K₂CO₃ (126.69 mg, 0.917 mmol) and KI (7.61 mg, 0.046 mmol) in one portion at 25° C. The mixture was stirred at 110° C. for 1 hour. The resulting product was cooled to RT, then dissolved in DMF (3 mL) and filtered to remove the insoluble. The filtrate was purified by prep. HPLC (Column: Welch Xtimate 75*40 mm*3 μm, Mobile Phase A: water (0.225% FA), Mobile Phase B: acetonitrile, Flow rate: 25 mL/min, gradient condition from 55% B to 75%). The pure fractions were collected and the volatiles were removed under vacuum. The residue was partitioned between acetonitrile (2 mL) and water (10 mL). The solution was lyophilized to dryness to give 3-[1-[(6-chloropyridin-3-yl)methyl]-7-fluoro-benzimidazol-2-yl]-4-methyl-1,2,5-oxadiazole (33.27 mg, 0.95 mmol, 20.8% yield, 98.7% purity) as a white solid.

MS ES⁺: 344.2

¹H NMR (400 MHz, DMSO-d₆) 8.44-8.37 (m, J=2.3 Hz, 1H), 7.68-7.58 (m, 2H), 7.47-7.39 (m, 2H), 7.27-7.18 (m, J=11.0 Hz, 1H), 5.97-5.94 (m, 2H), 2.80-2.78 (m, 3H).

### Example 39: 3-[1-[(6-chloropyridin-3-yl)methyl]-4-fluoro-benzimidazol-2-yl]-4-methyl-1,2,5-oxadiazole

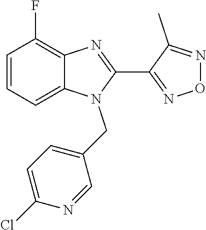 [SMILES: Cc1nonc1-c1nc2c(F)cccc2n1Cc1ccc(Cl)nc1]

Prepared as described for Example 38 using 2-chloro-5-(chloromethyl)pyridine (74.26 mg, 0.458 mmol) and 3-(7-fluoro-benzimidazol-2-yl)-4-methyl-1,2,5-oxadiazole (Intermediate 1) (100 mg, 0.458 mmol) to give 3-[1-[(6-chloropyridin-3-yl)methyl]-4-fluoro-benzimidazol-2-yl]-4-methyl-1,2,5-oxadiazole (25.81 mg, 0.074 mmol, 16.2% yield, 98.9% purity) as a white solid.

MS ES⁺: 344.2

¹H NMR (400 MHz, DMSO-d₆) 8.44-8.29 (m, 1H), 7.79-7.70 (m, J=8.1 Hz, 1H), 7.68-7.56 (m, 1H), 7.53-7.41 (m, J=8.1 Hz, 1H), 7.39-7.30 (m, J=8.1, 8.1 Hz, 1H), 7.29-7.17 (m, 1H), 6.06-5.85 (m, 2H), 2.84-2.72 (m, 3H).

### Example 40: 5-[[4-fluoro-2-(4-methyl-1,2,5-oxadiazol-3-yl)benzimidazol-1-yl]methyl]pyridine-2-carbonitrile

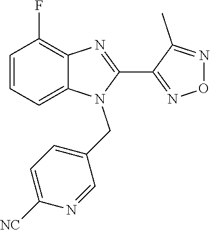 [SMILES: Cc1nonc1-c1nc2c(F)cccc2n1Cc1ccc(C#N)nc1]

Prepared as described for Example 38 using 3-(7-fluoro-benzimidazol-2-yl)-4-methyl-1,2,5-oxadiazole (Intermediate 1) (100 mg, 0.458 mmol) and 5-(bromomethyl)pyridine-2-carbonitrile (prepared as described for WO2007/28083) (135.46 mg, 0.687 mmol) to give 5-[[4-fluoro-2-(4-methyl-1,2,5-oxadiazol-3-yl)benzimidazol-1-yl]methyl]pyridine-2-carbonitrile (7.98 mg, 0.023 mmol, 5.1% yield, 97.1% purity) as an off-white solid.

MS ES⁺: 335.3

¹H NMR (400 MHz, DMSO-d₆) 8.81-8.78 (m, 1H), 8.04-8.01 (m, 1H), 7.81-7.76 (m, 1H), 7.66-7.63 (m, 1H), 7.48 (d, J=4.9 Hz, 1H), 7.33-7.26 (m, 1H), 6.13 (s, 2H), 2.85 (s, 3H).

### Example 41: 5-[[7-fluoro-2-(4-methyl-1,2,5-oxadiazol-3-yl)benzimidazol-1-yl]methyl]pyrazine-2-carbonitrile

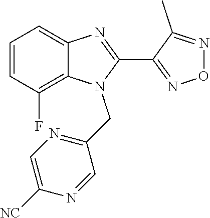 [SMILES: Cc1nonc1-c1nc2cccc(F)c2n1Cc1cnc(C#N)cn1]

Prepared as described for Example 38 using 5-(chloromethyl)pyrazine-2-carbonitrile (70.38 mg, 0.458 mmol) and 3-(7-fluoro-benzimidazol-2-yl)-4-methyl-1,2,5-oxadiazole (Intermediate 1) (100 mg, 0.458 mmol) to give 5-[[7-fluoro-2-(4-methyl-1,2,5-oxadiazol-3-yl)benzimidazol-1-yl]methyl]pyrazine-2-carbonitrile (9.94 mg, 0.029 mmol, 6.4% yield, 98.7% purity) as an off-white powder.

MS ES⁺: 336.3

¹H NMR (400 MHz, DMSO-d₆) 8.99 (s, 2H), 7.68-7.77 (m, 1H), 7.19-7.40 (m, 2H), 6.20-6.27 (m, 2H), 2.75 (s, 3H).

### Example 42: 5-[[4-fluoro-2-(4-methyl-1,2,5-oxadiazol-3-yl)benzimidazol-1-yl]methyl]pyrazine-2-carbonitrile

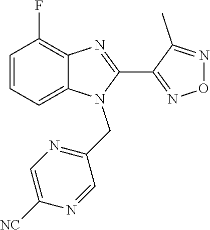 [SMILES: Cc1nonc1-c1nc2c(F)cccc2n1Cc1cnc(C#N)cn1]

Prepared as described for Example 38 using 5-(chloromethyl)pyrazine-2-carbonitrile (70.38 mg, 0.458 mmol) and 3-(7-fluoro-benzimidazol-2-yl)-4-methyl-1,2,5-oxadiazole (Intermediate 1) (100 mg, 0.458 mmol) to give 5-[[4-fluoro-2-(4-methyl-1,2,5-oxadiazol-3-yl)benzimidazol-1-yl]methyl]pyrazine-2-carbonitrile (20.53 mg, 0.061 mmol, 13.3% yield, 99.3% purity) as an off-white powder.

MS ES⁺: 336.3

¹H NMR (400 MHz, DMSO-d₆) 9.01 (s, 2H), 7.56-7.67 (m, 1H), 7.35-7.46 (m, 1H), 7.12-7.26 (m, 1H), 6.24 (s, 2H), 2.77 (s, 3H).

### Example 43: 3-[7-fluoro-1-[(6-methoxypyridin-3-yl)methyl]benzimidazol-2-yl]-4-methyl-1,2,5-oxadiazole

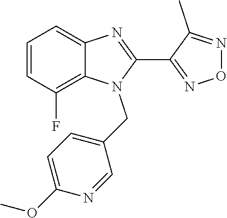 [SMILES: COc1ccc(Cn2c(-c3nonc3C)nc3cccc(F)c32)cn1]

Prepared as described for Example 38 using 5-(chloromethyl)-2-methoxy-pyridine (72.23 mg, 0.458 mmol) and 3-(7-fluoro-benzimidazol-2-yl)-4-methyl-1,2,5-oxadiazole (Intermediate 1) (100 mg, 0.458 mmol) to give 3-[7-fluoro-1-[(6-methoxypyridin-3-yl)methyl]benzimidazol-2-yl]-4-methyl-1,2,5-oxadiazole (30.53 mg, 0.089 mmol, 19.5% yield, 99.3% purity) as a white powder.

MS ES⁺: 340.3

¹H NMR (400 MHz, DMSO-d₆) 8.04 (s, 1H), 7.67-7.76 (m, 1H), 7.45-7.59 (m, 1H), 7.30-7.37 (m, 1H), 7.21-7.28 (m, 1H), 6.72-6.83 (m, 1H), 5.88 (s, 2H), 3.79 (s, 3H), 2.76 (s, 3H).

### Example 44: 5-[[7-fluoro-2-(4-methyl-1,2,5-oxadiazol-3-yl)benzimidazol-1-yl]methyl]pyrimidine-2-carbonitrile

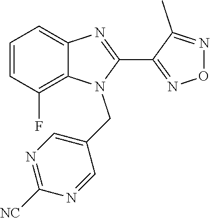 [SMILES: Cc1nonc1-c1nc2cccc(F)c2n1Cc1cnc(C#N)nc1]

Prepared as described for Example 38 using 5-(bromomethyl)pyrimidine-2-carbonitrile (42.00 mg, 0.212 mmol) and 3-(7-fluoro-benzimidazol-2-yl)-4-methyl-1,2,5-oxadiazole (Intermediate 1) (46.28 mg, 0.212 mmol) to give 5-[[7-fluoro-2-(4-methyl-1,2,5-oxadiazol-3-yl)benzimidazol-1-yl]methyl]pyrimidine-2-carbonitrile (20.39 mg, 0.061 mmol, 28.6% yield, 99.7% purity) as a yellow solid.

MS ES⁺: 336.3

¹H NMR (400 MHz, DMSO-d₆) 8.96 (s, 2H), 7.74 (d, J=8.1 Hz, 1H), 7.40-7.33 (m, 1H), 7.29-7.22 (m, 1H), 6.08 (s, 2H), 2.78 (s, 3H).

### Example 45: 5-[[4-fluoro-2-(4-methyl-1,2,5-oxadiazol-3-yl)benzimidazol-1-yl]methyl]pyrimidine-2-carbonitrile

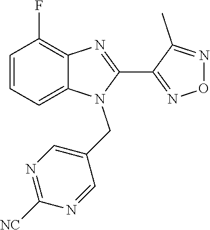 [SMILES: Cc1nonc1-c1nc2c(F)cccc2n1Cc1cnc(C#N)nc1]

Prepared as described for Example 38 using 5-(bromomethyl)pyrimidine-2-carbonitrile (42.00 mg, 0.212 mmol) and 3-(7-fluoro-benzimidazol-2-yl)-4-methyl-1,2,5-oxadiazole (Intermediate 1) (46.28 mg, 0.212 mmol) to give 5-[[4-fluoro-2-(4-methyl-1,2,5-oxadiazol-3-yl)benzimidazol-1-yl]methyl]pyrimidine-2-carbonitrile (11.45 mg, 0.034 mmol, 16.1% yield, 99.7% purity) as a yellow solid.

MS ES⁺: 336.3

¹H NMR (400 MHz, DMSO-d₆) 8.91 (s, 2H), 7.62 (d, J=8.1 Hz, 1H), 7.46-7.39 (m, 1H), 7.26-7.20 (m, 1H), 6.07 (s, 2H), 2.79 (s, 3H).

### Example 46: 6-[[7-fluoro-2-(4-methyl-1,2,5-oxadiazol-3-yl)benzimidazol-1-yl]methyl]pyridazine-3-carbonitrile

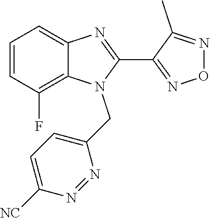 [SMILES: Cc1nonc1-c1nc2cccc(F)c2n1Cc1ccc(C#N)nn1]

Prepared as described for Example 38 using 6-(bromomethyl)pyridazine-3-carbonitrile (52 mg, 0.263 mmol) and 3-(7-fluoro-benzimidazol-2-yl)-4-methyl-1,2,5-oxadiazole (Intermediate 1) (57.30 mg, 0.263 mmol) to give 6-[[7-fluoro-2-(4-methyl-1,2,5-oxadiazol-3-yl)benzimidazol-1-yl]methyl]pyridazine-3-carbonitrile (1.77 mg, 0.005 mmol, 2.0% yield, 100% purity) as an off-white solid.

MS ES⁺: 336.3

¹H NMR (400 MHz, CDCl₃) 7.80 (d, J=8.8 Hz, 1H), 7.71 (d, J=8.4 Hz, 1H), 7.46 (d, J=8.8 Hz, 1H), 7.34-7.28 (m, 1H), 7.12-7.05 (m, 1H), 6.45 (s, 2H), 2.87 (s, 3H).

### Example 47: 6-[[4-fluoro-2-(4-methyl-1,2,5-oxadiazol-3-yl)benzimidazol-1-yl]methyl]pyridazine-3-carbonitrile

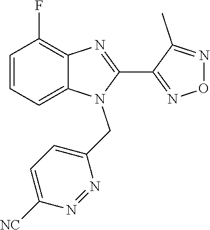 [SMILES: Cc1nonc1-c1nc2c(F)cccc2n1Cc1ccc(C#N)nn1]

Prepared as described for Example 38 using 6-(bromomethyl)pyridazine-3-carbonitrile (52 mg, 0.263 mmol) and 3-(7-fluoro-benzimidazol-2-yl)-4-methyl-1,2,5-oxadiazole (Intermediate 1) (57.30 mg, 0.263 mmol) to give 6-[[4-fluoro-2-(4-methyl-1,2,5-oxadiazol-3-yl)benzimidazol-1-yl]methyl]pyridazine-3-carbonitrile (10.61 mg, 0.032 mmol, 12.0% yield, 99.9% purity) as an off-white solid.

MS ES⁺: 336.2

¹H NMR (400 MHz, CDCl₃) 7.80 (d, J=8.8 Hz, 1H), 7.61 (d, J=8.8 Hz, 1H), 7.39-7.32 (m, 2H), 7.14-7.05 (m, 1H), 6.28 (s, 2H), 2.91 (s, 3H).

### Example 48: 3-[1-[(6-ethoxypyridin-3-yl)methyl]-4-fluoro-benzimidazol-2-yl]-4-methyl-1,2,5-oxadiazole

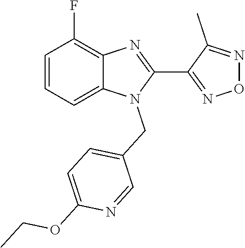 [SMILES: CCOc1ccc(Cn2c(-c3nonc3C)nc3c(F)cccc32)cn1]

Prepared as described for Example 38 using 5-(chloromethyl)-2-ethoxy-pyridine (80 mg, 0.466 mmol) (prepared as described for Journal of Medicinal Chemistry, 2000, vol. 43, no. 18, pages 3386-3399) and 3-(7-fluoro-benzimidazol-2-yl)-4-methyl-1,2,5-oxadiazole (Intermediate 1) (81.36 mg, 0.373 mmol) to give 3-[1-[(6-ethoxypyridin-3-yl)methyl]-4-fluoro-benzimidazol-2-yl]-4-methyl-1,2,5-oxadiazole (11.26 mg, 0.032 mmol, 6.8% yield, 99.0% purity) as a white powder.

MS ES⁺: 354.3

¹H NMR (400 MHz, DMSO-d₆) 8.16 (s, 1H), 7.59-7.65 (m, 1H), 7.52-7.58 (m, 1H), 7.35-7.45 (m, 1H), 7.15-7.24 (m, 1H), 6.69-6.75 (m, 1H), 5.85 (s, 2H), 4.18-4.29 (m, 2H), 2.77 (s, 3H), 1.22-1.31 (m, 3H).

### Example 49: 3-[7-fluoro-1-(pyrimidin-5-ylmethyl)benzimidazol-2-yl]-4-methyl-1,2,5-oxadiazole

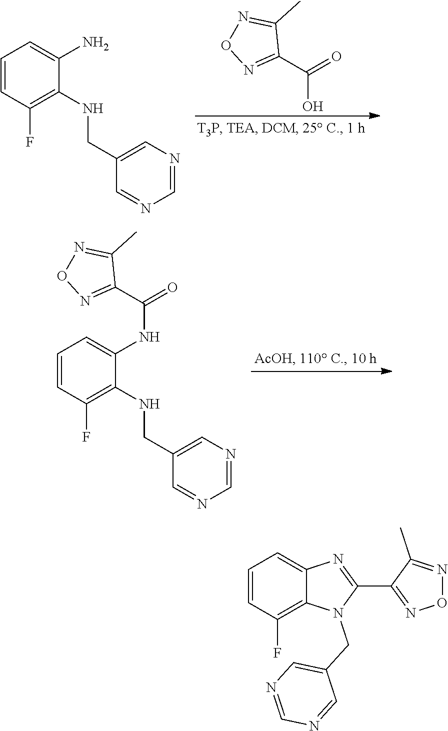

Step 1: A mixture of 4-methyl-1,2,5-oxadiazole-3-carboxylic acid (Intermediate 2) (46.95 mg, 0.367 mmol), 3-fluoro-N2-(pyrimidin-5-ylmethyl)benzene-1,2-diamine (80 mg, 0.367 mmol), TEA (111.28 mg, 1.10 mmol) and T₃P (174.96 mg, 0.550 mmol, 50% in ethyl acetate) in DCM (1 mL) was stirred at 25° C. for 1 hour. The mixture was extracted with ethyl acetate (5 mL×3). The combined organic phases were washed with brine (5 mL), dried over anhydrous Na₂SO₄, and concentrated in vacuum to dryness to give N-[3-fluoro-2-(pyrimidin-5-ylmethylamino)phenyl]-4-methyl-1,2,5-oxadiazole-3-carboxamide (85 mg, 0.259 mmol, 70.6% yield) as a yellow oil which was used for the next step without purification.

Step 2: A solution of N-[3-fluoro-2-(pyrimidin-5-ylmethylamino)phenyl]-4-methyl-1,2,5-oxadiazole-3-carboxamide (85 mg, 0.259 mmol) in AcOH (5 mL) was stirred at 110° C. for 10 hours. Then the reaction mixture was cooled to RT and reduced under vacuum. The residue was dissolved in DMF (3 mL) and filtered to remove the insoluble.

The filtrate was concentrated in vacuo. The residue was purified by prep. HPLC (Column: Phenomenex Luna C₁₈ 75*30 mm*3 μm, Mobile Phase A: water (0.225% FA), Mobile Phase B: acetonitrile, Flow rate: 30 mL/min, gradient condition from 21% B to 51%). The pure fractions were collected and the volatiles were removed under vacuum.

The residue was partitioned between acetonitrile (2 mL) and water (10 mL). The solution was lyophilized to dryness to give 3-[7-fluoro-1-(pyrimidin-5-ylmethyl)benzimidazol-2-yl]-4-methyl-1,2,5-oxadiazole (15.65 mg, 0.048 mmol, 18.7% yield, 96.0% purity) as a white powder.

MS ES⁺: 311.3

¹H NMR (400 MHz, DMSO-d₆) 9.13 (s, 1H), 8.71 (s, 2H), 7.74 (d, J=70.50 Hz, 1H), 7.21-7.37 (m, 2H), 5.99 (s, 2H), 2.78 (s, 3H).

### Example 50: 5-[[2-(4-methyl-1,2,5-oxadiazol-3-yl)imidazo[4,5-b]pyridin-3-yl]methyl]pyridine-2-carbonitrile

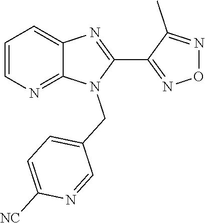 [SMILES: Cc1nonc1-c1nc2cccnc2n1Cc1ccc(C#N)nc1]

Prepared as described for Example 24 using 2-fluoro-3-nitro-pyridine (500 mg, 3.52 mmol) and 5-(aminomethyl)pyridine-2-carbonitrile hydrochloride (prepared as described for Journal of Medicinal Chemistry, 2003, vol. 46, no. 17, pages 3612-3622) (562.26 mg, 4.22 mmol) to give 5-[[2-(4-methyl-1,2,5-oxadiazol-3-yl)imidazo[4,5-b]pyridin-3-yl]methyl]pyridine-2-carbonitrile (21.85 mg, 0.066 mmol, 1.9% yield, 95.2% purity) as a white solid.

MS ES⁺: 318.1

¹H NMR (400 MHz, DMSO-d₆) 8.77 (d, J=1.6 Hz, 1H), 8.55 (dd, J=1.2, 4.8 Hz, 1H), 8.37 (dd, J=1.2, 8.4 Hz, 1H), 7.95 (d, J=8.0 Hz, 1H), 7.83 (dd, J=2.4, 8.0 Hz, 1H), 7.49 (dd, J=4.8, 8.0 Hz, 1H), 6.00 (s, 2H), 2.77 (s, 3H).

### Example 51: 5-[[2-(4-amino-1,2,5-oxadiazol-3-yl)imidazo[4,5-b]pyridin-3-yl]methyl]pyridine-2-carbonitrile

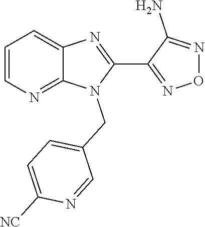 [SMILES: N#Cc1ccc(Cn2c(-c3nonc3N)nc3cccnc32)cn1]

Prepared as described for Example 23 using 2-fluoro-3-nitro-pyridine (200 mg, 1.41 mmol) and 5-(aminomethyl)pyridine-2-carbonitrile hydrochloride (prepared as described for Journal of Medicinal Chemistry, 2003, vol. 46, no. 17, pages 3612-3622) (238.74 mg, 1.41 mmol) to give 5-[[2-(4-amino-1,2,5-oxadiazol-3-yl)imidazo[4,5-b]pyridin-3-yl]methyl]pyridine-2-carbonitrile (2.79 mg, 0.009 mmol, 0.6% yield, 98.9% purity) as an off-white solid.

MS ES⁺: 319.0

¹H NMR (400 MHz, DMSO-d₆) 8.78 (d, J=1.6 Hz, 1H), 8.56 (dd, J=1.4, 4.8 Hz, 1H), 8.35 (dd, J=1.4, 8.1 Hz, 1H), 7.95 (d, J=8.1 Hz, 1H), 7.82 (dd, J=2.1, 8.1 Hz, 1H), 7.51 (dd, J=4.8, 8.1 Hz, 1H), 6.97 (s, 2H), 6.03 (s, 2H).

### Example 52: 5-[[7-fluoro-2-(4-methyl-1,2,5-oxadiazol-3-yl)benzimidazol-1-yl]methyl]pyridine-2-carbonitrile

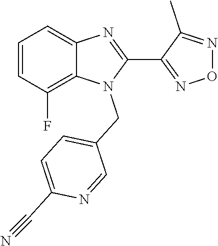 [SMILES: Cc1nonc1-c1nc2cccc(F)c2n1Cc1ccc(C#N)nc1]

Prepared as described for Example 49 using 1,2-difluoro-3-nitro-benzene (562.78 mg, 3.54 mmol) and 5-(aminomethyl)pyridine-2-carbonitrile hydrochloride (600 mg, 3.54 mmol) to give 5-[[7-fluoro-2-(4-methyl-1,2,5-oxadiazol-3-yl)benzimidazol-1-yl]methyl]pyridine-2-carbonitrile (21.2 mg, 0.062 mmol, 1.8% yield, 98.3% purity) as an off-white solid.

MS ES⁺: 334.9

¹H NMR (400 MHz, DMSO-d₆) 8.75-8.68 (m, 1H), 8.03-7.96 (m, 1H), 7.80-7.72 (m, 2H), 7.37 (dt, J=5.1, 8.1 Hz, 1H), 7.28-7.22 (m, 1H), 6.07 (s, 2H), 2.78 (s, 3H).

### Example 53: 3-[1-[(6-methoxypyridin-3-yl)methyl]benzimidazol-2-yl]-4-methyl-1,2,5-oxadiazole

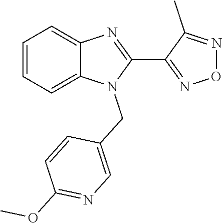 [SMILES: COc1ccc(Cn2c(-c3nonc3C)nc3ccccc32)cn1]

Prepared as described for Example 37 using 3-(1H-benzimidazol-2-yl)-4-methyl-1,2,5-oxadiazole (100 mg, 0.500 mmol) and 5-(chloromethyl)-2-methoxy-pyridine (78.72 mg, 0.500 mmol) to give 3-[l-[(6-methoxypyridin-3-yl)methyl]benzimidazol-2-yl]-4-methyl-1,2,5-oxadiazole (20 mg, 0.060 mmol, 12.0% yield, 96.3% purity) as a white powder.

MS ES⁺: 322.2

¹H NMR (400 MHz, DMSO-d₆) 8.16 (s, 1H), 7.70-7.93 (m, 2H), 7.29-7.60 (m, 3H), 6.74 (d, J=7.60 Hz, 1H), 5.85 (s, 2H), 3.79 (s, 3H), 2.77 (s, 3H).

### Example 54: 5-[[2-(4-methyl-1,2,5-oxadiazol-3-yl)benzimidazol-1-yl]methyl]pyridin-2-ol

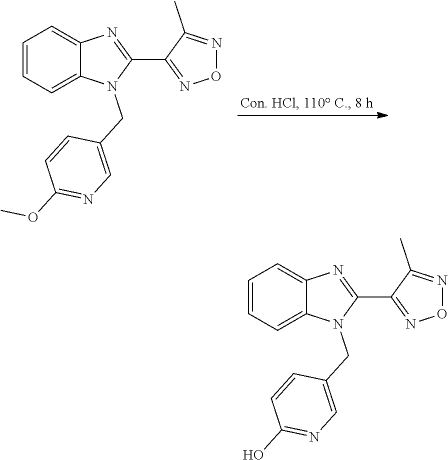 [SMILES: COc1ccc(Cn2c(-c3nonc3C)nc3ccccc32)cn1 ; Cc1nonc1-c1nc2ccccc2n1Cc1ccc(O)nc1]

A solution of 3-[l-[(6-methoxypyridin-3-yl)methyl]benzimidazol-2-yl]-4-methyl-1,2,5-oxadiazole (Example 53) (15 mg, 0.047 mmol) in conc. HCl (5 mL) was stirred at 110° C. for 8 hours. The mixture was evaporated to dryness. The residue was purified by prep. HPLC (Column: Phenomenex Luna C₁₈ 100*40 mm*3 μm, Mobile Phase A: water (0.225% FA), Mobile Phase B: acetonitrile, Flow rate: 25 mL/min, gradient condition from 16% B to 56%). The pure fractions were collected and the volatiles were removed under vacuum. The residue was partitioned between acetonitrile (2 mL) and water (10 mL). The solution was lyophilized to dryness to give 5-[[2-(4-methyl-1,2,5-oxadiazol-3-yl)benzimidazol-1-yl]methyl]pyridin-2-ol (14 mg, 0.046 mmol, 97.6% yield, 100% purity) as white solid.

MS ES⁺: 307.9

### Example 55: 5-[[6-fluoro-2-(4-methyl-1,2,5-oxadiazol-3-yl)benzimidazol-1-yl]methyl]pyridine-2-carbonitrile

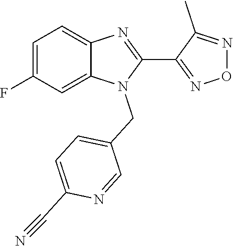 [SMILES: Cc1nonc1-c1nc2ccc(F)cc2n1Cc1ccc(C#N)nc1]

Prepared as described for Example 49 using 2,4-difluoro-1-nitro-benzene (1 g, 6.29 mmol) and 5-(aminomethyl)pyridine-2-carbonitrile (1.07 g, 6.29 mmol) to give 5-[[6-fluoro-2-(4-methyl-1,2,5-oxadiazol-3-yl)benzimidazol-1-yl]methyl]pyridine-2-carbonitrile (29.73 mg, 0.089 mmol, 1.4% yield, 99.8% purity) as a white powder.

MS ES⁺: 335.3

¹H NMR (400 MHz, DMSO-d₆) 8.72 (s, 1H), 7.88-8.01 (m, 2H), 7.63-7.78 (m, 2H), 7.27 (s, 1H), 6.02 (s, 2H), 2.77 (s, 3H).

### Example 56: 3-[6,7-difluoro-1-(pyridin-3-ylmethyl)benzimidazol-2-yl]-4-methyl-1,2,5-oxadiazole

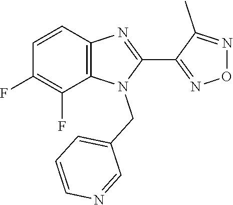 [SMILES: Cc1nonc1-c1nc2ccc(F)c(F)c2n1Cc1cccnc1]

Prepared as described for Example 49 using 1,2,3-trifluoro-4-nitro-benzene (2 g, 11.29 mmol) and pyridin-3-ylmethanamine (1.22 g, 11.29 mmol) to give 3-[6,7-difluoro-1-(pyridin-3-ylmethyl)benzimidazol-2-yl]-4-methyl-1,2,5-oxadiazole (19.83 mg, 0.059 mmol, 0.5% yield, 97.5% purity) as an off-white powder.

MS ES⁺: 327.9

¹H NMR (400 MHz, DMSO-d₆) 8.53-8.41 (m, 2H), 7.78-7.72 (m, 1H), 7.59-7.53 (m, 1H), 7.49-7.40 (m, 1H), 7.38-7.32 (m, 1H), 5.98 (s, 2H), 2.76 (s, 3H).

### Example 57: 3-methyl-4-[1-(pyrimidin-5-ylmethyl)benzimidazol-2-yl]-1,2,5-oxadiazole

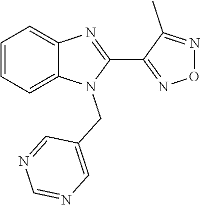 [SMILES: Cc1nonc1-c1nc2ccccc2n1Cc1cncnc1]

Prepared as described for Example 37 using pyrimidin-5-ylmethyl 4-methylbenzenesulfonate (13.20 mg, 0.050 mmol) (prepared as described for WO2009/45381) and 3-(1H-benzimidazol-2-yl)-4-methyl-1,2,5-oxadiazole (10 mg, 0.050 mmol) to give 3-methyl-4-[1-(pyrimidin-5-ylmethyl)benzimidazol-2-yl]-1,2,5-oxadiazole (9.47 mg, 0.030 mmol, 60.6% yield, 93.4% purity) as a white powder.

MS ES⁺: 293.2

### Example 58: 3-methyl-4-[1-[(6-(methylsulfonyl)pyridin-3-yl)methyl]benzimidazol-2-yl]-,2,5-oxadiazole

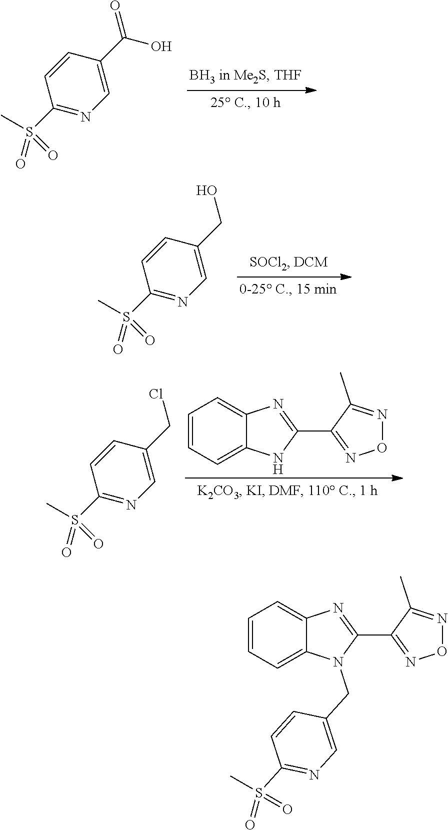

Step 1: To a mixture of 6-(methylsulfonyl)pyridine-3-carboxylic acid (500 mg, 2.49 mmol) in THF (5 mL) was added BH₃ in Me₂S (10M, 1.24 mL, 5 eq) dropwise at 0° C. under N₂. The mixture was warmed to 25° C. and stirred for 10 hours. MeOH (10 mL) was added dropwise slowly at ° C. to quench the reaction. The mixture was stirred at 25° C. for 30 min. The mixture was concentrated in vacuo to afford (6-(methylsulfonyl)pyridin-3-yl)methanol (300 mg, 1.60 mmol, 64.5% yield) as a yellow oil which was used for the next step without further purification.

Step 2: To a solution of (6-(methylsulfonyl)pyridin-3-yl)methanol (300 mg, 1.60 mmol) in DCM (1.5 mL) was added SOCl₂ (953.21 mg, 8.01 mmol) at ° C. The mixture was stirred at 0-25° C. for 15 min. Then the mixture was concentrated in vacuum to afford 5-(chloromethyl)-2-(methylsulfonyl)-pyridine (200 mg, 0.486 mmol, 30.3% yield, 50% purity) as a yellow liquid which was used for the next step without purification.

MS ES⁺: 206.1

Step 3: A mixture of 3-(1H-benzimidazol-2-yl)-4-methyl-1,2,5-oxadiazole (20 mg, 0.100 mmol), 5-(chloromethyl)-2-(methylsulfonyl)-pyridine (20.55 mg, 0.100 mmol), K₂CO₃ (27.61 mg, 0.200 mmol) and KI (1.66 mg, 0.010 mmol) in DMF (1 mL) was stirred at 110° C. for 1 hour. The resulting mixture was cooled down to RT. Then the mixture was dissolved in DMF (3 mL) and filtered to remove the insoluble. The filter liquor was concentrated in vacuo. The residue was purified by prep. HPLC (Column: Phenomenex Gemini-NX C₁₈ 75*30 mm*3 μm, Mobile Phase A: water (0.05% NH₃·H₂O+10 mM NH₄HCO₃), Mobile Phase B: acetonitrile, Flow rate: 14 mL/min, gradient condition from 17% B to 67%). The pure fractions were collected and the volatiles were removed under vacuum. The residue was partitioned between acetonitrile (2 mL) and water (10 mL). The solution was lyophilized to dryness to give 3-methyl-4-[1-[(6-(methylsulfonyl)pyridin-3-yl)methyl]benzimidazol-2-yl]-1,2,5-oxadiazole (13.64 mg, 0.036 mmol, 36.3% yield, 98.1% purity) as a white powder.

MS ES⁺: 370.1

¹H NMR (400 MHz, DMSO-d₆) 8.73-8.76 (m, 1H), 7.95-7.99 (m, 1H), 7.89-7.93 (m, 1H), 7.74-7.80 (m, 2H), 7.38-7.44 (m, 2H), 6.08 (s, 2H), 3.26 (s, 3H), 2.78-2.82 (m, 3H).

### Example 59: 4-[3-(pyridin-3-ylmethyl)imidazo[4,5-b]pyridin-2-yl]-1,2,5-oxadiazol-3-amine

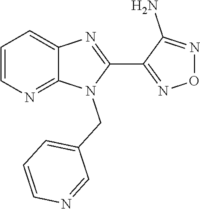 [SMILES: Nc1nonc1-c1nc2cccnc2n1Cc1cccnc1]

Prepared as described for Example 23 using 2-fluoro-3-nitro-pyridine (300 mg, 2.11 mmol) and pyridin-3-ylmethanamine (228.33 mg, 2.11 mmol) to give 4-[3-(pyridin-3-ylmethyl)imidazo[4,5-b]pyridin-2-yl]-1,2,5-oxadiazol-3-amine (23.46 mg, 0.080 mmol, 3.8% yield, 99.8% purity) as a yellow solid.

MS ES⁺: 294.1

¹H NMR (400 MHz, DMSO-d₆) 8.59-8.54 (m, 2H), 8.49-8.44 (m, 1H), 8.36-8.30 (m, 1H), 7.64-7.59 (m, 1H), 7.52-7.47 (m, 1H), 7.34-7.30 (m, 1H), 6.98 (s, 2H), 5.95 (s, 2H).

### Example 60: 3-[1-[(6-chloropyridin-3-yl)methyl]benzimidazol-2-yl]-4-methyl-1,2,5-oxadiazole

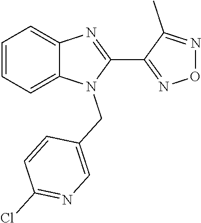 [SMILES: Cc1nonc1-c1nc2ccccc2n1Cc1ccc(Cl)nc1]

Prepared as described for Example 37 using 2-chloro-5-(chloromethyl)pyridine (16.19 mg, 0.100 mmol) and 3-(1H-benzimidazol-2-yl)-4-methyl-1,2,5-oxadiazole (20 mg, 0.100 mmol) to give 3-[1-[(6-chloropyridin-3-yl)methyl]benzimidazol-2-yl]-4-methyl-1,2,5-oxadiazole (18.14 mg, 0.055 mmol, 54.9% yield, 98.5% purity) as a white powder.

MS ES⁺: 326.2

### Example 61: 5-[[4-chloro-2-(4-methyl-1,2,5-oxadiazol-3-yl)benzimidazol-1-yl]methyl]pyridine-2-carbonitrile

### Example 62: 5-[[7-chloro-2-(4-methyl-1,2,5-oxadiazol-3-yl)benzimidazol-1-yl]methyl]pyridine-2-carbonitrile

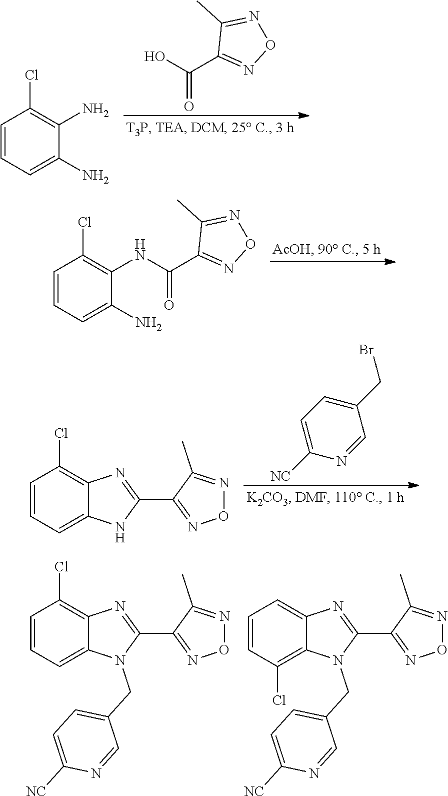

Step 1: To a mixture of 3-chlorobenzene-1,2-diamine (500 mg, 3.51 mmol), 4-methyl-1,2,5-oxadiazole-3-carboxylic acid (Intermediate 2) (359.32 mg, 2.81 mmol) and TEA (1.06 g, 10.52 mmol) in DCM (5 mL) was added dropwise T₃P (4.46 g, 7.01 mmol, 4.17 mL, 50% purity in ethyl acetate) at ° C. Then the mixture was stirred at 25° C. for 3 hours. The mixture was poured into water (10 mL) and extracted with DCM (10 mL×3).

The combined organic layers were concentrated to afford N-(2-amino-6-chloro-phenyl)-4-methyl-1,2,5-oxadiazole-3-carboxamide (800 mg, 3.17 mmol, 90.3% yield) as a black solid which was used for the next step without further purification.

MS ES⁺: 253.1

Step 2: N-(2-amino-6-chloro-phenyl)-4-methyl-1,2,5-oxadiazole-3-carboxamide (800 mg, 3.17 mmol) was added into AcOH (5 mL) in one portion at 25° C. The mixture was stirred at 90° C. for 5 hours. The mixture was concentrated, and then adjusted to pH=8 with sat. NaHCO₃ (aq.) (10 mL) and extracted with ethyl acetate (10 mL×3). The combined organic layers were dried with Na₂SO₄ and concentrated to afford the crude product which was purified by flash column chromatography (ISCO®; 12 g SepaFlash® Silica Flash Column, Eluent of 0-15% ethyl acetate/petroleum ether gradient @ 30 mL/min) to give 3-(4-chloro-1H-benzimidazol-2-yl)-4-methyl-1,2,5-oxadiazole (470 mg, 1.83 mmol, 57.7% yield, 91.2% purity) as a white solid.

MS ES⁺: 235.2

Step 3: To a mixture of 3-(4-chloro-1H-benzimidazol-2-yl)-4-methyl-1,2,5-oxadiazole (200 mg, 0.852 mmol) and 5-(bromomethyl)pyridine-2-carbonitrile (167.94 mg, 0.852 mmol) in DMF (5 mL) was added K₂CO₃ (235.60 mg, 1.70 mmol) in one portion at 25° C. Then the mixture was heated to 110° C. and stirred for 1 hour. The mixture was cooled to RT and filtered. Then the filtrate was purified by prep. HPLC (Column: Phenomenex Gemini-NX C₁₈ 75*30 mm*3 μm, Mobile Phase A: water (10 mM NH₄HCO₃), Mobile Phase B: MeCN, Flow rate: 25 mL/min, gradient condition from 33% B to 73%). The pure fractions were collected and the volatiles were removed under vacuum. The residue was partitioned between acetonitrile (2 mL) and water (10 mL). The solution was lyophilized to dryness to give Peak 1 (5-[[4-chloro-2-(4-methyl-1,2,5-oxadiazol-3-yl)benzimidazol-1-yl]methyl]pyridine-2-carbonitrile) (49-53 mg, 0.139 mmol, 98.7% yield, 98.7% purity) as a brown powder and Peak 2 (5-[[7-chloro-2-(4-methyl-1,2,5-oxadiazol-3-yl)benzimidazol-1-yl]methyl]pyridine-2-carbonitrile) (8.19 mg, 0.023 mmol, 98.6% yield, 98.6% purity) as a brown powder.

### Example 61 (Peak 1)

MS ES⁺: 351.2

¹H NMR (400 MHz, DMSO-d₆) 8.75 (d, J=1.8 Hz, 1H), 7.96 (d, J=8.2 Hz, 1H), 7.73 (d, J=8.2 Hz, 2H), 7.54-7.48 (m, 1H), 7.47-7.33 (m, 1H), 6.07 (s, 2H), 2.82 (s, 3H).

### Example 62 (Peak 2)

MS ES⁺: 351.2

¹H NMR (400 MHz, DMSO-d₆) 8.67 (d, J=1.6 Hz, 1H), 7.98 (d, J=8.0 Hz, 1H), 7.92 (d, J=8.0 Hz, 1H), 7.72-7.64 (m, 1H), 7.50-7.45 (m, 1H), 7.44-7.37 (m, 1H), 6.24 (s, 2H), 2.77 (s, 3H).

### Example 63: 3-methyl-4-[1-[(6-methylpyridin-3-yl)methyl]benzimidazol-2-yl]-1,2,5-oxadiazole

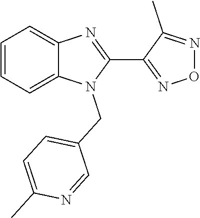 [SMILES: Cc1ccc(Cn2c(-c3nonc3C)nc3ccccc32)cn1]

Prepared as described for Example 37 using 3-(1H-benzimidazol-2-yl)-4-methyl-1,2,5-oxadiazole (10 mg, 0.050 mmol) and (6-methylpyridin-3-yl)methyl 4-methylbenzenesulfonate (13.85 mg, 0.050 mmol) to give 3-methyl-4-[1-[(6-methylpyridin-3-yl)methyl]benzimidazol-2-yl]-1,2,5-oxadiazole (3.76 mg, 0.012 mmol, 24.4% yield, 99.0% purity) as a white powder.

MS ES⁺: 306.1

### Example 64: 3-methyl-4-[1-[(2-methylpyrimidin-5-yl)methyl]benzimidazol-2-yl]-1,2,5-oxadiazole

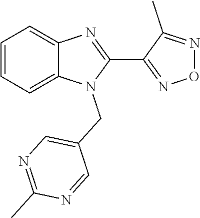 [SMILES: Cc1ncc(Cn2c(-c3nonc3C)nc3ccccc32)cn1]

Prepared as described for Example 37 using 3-(1H-benzimidazol-2-yl)-4-methyl-1,2,5-oxadiazole (10 mg, 0.050 mmol) and (2-methylpyrimidin-5-yl)methyl 4-methylbenzenesulfonate (13.90 mg, 0.050 mmol) to give 3-methyl-4-[1-[(2-methylpyrimidin-5-yl)methyl]benzimidazol-2-yl]-1,2,5-oxadiazole (10.24 mg, 0.033 mmol, 66.7% yield, 99.7% purity) as a white powder.

MS ES⁺: 307.3

### Example 65: 3-[4,7-difluoro-1-(pyridin-3-ylmethyl)benzimidazol-2-yl]-4-methyl-1,2,5-oxadiazole

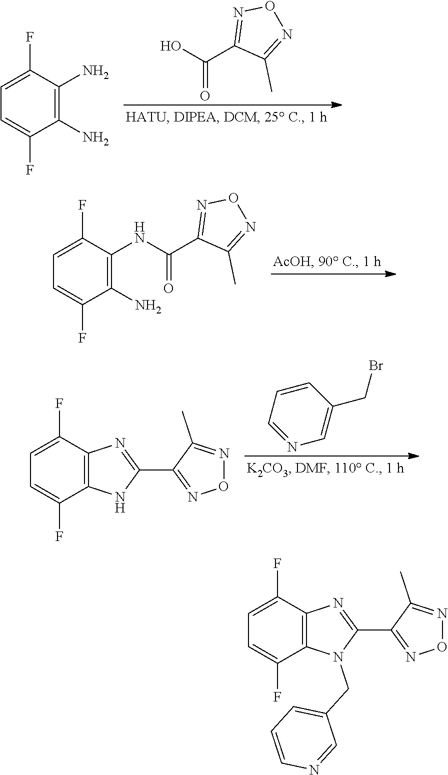

Step 1: To a mixture of 3,6-difluorobenzene-1,2-diamine (280 mg, 1.94 mmol) and 4-methyl-1,2,5-oxadiazole-3-carboxylic acid (Intermediate 2) (248.85 mg, 1.94 mmol) in DCM (4 mL) was added DIPEA (753.28 mg, 5.83 mmol) and HATU (1.48 g, 3.89 mmol) at 25° C. The mixture was stirred at 25° C. for 1 hour. Then the mixture was poured into water (15 mL) and extracted with DCM (15 mL×3). The combined organic layers were dried over Na₂SO₄ and filtered. The filtrate was concentrated to afford N-(2-amino-3,6-difluoro-phenyl)-4-methyl-1,2,5-oxadiazole-3-carboxamide (450 mg, 1.77 mmol, 91.1% yield) as a black solid which was used for the next step directly.

Step 2: N-(2-amino-3,6-difluoro-phenyl)-4-methyl-1,2,5-oxadiazole-3-carboxamide (450 mg, 1.77 mmol) was added into AcOH (4 mL) in one portion at 25° C. The mixture was stirred at 90° C. for 1 hour. Then the mixture was adjusted to pH=9 with sat. NaHCO₃ (aq.) (10 mL), then extracted with ethyl acetate (10 mL×3). The combined organic layers were dried with anhydrous Na₂SO₄ and concentrated to dryness. The residue was purified by flash silica gel chromatography (ISCO®; 4 g SepaFlash® Silica Flash Column, eluent of petroleum ether: ethyl acetate=3:1 @ 35 mL/min) to give 3-(4,7-difluoro-1H-benzimidazol-2-yl)-4-methyl-1,2,5-oxadiazole (50 mg, 0.190 mmol, 10.7% yield, 89.8% purity) as a white solid.

MS ES⁺: 237.0

Step 3: A mixture of 3-(4,7-difluoro-1H-benzimidazol-2-yl)-4-methyl-1,2,5-oxadiazole (28 mg, 0.119 mmol), 3-(bromomethyl)pyridine (20.39 mg, 0.119 mmol) and K₂CO₃ (32.7, 0.237 mmol) in DMF (3 mL) was stirred at 110° C. for 1 hour. The resulting mixture was cooled to RT and filtered to remove the insoluble. The filter liquor was concentrated in vacuo. The crude product was purified by prep. HPLC (Column: Phenomenex Gemini-NX C₁₈ 75*30 mm*3 μm, Mobile Phase A: water (0.04% NH₃·H₂O +10 mM NH₄HCO₃), Mobile Phase B: acetonitrile, Flow rate: 25 mL/min, gradient condition from 34% B to 84%). The pure fractions were collected and the volatiles were removed under vacuum. The residue was partitioned between acetonitrile (2 mL) and water (1 mL). The solution was lyophilized to dryness to give 3-[4,7-difluoro-1-(pyridin-3-ylmethyl)benzimidazol-2-yl]-4-methyl-1,2,5-oxadiazole (22 mg, 0.067 mmol, 56.3% yield, 99.2% purity) as a white powder.

MS ES⁺: 328.3

¹H NMR (400 MHz, DMSO-d₆) 8.54-8.43 (m, 2H), 7.58-7.51 (m, 1H), 7.35 (d, J=7.6 Hz, 1H), 7.30-7.16 (m, 2H), 5.98 (s, 2H), 2.78 (s, 3H).

### Example 66: 3-[1-[(2-methoxypyridin-4-yl)methyl]benzimidazol-2-yl]-4-methyl-1,2,5-oxadiazole

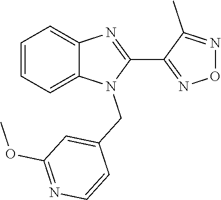 [SMILES: COc1cc(Cn2c(-c3nonc3C)nc3ccccc32)ccn1]

Prepared as described for Example 37 using (2-methoxypyridin-4-yl)methyl 4-methylbenzenesulfonate (14.65 mg, 0.050 mmol) and 3-(1H-benzimidazol-2-yl)-4-methyl-1,2,5-oxadiazole (10 mg, 0.050 mmol) to give 3-[1-[(2-methoxypyridin-4-yl)methyl]benzimidazol-2-yl]-4-methyl-1,2,5-oxadiazole (9.7 mg, 0.030 mmol, 60.4% yield, 100% purity) as a white powder.

MS ES⁺: 322.3

### Example 67: 3-[[2-(4-methyl-1,2,5-oxadiazol-3-yl)benzimidazol-1-yl]methyl]pyridine-2-carbonitrile

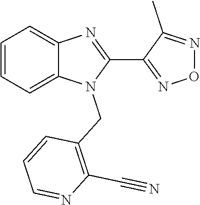 [SMILES: Cc1nonc1-c1nc2ccccc2n1Cc1cccnc1C#N]

Prepared as described for Example 37 using 3-(bromoethyl)pyridine-2-carbonitrile (19.68 mg, 0.100 mmol) and 3-(1H-benzimidazol-2-yl)-4-methyl-1,2,5-oxadiazole (20 mg, 0.100 mmol) to give 3-[[2-(4-methyl-1,2,5-oxadiazol-3-yl)benzimidazol-1-yl]methyl]pyridine-2-carbonitrile (21.34 mg, 0.067 mmol, 67.3% yield, 99.6% purity) as a white powder.

MS ES⁺: 317.2

### Example 68: 5-[7-fluoro-1-(pyridin-3-ylmethyl)benzimidazol-2-yl]-1,2,3-thiadiazole

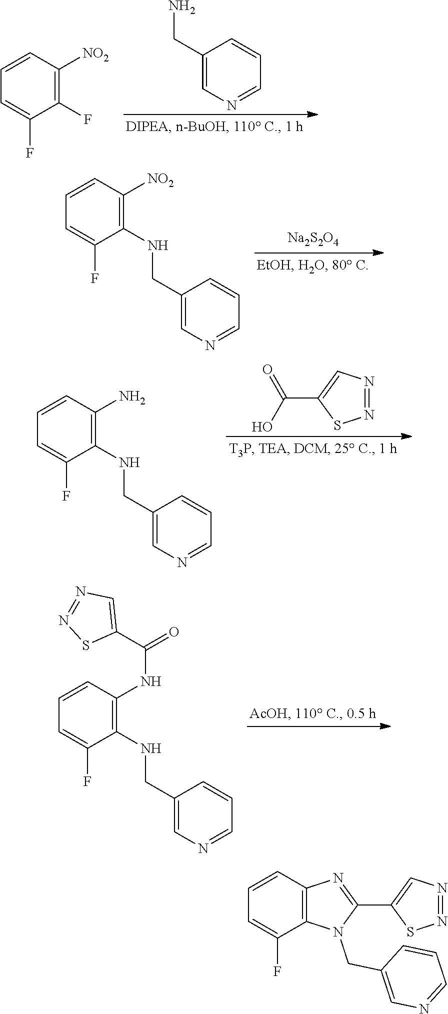

Step 1: A solution of 1,2-difluoro-3-nitro-benzene (10 g, 62.86 mmol), pyridin-3-ylmethanamine (6.80 g, 62.86 mmol) and DIPEA (16.25 g, 125.72 mmol) in n-BuOH (50 mL) was stirred at 110° C. for 1 hour. The mixture was cooled down to RT and a yellow precipitation formed. The precipitation was collected and washed with EtOH (20 mL) to give 2-fluoro-6-nitro-N-(pyridin-3-ylmethyl)aniline (10 g, 36.40 mmol, 57.9% yield, 90% purity) as a yellow solid which was used for the next step directly.

MS ES⁺: 248.1

¹H NMR (400 MHz, DMSO-d₆) 8.53 (d, J=1.4 Hz, 1H), 8.44 (dd, J=1.4, 4.8 Hz, 1H), 8.16 (t, J=50.6 Hz, 1H), 7.93-7.79 (m, 1H), 7.72 (d, J=7.9 Hz, 1H), 7.49-7.30 (m, 2H), 6.72 (dt, J=40.8, 8.3 Hz, 1H), 4.71 (dd, J=40.4, 6.5 Hz, 2H).

Step 2: A solution of 2-fluoro-6-nitro-N-(pyridin-3-ylmethyl)aniline (2 g, 8.09 mmol) in EtOH (15 mL) was heated to 80° C. and stirred for 0.5 hour. Then a solution of sodium hydrosulfite (7.04 g, 40.45 mmol) in water (20 mL) was added into the reaction mixture and stirred until the mixture turned from yellow to colourless. Then the mixture was cooled down to RT and extracted with DCM (40 mL×2). The combined organic layers were washed with brine (40 mL×2), dried over Na₂SO₄ and filtered. The filtrate was evaporated to dryness to give 3-fluoro-N2-(pyridin-3-ylmethyl)benzene-1,2-diamine (0.92 g, 4.23 mmol, 52.4% yield) as a yellow oil which was used for the next step without further purification.

Step 3: To a solution of 3-fluoro-N2-(pyridin-3-ylmethyl)benzene-1,2-diamine (50.08 mg, 0.231 mmol), thiadiazole-5-carboxylic acid (30 mg, 0.231 mmol) and TEA (69.99 mg, 0.692 mmol) in DCM (1 mL) was added T₃P (220.07 mg, 0.346 mmol, 50% purity in ethyl acetate) at ° C. Then the mixture was stirred at 25° C. for 1 hour. The reaction mixture was diluted with DCM (10 mL) and washed with H₂O (5 mL×2). The separated organic layer was dried over Na₂SO₄, filtered and concentrated under reduced pressure to give N-[3-fluoro-2-(pyridin-3-ylmethylamino)phenyl]-1,2,3-thiadiazole-5-carboxamide (76 mg) as a yellow solid which used into the next step without further purification.

Step 4: A solution of N-[3-fluoro-2-(pyridin-3-ylmethylamino)phenyl]-1,2,3-thiadiazole-5-carboxamide (76 mg, 0.231 mmol) and AcOH (1 mL) was stirred at 110° C. for 0.5 hour. The reaction mixture was concentrated under reduced pressure to give a residue which was purified by prep. HPLC (Column: Phenomenex Luna C₁₈ 100*40 mm*3 μm, Mobile Phase A: water (0.225% FA), Mobile Phase B: acetonitrile, Flow rate: 25 mL/min, gradient condition from 0% B to 40%) to give 5-[7-fluoro-1-(pyridin-3-ylmethyl)benzimidazol-2-yl]-1,2,3-thiadiazole (7.12 mg, 0.023 mmol, 9.8% yield, 98.6% purity) as an off-white solid.

MS ES⁺: 312.0

¹H NMR (400 MHz, DMSO-d₆) 9.50 (s, 1H), 8.51-8.47 (m, 1H), 8.44-8.40 (m, 1H), 7.69-7.64 (m, 1H), 7.45-7.40 (m, 1H), 7.36-7.30 (m, 2H), 7.25-7.18 (m, 1H), 5.91 (s, 2H).

### Example 69: 3-methyl-4-[1-[(3-methylpyridin-2-yl)methyl]benzimidazol-2-yl]-1,2,5-oxadiazole

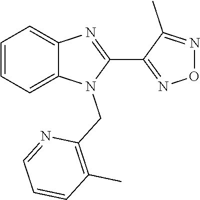 [SMILES: Cc1cccnc1Cn1c(-c2nonc2C)nc2ccccc21]

Prepared as described for Example 37 using 2-(chloromethyl)-3-methyl-pyridine (14.15 mg, 0.100 mmol) and 3-(1H-benzimidazol-2-yl)-4-methyl-1,2,5-oxadiazole (20 mg, 0.100 mmol) to give 3-methyl-4-[1-[(3-methylpyridin-2-yl)methyl]benzimidazol-2-yl]-1,2,5-oxadiazole (2.93 mg, 0.009 mmol, 14.6% yield, 99.6% purity) as a white powder.

MS ES⁺: 306.3

### Example 70: 3-[7-ethoxy-1-(pyridin-4-ylmethyl)benzimidazol-2-yl]-4-methyl-1,2,5-oxadiazole

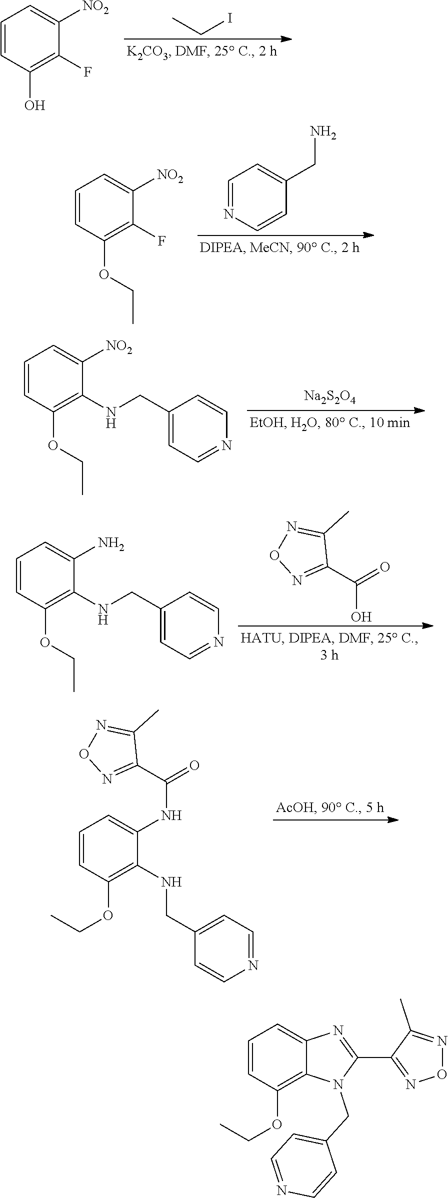

Step 1: To a mixture of 2-fluoro-3-nitro-phenol (200 mg, 1.27 mmol) and K₂CO₃ (351.90 mg, 2.55 mmol) in DMF (2 mL) was added dropwise iodoethane (397.11 mg, 2.55 mmol) at 25° C. The mixture was stirred at 25° C. for 2 hours. The mixture was poured into water (10 mL) and extracted with ethyl acetate (10 mL×3). The combined organic phases were washed with brine (10 mL×3), dried with anhydrous Na₂SO₄, filtered and concentrated in vacuum to afford 1-ethoxy-2-fluoro-3-nitro-benzene (250 mg, 1.08 mmol, 84.9% yield, 80% purity) as a brown oil which was used in the next step without further purification.

¹H NMR (400 MHz, DMSO-d₆) 7.68-7.52 (m, 2H), 7.34 (dt, J=1.9, 8.4 Hz, 1H), 4.20 (q, J=70.0 Hz, 2H), 1.37 (t, J=7.0 Hz, 3H).

Step 2: To a mixture of 1-ethoxy-2-fluoro-3-nitro-benzene (250 mg, 1.35 mmol) and pyridin-4-ylmethanamine (219.02 mg, 2.03 mmol) in MeCN (3 mL) was added DIPEA (523.53 mg, 4.05 mmol) in one portion at 25° C. The mixture was stirred at 90° C. for 2 hours. The mixture was concentrated to afford the crude product which was purified by flash column chromatography (ISCO®; 4 g SepaFlash® Silica Flash Column, eluent of 030% ethyl acetate/petroleum ether gradient @ 20 mL/min) to give 2-ethoxy-6-nitro-N-(pyridin-4-ylmethyl)aniline (250 mg, 0.869 mmol, 64.4% yield, 95% purity) as a yellow solid.

MS ES⁺: 274.0

¹H NMR (400 MHz, DMSO-d₆) 8.55-8.38 (m, 2H), 7.94 (t, J=6.8 Hz, 1H), 7.57 (dd, J=1.3, 8.6 Hz, 1H), 7.23 (d, J=5.8 Hz, 2H), 7.11 (d, J=8.0 Hz, 1H), 6.72 (t, J=8.3 Hz, 1H), 4.75 (d, J=6.9 Hz, 2H), 3.94 (q, J=6.9 Hz, 2H), 1.15 (t, J=6.9 Hz, 3H).

Step 3: A mixture of Na₂S₂O₄ (796.36 mg, 4.57 mmol) in H₂O (1 mL) was added into a mixture of 2-ethoxy-6-nitro-N-(pyridin-4-ylmethyl)aniline (250 mg, 0.915 mmol) in EtOH (2 mL) at 8W° C. The mixture was stirred at 80° C. for 10 min. The mixture was concentrated and the aqueous phase was extracted with ethyl acetate (20 mL×3). The combined organic layers were dried with Na₂SO₄ and concentrated to afford 3-ethoxy-N²-(pyridin-4-ylmethyl)benzene-1,2-diamine (240 mg, 0.789 mmol, 86.3% yield, 80% purity) as a brown solid which was used in the next step without further purification.

¹H NMR (400 MHz, DMSO-d₆) 8.52-8.38 (m, 2H), 7.38-7.26 (m, 2H), 6.61 (t, J=8.1 Hz, 1H), 6.29 (dd, J=1.3, 8.0 Hz, 1H), 6.17 (dd, J=1.1, 8.1 Hz, 1H), 4.10 (s, 2H), 3.84 (q, J=70.0 Hz, 2H), 1.23 (t, J=6.9 Hz, 3H).

Step 4: To a mixture of 3-ethoxy-N²-(pyridin-4-ylmethyl)benzene-1,2-diamine (100 mg, 0.411 mmol) and 4-methyl-1,2,5-oxadiazole-3-carboxylic acid (Intermediate 2) (78.97 mg, 0.617 mmol) in DMF (1 mL) was added HATU (312.56 mg, 0.822 mmol) and DIPEA (159.36 mg, 1.23 mmol) in one portion at 25° C. The mixture was stirred at 25° C. for 3 hours. The mixture was poured into water (20 mL) and extracted with ethyl acetate (20 mL×3). The combined organic layers were dried with anhydrous Na₂SO₄, and concentrated to afford N-[3-ethoxy-2-(pyridin-4-ylmethylamino)phenyl]-4-methyl-1,2,5-oxadiazole-3-carboxamide (100 mg, 0.283 mmol, 68.9% yield) as a yellow oil which was used in the next step without further purification.

Step 5: N-[3-ethoxy-2-(pyridin-4-ylmethylamino)phenyl]-4-methyl-1,2,5-oxadiazole-3-carboxamide (so mg, 0.141 mmol) was added into AcOH (1 mL) in one portion at 25° C. The mixture was stirred at 90° C. for 5 hours. The mixture was concentrated to afford the crude product which was purified by prep. HPLC (Column: Phenomenex Gemini-NX C₁₈ 75*30 mm*3 μm, Mobile Phase A: water (0.04% NH₃·H₂O+10 mM NH₄HCO₃), Mobile Phase B: acetonitrile, Flow rate: 25 mL/min, gradient condition from 24% B to 54%). The pure fractions were collected and the volatiles were removed under vacuum. The residue was partitioned between acetonitrile (2 mL) and water (10 mL). The solution was lyophilized to dryness to give 3-[7-ethoxy-1-(pyridin-4-ylmethyl)benzimidazol-2-yl]-4-methyl-1,2,5-oxadiazole (2.58 mg, 0.008 mmol, 5.4% yield, 99.0% purity) as an off-white powder.

MS ES⁺: 336.1

¹H NMR (400 MHz, DMSO-d₆) 8.50-8.43 (m, 2H), 7.44 (d, J=8.2 Hz, 1H), 7.25 (t, J=8.2 Hz, 1H), 7.01 (d, J=5.8 Hz, 2H), 6.89 (d, J=7.8 Hz, 1H), 6.04 (s, 2H), 4.02 (q, J=6.8 Hz, 2H), 2.75 (s, 3H), 1.10 (t, J=6.8 Hz, 3H).

### Example 71: 4-[1-(pyridin-4-ylmethyl)benzimidazol-2-yl]-1,2,5-oxadiazol-3-amine

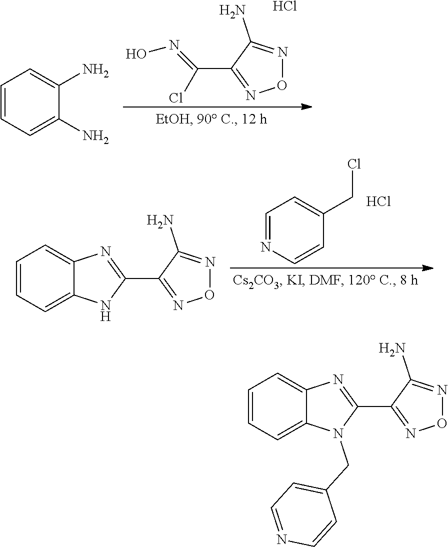

Step 1: A mixture of benzene-1,2-diamine (10 g, 92.47 mmol) and (3Z)-4-amino-N-hydroxy-1,2,5-oxadiazole-3-carboximidoyl chloride hydrochloride (18.40 g, 92.47 mmol) in EtOH (300 mL) was stirred at 90° C. for 12 hours. The mixture was cooled down to RT and an off-white precipitation formed. The precipitation was collected to give 4-(1H-benzimidazol-2-yl)-1,2,5-oxadiazol-3-amine (11 g, 54.35 mmol, 58.8% yield, 99.4% purity) as an off-white solid which was used for the next step directly.

MS ES⁺: 202.1

¹H NMR (400 MHz, DMSO-d₆) 13.69 (br s, 1H), 7.98-7.50 (m, 2H), 7.33 (dd, J=70.2, 17.3 Hz, 2H), 6.83 (s, 2H).

Step 2: To a solution of 4-(1H-benzimidazol-2-yl)-1,2,5-oxadiazol-3-amine (2 g, 9.94 mmol) in DMF (15 mL) was added 4-(chloromethyl)pyridine hydrochloride (1.63 g, 9.94 mmol), Cs₂CO₃ (9.72 g, 29.82 mmol) and KI (1.65 g, 9.94 mmol). The mixture was stirred at 120° C. for 8 hours. The reaction mixture was cooled down to RT, at which point water (30 mL) was added to the mixture and an off-white precipitation formed.

The precipitation was collected to give the crude product. The crude product was triturated with EtOH (10 mL) at 25° C. for 1 hour, then with ethyl acetate (10 mL) at 25° C. for 1 hour, and then with MeOH (10 mL) at 25° C. for 8 hours to give 4-[1-(pyridin-4-ylmethyl)benzimidazol-2-yl]-1,2,5-oxadiazol-3-amine (1.1 g, 3.76 mmol, 37.9% yield) as an off-white solid. The crude product was triturated with H₂O (30 mL) at 25° C. for 8 hours. Then the crude product was extracted with CHCl₃ (50 mL) at 80° C. and sat. LiCl (aq) (250 mL). The separated organic layer was dried over Na₂SO₄ and filtered. The filtrate was evaporated to dryness to give 4-[1-(pyridin-4-ylmethyl)benzimidazol-2-yl]-1,2,5-oxadiazol-3-amine (930 mg, 3.05 mmol, 81.2% yield, 96% purity) as a yellow solid.

MS ES⁺: 293.3

¹H NMR (400 MHz, DMSO-d₆) 8.52-8.45 (m, 2H), 7.93-7.87 (m, 1H), 7.74-7.69 (m, 1H), 7.46-7.37 (m, 2H), 7.10-7.06 (m, 2H), 7.04-6.99 (m, 2H), 6.03-5.99 (m, 2H).

Melting Point (° C.): 240.0-240.5

### Example 72: 2-(4-methyl-1,2,5-oxadiazol-3-yl)-3-(pyridin-3-ylmethyl) benzimidazol-4-amine

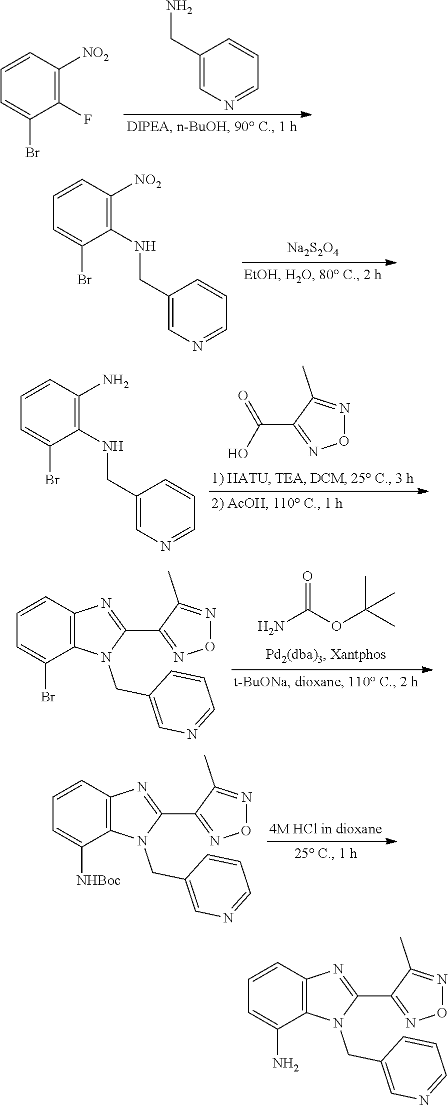

Step 1: A solution of 1-bromo-2-fluoro-3-nitro-benzene (5 g, 22.73 mmol), pyridin-3-ylmethanamine (2.46 g, 22.73 mmol) and DIPEA (5.87 g, 45.46 mmol) in n-BuOH (50 mL) was stirred at 90° C. for 1 hour. The mixture was concentrated to afford the crude product which was purified by column chromatography to give 2-bromo-6-nitro-N-(pyridin-3-ylmethyl)aniline (6.4 g, 20.71 mmol, 91.1% yield, 99.7% purity) as a yellow oil.

MS ES⁺: 307.9

Step 2: A mixture of 2-bromo-6-nitro-N-(pyridin-3-ylmethyl)aniline (2 g, 6.49 mmol), Na₂S₂O₄ (5.65 g, 32-45 mmol) in EtOH (20 mL) and H₂O (6 mL) was stirred at 80° C. for 2 hours. The mixture was poured into water (50 mL) and extracted with DCM (100 mL×3). The combined organic layers were dried with Na₂SO₄ and filtered. The filtrate was concentrated to afford 3-bromo-N²-(pyridin-3-ylmethyl)benzene-1,2-diamine (1.5 g, 5.39 mmol, 83.1% yield) as a yellow oil.

¹H NMR (400 MHz, DMSO-d₆) 8.56-8.50 (m, 1H), 8.48-8.40 (m, 1H), 7.79-7.71 (m, 1H), 7.36-7.27 (m, 1H), 6.71-6.63 (m, 3H), 5.12 (s, 2H), 4.20-4.13 (m, 1H), 4.11-4.06 (m, 2H).

Step 3: To a solution of 3-bromo-N²-(pyridin-3-ylmethyl)benzene-1,2-diamine (1 g, 3.60 mmol), TEA (1.09 g, 10.79 mmol) and 4-methyl-1,2,5-oxadiazole-3-carboxylic acid (Intermediate 2) (460.50 mg, 3.60 mmol) in DCM (10 mL) was added HATU (2.73 g, 7.19 mmol). The reaction mixture was stirred at 25° C. for 3 hours. The mixture was poured into water (10 mL) and extracted with DCM (10 mL×2). The combined organic layers were washed with brine (10 mL×2), dried with anhydrous Na₂SO₄ and filtered. The filtrate was evaporated to dryness as a yellow solid. Then the residue was taken into AcOH (10 mL) and stirred at 110° C. for 1 hour. The mixture was evaporated to dryness. The residue was extracted with DCM (10 mL×3). Then combined organic layers were washed with sat. NaHCO₃ (aq.) (50 mL), dried over Na₂SO₄ and filtered. The filtrate was evaporated to dryness. The residue was purified by column chromatography to afford 3-[7-bromo-1-(pyridin-3-ylmethyl)benzimidazol-2-yl]-4-methyl-1,2,5-oxadiazole (1 g, 2.70 mmol, 87.4% yield) as an off-white solid.

MS ES⁺: 372.1

¹H NMR (400 MHz, DMSO-d₆) 8.92-8.64 (m, 1H), 8.59-8.30 (m, 2H), 8.05-7.93 (m, 1H), 7.73-7.58 (m, 1H), 7.45-7.27 (m, 2H), 6.21 (s, 2H), 2.76 (s, 3H).

Step 4: To a solution of 3-[7-bromo-1-(pyridin-3-ylmethyl)benzimidazol-2-yl]-4-methyl-1,2,5-oxadiazole (200 mg, 0.540 mmol), tert-butyl carbamate (63.29 mg, 0.540 mmol), t-BuONa (155.76 mg, 1.62 mmol) and Xantphos (62.52 mg, 0.108 mmol) was added Pd₂(dba)₃ (98.94 mg, 0.108 mmol) in 1,4-dioxane (2 mL) at 25° C. Then the mixture was heated to 110° C. and stirred for 2 hours under N₂. The mixture was cooled to 25° C. and poured into water (10 mL) and stirred for 3 min. The aqueous phase was extracted with DCM (10 mL×2). The combined organic phases were washed with brine (10 mL×2), dried with anhydrous Na₂SO₄, filtered and concentrated in vacuum. The crude product was purified by column chromatography to give tert-butyl N-[2-(4-methyl-1,2,5-oxadiazol-3-yl)-3-(pyridin-3-ylmethyl)benzimidazol-4-yl]carbamate (40 mg, 0.075 mmol, 13.9% yield, 76.2% purity) as a white solid.

MS ES⁺: 407.0

Step 5: A solution of tert-butyl N-[2-(4-methyl-1,2,5-oxadiazol-3-yl)-3-(pyridin-3-ylmethyl)benzimidazol-4-yl]carbamate (40 mg, 0.075 mmol, 76.2% purity) in HCl/dioxane (4M, 5 mL) was stirred for 1 hour at 25° C. The reaction mixture was concentrated under reduced pressure to afford the crude product which was purified by prep. HPLC (Column: Phenomenex Gemini-NX C₁₈ 75*30 mm*3 μm, Mobile Phase A: water (10 mM NH₄HCO₃), Mobile Phase B: acetonitrile, Flow rate: 25 mL/min, gradient condition from 12% B to 72%). The pure fractions were collected, and the volatiles were removed under vacuum. The residue was partitioned between acetonitrile (2 mL) and water (10 mL). The solution was lyophilized to dryness to give 2-(4-methyl-1,2,5-oxadiazol-3-yl)-3-(pyridin-3-ylmethyl)benzimidazol-4-amine (2.62 mg, 0.008 mmol, 8.5% yield, 98.0% purity) as a white powder.

MS ES⁺: 307.3

¹H NMR (400 MHz, DMSO-d₆) 8.41-8.45 (m, 1H), 8.34 (d, J=1.60 Hz, 1H), 7.33-7.38 (m, 1H), 7.27-7.32 (m, 1H), 7.13-7.17 (m, 1H), 7.04-7.10 (m, 1H), 6.67 (d, J=7.60 Hz, 1H), 6.10 (s, 2H), 5.17 (s, 2H), 2.71 (s, 3H).

### Example 73: N-methyl-5-[[2-(4-methyl-1,2,5-oxadiazol-3-yl)benzimidazol-1-yl]methyl]pyridin-2-amine

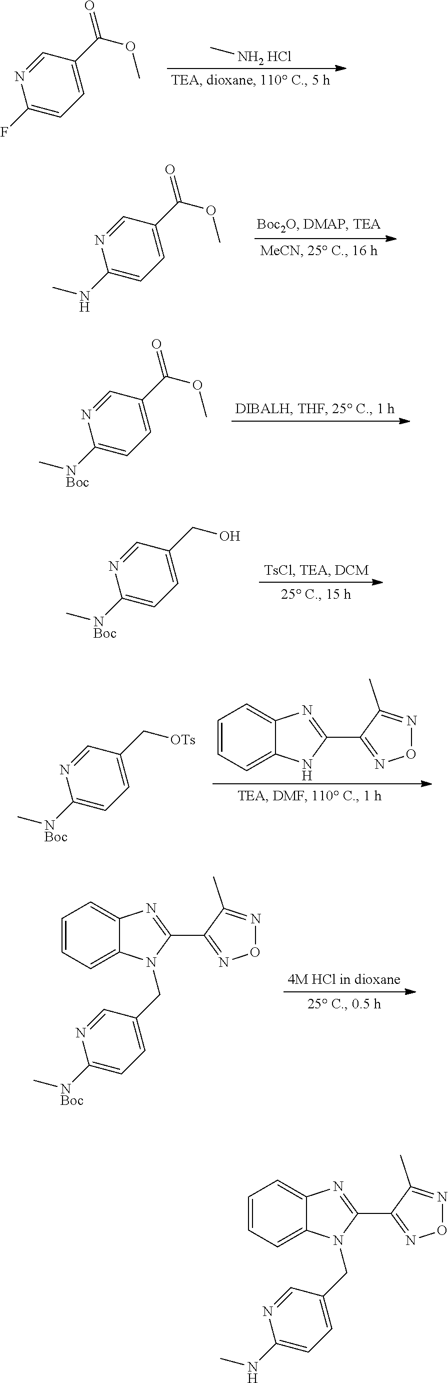

Step 1: A mixture of methyl 6-fluoropyridine-3-carboxylate (1 g, 6.45 mmol), methanamine hydrochloride (2.18 g, 32.23 mmol) and TEA (1.63 g, 16.12 mmol) in 1,4-dioxane (5 mL) was stirred at 110° C. for 5 hours. The reaction mixture cooled down to RT and poured into H₂O (10 mL). The mixture was extracted with ethyl acetate (10 mL ×3). The combined organic phases were washed with brine (10 mL, dried over anhydrous Na₂SO₄ and filtered. The filtrate was concentrated in vacuum to give methyl 6-(methylamino)pyridine-3-carboxylate (800 mg, 4.81 mmol, 74.7% yield) as a white solid.

MS ES⁺: 167.1

¹H NMR (400 MHz, DMSO-d₆) 8.55-8.60 (m, 1H), 7.76-7.85 (m, 1H), 7.33-7.41 (m, 1H), 6.44-6.50 (m, 1H), 3.75-3.77 (m, 3H), 2.81-2.84 (m, 3H).

Step 2: A mixture of methyl 6-(methylamino)pyridine-3-carboxylate (700 mg, 4.21 mmol), tert-butoxycarbonyl tert-butyl carbonate (1.10 g, 5.05 mmol), DMAP (51.46 mg, 0.421 mmol) and TEA (511.50 mg, 5.05 mmol) in MeCN (10 mL) was stirred at 25° C. for 16 hours. The reaction mixture cooled down to RT and poured into H₂O (10 mL). The mixture was extracted with ethyl acetate (10 mL×3). The combined organic phases were washed with brine (10 mL), dried over anhydrous Na₂SO₄, and concentrated in vacuum to give a residue. The residue was purified by column chromatography to afford methyl 6-[tert-butoxycarbonyl(methyl)amino]pyridine-3-carboxylate (1.0 g, 3.76 mmol, 89.2% yield) as a yellow oil.

MS ES⁺: 267.1

Step 3: To a mixture of methyl 6-[tert-butoxycarbonyl(methyl)amino]pyridine-3-carboxylate (200 mg, 0.751 mmol) in THF (1 mL) was added dropwise DIBALH (1M in toluene, 751.05 μL). Then the mixture was stirred at 25° C. for 1 hour. The reaction mixture was quenched with sat. NH₄Cl (aq.) (3 mL). The mixture was poured into H₂O (5 mL), filtered through a Celite® pad and washed with ethyl acetate (10 mL). The mixture was extracted with ethyl acetate (5 mL×3). The combined organic phases were washed with brine (5 mL), dried over anhydrous Na₂SO₄, and concentrated in vacuum to give tert-butyl N-[5-(hydroxymethyl)pyridin-2-yl]-N-methyl-carbamate (100 mg, 0.420 mmol, 55.9% yield) as a yellow oil which was used for the next step directly.

MS ES⁺: 239.2

Step 4: A mixture of tert-butyl N-[5-(hydroxymethyl)pyridin-2-yl]-N-methyl-carbamate (100 mg, 0.420 mmol), 4-toluenesulfonyl chloride (96.01 mg, 0.504 mmol) and TEA (84.93 mg, 0.839 mmol) in DCM (1.5 mL) was stirred at 25° C. for 15 min. The mixture was extracted with DCM (5 mL×3). The combined organic phases were washed with brine (5 mL×3), dried with anhydrous Na₂SO₄, filtered and concentrated in vacuum. The residue was purified by column chromatography to afford [6-[tert-butoxycarbonyl(methyl)amino]pyridin-3-yl]methyl 4-methylbenzenesulfonate (10 mg, 0.025 mmol, 5.7% yield) as a yellow oil.

Step 5: A mixture of [6-[tert-butoxycarbonyl(methyl)amino]pyridin-3-yl]methyl 4-methylbenzenesulfonate (10 mg, 0.025 mmol), 3-(1H-benzimidazol-2-yl)-4-methyl-1,2,5-oxadiazole (5.10 mg, 0.025 mmol) and K₂CO₃ (3.52 mg, 0.025 mmol) in DMF (1 mL) was stirred at 110° C. for 1 hour. The mixture was poured into water (2 mL) and extracted with ethyl acetate (2 mL×2). Then combined organic layers were washed with brine (2 mL×2), dried with anhydrous Na₂SO₄, filtered and concentrated in vacuum to afford tert-butyl N-methyl-N-[5-[[2-(4-methyl-1,2,5-oxadiazol-3-yl)benzimidazol-1-yl]methyl]pyridin-2-yl]carbamate (10 mg, 0.024 mmol, 93.3% yield) as a yellow oil which was used for the next step directly.

MS ES⁺: 421.1

Step 6: A mixture of tert-butyl N-methyl-N-[5-[[2-(4-methyl-1,2,5-oxadiazol-3-yl)benzimidazol-1-yl]methyl]pyridin-2-yl]carbamate (10 mg, 0.024 mmol) in 4M HCl in 1,4-dioxane (5 mL) was stirred at 25° C. for 30 min. The resulting mixture was dissolved in DMF (3 mL) and filtered. The filter liquor was concentrated in vacuo. The crude product was purified by prep. HPLC (Column: Xtimate C₁₈ 100*30 mm*10 μm, Mobile Phase A: water (0.225% FA), Mobile Phase B: acetonitrile, Flow rate: 10 mL/min, gradient condition from 10% B to 40%). The pure fractions were collected and the volatiles were removed under vacuum. The residue was partitioned between acetonitrile (2 mL) and water (10 mL). The solution was lyophilized to dryness to give N-methyl-5-[[2-(4-methyl-1,2,5-oxadiazol-3-yl)benzimidazol-1-yl]methyl]pyridin-2-amine (1.36 mg, 0.004 mmol, 17.7% yield, 99.1% purity) as a white powder.

MS ES⁺: 321.3

¹H NMR (400 MHz, DMSO-d₆) 8.01 (d, J=2.00 Hz, 1H), 7.84 (d, J=8.00 Hz, 1H), 7.77 (d, J=8.00 Hz, 1H), 7.30-7.45 (m, 2H), 7.24 (dd, J=8.69, 2.31 Hz, 1H), 6.51 (d, J=40.13 Hz, 1H), 6.33 (d, J=8.63 Hz, 1H), 5.71 (s, 2H), 2.74-2.79 (m, 3H), 2.69 (d, J=40.88 Hz, 3H).

### Example 74: 3-methyl-4-[1-[(2-methylpyridin-4-yl)methyl]benzimidazol-2-yl]-1,2,5-oxadiazole

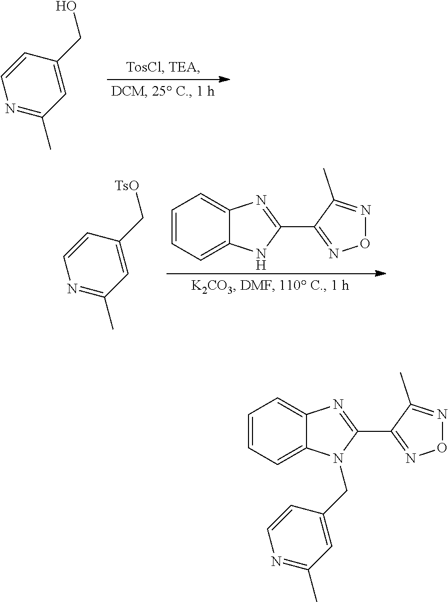

Step 1: A mixture of (2-methylpyridin-4-yl)methanol (250 mg, 2.03 mmol), 4-toluenesulfonyl chloride (387.02 mg, 2.03 mmol) and TEA (410.83 mg, 4.06 mmol) in DCM (5 mL) was stirred at 25° C. for 1 hour. The reaction was quenched by the addition of water (10 mL), the phases separated and the aqueous phase was extracted with DCM (5 mL×3). The combined organic phases were washed with brine (5 mL×3), dried with anhydrous Na₂SO₄, filtered and concentrated in vacuum. The residue was purified by silica gel chromatography (petroleum ether: ethyl acetate=1:0 to 1:5) to afford (2-methylpyridin-4-yl)methyl 4-methylbenzenesulfonate (56 mg, 0.202 mmol, 10.0% yield) as a red liquid.

MS ES⁺: 278.1

Step 2: A mixture of (2-methylpyridin-4-yl)methyl 4-methylbenzenesulfonate (13.85 mg, 0.050 mmol), 3-(1H-benzimidazol-2-yl)-4-methyl-1,2,5-oxadiazole (10 mg, 0.050 mmol) and K₂CO₃ (13.81 mg, 0.100 mmol) in DMF (0.5 mL) was stirred at 110° C. for 1 hour. The resulting mixture was cooled to RT, and then dissolved in DMF (3 mL) and filtered to remove the insoluble. The filter liquor was concentrated in vacuo. The crude product was further purified by prep. HPLC (Column: Welch Xtimate 75*40 mm*3 μm, Mobile Phase A: water (0.225% FA), Mobile Phase B: acetonitrile, Flow rate: 25 mL/min, gradient condition from 5% B to 45%). The pure fractions were collected and the volatiles were removed under vacuum. The residue was partitioned between acetonitrile (2 mL) and water (10 mL). The solution was lyophilized to dryness to give 3-methyl-4-[1-[(2-methylpyridin-4-yl)methyl]benzimidazol-2-yl]-1,2,5-oxadiazole (4.38 mg, 0.014 mmol, 27.3% yield, 95.1% purity) as a white powder.

MS ES⁺: 306.3

### Example 75: 3-[1-[(4,6-dimethylpyridin-2-yl)methyl]benzimidazol-2-yl]-4-methyl-1,2,5-oxadiazole

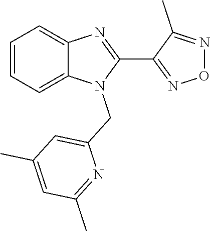 [SMILES: Cc1cc(C)nc(Cn2c(-c3nonc3C)nc3ccccc32)c1]

Prepared as described for Example 37 using 2-(bromomethyl)-4,6-dimethyl-pyridine (9.99 mg, 0.050 mmol) and 3-(1H-benzimidazol-2-yl)-4-methyl-1,2,5-oxadiazole (10 mg, 0.050 mmol) to give 3-[1[(4,6-dimethylpyridin-2-yl)methyl]benzimidazol-2-yl]-4-methyl-1,2,5-oxadiazole (11.71 mg, 0.037 mmol, 73.4% yield, 100% purity) as a white powder.

MS ES⁺: 320.3

### Example 76: 3-methyl-4-[1-[(1-oxidopyridin-1-ium-3-yl)methyl]benzimidazol-2-yl]-1,2,5-oxadiazole

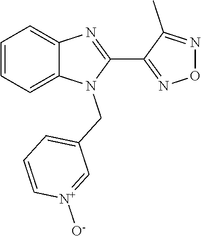 [SMILES: Cc1nonc1-c1nc2ccccc2n1Cc1ccc[n+]([O-])c1]

Prepared as described for Example 37 using 3-(chloromethyl)-1-oxido-pyridin-1-ium (prepared as described for WO2004/46113) (50 mg, 0.348 mmol) and 3-(1H-benzimidazol-2-yl)-4-methyl-1,2,5-oxadiazole (69.72 mg, 0.348 mmol) to give 3-methyl-4-[1-[(1-oxidopyridin-1-ium-3-yl)methyl]benzimidazol-2-yl]-1,2,5-oxadiazole (18.83 mg, 0.060 mmol, 17.1% yield, 97.4% purity) as a white powder.

MS ES⁺: 308.0

¹H NMR (400 MHz, DMSO-d₆) 8.17 (s, 1H), 8.13 (d, J=6.4 Hz, 1H), 7.90 (d, J=7.2 Hz, 1H), 7.76 (d, J=7.8 Hz, 1H), 7.47-7.36 (m, 2H), 7.33 (dd, J=6.4, 7.8 Hz, 1H), 7.03 (d, J 20=8.0 Hz, 1H), 5.89 (s, 2H), 2.79 (s, 3H).

### Example 77: 3-methyl-4-[1-[(1-oxidopyridin-1-ium-4-yl)methyl]benzimidazol-2-yl]-1,2,5-oxadiazole

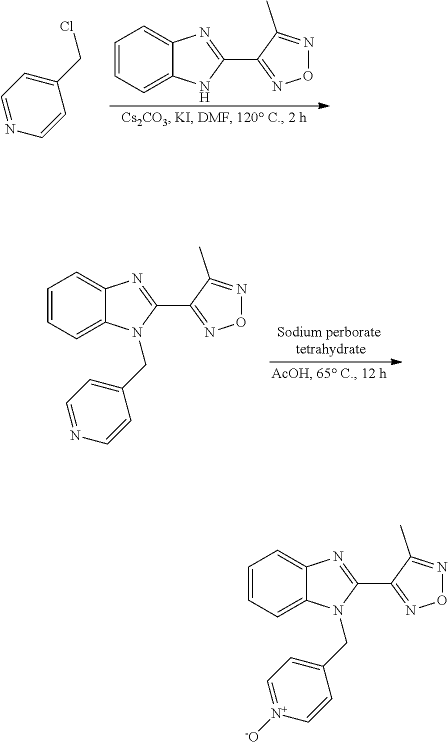

Step 1: To a solution of 3-(1H-benzimidazol-2-yl)-4-methyl-1,2,5-oxadiazole (500 mg, 2.50 mmol) in DMF (5 mL) was added 4-(chloromethyl)pyridine (318.62 mg, 2.50 mmol), Cs₂CO₃ (1.63 g, 5.00 mmol) and KI (82.92 mg, 0.500 mmol). The mixture was stirred at 120° C. for 2 hours under microwave radiation. The reaction mixture was diluted with ethyl acetate (50 mL). The separated organic layer was washed with H₂O (30 mL×3), dried over Na₂SO₄, filtered and concentrated under reduced pressure to give a residue. The residue was purified by column chromatography (SiO₂, petroleum ether ethyl acetate=1:0 to 1:1) to give 3-methyl-4-[1-(pyridin-4-ylmethyl)benzimidazol-2-yl]-1,2,5-oxadiazole (526 mg, 1.80 mmol, 72.2% yield, 99.9% purity) as an off-white solid.

MS ES⁺: 291.9

¹H NMR (400 MHz, DMSO-d₆) 8.52-8.45 (m, 2H), 7.95-7.87 (m, 1H), 7.70-7.64 (m, 1H), 7.45-7.35 (m, 2H), 7.11-7.07 (m, 2H), 5.95 (s, 2H), 2.79 (s, 3H).

Step 2: To a solution of 3-methyl-4-[1-(pyridin-4-ylmethyl)benzimidazol-2-yl]-1,2,5-oxadiazole (50 mg, 0.172 mmol) in AcOH (1 mL) was added sodium perborate tetrahydrate (29.05 mg, 0.189 mmol) at 65° C. The mixture was stirred at 65° C. for 12 hours, at which point the reaction mixture was concentrated under reduced pressure to dryness. The residue was purified by prep. HPLC (column: Phenomenex Luna C₁₈ 75*30 mm*3 μm; mobile phase A: water (0.225% FA), mobile phase B: MeCN; Flow rate: 25 mL/min, gradient condition from 30% B to 60%). The pure fractions were collected and the volatiles were removed under vacuum. The residue was partitioned between acetonitrile (2 mL) and water (10 mL). The solution was lyophilized to dryness to give 3-methyl-4-[1-[(1-oxidopyridin-1-ium-4-yl)methyl]benzimidazol-2-yl]-1,2,5-oxadiazole (20.78 mg, 0.068 mmol, 39.4% yield, 99.9% purity) as a white solid.

MS ES⁺: 308.3

¹H NMR (400 MHz, DMSO-d₆) 8.17-8.11 (m, 2H), 7.93-7.87 (m, 1H), 7.74-7.68 (m, 1H), 7.48-7.35 (m, 2H), 7.22-7.14 (m, 2H), 5.88 (s, 2H), 2.78 (s, 3H).

### Example 78: 3-methyl-4-[1-[(6-methylpyridin-2-yl)methyl]benzimidazol-2-yl]-1,2,5-oxadiazole

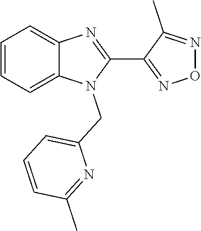 [SMILES: Cc1cccc(Cn2c(-c3nonc3C)nc3ccccc32)n1]

Prepared as described for Example 37 using 2-(bromomethyl)-6-methyl-pyridine (18.59 mg, 0.100 mmol) and 3-(1H-benzimidazol-2-yl)-4-methyl-1,2,5-oxadiazole (20 mg, 0.100 mmol) to give 3-methyl-4-[1-[(6-methylpyridin-2-yl)methyl]benzimidazol-2-yl]-1,2,5-oxadiazole (17.25 mg, 0.056 mmol, 56.0% yield, 99.1% purity) as a white powder.

MS ES⁺: 306.3

### Example 79: 3-methyl-4-[1-(pyridin-2-ylmethyl)benzimidazol-2-yl]-1,2,5-oxadiazole

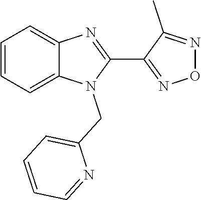 [SMILES: Cc1nonc1-c1nc2ccccc2n1Cc1ccccn1]

Prepared as described for Example 74 using pyridin-2-ylmethyl 4-methylbenzenesulfonate (13.15 mg, 0.050 mmol) and 3-(1H-benzimidazol-2-yl)-4-methyl-1,2,5-oxadiazole (10 mg, 0.050 mmol) to give 3-methyl-4-[1-(pyridin-2-ylmethyl)benzimidazol-2-yl]-1,2,5-oxadiazole (12.86 mg, 0.044 mmol, 87.7% yield, 99.2% purity) as a white powder.

MS ES⁺: 292.3

### Example 80: 5-[[6-fluoro-2-(4-methyl-1,2,5-thiadiazol-3-yl)benzimidazol-1-yl]methyl]pyridine-2-carbonitrile

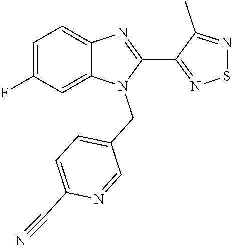 [SMILES: Cc1nsnc1-c1nc2ccc(F)cc2n1Cc1ccc(C#N)nc1]

Prepared as described for Example 49 using 2,4-difluoro-1-nitro-benzene (1 g, 6.29 mmol) and 5-(aminomethyl)pyridine-2-carbonitrile hydrochloride (prepared as described for Journal of Medicinal Chemistry, 2003, vol. 46, no. 17, pages 3612-3622) (1.07 g, 6.29 mmol) to give 5-[[6-fluoro-2-(4-methyl-1,2,s-thiadiazol-3-yl)benzimidazol-1-yl]methyl]pyridine-2-carbonitrile (16.48 mg, 0.047 mmol, 0.7% yield, 99.1% purity) as a white powder.

MS ES⁺: 351.2

¹H NMR (400 MHz, DMSO-d₆) 8.71 (s, 1H), 7.85-7.99 (m, 2H), 7.61-7.74 (m, 2H), 7.24 (s, 1H), 6.04 (s, 2H), 2.95 (s, 3H).

### Example 81: N-methyl-2-(4-methyl-1,2,5-oxadiazol-3-yl)-3-(pyridin-3-ylmethyl)benzimidazol-4-amine

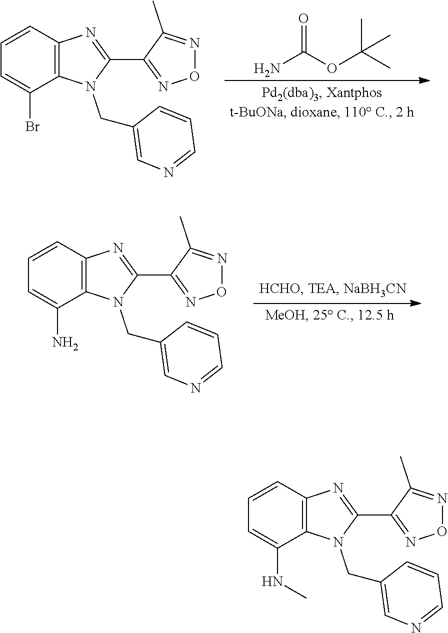

Step 1: To a solution of 3-[7-bromo-1-(pyridin-3-ylmethyl)benzimidazol-2-yl]-4-methyl-1,2,5-oxadiazole (100 mg, 0.270 mmol), tert-butyl carbamate (31.64 mg, 0.270 mmol), Xantphos (31.26 mg, 0.054 mmol) and t-BuONa (77.88 mg, 0.810 mmol) in 1,4-dioxane (1.5 mL) was added Pd₂(dba)₃ (49.47 mg, 0.054 mmol) under N₂. The mixture was stirred at 110° C. for 2 hours. Then the reaction mixture was diluted with H₂O (20 mL) and extracted with ethyl acetate (20 mL×3). The combined organic phase was concentrated under reduced pressure to give 2-(4-methyl-1,2,5-oxadiazol-3-yl)-3-(pyridin-3-ylmethyl)benzimidazol-4-amine (72 mg) as a yellow solid which was used into the next step without further purification.

MS ES⁺: 307.1

Step 2: To a solution of 2-(4-methyl-1,2,5-oxadiazol-3-yl)-3-(pyridin-3-ylmethyl)benzimidazol-4-amine (72 mg, 0.235 mmol), formaldehyde (10.59 mg, 0.353 mmol, 37% in water) and MeOH (1.5 mL) was added TEA (71.35 mg, 0.705 mmol). The mixture was stirred at 25° C. for 0.5 hour. Then NaBH₃CN (44.31 mg, 0.705 mmol) was added in portions. The resulting mixture was stirred at 25° C. for 12.5 hours, at which point the reaction mixture was diluted with H₂O (10 mL) and extracted with DCM (20 mL). The separated organic layer was dried over Na₂SO₄, filtered and concentrated under reduced pressure to give a residue which was purified by prep. HPLC (column: Welch Xtimate 75*40 mm*3 μm; Mobile Phase A: water (0.225% FA), Mobile Phase B: acetonitrile, Flow rate: 25 mL/min, gradient condition from 25% B to 55%) to give N-methyl-2-(4-methyl-1,2,5-oxadiazol-3-yl)-3-(pyridin-3-ylmethyl)benzimidazol-4-amine (1.01 mg, 0.003 mmol, 1.2% yield, 91.0% purity) as a white solid.

MS ES⁺: 321.3

¹H NMR (400 MHz, DMSO-d₆) 8.45-8.40 (m, 1H), 8.29 (s, 1H), 7.31-7.26 (m, 2H), 7.22-7.15 (m, 2H), 6.56-6.53 (m, 1H), 6.14 (s, 2H), 5.50-5.43 (m, 1H), 2.75-2.68 (m, 6H).

### Example 82: 3-[1-[(3-fluoropyridin-2-yl)methyl]benzimidazol-2-yl]-4-methyl-1,2,5-oxadiazole

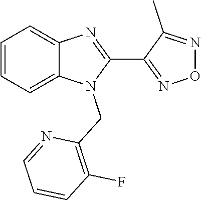 [SMILES: Cc1nonc1-c1nc2ccccc2n1Cc1ncccc1F]

Prepared as described for Example 37 using 3-(1H-benzimidazol-2-yl)-4-methyl-1,2,5-oxadiazole (20 mg, 0.100 mmol) and 2-(chloromethyl)-3-fluoro-pyridine (14.54 mg, 0.100 mmol) to give 3-[1-[(3-fluoropyridin-2-yl)methyl]benzimidazol-2-yl]-4-methyl-1,2,5-oxadiazole (9.41 mg, 0.030 mmol, 29.6% yield, 97.2% purity) as a yellow powder.

MS ES⁺: 310.2

### Example 83: 5-[[4,7-difluoro-2-(4-methyl-1,2,5-oxadiazol-3-yl)benzimidazol-1-yl]methyl]pyrimidine-2-carbonitrile

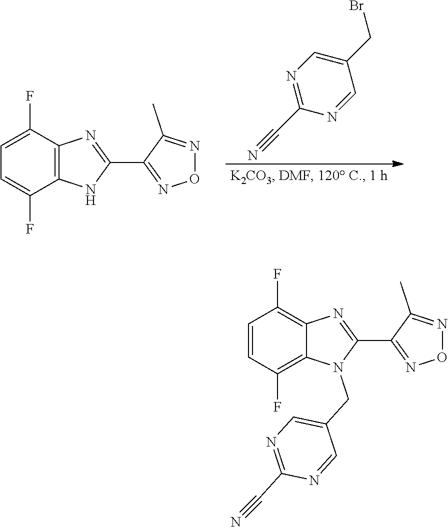 [SMILES: Cc1nonc1-c1nc2c(F)ccc(F)c2[nH]1 ; N#Cc1ncc(CBr)cn1 ; Cc1nonc1-c1nc2c(F)ccc(F)c2n1Cc1cnc(C#N)nc1]

To a solution of 3-(4,7-difluoro-1H-benzimidazol-2-yl)-4-methyl-1,2,5-oxadiazole (30 mg, 0.127 mmol) in DMF (1 mL) was added 5-(bromomethyl)pyrimidine-2-carbonitrile (prepared as described for US2018/079742) (25.15 ing, 0.127 mmol) and K₂CO₃(52.67 ng, 0-381 mmol). The mixture was stirred at 120° C. for 1 hour. The reaction mixture was cooled down and evaporated to dryness. The residue was purified by prep. HPLC (Column: Xtimate C₁₈ 100*30 mm*10 μm, Mobile Phase A: water (0.225%0 FA), Mobile Phase B: acetonitrile, Flow rate: 25 mL/min, gradient condition from 5500 B to 8500). The pure fractions were collected and the volatiles were removed under vacuum. The residue was partitioned between acetonitrile (2 mL) and water (10 mL). The solution was lyophilized to dryness to give 5-[[4,7-difluoro-2-(4-methyl-1,2,5-oxadiazol-3-yl)benzimidazol-1-yl]methyl]pyrimidine-2-carbonitrile (10.99 mg, 0.031 mmol, 24.2% yield, 98.9% purity) as an off-white solid.

MS ES⁺: 354.3

¹H NMR (400 MHz, DMSO-d₆) 8.98 (s, 2H), 7.37-7.15 (m, 2H), 6.07 (s, 2H), 2.78 (s, 3H).

### Example 84: 3-[4,7-difluoro-1-(pyrimidin-5-ylmethyl)benzimidazol-2-yl]-4-methyl-1,2,5-oxadiazole

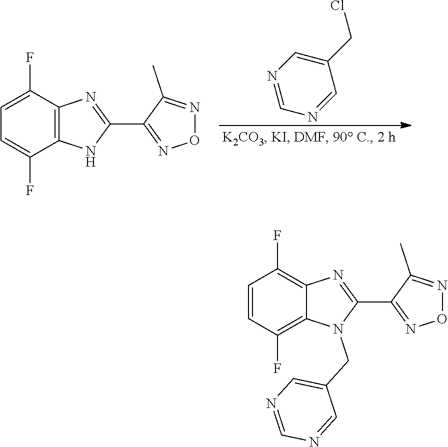 [SMILES: Cc1nonc1-c1nc2c(F)ccc(F)c2[nH]1 ; ClCc1cncnc1 ; Cc1nonc1-c1nc2c(F)ccc(F)c2n1Cc1cncnc1]

To a solution of 3-(4,7-difluoro-1H-benzimidazol-2-yl)-4-methyl-1,2,5-oxadiazole (30 mg, 0.127 mmol) in DMF (1 mL) was added 5-(chloromethyl)pyrimidine (16.33 mg, 0.127 mmol), K₂CO₃ (52.67 mg, 0.381 mmol) and KI (4.22 mg, 0.025 mmol). The mixture was stirred at 90° C. for 2 hours. The reaction mixture was cooled down and evaporated to dryness. The residue was purified by prep. HPLC (Column: Xtimate C₁₈ 100*30 mm*10 μm, Mobile Phase A: water (0.225% FA), Mobile Phase B: acetonitrile, Flow rate: 25 mL/min, gradient condition from 45% B to 75%). The pure fractions were collected and the volatiles were removed under vacuum. The residue was partitioned between acetonitrile (2 mL) and water (10 mL). The solution was lyophilized to dryness to give 3-[4,7-difluoro-1-(pyrimidin-5-ylmethyl)benzimidazol-2-yl]-4-methyl-1,2,5-oxadiazole (16 mg, 0.049 mmol, 38.4% yield, 100% purity) as a white solid.

MS ES⁺: 329.3

¹H NMR (400 MHz, DMSO-d₆) 9.14 (s, 1H), 8.73 (s, 2H), 7.37-7.12 (m, 2H), 5.99 (s, 2H), 2.78 (s, 3H).

### Example 85: rac-4-[1-[1-(pyridin-3-yl)ethyl]benzimidazol-2-yl]-1,2,5-oxadiazol-3-amine

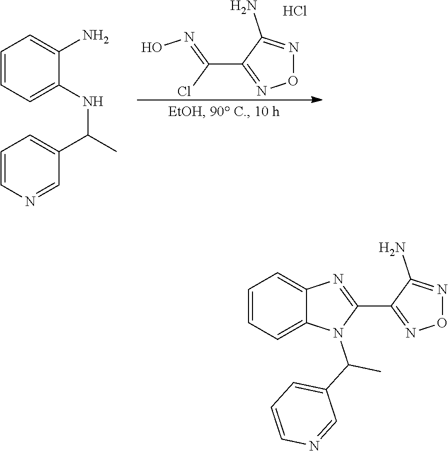 [SMILES: CC(Nc1ccccc1N)c1cccnc1 ; Nc1nonc1/C(Cl)=N/O ; Cl ; CC(c1cccnc1)n1c(-c2nonc2N)nc2ccccc21]

A mixture of N²-[1-(pyridin-3-yl)ethyl]benzene-1,2-diamine (85 mg, 0.399 mmol) and (Z)-4-amino-N-hydroxy-1,2,5-oxadiazole-3-carbimidoyl chloride hydrochloride (64.78 mg, 0.326 mmol) in EtOH (3 mL) was stirred at 90° C. for 10 hours. The mixture was concentrated to dryness. The residue was purified by prep. HPLC (Column: Welch Xtimate 75*40 mm*3 μm, Mobile Phase A: water (0.225% FA), Mobile Phase B: MeCN, Flow rate: 25 mL/min, gradient condition from 20% B to 50%). The pure fractions were collected and the volatiles were removed under vacuum. The residue was partitioned between acetonitrile (2 mL) and water (10 mL). The solution was lyophilized to dryness to give rac-4-[1-[1-(pyridin-3-yl)ethyl]benzimidazol-2-yl]-1,2,5-oxadiazol-3-amine (31.13 mg, 0.101 mmol, 25.3% yield, 99.5% purity) as a brown powder.

MS ES⁺: 307.1

¹H NMR (400 MHz, DMSO-d₆) 8.59 (d, J=1.8 Hz, 1H), 8.51 (d, J=3.6 Hz, 1H), 7.85 (d, J=8.2 Hz, 1H), 7.74 (d, J=8.4 Hz, 1H), 7.38 (dd, J=4.6, 7.8 Hz, 1H), 7.34-7.20 (m, 3H), 7.02 (s, 2H), 6.92 (q, J=7.6 Hz, 1H), 2.07 (d, J=7.2 Hz, 3H).

SFC: Rt=4.764 min, 6.948 min; 50-46%, 49-54%

### Example 86: 4-[7-fluoro-1-(pyridin-3-ylmethyl)benzimidazol-2-yl]-1,2,5-oxadiazol-3-amine

### Example 87: 4-[4-fluoro-1-(pyridin-3-ylmethyl)benzimidazol-2-yl]-1,2,5-oxadiazol-3-amine

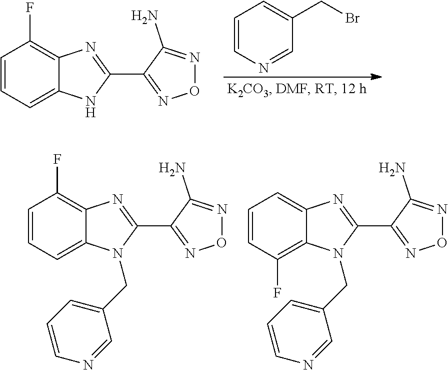

To a stirred solution of 4-(4-fluoro-benzimidazol-2-yl)-1,2,5-oxadiazol-3-amine (Intermediate 4) (0.2 g, 0.9 mmol) in DMF (8 mL) was added potassium carbonate (0.378 g, 2.7 mmol) and 3-(bromomethyl)pyridine (0.236 g, 1.4 mmol) and the resulting reaction mixture was stirred for 12 hours at RT. After completion of the reaction, the mixture was diluted with water (so mL) and extracted with ethyl acetate (2×50 mL). The organic layer was dried over Na₂SO₄ and concentrated under reduced pressure to obtain an isomeric mixture of desired products. The crude mixture was purified by SFC (Column/dimensions: Chiralcel-OJ-H (30×250)mm, 5 μm; % CO₂: 70%; % Co-solvent: 30% (MeOH); Total Flow: 100.0 g/min; Back Pressure: 100 bar; Temperature: 30° C.; UV: 220 nm) to afford Peak 2 (4-[7-fluoro-1-(pyridin-3-ylmethyl)benzimidazol-2-yl]-1,2,5-oxadiazol-3-amine) (0.04 g, 14% yield) as an off-white solid and Peak 1 (4-[4-fluoro-1-(pyridin-3-ylmethyl)benzimidazol-2-yl]-1,2,5-oxadiazol-3-amine) (0.06 g, 21% yield) as an off-white solid.

### Example 86 (Peak 2)

MS ES⁺: 311.13

¹H NMR (400 MHz, DMSO-d₆) 8.50-8.45 (m, 2H), 7.73 (d, J=8.4 Hz, 1H), 7.51 (d, J=8.0 Hz, 1H), 7.39-7.30 (m, 2H), 7.28-7.22 (m, 1H), 6.98 (s, 2H), 6.04 (s, 2H).

### Example 87 (Peak 1)

MS ES⁺: 311.13

¹H NMR (400 MHz, DMSO-d6) 8.54 (s, 1H), 8.48 (d, J=3.60 Hz, 1H), 7.64 (d, J=8.40 Hz, 1H), 7.52 (d, J=7.60 Hz, 1H), 7.45-7.39 (m, 1H), 7.35-7.29 (m, 1H), 7.26-7.20 (m, 1H), 6.94 (s, 2H), 6.01 (s, 2H).

### Example 88: 4-(1-((6-bromopyridin-3-yl)methyl)benzimidazol-2-yl)-1,2,5-oxadiazol-3-amine

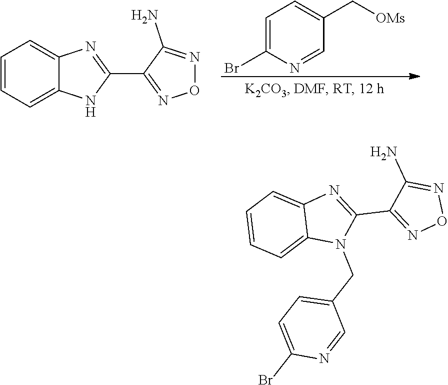

Following the procedure employed for Example 86 using 4-(benzimidazol-2-yl)-1,2,5-oxadiazol-3-amine (Intermediate 5) (0.184 g, 0.91 mmol) and (6-bromopyridin-3-yl)methyl methanesulfonate (Intermediate 6) (0.172 g, 1.4 mmol), gave 4-(1-((6-bromopyridin-3-yl)methyl)benzimidazol-2-yl)-1,2,5-oxadiazol-3-amine (0.105 g, 31% yield) as an off-white solid.

MS ES⁺: 371.35

¹H NMR (400 MHz, DMSO-d₆) 8.38 (d, J=2.0 Hz, 1H), 7.88 (d, J=8.0 Hz, 1H), 7.78 (d, J=8.0 Hz, 1H), 7.57 (d, J=8.4 Hz, 1H), 7.48-7.38 (m, 3H), 7.00 (s, 2H), 5.97 (s, 2H).

### Example 89: 3-[1-(pyridin-3-ylmethyl)benzimidazol-2-yl]-4-vinyl-1,2,5-thiadiazole

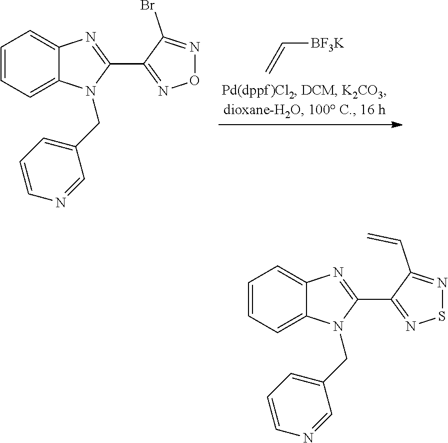

A solution of 3-bromo-4-(1-(pyridin-3-ylmethyl)benzimidazol-2-yl)-1,2,5-thiadiazole (Example 126) (80 mg, 0.21 mmol), potassium vinyl trifluoroborane (40 mg, 0.3 mmol) and K₂CO₃ (70 mg, 0.5 mmol) in 1,4-dioxane (1.8 mL) and water (0.2 mL) was purged with N₂ for 10 min. At this point, PdCl₂(dppf)·CH₂Cl₂ (16 mg, 0.02 mmol) was added under N₂ atmosphere and the reaction mixture was stirred at 100° C. for 16 hours in a sealed tube. After completion of the reaction, the reaction mass was diluted with ethyl acetate (20 mL), filtered through a Celite® bed which was washed thoroughly with ethyl acetate (2×20 mL). Filtrate and washings were combined and washed with water (100 mL). The organic layer was dried over Na₂SO₄ and concentrated under reduced pressure. The crude material was purified by reverse phase column chromatography (0-60% methanol in water) to afford 3-[1-(pyridin-3-ylmethyl)benzimidazol-2-yl]-4-vinyl-1,2,5-thiadiazole as a pale yellow solid (50 mg, 73% yield).

MS ES⁺: 320.19

¹H NMR (400 MHz, DMSO-d₆) 8.50 (s, 1H), 8.45 (d, J=3.9 Hz, 1H), 7.93-7.86 (m, 2H), 7.68 (d, J=7.2 Hz, 1H), 7.52 (d, J=8.0 Hz, 1H), 7.40-7.35 (m, 2H), 7.32-7.27 (m, 1H), 6.35 (d, J=17.2 Hz, 1H), 5.96 (s, 2H), 5.77 (t, J=11.6 Hz, 1H).

### Example 90: 4-[1-(pyridin-3-ylmethyl)benzimidazol-2-yl]-1,2,5-oxadiazol-3-amine

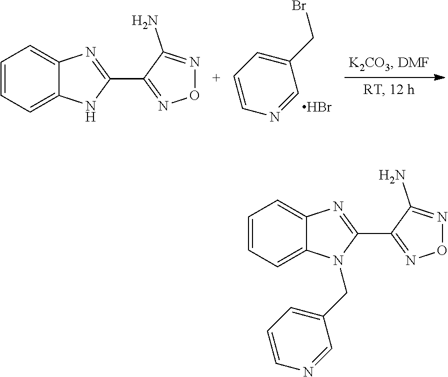

Following the procedure employed for Example 86 using 4-(benzimidazol-2-yl)-1,2,5-oxadiazol-3-amine (Intermediate 5) (0.1 g, 0.5 mmol) and 3-(bromomethyl)pyridine hydrobromide (0.15 g, 0.6 mmol), gave 4-[1-(pyridin-3-ylmethyl)benzimidazol-2-yl]-1,2,5-oxadiazol-3-amine (0.09 g, 61% yield) as an off-white solid.

MS ES⁺: 293.33

¹H NMR (400 MHz, DMSO) 8.53 (d, J=1.6 Hz, 1H), 8.48-8.46 (dd, J=1.2 Hz and 4.4 Hz, 1H), 7.88 (d, J=7.2 Hz, 1H), 7.79 (d, J=7.6 Hz, 1H), 7.52 (d, J=8.0 Hz, 1H), 7.48-7.35 (m, 2H), 7.35-7.28 (m, 1H), 7.01 (s, 2H), 6.01 (s, 2H).

### Example 91: 4-[6-fluoro-1-(pyridin-4-ylmethyl)benzimidazol-2-yl]-1,2,5-oxadiazol-3-amine

### Example 92: 4-[5-fluoro-1-(pyridin-4-ylmethyl)benzimidazol-2-yl]-1,2,5-oxadiazol-3-amine

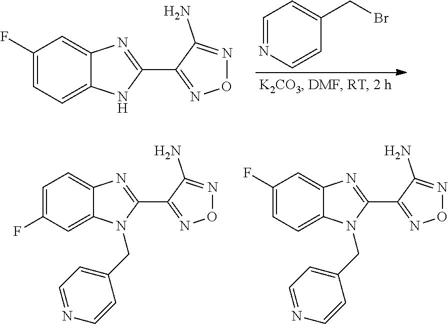

Following the procedure employed for Example 86 using 4-(5-fluoro-benzimidazol-2-yl)-1,2,5-oxadiazol-3-amine (Intermediate 8) (0.15 g, 0.685 mmol) and 4-(bromomethyl)pyridine (0.172 g, 1.0 mmol) followed by separation of isomers by SFC, gave 4-[6-fluoro-1-(pyridin-4-ylmethyl)benzimidazol-2-yl]-1,2,5-oxadiazol-3-amine (20 mg, 35% yield) as a pale brown solid and 4-[5-fluoro-1-(pyridin-4-ylmethyl)benzimidazol-2-yl]-1,2,5-oxadiazol-3-amine (20 mg, 35% yield) as a pale brown solid.

### Example 91

MS ES⁺: 311.13

¹H NMR (400 MHz, DMSO-d₆) 8.49 (d, J=5.6 Hz, 2H), 7.95-7.91 (m, 1H), 7.71-7.68 (dd, J=2.0 Hz and 9.2 Hz, 1H), 7.27 (m, 1H), 7.07 (d, J=5.6 Hz, 2H), 6.97 (s, 2H), 5.97 (s, 2H).

### Example 92

MS ES⁺: 311.17

¹H NMR (400 MHz, DMSO) 8.48 (d, J=6.0 Hz, 2H), 7.74 (m, J=4.2 Hz, 2H), 7.33 (m, J=4.2 Hz, 1H), 7.08 (d, J=5.8 Hz, 2H), 6.98 (s, 2H), 6.00 (s, 2H).

### Example 93: 4-[I-[(2-methoxypyridin-4-yl)methyl]benzimidazol-2-yl]-1,2,5-oxadiazol-3-amine

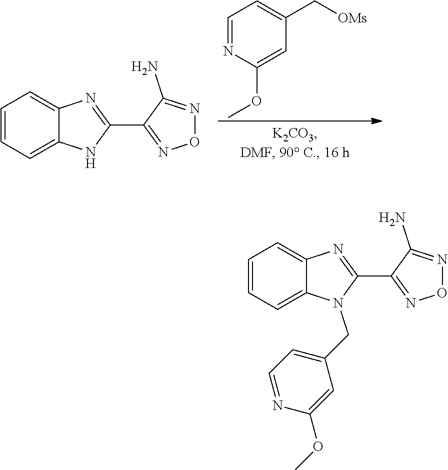

Following the procedure employed for Example 86 using 4-(benzimidazol-2-yl)-1,2,5-oxadiazol-3-amine (Intermediate 5) (50 mg, 0.25 mmol) and (2-methoxypyridin-4-yl)methyl methanesulfonate (Intermediate 13) (64 mg, 0.3 mmol), gave 4-[1-[(2-methoxypyridin-4-yl)methyl]benzimidazol-2-yl]-1,2,5-oxadiazol-3-amine (37 mg, 46% yield) as a white solid.

MS ES⁺: 323.18

¹H NMR (400 MHz, DMSO-d₆) 8.07 (dd, J=0.80, 5.40 Hz, 1H), 7.88-7.89 (m, 1H), 7.69-7.69 (m, 1H), 7.39-7.40 (m, 2H), 7.00 (s, 2H), 6.70 (dd, J=1.60, 5.40 Hz, 1H), 6.42 (d, J=0.40 Hz, 1H), 5.96 (s, 2H), 3.78 (s, 3H).

### Example 94: 4-[1-[[2-(trifluoromethyl)pyridin-4-yl]methyl]benzimidazol-2-yl]-1,2,5-oxadiazol-3-amine

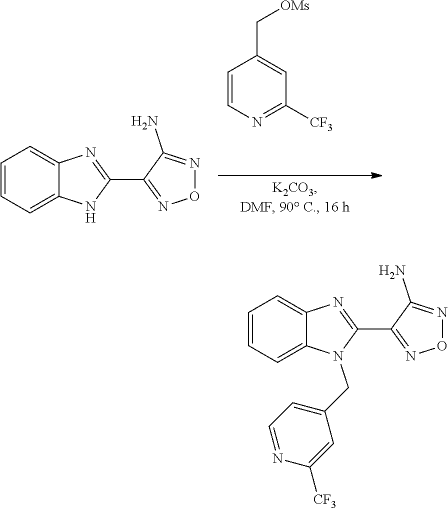 [SMILES: Nc1nonc1-c1nc2ccccc2[nH]1 ; CS(=O)(=O)OCc1ccnc(C(F)(F)F)c1 ; Nc1nonc1-c1nc2ccccc2n1Cc1ccnc(C(F)(F)F)c1]

Following the procedure employed for Example 86 using 4-(benzimidazol-2-yl)-1,2,5-oxadiazol-3-amine (Intermediate 5) (0.3 g, 1.66 mmol) and (2-(trifluoromethyl)pyridin-4-yl)methyl methanesulfonate (Intermediate 24) (0.19 g, 1.1 mmol), gave 4-[1-[[2-(trifluoromethyl)pyridin-4-yl]methyl]benzimidazol-2-yl]-1,2,5-oxadiazol-3-amine (0.105 g, 18% yield) as an off-white solid.

MS ES⁺: 361.19

¹H NMR (400 MHz, DMSO-d₆) 8.64 (d, J=5.0 Hz, 1H), 7.92 (q, J=2.9 Hz, 1H), 7.83 (s, 1H), 7.73 (q, J=2.9 Hz, 1H), 7.43 (m, 2H), 7.21 (d, J=4.7 Hz, 1H), 7.00 (s, 2H), 6.12 (s, 2H).

### Example 95: 4-[i-[[5-(trifluoromethyl)pyridin-3-yl]methyl]benzimidazol-2-yl]-1,2,5-oxadiazol-3-amine

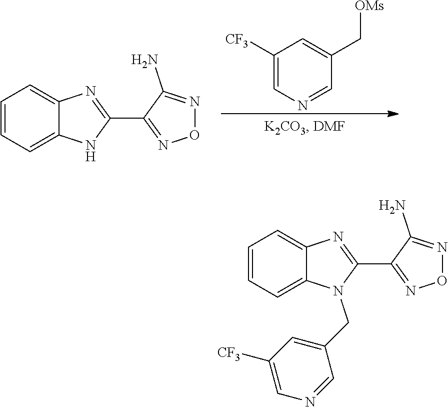 [SMILES: Nc1nonc1-c1nc2ccccc2[nH]1 ; CS(=O)(=O)OCc1cncc(C(F)(F)F)c1 ; Nc1nonc1-c1nc2ccccc2n1Cc1cncc(C(F)(F)F)c1]

Following the procedure employed for Example 86 using 4-(benzimidazol-2-yl)-1,2,5-oxadiazol-3-amine (Intermediate 5) (0.042 g, 0.2 mmol) and (5-(trifluoromethyl)pyridin-3-yl)methyl methanesulfonate (0.076 g, 0.3 mmol), gave 4-[1-[[5-(trifluoromethyl)pyridin-3-yl]methyl]benzimidazol-2-yl]-1,2,5-oxadiazol-3-amine (0.042 g, 58% yield) as a pale yellow solid.

MS ES⁺: 361.2

¹H NMR (400 MHz, DMSO-d₆) 8.91 (s, 1H), 8.69 (s, 1H), 8.08 (s, 1H), 7.90 (d, J=7.20 Hz, 1H), 7.81 (d, J=8.00 Hz, 1H), 7.39-7.40 (m, 2H), 6.99 (s, 2H), 6.10 (s, 2H).

### Example 96: N-methyl-4-[1-(pyridin-3-ylmethyl)benzimidazol-2-yl]-1,2,5-thiadiazol-3-amine

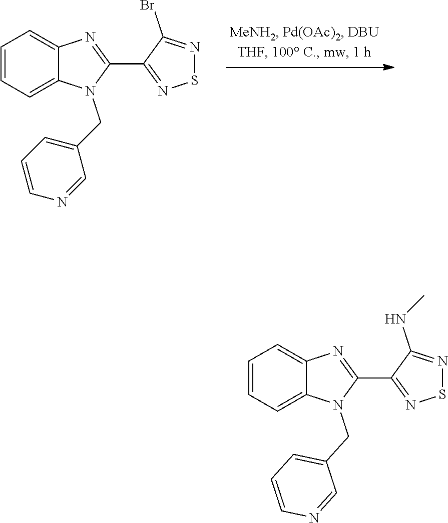 [SMILES: Brc1nsnc1-c1nc2ccccc2n1Cc1cccnc1 ; CNc1nsnc1-c1nc2ccccc2n1Cc1cccnc1]

To a solution of 3-bromo-4-(1-(pyridin-3-ylmethyl)benzimidazol-2-yl)-1,2,5-thiadiazole (Example 126) (0.051 g, 0.14 mmol) in 2M methylamine solution in THF (2.6 mL) was added DBU (0.05 g, 0.3 mmol) and palladium acetate (0.001 g, 0.01 mmol). The reaction was heated at 100° C. under microwave irradiation for 1 hour. The reaction mixture was then concentrated in vacuo and the crude product was purified by reverse phase chromatography using 65% methanol in water to afford N-methyl-4-[1-(pyridin-3-ylmethyl)benzimidazol-2-yl]-1,2,5-thiadiazol-3-amine (16 mg, 36% yield) as a pale brown solid.

MS ES⁺: 323.16

¹H NMR (400 MHz, DMSO-d₆) 8.53 (d, J=1.9 Hz, 2H), 8.44 (q, J=2.0 Hz, 1H), 7.86 (q, J=2.9 Hz, 1H), 7.75 (q, J=2.9 Hz, 1H), 7.51 (d, J=8.0 Hz, 1H), 7.38 (m, 2H), 7.29 (q, J=4.2 Hz, 1H), 6.19 (s, 2H), 3.13 (d, J=4.9 Hz, 3H).

### Example 97: 4-[1-[[6-(trifluoromethyl)pyridin-3-yl]methyl]benzimidazol-2-yl]-1,2,5-oxadiazol-3-amine

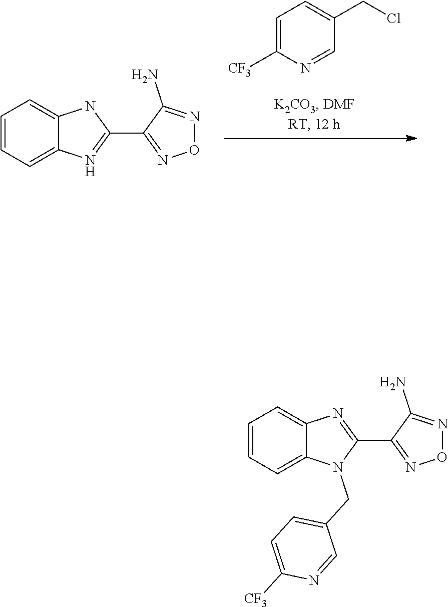 [SMILES: FC(F)(F)c1ccc(CCl)cn1 ; Nc1nonc1-c1nc2ccccc2[nH]1 ; Nc1nonc1-c1nc2ccccc2n1Cc1ccc(C(F)(F)F)nc1]

Following the procedure employed for Example 86 using 4-(benzimidazol-2-yl)-1,2,5-oxadiazol-3-amine (Intermediate 5) (0.15 g, 0.7 mmol) and 5-(chloromethyl)-2-(trifluoromethyl)pyridine (0.163 g, 0.84 mmol), gave 4-[1-[[6-(trifluoromethyl)pyridin-3-yl]methyl]benzimidazol-2-yl]-1,2,5-oxadiazol-3-amine (0.21 g, 82% yield) as an off-white solid.

MS ES⁺: 361.15

¹H NMR (400 MHz, DMSO-d₆) 8.75 (s, 1H), 7.90 (d, J=7.4 Hz, 1H), 7.81 (t, J=9.4 Hz, 2H), 7.71 (d, J=8.0 Hz, 1H), 7.45-7.43 (m, 2H), 7.00 (s, 2H), 6.12 (s, 2H).

### Example 98: 3-methyl-4-[1-(pyridin-4-ylmethyl)benzimidazol-2-yl]-1,2,5-oxadiazole

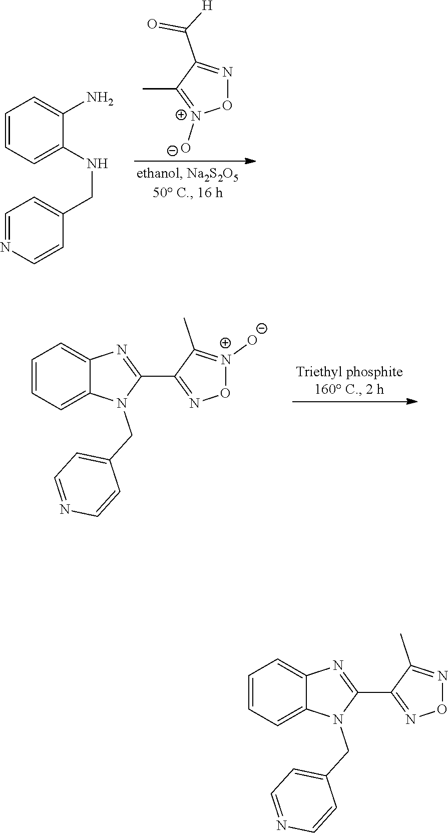

Step 1: To a stirred solution of N¹-(pyridin-4-ylmethyl)benzene-1,2-diamine (Intermediate 16) (150 mg, 0.7527 mmol) and 4-formyl-3-methyl-1,2,5-oxadiazole 2-oxide (Intermediate 15) (134 mg, 1.505 mmol) in ethanol, sodium metabisulfite (357 mg, 1.881 mmol) was added and heated to 50° C. for 16 hours. Upon completion, the reaction was quenched with ice cold water and extracted with ethyl acetate (2×100 mL), dried over Na₂SO₄, filtered and evaporated. The crude product was purified by reverse phase chromatography using aq. ammonium bicarbonate and methanol to yield 3-methyl-4-(1-(pyridin-4-ylmethyl)benzimidazol-2-yl)-1,2,5-oxadiazole 2-oxide (150 mg, 64.9% yield).

MS ES⁺: 308.14

Step 2: A stirred solution of 3-methyl-4-(1-(pyridin-4-ylmethyl)benzimidazol-2-yl)-1,2,5-oxadiazole 2-oxide (80 mg, 0.26 mmol) in triethyl phosphite (5 mL) was heated to 160° C. for 2 hours. After completion, the reaction was quenched with ice cold water and extracted with ethyl acetate (2×20 mL), dried over Na₂SO₄, filtered and evaporated. The crude product was purified by reverse phase chromatography to afford 3-methyl-4-[1-(pyridin-4-ylmethyl)benzimidazol-2-yl]-1,2,5-oxadiazole (25.8 mg, 34% yield) as an off-white solid.

MS ES⁺: 292.18

¹H NMR (400 MHz, DMSO-d₆) 8.49 (d, J=4.1 Hz, 2H), 7.91 (d, J=7.3 Hz, 1H), 7.67 (d, J=7.4 Hz, 2H), 7.40 (t, J=6.5 Hz, 2H), 7.09 (d, J=4.3 Hz, 1H), 5.96 (s, 2H), 2.79 (s, 3H).

### Example 99: 4-[1-(pyridin-3-ylmethyl)benzimidazol-2-yl]-1,2,5-thiadiazole-3-carbonitrile

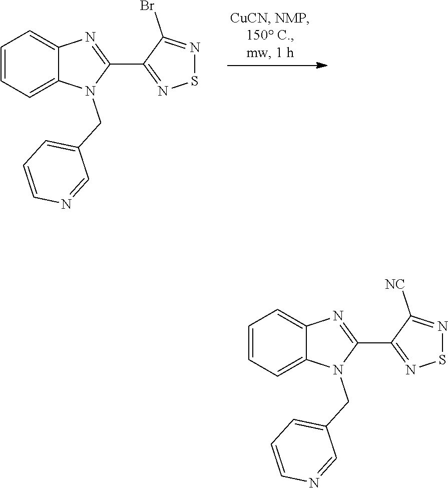 [SMILES: Brc1nsnc1-c1nc2ccccc2n1Cc1cccnc1 ; N#Cc1nsnc1-c1nc2ccccc2n1Cc1cccnc1]

3-Bromo-4-(1-(pyridin-3-ylmethyl)benzimidazol-2-yl)-1,2,5-thiadiazole (Example 126) (0.05 g, 0.1 mmol) was dissolved in NMP (5 mL). Copper(I) cyanide (0.01 g, 0.1 mmol) was added and the reaction mixture was heated at 150° C. under microwave irradiation for 1 hour. After this time, the reaction mixture was diluted with ethyl acetate and filtered through a pad of Celite®. The filtrate was washed with water and brine respectively. The organic layer was separated, dried (Na₂SO₄) and concentrated under vacuum to obtain crude product, which was purified by reverse phase column chromatography using 60% methanol in water to afford 4-[1-(pyridin-3-ylmethyl)benzimidazol-2-yl]-1,2,5-thiadiazole-3-carbonitrile (20 mg, 42% yield) as an off-white solid.

MS ES⁺: 319.12

¹H NMR (400 MHz, DMSO-d₆) 8.61 (s, 1H), 8.47 (d, J=4.1 Hz, 1H), 7.90 (d, J=7.4 Hz, 1H), 7.71 (d, J=7.7 Hz, 1H), 7.62 (d, J=7.8 Hz, 1H), 7.43-7.36 (m, 2H), 7.31 (q, J=4.2 Hz, 1H), 6.03 (s, 2H).

### Example 100: 2-(3-methylthiophen-2-yl)-1-(pyridin-3-ylmethyl) benzimidazole

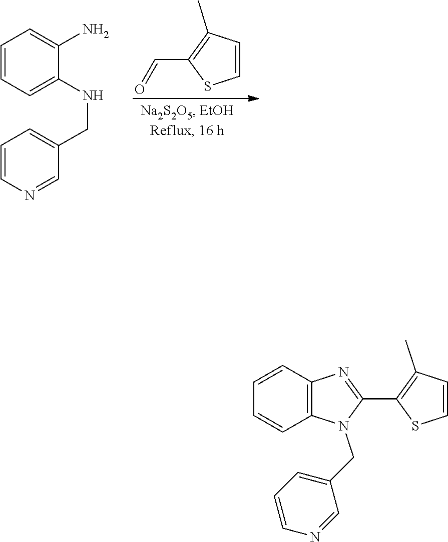

To a mixture of N¹-(pyridin-3-ylmethyl)benzene-1,2-diamine (Intermediate 14) (0.1 g, 0.5 mmol) and 3-methylthiophene-2-carboxaldehyde (0.064 g, 0.5 mmol) in ethanol (10 mL) was added sodium metabisulfite (0.238 g, 1.25 mmol) and the mixture was refluxed for 16 hours. Upon completion, solvent was evaporated, and the residue was diluted with water and extracted with ethyl acetate (2×20 mL). The organic layer was dried over Na₂SO₄, concentrated and purified by chromatography to afford 2-(3-methylthiophen-2-yl)-1-(pyridin-3-ylmethyl)benzimidazole (0.07 g, 40% yield) as an off-white solid.

MS ES⁺: 306.23

¹H NMR (401 MHz, DMSO) 8.43 (t, J=3.2 Hz, 1H), 8.23 (s, 1H), 7.73 (m, 2H), 7.59 (t, J=4.6 Hz, 1H), 7.28 (m, 4H), 7.11 (d, J=5.1 Hz, 1H), 5.55 (s, 2H), 2.22 (s, 3H).

### Example 101: 3-methyl-4-[1-(pyridin-3-ylmethyl)benzimidazol-2-yl]-1,2,5-oxadiazole

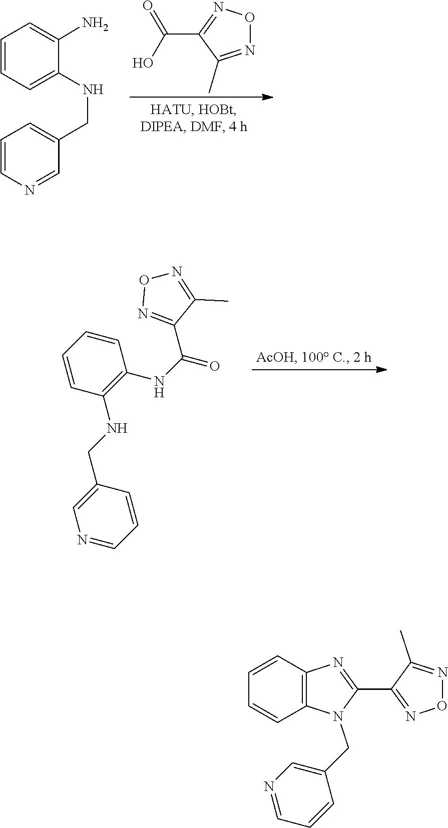

Following the general procedure employed for Example 126 using 4-methyl-1,2,5-oxadiazole-3-carboxylic acid (Intermediate 2) (0.12 g, 1 mmol) and N¹-(pyridin-3-ylmethyl)benzene-1,2-diamine (Intermediate 14) (0.1 g, 0.5 mmol), gave 3-methyl-4-[1-(pyridin-3-ylmethyl)benzimidazol-2-yl]-1,2,5-oxadiazole (0.040 g, 43% yield) as an off-white solid.

MS ES⁺: 292.14

¹H NMR (401 MHz, DMSO) 8.53 (d, J=1.5 Hz, 1H), 8.47 (d, J=4.5 Hz, 1H), 7.89 (d, J=7.7 Hz, 1H), 7.74 (d, J=7.9 Hz, 1H), 7.53 (d, J=7.9 Hz, 1H), 7.40 (m, 2H), 7.32 (q, J =4.2 Hz, 1H), 5.96 (s, 2H), 2.79 (s, 3H).

### Example 102: 5-methyl-4-[1-(pyridin-3-ylmethyl)benzimidazol-2-yl]-1,2,3-thiadiazole

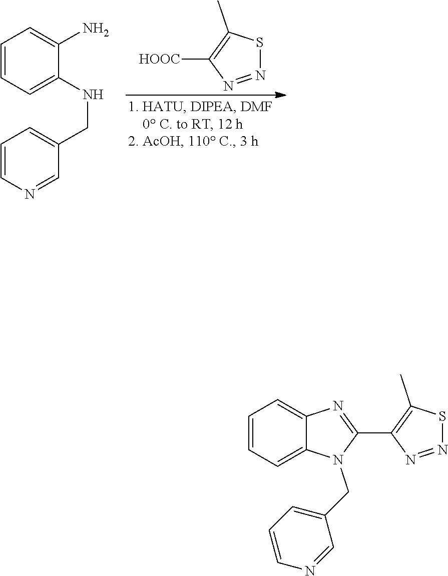

Following the procedure employed for Example 126 using N¹-(pyridin-3-ylmethyl)benzene-1,2-diamine (Intermediate 14) (218 mg, 1.1 mmol) and 5-methyl-1,2,3-thiadiazole-4-carboxylic acid (Intermediate 3) (150 mg, 1 mmol), gave 5-methyl-4-[1-(pyridin-3-ylmethyl)benzimidazol-2-yl]-1,2,3-thiadiazole (60 mg, 22% yield) as a pale yellow solid.

MS ES⁺: 308.24

¹H NMR (400 MHz, DMSO-d6) 8.44 (d, J=4.40 Hz, 2H), 7.82 (d, J=2.00 Hz, 1H), 7.68 (d, J=2.40 Hz, 1H), 7.48 (d, J=8.00 Hz, 1H), 7.28-7.29 (m, 3H), 5.93 (s, 2H), 2.94 (s, 3H).

### Example 103: 5-methyl-4-[1-(pyridin-3-ylmethyl)benzimidazol-2-yl]oxazole

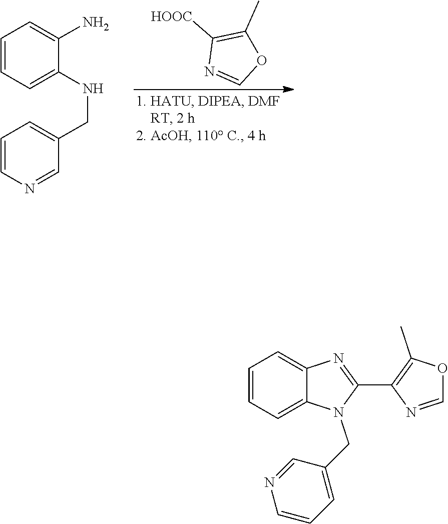

Following the procedure employed for Example 126 using N¹-(pyridin-3-ylmethyl)benzene-1,2-diamine (Intermediate 14) (0.2 g, 1 mmol) and 5-methyl-oxazole-4-carboxylic acid (0.127 g, 1 mmol), gave 5-methyl-4-[1-(pyridin-3-ylmethyl)benzimidazol-2-yl]oxazole (0.088 g, 30% yield) as an off-white solid.

MS ES⁺: 291.17

¹H NMR (400 MHz, DMSO-d₆) 8.50 (s, 2H), 8.44 (q, J=2.1 Hz, 1H), 7.70 (m, J=2.3 Hz, 1H), 7.58 (m, 1H), 7.53 (t, J=11.7 Hz, 1H), 7.27 (m, 3H), 6.03 (s, 2H), 2.77 (s, 3H).

### Example 104: 4-methyl-3-[1-(pyridin-3-ylmethyl)benzimidazol-2-yl]isoxazole

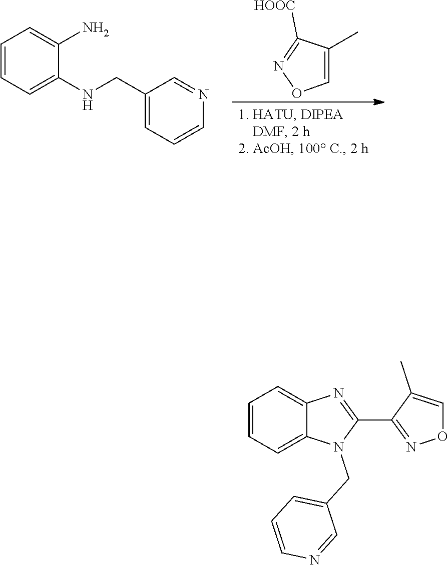

Following the procedure employed for Example 126 using N1-(pyridin-3-ylmethyl)benzene-1,2-diamine (Intermediate 14) (100 mg, 0.5 mmol) and 4-methyl-isoxazole-3-carboxylic acid (64 mg, 0.5 mmol), gave 4-methyl-3-[1-(pyridin-3-ylmethyl)benzimidazol-2-yl]isoxazole (76 mg, 71% yield) as a pale brown solid.

MS ES⁺: 291.13

¹H NMR (400 MHz, DMSO-d₆) 8.99 (s, 1H), 8.46 (t, J=4.7 Hz, 2H), 7.83 (d, J=7.5 Hz, 1H), 7.69 (d, J=7.6 Hz, 1H), 7.48 (d, J=7.9 Hz, 1H), 7.34 (m, 3H), 5.94 (s, 2H), 2.34 (s, 3H).

### Example 105: 3-ethyl-4-[1-(pyridin-3-ylmethyl)benzimidazol-2-yl]-1,2,5-thiadiazole

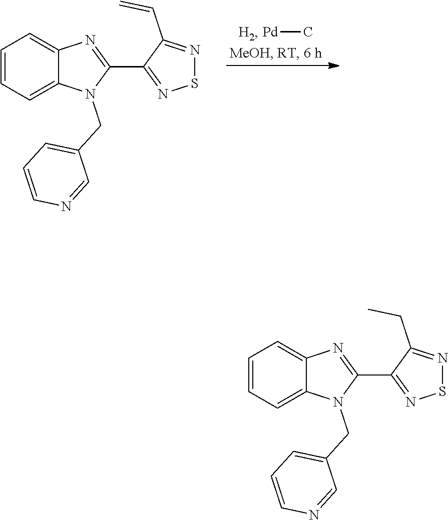

To a solution of 3-[1-(pyridin-3-ylmethyl)benzimidazol-2-yl]-4-vinyl-1,2,5-thiadiazole (Example 89) (30 mg, 0.1 mmol) in dry MeOH (5 mL) was added 10% Pd-C(5 mg).

The solution was transferred to a steel bomb and hydrogenated at RT at 100 psi for 6 hours. The reaction mixture was then filtered through a pad of Celite® and the filtrate was evaporated to dryness to give the crude product, which was purified by reverse phase chromatography using 0-60% methanol in water to afford 3-ethyl-4-[1-(pyridin-3-ylmethyl)benzimidazol-2-yl]-1,2,5-thiadiazole (20 mg, 70% yield) as an off-white solid.

MS ES⁺: 322.2

¹H NMR (400 MHz, DMSO-d₆) 8.45 (t, J=4.8 Hz, 2H), 7.85 (d, J=7.5 Hz, 1H), 7.69 (d, J=7.8 Hz, 1H), 7.49 (d, J=7.8 Hz, 1H), 7.39-7.27 (m, 3H), 5.96 (s, 2H), 3.39 (q, J=7.5 Hz, 2H), 1.30 (t, J=7.4 Hz, 3H).

### Example 106: 4-[1-(pyrimidin-4-ylmethyl)benzimidazol-2-yl]-1,2,5-oxadiazol-3-amine

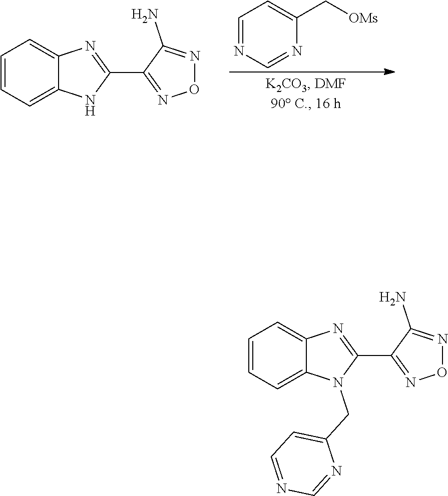

A solution of 4-(benzimidazol-2-yl)-1,2,5-oxadiazol-3-amine (Intermediate 5) (70 mg, 0.3 mmol), pyrimidin-4-ylmethyl methanesulfonate (Intermediate 18) (56 mg, 0.298 mmol) and K₂CO₃ (85 mg, 0.45 mmol) in DMF (3 mL) was stirred at 90° C. for 16 hours. After completion of reaction, the reaction mixture was allowed to cool to RT, diluted with ice cold water (30 mL), and stirred for 15 min. The precipitated solid was filtered and washed with water (3×25 mL). Drying under vacuum to afforded 4-[1-(pyrimidin-4-ylmethyl)benzimidazol-2-yl]-1,2,5-oxadiazol-3-amine (81 mg, 80% yield) as a faint brown solid.

MS ES⁺: 294.16

¹H NMR (400 MHz, DMSO-d₆) 9.00 (d, J=0.80 Hz, 1H), 8.75 (d, J=5.20 Hz, 1H), 7.89 (dd, J=1.60 Hz and 6.60 Hz, 1H), 7.73 (dd, J=2.00, 6.60 Hz, 1H), 7.43-7.36 (m, 3H), 7.00 (s, 2H), 6.09 (s, 2H).

### Example 107: 4-[1-(pyridazin-4-ylmethyl)benzimidazol-2-yl]-1,2,5-oxadiazol-3-amine

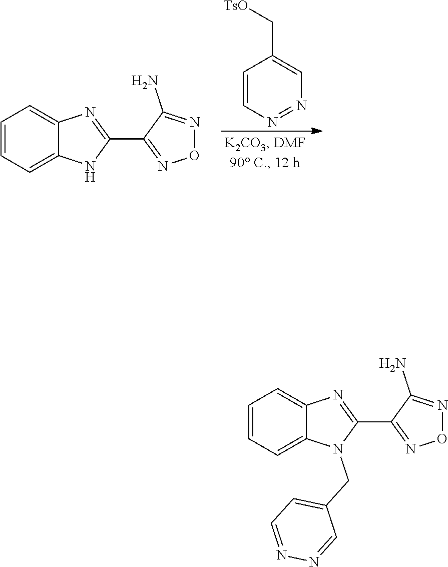 [SMILES: Nc1nonc1-c1nc2ccccc2[nH]1 ; Cc1ccc(S(=O)(=O)OCc2ccnnc2)cc1 ; Nc1nonc1-c1nc2ccccc2n1Cc1ccnnc1]

Following the procedure employed for Example 106 using 4-(benzimidazol-2-yl)-1,2,5-oxadiazol-3-amine (Intermediate 5) (273 mg, 1.3584 mmol) and pyridazin-4-ylmethyl 4-methylbenzenesulfonate (Intermediate 19) (400 mg, 1.509433 mmol) followed by purification by prep. HPLC gave 4-[1-(pyridazin-4-ylmethyl)benzimidazol-2-yl]-1,2,5-oxadiazol-3-amine (0.25 g, 42% yield) as a yellow solid.

MS ES⁺: 294.16

¹H NMR (400 MHz, DMSO-d₆) 9.21 (s, 1H), 9.09 (d, J=5.3 Hz, 1H), 7.91 (d, J=7.2 Hz, 1H), 7.76 (d, J=7.2 Hz, 2H), 7.45-7.43 (m, 1H), 7.21 (q, J=2.4 Hz, 1H), 6.99 (s, 2H), 6.05 (s, 2H).

### Example 108: 3-fluoro-4-[1-(pyridin-3-ylmethyl)benzimidazol-2-yl]-1,2,5-thiadiazole

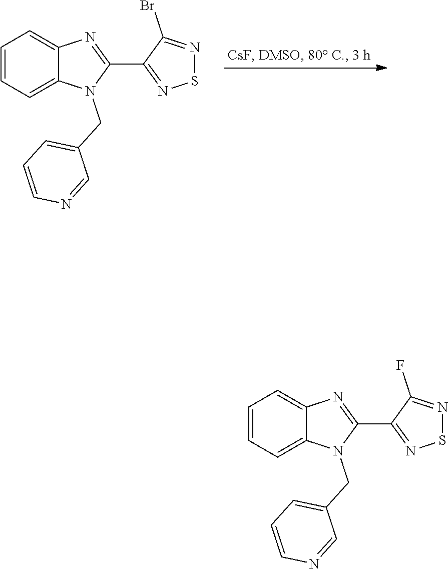 [SMILES: Brc1nsnc1-c1nc2ccccc2n1Cc1cccnc1 ; Fc1nsnc1-c1nc2ccccc2n1Cc1cccnc1]

3-Bromo-4-(1-(pyridin-3-ylmethyl)benzimidazol-2-yl)-1,2,5-thiadiazole (Example 126) (0.06 g, 0.2 mmol) and caesium fluoride (6 g, 0.4 mmol) in DMSO (5 mL) were placed in a sealed tube and stirred for 3 hours at 80° C. The reaction was then quenched by adding ice water and extracted with ethyl acetate (2×10 mL). The organic layer was dried over Na₂SO₄, concentrated and purified by reverse phase chromatography using 55% methanol in water to afford 3-fluoro-4-[1-(pyridin-3-ylmethyl)benzimidazol-2-yl]-1,2,5-thiadiazole (16 mg, 48% yield) as a yellow solid.

MS ES⁺: 312.18

¹H NMR (400 MHz, DMSO-d₆) 8.57 (d, J=1.5 Hz, 1H), 8.47 (d, J=3.6 Hz, 1H), 7.86 (d, J=7.2 Hz, 1H), 7.68 (d, J=7.3 Hz, 1H), 7.58 (d, J=7.9 Hz, 1H), 7.35 (m, 3H), 6.00 (s, 2H).

### Example 109: 4-[I-[[2-(trifluoromethyl)pyridin-3-yl]methyl]benzimidazol-2-yl]-1,2,5-oxadiazol-3-amine

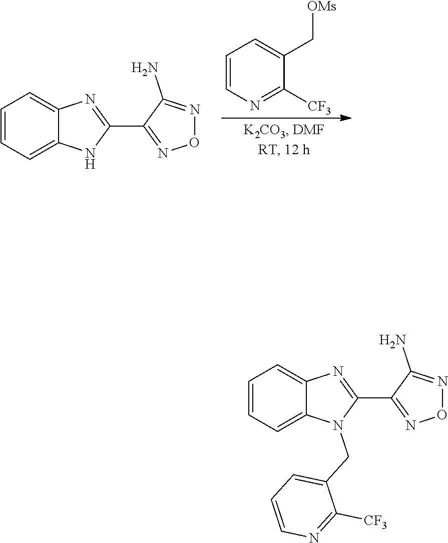

Following the procedure employed for Example 86 using 4-(benzimidazol-2-yl)-1,2,5-oxadiazol-3-amine (Intermediate 5) (0.10 g, 0.5 mmol) and (2-(trifluoromethyl)pyridin-3-yl)methyl methanesulfonate (Intermediate 20) (0.153 g, 0.6 mmol), gave 4-[1-[[2-(trifluoromethyl)pyridin-3-yl]methyl]benzimidazol-2-yl]-1,2,5-oxadiazol-3-amine (0.020 g, 11% yield) as an off-white solid.

MS ES⁺: 361.18

¹H NMR (400 MHz, DMSO-d₆) 8.63 (d, J=4.4 Hz, 1H), 7.94 (m, 1H), 7.68 (m, J=2.3 Hz, 1H), 7.51 (q, J=4.2 Hz, 1H), 7.44 (m, 2H), 6.98 (s, 2H), 6.94 (d, J=7.9 Hz, 1H), 6.15 (s. 2H).

### Example 110: 3-methyl-4-[3-(pyridin-3-ylmethyl)imidazo[4,5-c]pyridin-2-yl]-1,2,5-oxadiazole

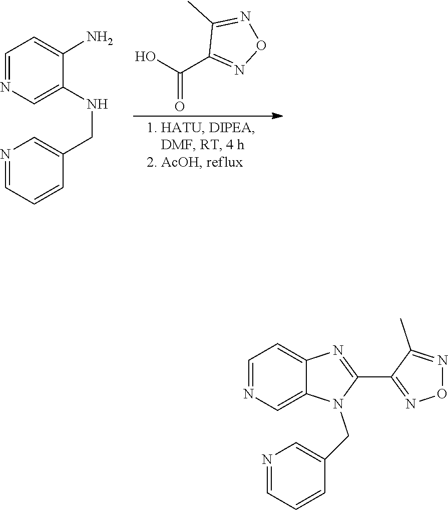

Following the procedure employed for Example 126 using N3-(pyridin-3-ylmethyl)pyridine-3,4-diamine (Intermediate 21) (100 mg, 0.50 mmol) and 4-methyl-1,2,5-oxadiazole-3-carboxylic acid (Intermediate 2) (44 mg, 0.35 mmol), gave 3-methyl-4-[3-(pyridin-3-ylmethyl)imidazo[4,5-c]pyridin-2-yl]-1,2,5-oxadiazole (62 mg, 33% yield) as a white solid.

MS ES⁺: 293.16

¹H NMR (400 MHz, DMSO-d₆) 9.15 (s, 1H), 8.59 (d, J=2.00 Hz, 1H), 8.49 (d, J=5.60 Hz, 2H), 7.89 (d, J=5.60 Hz, 1H), 7.61 (d, J=8.00 Hz, 1H), 7.33-7.34 (m, 1H), 6.04 (s, 2H), 2.78 (s, 3H).

### Example 111: 4,5-dimethyl-3-[1-(pyridin-3-ylmethyl)benzimidazol-2-yl]isoxazole

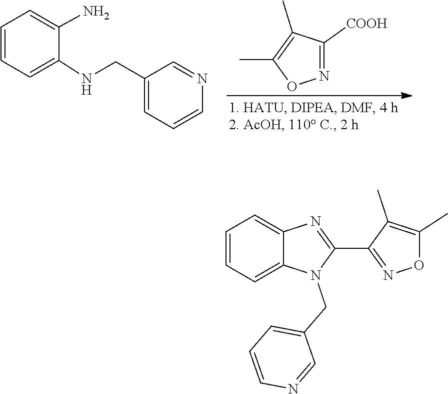

Following the procedure employed for Example 126 using N¹-(pyridin-3-ylmethyl)benzene-1,2-diamine (Intermediate 14) (120 mg, 0.6 mmol) and 4,5-dimethylisoxazole-3-carboxylic acid (85 mg, 0.6 mmol), gave 4,5-dimethyl-3-[1-(pyridin-3-ylmethyl)benzimidazol-2-yl]isoxazole (130 mg, 85% yield) as a pale brown solid.

MS ES⁺: 305.34

¹H NMR (400 MHz, DMSO-d₆) 8.45 (m, 2H), 7.82 (d, J=7.4 Hz, 1H), 7.67 (d, J=7.5 Hz, 1H), 7.48 (d, J=7.9 Hz, 1H), 7.35-7.33 (m, 3H), 5.93 (s, 2H), 2.45 (s, 3H), 2.26 (s, 3H).

### Example 112: 3-methyl-4-[1-(pyridin-3-ylmethyl)benzimidazol-2-yl]isoxazole

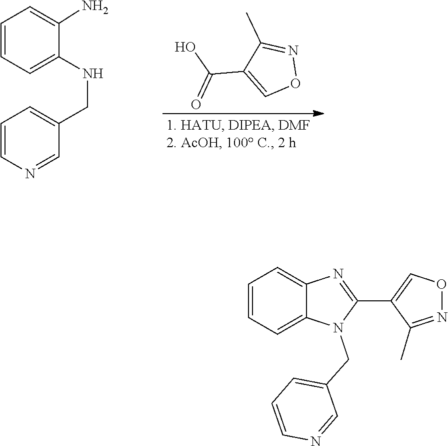

Following the procedure employed for Example 126 using N¹-(pyridin-3-ylmethyl)benzene-1,2-diamine (Intermediate 14) (0.1 g, 0.5 mmol) and 3-methylisoxazole-4-carboxylic acid (0.063 g, 0.5 mmol), gave 3-methyl-4-[1-(pyridin-3-ylmethyl)benzimidazol-2-yl]isoxazole (0.08 g, 79.6% yield) as a white solid.

MS ES⁺: 291.33

¹H NMR (401 MHz, DMSO-d₆) 9.38 (s, 1H), 8.45 (t, J=3.1 Hz, 1H), 8.33 (s, 1H), 7.75 (q, J=3.0 Hz, 1H), 7.61 (t, J=4.5 Hz, 1H), 7.29 (t, J=3.9 Hz, 4H), 5.70 (s, 2H), 2.43 (s, 3H).

### Example 113: 2-(1,4-dimethylpyrazol-3-yl)-1-(pyridin-3-ylmethyl) benzimidazole

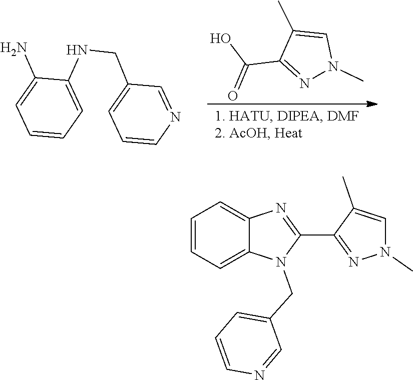

Following the procedure employed for Example 126 using N¹-(pyridin-3-ylmethyl)benzene-1,2-diamine (Intermediate 14) (200 mg, 1 mmol) and 1,4-dimethyl-1H-pyrazole-3-carboxylic acid (169 mg, 1.2 mmol), gave 2-(1,4-dimethylpyrazol-3-yl)-1-(pyridin-3-ylmethyl)benzimidazole (83 mg, 28% yield) as an off-white solid.

MS ES⁺: 304.27

¹H NMR (400 MHz, DMSO-d₆) 8.49 (s, 1H), 8.42 (d, J=4.3 Hz, 1H), 7.69 (d, J=5.8 Hz, 2H), 7.54 (m, 2H), 7.26 (m, 3H), 6.00 (s, 2H), 3.88 (s, 3H), 2.35 (s, 3H).

### Example 114: 2-(1-methylpyrazol-5-yl)-1-(pyridin-3-ylmethyl) benzimidazole

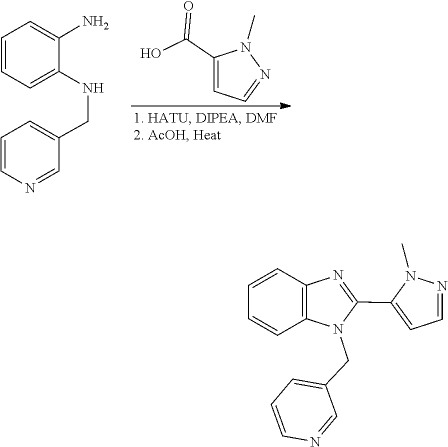

Following the procedure employed for Example 126 using N¹-(pyridin-3-ylmethyl)benzene-1,2-diamine (Intermediate 14) (100 mg, 0.50 mmol) and 1-methyl-1H-pyrazole-5-carboxylic acid (63 mg, 0.50 mmol), gave 2-(1-methylpyrazol-5-yl)-1-(pyridin-3-ylmethyl)benzimidazole (55 mg, 39% yield) as an off-white solid.

MS ES⁺: 290.17

¹H NMR (400 MHz, DMSO-d₆) 8.46 (dd, J=2.00, 4.40 Hz, 1H), 8.30 (s, 1H), 7.79-7.79 (m, 1H), 7.64-7.64 (m, 1H), 7.61 (d, J=2.00 Hz, 1H), 7.29-7.30 (m, 4H), 6.65 (d, J=2.00 Hz, 1H), 5.66 (s, 2H), 3.98 (s, 3H).

### Example 115: 4-[1-(pyridin-2-ylmethyl)benzimidazol-2-yl]-1,2,5-oxadiazol-3-amine

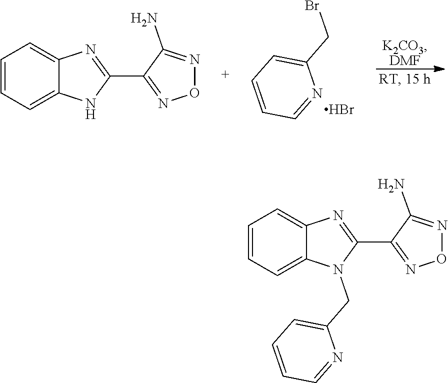

Following the procedure employed for Example 86 using 4-(benzimidazol-2-yl)-1,2,5-oxadiazol-3-amine (Intermediate 5) (0.1 g, 0.5 mmol) and 2-(bromomethyl)pyridine hydrobromide (0.15 g, 0.6 mmol), gave 4-[1-(pyridin-2-ylmethyl)benzimidazol-2-yl]-1,2,5-oxadiazol-3-amine (0.08 g, 57% yield) as an off-white solid.

MS ES⁺: 293.29

¹H NMR (400 MHz, DMSO-d₆) 8.39 (t, J=2.8 Hz, 1H), 7.86 (q, J=2.9 Hz, 1H), 7.77-7.68 (m, 2H), 7.40-7.34 (m, 2H), 7.25 (q, J=3.9 Hz, 2H), 7.01 (s, 2H), 6.06 (s, 2H).

### Example 116: 3-ethyl-4-[1-(pyridin-3-ylmethyl)benzimidazol-2-yl]-1,2,5-oxadiazole

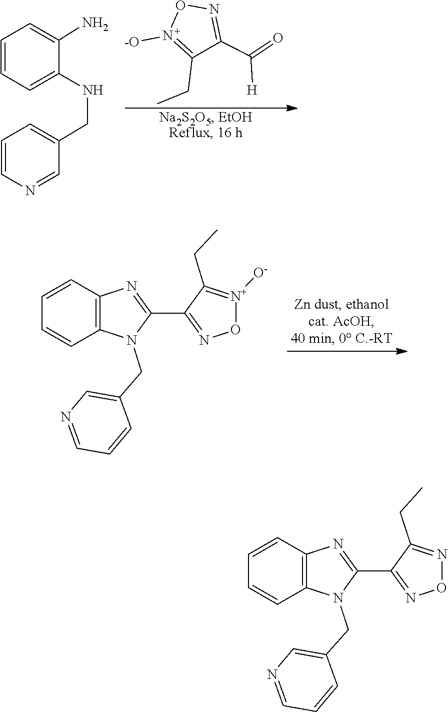

Step 1: To a mixture of N¹-(pyridin-3-ylmethyl)benzene-1,2-diamine (Intermediate 14) (1.2 g, 6 mmol) and 3-ethyl-4-formyl-1,2,5-oxadiazole 2-oxide (0.85 g, 6 mmol) in EtOH (10 mL), sodium metabisulfite (2.8 g, 15 mmol) was added slowly. The resulting mixture was allowed to stir at 50° C. for 16 hours. After this time, the crude reaction mixture was evaporated under reduced pressure and the residue was extracted with ethyl acetate. The organic layer was dried over sodium sulfate and concentrated in vacuum. The crude product was purified by column chromatography to afford 3-ethyl-4-(1-(pyridin-3-ylmethyl)-benzimidazol-2-yl)-1,2,5-oxadiazole 2-oxide (0.50 g, 26% yield) as an off-white solid.

Step 2: To a mixture of 3-ethyl-4-(1-(pyridin-3-ylmethyl)-benzimidazol-2-yl)-1,2,5-oxadiazole 2-oxide (0.10 g, 0.3 mmol) in ethanol (4 mL), was added acetic acid (0.03 mL) and Zn dust (0.03 g, 0.6 mmol) slowly at 0° C. The resulting mixture was stirred at 0° C. to RT for 40 min. After completion of the reaction, the mixture was basified with sodium bicarbonate and extracted with ethyl acetate. The organic layer was dried over Na₂SO₄ and concentrated under vacuum. The crude product was purified by prep. HPLC to obtain 3-ethyl-4-[1-(pyridin-3-ylmethyl)benzimidazol-2-yl]-1,2,5-oxadiazole (0.010 g, 11% yield) as an off-white solid.

MS ES⁺: 306.26

¹H NMR (400 MHz, DMSO-d₆) 8.53-8.45 (m, 2H), 7.88 (d, J=8.0 Hz, 1H), 7.75 (d, J=8.0 Hz, 1H), 7.51 (d, J=7.6 Hz, 1H), 7.45-7.28 (m, 3H), 5.95 (s, 2H), 3.40-3.30 (m, 2H), 1.36 (t, J=7.6 Hz, 3H).

### Example 117: 2-(furan-2-yl)-1-(pyridin-4-ylmethyl)benzimidazole

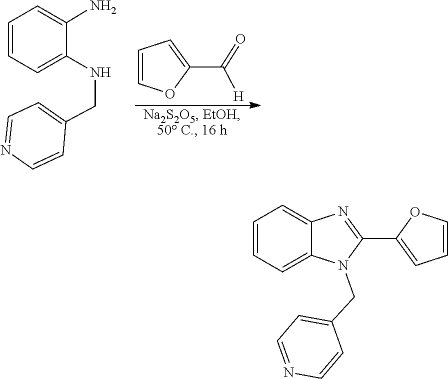

A mixture of furan-2-carboxaldehyde (0.108 g, 1.12 mmol), N¹-(pyridin-4-ylmethyl)benzene-1,2-diamine (Intermediate 16) (0.15 g, 0.75 mmol) and sodium metabisulfite (0.285 g, 1.5 mmol) was suspended in ethanol (8 mL). The resultant mixture was stirred at 50° C. for 16 hours. After completion of the reaction, the solvent was evaporated and the residue was suspended in ethyl acetate (50 mL) and washed with sat. NaHCO₃, water and brine sequentially. The organic layer was dried over Na₂SO₄ and concentrated in vacuum. The residue was purified by chromatography to afford 2-(furan-2-yl)-1-(pyridin-4-ylmethyl)benzimidazole (35 mg, 17% yield) as an off-white solid.

MS ES⁺: 276.22

¹H NMR (400 MHz, DMSO-d₆) 8.48 (q, J=2.0 Hz, 2H), 7.90 (q, J=0.8 Hz, 1H), 7.72 (m, 1H), 7.58 (m, 1H), 7.28 (m, 2H), 7.14 (q, J=1.4 Hz, 1H), 7.04 (q, J=2.0 Hz, 2H), 6.70 (q, J=1.8 Hz, 1H), 5.85 (s, 2H).

### Example 118: 4-[6-fluoro-1-(pyridin-3-ylmethyl)benzimidazol-2-yl]-1,2,5-oxadiazol-3-amine

### Example 119: 4-[5-fluoro-1-(pyridin-3-ylmethyl)benzimidazol-2-yl]-1,2,5-oxadiazol-3-amine

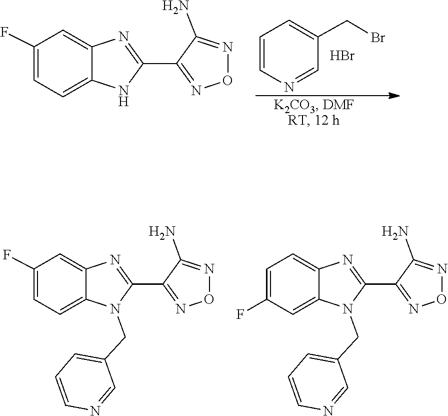

Following the procedure employed for Example 86 using 4-(5-fluoro-benzimidazol-2-yl)-1,2,5-oxadiazol-3-amine (Intermediate 8) (0.2 g, 0.91 mmol) and 3-(bromomethyl)pyridine hydrobromide (0.372 g, 2.7 mmol) an isomeric mixture of desired products was obtained. The crude mixture was separated by SFC (Column/dimensions: Chiralcel-OD-3 (4.6×150)mm, 3 μm; % CO₂: 70%; % Co-solvent: 30% (MeOH); Total Flow: 3.00 g/min; Back Pressure: 1500 bar; Temperature: 30° C.; UV: 220 nm) to afford Peak 2 (4-[6-fluoro-1-(pyridin-3-ylmethyl)benzimidazol-2-yl]-1,2,5-oxadiazol-3-amine) (0.09 g, 48% yield) as an off-white solid and Peak 1 (4-[5-fluoro-1-(pyridin-3-ylmethyl)benzimidazol-2-yl]-1,2,5-oxadiazol-3-amine) (0.066 g, 28% yield) as an off-white solid.

### Example 118 (Peak 2)

MS ES⁺: 311.17

¹H NMR (400 MHz, DMSO-d₆) 8.52 (d, J=1.6 Hz, 1H), 8.48 (t, J=4.4 Hz, 1H), 7.95-7.88 (m, 1H), 7.80-7.76 (dd, J=2.4 Hz and 9.2 Hz, 1H), 7.51 (d, J=8.0 Hz, 1H), 7.35-7.22 (m, 2H), 6.97 (s, 2H), 5.97 (s, 2H).

### Example 119 (Peak 1)

MS ES⁺: 311-13

¹H NMR (400 MHz, DMSO-d₆) 8.53 (d, J=1.6 Hz, 1H), 8.48 (d, J=3.6 Hz, 1H), 7.85-7.80 (m, 1H), 7.72-7.68 (dd, J=2.4 Hz and 9.2 Hz, 1H), 7.51 (d, J=8.0 Hz, 1H), 7.35-7.28 (m, 2H), 6.98 (s, 2H), 6.01 (s, 2H).

### Example 120: 4-[7-fluoro-1-(pyridin-4-ylmethyl)benzimidazol-2-yl]-1,2,5-oxadiazol-3-amine

### Example 121: 4-[4-fluoro-1-(pyridin-4-ylmethyl)benzimidazol-2-yl]-1,2,5-oxadiazol-3-amine

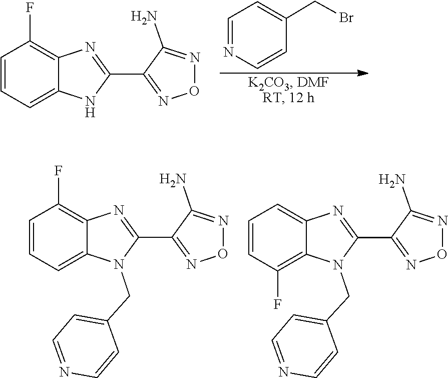

Following the procedure employed for Example 86 using 4-(4-fluoro-benzimidazol-2-yl)-1,2,5-oxadiazol-3-amine (Intermediate 4) (0.25 g, 1.1 mmol) and 4-(bromomethyl)pyridine (0.295 g, 1.7 mmol) an isomeric mixture of desired products was obtained. The crude mixture was separated by SFC (Column/dimensions: Chiralcel-OJ-H (30×250)mm, 5 μm; % CO₂: 70%; % Co-solvent: 30% (MeOH); Total Flow: 100.0 g/min; Back Pressure: 100 bar; Temperature: 30° C.; UV: 220 nm) to afford Peak 2 (4-[7-fluoro-1-(pyridin-4-ylmethyl)benzimidazol-2-yl]-1,2,5-oxadiazol-3-amine) (0.07 g, 20% yield) as an off-white solid and Peak 1 (4-[4-fluoro-1-(pyridin-4-ylmethyl)benzimidazol-2-yl]-1,2,5-oxadiazol-3-amine) (0.066 g, 28% yield) as an off-white solid.

### Example 120 (Peak 2)

MS ES⁺: 311.17

¹HNMR (400 MHz, DMSO-d6) 8.50 (d, J=5.6 Hz, 2H), 7.75 (d, J=8.0 Hz, 1H), 7.40-7.35 (m, 1H), 7.28-7.22 (m, 1H), 7.10 (d, J=5.6 Hz, 2H), 6.98 (s, 2H), 6.04 (s, 2H).

### Example 121 (Peak 1)

MS ES⁺: 311-13

¹HNMR (400 MHz, DMSO-d6) 8.49 (d, J=5.6 Hz, 2H), 7.56 (d, J=8.0 Hz, 1H), 7.45-7.38 (m, 1H), 7.29-7.20 (m, 1H), 7.10 (d, J=5.6 Hz, 2H), 6.94 (s, 2H), 6.01 (s, 2H).

### Example 122: 3-[7-fluoro-1-(pyridin-3-ylmethyl)benzimidazol-2-yl]-4-methyl-1,2,5-oxadiazole

### Example 123: 3-[4-fluoro-1-(pyridin-3-ylmethyl)benzimidazol-2-yl]-4-methyl-1,2,5-oxadiazole

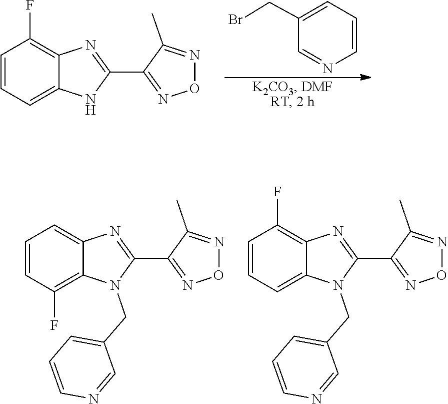

Following the procedure employed for Example 86 using 3-(4-fluoro-benzimidazol-2-yl)-4-methyl-1,2,5-oxadiazole (Intermediate 9) (0.28 g, 1.28 mmol) and 3-(bromomethyl)pyridine (0.329 g, 1.92 mmol) an isomeric mixture of desired products was obtained. The crude mixture was separated by SFC to afford Peak 1 (3-[7-fluoro-1-(pyridin-3-ylmethyl)benzimidazol-2-yl]-4-methyl-1,2,5-oxadiazole) (0.04 g, 20% yield) as an off-white solid and Peak 2 (3-[4-fluoro-1-(pyridin-3-ylmethyl)benzimidazol-2-yl]-4-methyl-1,2,5-oxadiazole) (0.03 g, 17% yield) as an off-white solid.

### Example 122 (Peak 1)

MS ES⁺: 310.34

¹H NMR (400 MHz, DMSO-d6) 8.50 (d, J=4.0 Hz, 1H), 8.47 (s, 1H), 7.74 (d, J=8.0 Hz, 1H), 7.53 (d, J=8.0 Hz, 1H), 7.38-7.32 (m, 2H), 7.28-7.20 (m, 1H), 5.98 (s, 2H), 2.78 (s, 3H).

### Example 123 (Peak 2)

MS ES⁺: 310.34

¹H NMR (400 MHz, DMSO-d6) 8.54 (s, 1H), 8.48 (d, J=4.0 Hz, 1H), 7.59 (d, J=8.0 Hz, 1H), 7.54 (d, J=8.0 Hz, 1H), 7.45-7.38 (m, 1H), 7.35-7.30 (m, 1H), 7.25-7.18 (m, 1H), 5.97 (s, 2H), 2.78 (s, 3H).

### Example 124: 3-[4-fluoro-1-(pyridin-3-ylmethyl)benzimidazol-2-yl]-4-methyl-1,2,5-thiadiazole

### Example 125: 3-[7-fluoro-1-(pyridin-3-ylmethyl)benzimidazol-2-yl]-4-methyl-1,2,5-thiadiazole

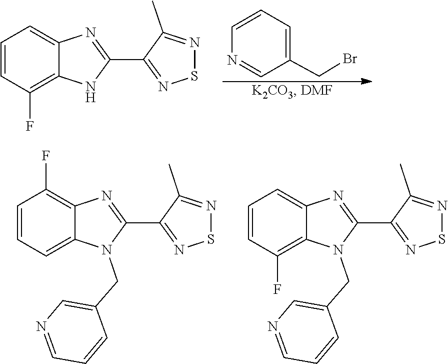

Following the procedure employed for Example 86 using 3-(7-fluoro-benzimidazol-2-yl)-4-methyl-1,2,s-thiadiazole (Intermediate 10) (0.15 g, 0.64 mmol) and 3-(bromomethyl)pyridine (0.5 g, 0.9 mmol) an isomeric mixture of desired products was obtained. The crude mixture was separated by SFC to afford Peak 1 (3-[4-fluoro-1-(pyridin-3-ylmethyl)benzimidazol-2-yl]-4-methyl-1,2,5-thiadiazole) (0.094 g, 45% yield) as an off-white solid and Peak 2 (3-[7-fluoro-1-(pyridin-3-ylmethyl)benzimidazol-2-yl]-4-methyl-1,2,5-thiadiazole) (0.062 g, 30% yield) as an off-white solid.

### Example 124 (Peak 1)

MS ES⁺: 326.17

¹H NMR (400 MHz, DMSO-d₆) 8.52 (s, 1H), 8.46 (d, J=4.80 Hz, 1H), 7.53 (d, J=8.00 Hz, 2H), 7.38-7.28 (m, 2H), 7.20-7.15 (m, 1H), 5.99 (s, 2H), 2.94 (s, 3H).

### Example 125 (Peak 2)

MS ES⁺: 326.27

¹H NMR (400 MHz, DMSO-d₆) 8.47 (d, J=4.4 Hz, 1H), 8.43 (s, 1H), 7.70 (d, J=8.00 Hz, 1H), 7.50 (d, J=8.0 Hz, 1H), 7.34-7.28 (m, 2H), 7.21-7.15 (m, 1H), 5.99 (s, 2H), 2.92 (s, 3H).

### Example 126: 3-bromo-4-(1-(pyridin-3-ylmethyl)benzimidazol-2-yl)-1,2,5-thiadiazole

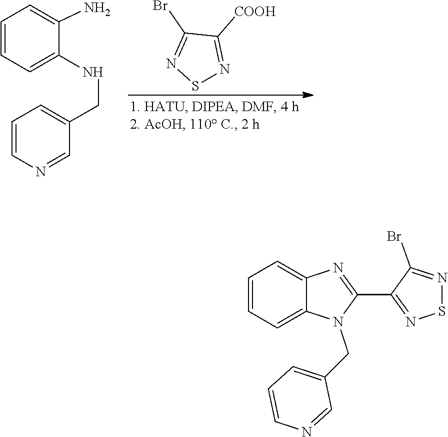 [SMILES: Nc1ccccc1NCc1cccnc1 ; O=C(O)c1nsnc1Br ; Brc1nsnc1-c1nc2ccccc2n1Cc1cccnc1]

To a solution of 4-bromo-1,2,5-thiadiazole-3-carboxylic acid (Intermediate 7) (0.9 g, 4.3 mmol) in DMF (20 mL) at 0° C. was added HATU (2.85 g, 7-5 mmol) and DIPEA (1.8 mL, 10.0 mmol), followed by the addition of N¹-(pyridin-3-ylmethyl)benzene-1,2-diamine (Intermediate 14) (1.0 g, 5.0 mmol). The resulting mixture was stirred at RT for 4 hours. After completion of the reaction, the reaction mixture was diluted with ice cooled water and extracted with ethyl acetate. The organic layer was dried over Na₂SO₄ and concentrated under reduced pressure. The resulting brown gummy material was dissolved in acetic acid (10 mL) and refluxed at 110° C. for 2 hours. The reaction mixture was diluted with water and extracted with ethyl acetate. The organic layer was dried over Na₂SO₄ and concentrated under reduced pressure. The crude product was purified by flash chromatography (0-60% EtOAc in petroleum ether) to afford 3-bromo-4-(1-(pyridin-3-ylmethyl)benzimidazol-2-yl)-1,2,5-thiadiazole (1.5 g, 94% yield) as a pale yellow solid.

MS ES⁺: 372.07

¹H NMR (400 MHz, DMSO-d₆) 8.46 (t, J=2.4 Hz, 2H), 7.86 (q, J=2.8 Hz, 1H), 7.68 (q, J=2.8 Hz, 1H), 7.51 (d, J=7.9 Hz, 1H), 7.35 (m, J=3.6 Hz, 3H), 5.79 (s, 2H).

### Example 127: 3-methyl-4-[1-(pyridin-3-ylmethyl)benzimidazol-2-yl]-1,2,5-thiadiazole

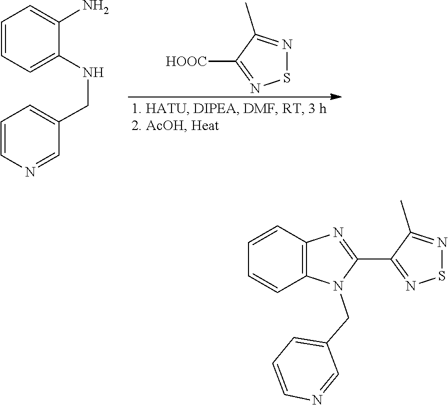

Following the procedure employed for Example 126 using 4-methyl-1,2,5-thiadiazole-3-carboxylic acid (0.051 g, 0.35 mmol) and N1-(pyridin-3-ylmethyl)benzene-1,2-diamine (Intermediate 14) (0.07 g, 0.35 mmol), gave 3-methyl-4-[1-(pyridin-3-ylmethyl)benzimidazol-2-yl]-1,2,5-thiadiazole (0.06 g, 55% yield) as an off-white solid.

MS ES⁺: 308.3

¹H NMR (400 MHz, DMSO-d₆) 8.50 (s, 1H), 8.45 (d, J=4.0 Hz, 1H), 7.86 (d, J=6.80 Hz, 1H), 7.68 (d, J=6.8 Hz, 1H), 7.51 (d, J=8.0 Hz, 1H), 7.40-7.25 (m, 3H), 5.98 (s, 2H), 2.94 (s, 3H).

### Example 128: 4-[7-fluoro-1-(pyridin-3-ylmethyl)benzimidazol-2-yl]-5-methyl-1,2,3-thiadiazole

### Example 129: 4-[4-fluoro-1-(pyridin-3-ylmethyl)benzimidazol-2-yl]-5-methyl-1,2,3-thiadiazole

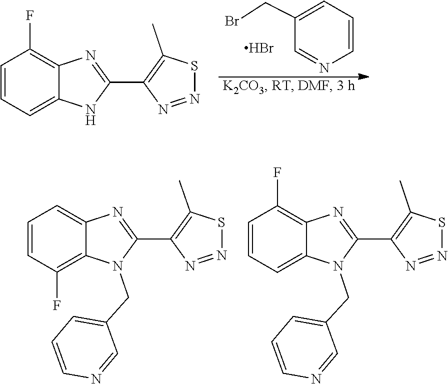

Following the procedure employed for Example 86 using 4-(4-fluoro-benzimidazol-2-yl)-5-methyl-1,2,3-thiadiazole (Intermediate 11) (0.25 g, 1.1 mmol) and 3-(bromomethyl)pyridine hydrobromide (0.333 g, 1.32 mmol) an isomeric mixture of desired products was obtained. The crude mixture was separated by SFC (Column/dimensions: Chiralcel-OJ-H (30×250)mm, 5 μm; % CO₂: 70%; % Co-solvent: 30% (MeOH); Total Flow: 100.0 g/min; Back Pressure: 100 bar; Temperature: 30° C.; UV: 220 nm) to afford Peak 1 (4-[7-fluoro-1-(pyridin-3-ylmethyl)benzimidazol-2-yl]-5-methyl-1,2,3-thiadiazole) (0.06 g, 19% yield) as an off-white solid and Peak 2 (4-[4-fluoro-1-(pyridin-3-ylmethyl)benzimidazol-2-yl]-5-methyl-1,2,3-thiadiazole) (0.065 g, 20% yield) as an off-white solid.

### Example 128 (Peak 1)

MS ES⁺: 326.31

¹H NMR (400 MHz, DMSO-d₆) 8.46 (d, J=3.8 Hz, 1H), 8.36 (s, 1H), 7.67 (d, J=8.0 Hz, 1H), 7.46 (d, J=8.0 Hz, 1H), 7.33-7.27 (m, 2H), 7.19-7.14 (m, 1H), 5.94 (s, 2H), 2.94 (s, 3H).

### Example 129 (Peak 2)

MS ES⁺: 326.31

¹H NMR (400 MHz, DMSO-d₆) 8.45 (br s, 2H), 7.53 (d, J=8.0 Hz, 1H), 7.48 (d, J=8.0 Hz, 1H), 7.36-7.27 (m, 2H), 7.18-7.13 (m, 1H), 5.94 (s, 2H), 2.95 (s, 3H).

### Example 130: 4-methyl-5-[1-(pyridin-3-ylmethyl)benzimidazol-2-yl]isoxazole

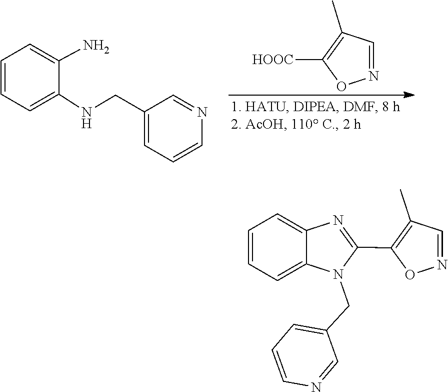

Following the procedure employed for Example 126 using 4-methylisoxazole-5-carboxylic acid (64 mg, 0.5 mmol) and N¹-(pyridin-3-ylmethyl)benzene-1,2-diamine (Intermediate 14) (100 mg, 0.5 mmol), gave 4-methyl-5-[1-(pyridin-3-ylmethyl)benzimidazol-2-yl]isoxazole (0.080 g, 75% yield) as a pale yellow solid.

MS ES⁺: 291.35

¹H NMR (400 MHz, DMSO-d₆) 8.75 (s, 1H), 8.48-8.42 (m, 2H), 7.82 (q, J=7.6 Hz, 1H), 7.76 (q, J=8.0 Hz, 1H), 7.46-7.28 (m, 4H), 5.86 (s, 2H), 2.38 (s, 3H).

### Example 131: 4-[4-fluoro-1-(pyrimidin-5-ylmethyl)benzimidazol-2-yl]-1,2,5-oxadiazol-3-amine

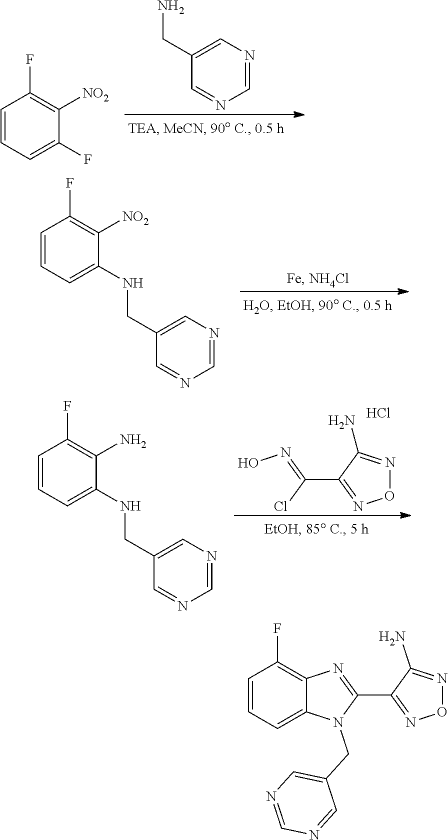

Step 1: A mixture of 1,3-difluoro-2-nitro-benzene (350 mg, 2.20 mmol), pyrimidin-5-ylmethanamine (240.08 mg, 2.20 mmol) and triethylamine (445.23 mg, 4.40 mmol) in acetonitrile (2 mL) was stirred at 90° C. for 0.5 hour. The mixture was concentrated to the dryness. The residue was purified by flash chromatography (ISCO®; 4 g SepaFlash® Silica Flash Column, eluent of 050% ethyl acetate in petroleum ether gradient @25 mL/min) to give 3-fluoro-2-nitro-N-(pyrimidin-5-ylmethyl)aniline (337 mg, 1.36 mmol, 61.7% yield) as a yellow solid.

MS ES⁺: 249.1

Step 2: To a mixture of 3-fluoro-2-nitro-N-(pyrimidin-5-ylmethyl)aniline (177 mg, 0.713 mmol) in H₂O (2.5 mL) and EtOH (2.5 mL) were added Fe (199.11 mg, 3.57 mmol) and NH₄Cl (190.72 mg, 3.57 mmol). The mixture was then stirred at 90° C. for 0.5 hour, at which point the mixture was cooled down to room temperature and filtered. The filtrate was concentrated to remove most of the EtOH, and then extracted with ethyl acetate (5 mL×3). The combined organic layers were dried with Na₂SO₄ and concentrated to afford 3-fluoro-N¹-(pyrimidin-5-ylmethyl)benzene-1,2-diamine (186 mg) as a black oil which was used in the next step without further purification.

MS ES⁺: 219.2

Step 3: A mixture of 3-fluoro-N1-(pyrimidin-5-ylmethyl)benzene-1,2-diamine (186 mg) and 4-amino-N-hydroxy-1,2,5-oxadiazole-3-carboximidoyl chloride hydrochloride (169.61 mg, 0.852 mmol) in EtOH (1.5 mL) was stirred at 85° C. for 5 hours. The reaction mixture was concentrated to afford the crude product which was purified by prep. HPLC (column: Phenomenex Luna C₁₈ 75*30 mm*3 μm, Mobile Phase A: water (0.225% FA), Mobile Phase B: MeCN, Flow rate: 25 mL/min, gradient condition from 10% B to 60%). The pure fractions were collected and the volatiles removed under vacuum. The residue was partitioned between acetonitrile (2 mL) and water (10 mL).

The solution was lyophilized to dryness to give the product which was further purified by prep. HPLC (column: Phenomenex Gemini-NX C₁₈ 75*30 mm*3 μm, Mobile Phase A: water (0.05% NH₃·H₂O+10 mM NH₄HCO₃), Mobile Phase B: acetonitrile, Flow rate: 25 mL/min, gradient condition from 17% B to 57%). The pure fractions were collected and the volatiles removed under vacuum. The residue was partitioned between acetonitrile (2 mL) and water (10 mL). The solution was lyophilized to dryness to give 4-[4-fluoro-1-(pyrimidin-5-ylmethyl)benzimidazol-2-yl]-1,2,5-oxadiazol-3-amine (5.5 mg, 0.017 mmol, 2.1% yield, 98.7% purity) as a white powder.

MS ES⁺: 312.3

¹H NMR (400 MHz, DMSO-d₆) 9.12 (s, 1H), 8.72 (s, 2H), 7.69 (d, J=8.4 Hz, 1H), 7.52-7.38 (m, 1H), 7.24 (d, J=10.8 Hz, 1H), 6.93 (s, 2H), 6.02 (s, 2H).

### Example 132: 4-(7-fluoro-1-(pyridazin-4-ylmethyl)-benzimidazol-2-yl)-1,2,5-oxadiazol-3-amine

### Example 133: 4-(4-fluoro-1-(pyridazin-4-ylmethyl)-benzimidazol-2-yl)-1,2,5-oxadiazol-3-amine

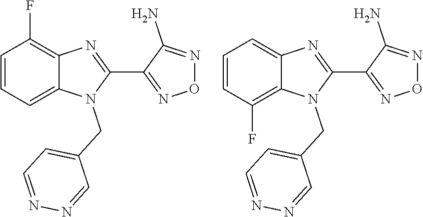

Step 1: A mixture of 3-fluorobenzene-1,2-diamine (4 g, 31.7 mmol) and 4-amino-N-hydroxy-1,2,5-oxadiazole-3-carboximidoyl chloride hydrochloride (6.31 g, 31.7 mmol) in EtOH (40 mL) was stirred at 85° C. for 24 hours and cooled to RT with grey precipitation forming. The precipitate was collected by filtration to give 4-(7-fluoro-1H-benzimidazol-2-yl)-1,2,5-oxadiazol-3-amine (4.0 g, 18 mmol, 57%) as a grey solid.

MS ES⁺: 220.1

¹H NMR (400 MHz, DMSO-d₆) 14.02 (br s, 1H), 7.46-7.41 (m, 1H), 7.36 (dt, J=4.8, 8.0 Hz, 1H), 7.14 (dd, J=7.9, 10.9 Hz, 1H), 6.79 (s, 2H).

Step 2: A mixture of 4-(7-fluoro-1H-benzimidazol-2-yl)-1,2,5-oxadiazol-3-amine (200 mg, 0.913 mmol), pyridazin-4-ylmethanol (141 mg, 1.28 mmol) and 2-(tributylphosphoranylidene)acetonitrile (440 mg, 1.83 mmol) in THF (1 mL) was stirred at 90° C. for 3 hours under microwave irradiation and concentrated to afford crude product, which was purified by flash chromatography (ISCO®; 20 g SepaFlash®, 0-800% EtOAc in petroleum ether) to give 4-(7-fluoro-1-(pyridazin-4-ylmethyl)-benzimidazol-2-yl)-1,2,5-oxadiazol-3-amine (94 mg, 32%) as a brown solid and 4-(4-fluoro-1-(pyridazin-4-ylmethyl)-benzimidazol-2-yl)-1,2,5-oxadiazol-3-amine (44 mg, 16%) as a white solid.

### Example 132 (Peak 1): 4-(7-fluoro-1-(pyridazin-4-ylmethyl)-benzimidazol-2-yl)-1,2,5-oxadiazol-3-amine

MS ES⁺: 312.3

¹H NMR (400 MHz, DMSO-d₆) 9.29 (d, J=0.8 Hz, 1H), 9.20-9.17 (m, 1H), 7.81 (d, J=8.2 Hz, 1H), 7.46-7.38 (m, 2H), 7.34-7.28 (m, 1H), 7.02 (s, 2H), 6.12 (s, 2H).

### Example 133 (Peak 2): 4-(4-fluoro-1-(pyridazin-4-ylmethyl)-benzimidazol-2-yl)-1,2,5-oxadiazol-3-amine

MS ES⁺: 312.3

¹H NMR (400 MHz, DMSO-d₆) 9.27-9.18 (m, 1H), 9.14-9.05 (m, 1H), 7.62 (d, J=8.4 Hz, 1H), 7.49-7.38 (m, 1H), 7.31-7.19 (m, 2H), 6.94 (s, 2H), 6.05 (s, 2H).

### Example 134: 4-(7-fluoro-1-(pyrimidin-4-ylmethyl)-benzimidazol-2-yl)-1,2,5-oxadiazol-3-amine

### Example 135: 4-(4-fluoro-1-(pyrimidin-4-ylmethyl)-benzimidazol-2-yl)-1,2,5-oxadiazol-3-amine

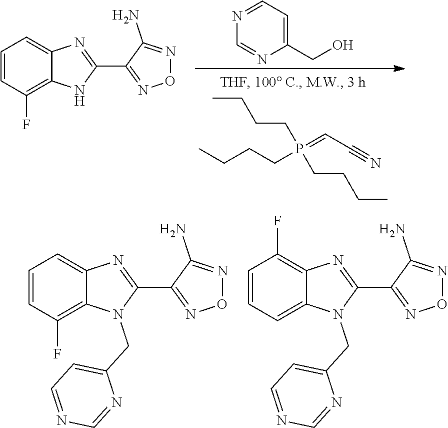

A mixture of 4-(7-fluoro-1H-benzimidazol-2-yl)-1,2,5-oxadiazol-3-amine (200 mg, 0.913 mmol), pyrimidin-4-ylmethanol (100 mg, 0.913 mmol) and 2-(tributylphosphoranylidene)acetonitrile (330 mg, 1.37 mmol) in THF (0.5 mL) was heated at 100° C. for 3 hours under microwave irradiation, poured into H₂O (10 mL) and extracted with EtOAc (10 mL×3). The combined organic layers were concentrated, and the residue purified by flash chromatography (ISCO®; 12 g SepaFlash®, 0-500% EtOAc in petroleum ether) to give crude product. Isomer 1 was further purified by prep. HPLC (column: Phenomenex Luna 30*30 mm*10 μm+YMC AQ 100*30*10 μm, Mobile Phase A: 0.225% aq. HCOOH, Mobile Phase B: CH₃CN, from 20% B to 80%). The pure fractions were collected and the volatiles were removed in vacuo. The residue was partitioned between CH₃CN (2 mL) and H₂O (10 mL). The solution was lyophilized to dryness to give 4-(7-fluoro-1-(pyrimidin-4-ylmethyl)-benzimidazol-2-yl)-1,2,5-oxadiazol-3-amine (65.8 mg, 23.1%) as a white solid. Isomer 2 was further purified by prep. HPLC (column: Phenomenex Luna 30*30 mm*10 μm+YMC AQ 100*30*10 μm, Mobile Phase A: 0.225% aq. FA, Mobile Phase B: CH₃CN, from 20% B to 80%). The pure fractions were collected and the volatiles were removed in vacuo. The residue was partitioned between CH₃CN (2 mL) and H₂O (10 mL). The solution was lyophilized to dryness to give 4-(4-fluoro-1-(pyrimidin-4-ylmethyl)-benzimidazol-2-yl)-1,2,5-oxadiazol-3-amine (117 mg, 40%) as a brown solid.

### Example 134 (Isomer 1): 4-(7-fluoro-1-(pyrimidin-4-ylmethyl)-benzimidazol-2-yl)-1,2,5-oxadiazol-3-amine

MS ES⁺: 312.3

¹H NMR (400 MHz, DMSO-d₆) 9.00 (d, J=1.4 Hz, 1H), 8.79 (d, J=5.4 Hz, 1H), 7.74 (d, J=8.2 Hz, 1H), 7.55-7.52 (m, 1H), 7.39-7.33 (m, 1H), 7.27-7.20 (m, 1H), 6.98 (s, 2H), 6.14 (s, 2H).

### Example 135 (Isomer 2): 4-(4-fluoro-1-(pyrimidin-4-ylmethyl)-benzimidazol-2-yl)-1,2,5-oxadiazol-3-amine

MS ES⁺: 312.3

¹H NMR (400 MHz, DMSO-d₆) 8.99 (d, J=1.2 Hz, 1H), 8.77 (d, J=5.4 Hz, 1H), 7.60 (d, J=8.4 Hz, 1H), 7.49-7.45 (m, 1H), 7.44-7.37 (m, 1H), 7.27-7.18 (m, 1H), 6.95 (s, 2H), 6.11 (s, 2H).

### Example 136: 4-(5,7-difluoro-1-(pyridin-4-ylmethyl)-benzimidazol-2-yl)-1,2,5-oxadiazol-3-amine

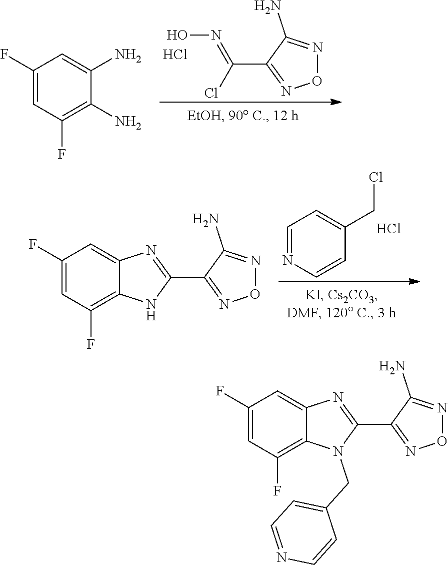

Step 1: A mixture of 3,5-difluorobenzene-1,2-diamine (500 mg, 3.47 mmol) and 4-amino-N-hydroxy-1,2,5-oxadiazole-3-carboximidoyl chloride hydrochloride (690 mg, 3.47 mmol) in EtOH (5 mL) was stirred at 90° C. for 12 hours and diluted with EtOH (10 mL) with yellow precipitation forming. The precipitate was collected by filtration to give 4-(5,7-difluoro-benzimidazol-2-yl)-1,2,5-oxadiazol-3-amine (1 g, crude) as a yellow solid.

MS ES⁺: 237.8

Step 2: A mixture of 4-(5,7-difluoro-benzimidazol-2-yl)-1,2,5-oxadiazol-3-amine (250 mg, 1.05 mmol), 4-(chloromethyl)pyridine hydrochloride (173 mg, 1.05 mmol), KI (175 mg, 1.05 mmol) and Cs₂CO₃ (1.03 g, 3.16 mmol) in DMF (2 mL) was stirred at 120° C. for 3 hours, cooled and extracted with EtOAc (10 mL×4). The combined organic layers were dried with anhydrous Na₂SO₄ and concentrated to afford crude product, which was purified by flash chromatography (ISCO®; 12 g SepaFlash®, 0-50% EtOAc in petroleum ether) to give the title compound (66.3 mg, 0.201 mmol, 19%) as a white solid.

MS ES⁺: 329.3

¹H NMR (400 MHz, DMSO-d₆) 8.54-8.47 (m, 2H), 7.67-7.59 (m, 1H), 7.41-7.34 (m, 1H), 7.12 (d, J=6.0 Hz, 2H), 6.97 (s, 2H), 6.02 (s, 2H).

### Example 137: 3-(7-fluoro-1-((6-(methylsulfonyl)pyridin-3-yl)methyl)-benzimidazol-2-yl)-4-methyl-1,2,5-oxadiazole

### Example 138: 3-(4-fluoro-1-((6-(methylsulfonyl)pyridin-3-yl)methyl)-benzimidazol-2-yl)-4-methyl-1,2,5-oxadiazole

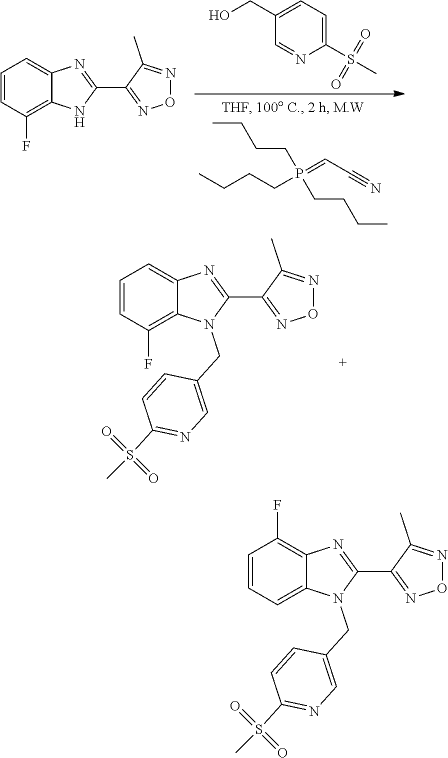 [SMILES: Cc1nonc1-c1nc2cccc(F)c2[nH]1 ; CCCCP(=CC#N)(CCCC)CCCC ; CS(=O)(=O)c1ccc(CO)cn1 ; Cc1nonc1-c1nc2cccc(F)c2n1Cc1ccc(S(C)(=O)=O)nc1 ; Cc1nonc1-c1nc2c(F)cccc2n1Cc1ccc(S(C)(=O)=O)nc1]

To a solution of 3-(7-fluoro-benzimidazol-2-yl)-4-methyl-1,2,5-oxadiazole (Intermediate 1) (117 mg, 0.534 mmol) and (6-methylsulfonyl-3-pyridyl)methanol (100 mg, 0.534 mmol) in THF (1 mL) was added 2-(tributylphosphoranylidene)acetonitrile (258 mg, 1.07 mmol) and the mixture was stirred at 100° C. for 2 hours under microwave irradiation. The mixture was concentrated under reduced pressure to give a residue, which was purified by prep. HPLC (column: Xtimate C₁₈ 100*30 mm*10 μm, Mobile Phase A: 0.225% aq. HCOOH, Mobile Phase B: CH₃CN, 40% B to 70%). The pure fractions were collected and the volatiles were removed in vacuo. The residue was partitioned between CH₃CN (2 mL) and H₂O (10 mL) and lyophilized to dryness to give the mixture product as a white solid, which was further purified by prep. HPLC (column: Phenomenex Luna C₁₈ 75*30 mm*3 μm, Mobile Phase A: 0.225% aq. HCOOH, Mobile Phase B: CH₃CN, 30% B to 70%). The pure fractions were collected and the volatiles were removed in vacuo. The residue was partitioned between CH₃CN (2 mL) and H₂O (10 mL) and lyophilized to dryness to give the mixture product as a white solid (51 mg), which was further purified by SFC (separation condition: DAICEL CHIRALCEL OJ (250 mm*30 mm, 10 μm); Mobile Phase: A: Supercritical CO₂, B: 0.1% NH₃·H₂O in EtOH, A:B=35:65) to give 3-(7-fluoro-1-((6-(methylsulfonyl)pyridin-3-yl)methyl)-benzimidazol-2-yl)-4-methyl-1,2,5-oxadiazole (isomer 1, 24 mg, 11%) as a white solid and 3-(4-fluoro-1-((6-(methylsulfonyl)pyridin-3-yl)methyl)-benzimidazol-2-yl)-4-methyl-1,2,5-oxadiazole (isomer 2, 7 mg, 3%) as a white solid.

### Example 137 (Isomer 1): 3-(7-fluoro-1-((6-(methylsulfonyl)pyridin-3-yl)methyl)-benzimidazol-2-yl)-4-methyl-1,2,5-oxadiazole

MS ES⁺: 388.3

¹H NMR (400 MHz, DMSO-d₆) 8.76-8.69 (m, 1H), 8.03-7.96 (m, 1H), 7.88-7.83 (m, 1H), 7.79-7.72 (m, 1H), 7.41-7.33 (m, 1H), 7.29-7.21 (m, 1H), 6.09 (s, 2H), 3.28 (s, 3H), 2.79 (s, 3H).

### Example 138 (Isomer 2): 3-(4-fluoro-1-((6-(methylsulfonyl)pyridin-3-yl)methyl)-benzimidazol-2-yl)-4-methyl-1,2,5-oxadiazole

MS ES⁺: 388.2

¹H NMR (400 MHz, DMSO-d₆) 8.79-8.74 (m, 1H), 8.02-7.96 (m, 1H), 7.85-7.77 (m, 1H), 7.65-7.58 (m, 1H), 7.47-7.40 (m, 1H), 7.29-7.20 (m, 1H), 6.13-6.06 (m, 2H), 3.27 (s, 3H), 2.81-2.79 (m, 3H).

### Example 139: 3-(7-fluoro-1-(pyridazin-3-ylmethyl)-benzimidazol-2-yl)-4-methyl-1,2,5-thiadiazole

### Example 140: 3-(4-fluoro-1-(pyridazin-3-ylmethyl)-benzimidazol-2-yl)-4-methyl-1,2,5-thiadiazole

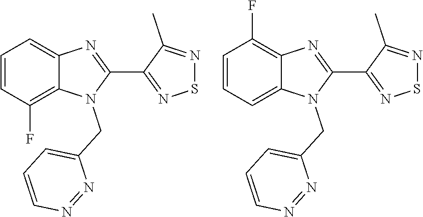

Step 1: A mixture of 3-fluorobenzene-1,2-diamine (350 mg, 2.77 mmol), 4-methyl-1,2,5-thiadiazole-3-carboxylic acid (400 mg, 2.77 mmol), triethylamine (842 mg, 8.32 mmol) and TP (2.65 g, 4.16 mmol, 50% purity in EtOAc) in CH₂Cl₂ (4 mL) was stirred at 25° C. for 1 hour, diluted with H₂O (30 mL) and extracted with EtOAc (10 mL×3). The combined organic layers were dried over Na₂SO₄, filtered and concentrated under reduced pressure to give a residue, which was purified by flash chromatography (ISCO®; 4 g SepaFlash®, 0-25% EtOAc in petroleum ether) to give N-(2-amino-3-fluorophenyl)-4-methyl-1,2,5-thiadiazole-3-carboxamide (350 mg, crude) as a yellow solid.

MS ES⁺: 253.2

¹H NMR (400 MHz, DMSO-d₆) 10.07 (s, 1H), 7.16-7.08 (m, 1H), 7.04-6.94 (m, 1H), 6.65-6.56 (m, 1H), 4.98 (s, 2H), 2.76 (s, 3H).

Step 2: A mixture of N-(2-amino-3-fluorophenyl)-4-methyl-1,2,5-thiadiazole-3-carboxamide (300 mg, 1.19 mmol) in AcOH (3 mL) was stirred at 110° C. for 1 hour, diluted with H₂O (30 mL) and extracted with EtOAc (10 mL×3). The combined organic layers were dried over Na₂SO₄, filtered and concentrated under reduced pressure to give a residue which was purified by flash chromatography (ISCO®; 4 g SepaFlash®, 0-10% EtOAc in petroleum ether) to give 3-(7-fluoro-benzimidazol-2-yl)-4-methyl-1,2,5-thiadiazole (100 mg, 0.427 mmol, 35.9%) as a white solid.

MS ES⁺: 235.1

Step 3: A mixture of 3-(7-fluoro-benzimidazol-2-yl)-4-methyl-1,2,5-thiadiazole (70 mg, 0.299 mmol), pyridazin-3-ylmethanol (33 mg, 0.299 mmol) and 2-(tributylphosphoranylidene)acetonitrile (144 mg, 0.598 mmol) in THF (1 mL) were heated at 100° C. for 3 hours under microwave irradiation, cooled, diluted with H₂O (30 mL) and extracted with EtOAc (10 mL×3). The combined organic layers were dried over Na₂SO₄, filtered and concentrated under reduced pressure to give a residue, which was purified by flash chromatography (ISCO®; 4 g SepaFlash®, 0-50% EtOAc in petroleum ether) to give 3-(7-fluoro-1-(pyridazin-3-ylmethyl)-benzimidazol-2-yl)-4-methyl-1,2,5-thiadiazole (11.7 mg, 11.9%) and 3-(4-fluoro-1-(pyridazin-3-ylmethyl)-benzimidazol-2-yl)-4-methyl-1,2,5-thiadiazole (19 mg, 20%) as white solids.

### Example 139: 3-(7-fluoro-1-(pyridazin-3-ylmethyl)-benzimidazol-2-yl)-4-methyl-1,2,5-thiadiazole

MS ES⁺: 326.9

¹HNMR (400 MHz, DMSO-d₆) 9.18-9.02 (m, 1H), 7.76-7.53 (m, 3H), 7.37-7.25 (m, 1H), 7.22-7.11 (m, 1H), 6.24 (s, 2H), 2.93 (s, 3H).

### Example 140: 3-(4-fluoro-1-(pyridazin-3-ylmethyl)-benzimidazol-2-yl)-4-methyl-1,2,5-thiadiazole

MS ES⁺: 327.0

¹H NMR (400 MHz, DMSO-d₆) 9.16-9.08 (m, 1H), 7.71-7.49 (m, 3H), 7.43-7.28 (m, 1H), 7.23-7.12 (m, 1H), 6.22 (s, 2H), 2.95 (s, 3H).

### Example 141: 4-(5,7-difluoro-1-(pyridazin-3-ylmethyl)-benzimidazol-2-yl)-1,2,5-oxadiazol-3-amine

### Example 142: 4-(4,6-difluoro-1-(pyridazin-3-ylmethyl)-benzimidazol-2-yl)-1,2,5-oxadiazol-3-amine

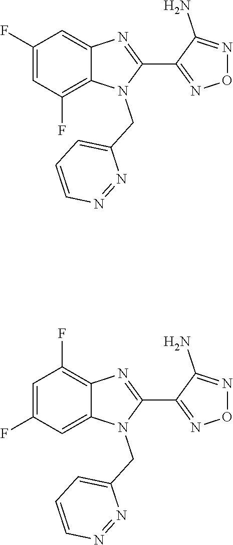 [SMILES: Nc1nonc1-c1nc2cc(F)cc(F)c2n1Cc1cccnn1 ; Nc1nonc1-c1nc2c(F)cc(F)cc2n1Cc1cccnn1]

Step 1: To a solution of pyridazin-3-ylmethanol (300 mg, 2.72 mmol) in CH₂Cl₂ (2 mL) was added SOCl₂ (1.30 g, 10.90 mmol) dropwise at ° C. The solution was stirred at 25° C. for 1 hour. Then the mixture was concentrated in vacuo to afford 3-(chloromethyl)pyridazine (300 mg, 86%) as a yellow solid which was used for the next step directly without further purification.

MS ES⁺: 129.1

Step 2: A mixture of 3,5-difluorobenzene-1,2-diamine (500 mg, 3.47 mmol) and 4-amino-N-hydroxy-1,2,5-oxadiazole-3-carboximidoyl chloride hydrochloride (690 mg, 3.47 mmol) in EtOH (5 mL) was stirred at 90° C. for 12 hours and diluted with EtOH (10 mL) with yellow precipitation forming. The precipitate was collected by filtration to give 4-(5,7-difluoro-benzimidazol-2-yl)-1,2,5-oxadiazol-3-amine (1 g, crude) as a yellow solid, which was used for the next step directly without further purification.

MS ES⁺: 237.8

Step 3: A mixture of 4-(5,7-difluoro-benzimidazol-2-yl)-1,2,5-oxadiazol-3-amine (250 mg, 1.05 mmol), 3-(chloromethyl)pyridazine (203 mg, 1.58 mmol), KI (175 mg, 1.05 mmol) and Cs₂CO₃ (1.03 g, 3.16 mmol) in DMF (2 mL) was stirred at 120° C. for 12 hours, cooled to RT, dissolved in DMF (3 mL) and filtered to remove the insoluble components. The filtrate was concentrated and the residue purified by prep. HPLC (column: Phenomenex Gemini-NX C₁₈ 75*30 mm*3 μm, Mobile Phase A: water (0.05% NH₃·H₂O+10 mM NH₄HCO₃), Mobile Phase B: CH₃CN, 18% B to 58%). The pure fractions were collected and the volatiles were removed in vacuo. The residue was partitioned between CH₃CN (2 mL) and H₂O (10 mL) and lyophilized to give the mixture product as a white powder (30 mg), which was further purified by SFC (separation condition: DAICEL CHIRALPAK AD (250 mm*30 mm, 10 μm); Mobile Phase: A: Supercritical CO₂, B: 0.1% NH₃·H₂O in EtOH, A:B=35:65) to give 4-(5,7-difluoro-1-(pyridazin-3-ylmethyl)-benzimidazol-2-yl)-1,2,5-oxadiazol-3-amine (3.8 mg, 1.1%) and 4-(4,6-difluoro-1-(pyridazin-3-ylmethyl)-benzimidazol-2-yl)-1,2,5-oxadiazol-3-amine (11.51 mg, 3.3%) as off-white solids.

### Example 141: 4-(5,7-difluoro-1-(pyridazin-3-ylmethyl)-benzimidazol-2-yl)-1,2,5-oxadiazol-3-amine

MS ES⁺: 330.0

¹H NMR (400 MHz, DMSO-d₆) 9.17-9.10 (m, 1H), 7.80-7.69 (m, 2H), 7.64-7.58 (m, 1H), 7.38-7.31 (m, 1H), 6.97 (s, 2H), 6.27 (s, 2H).

### Example 142: 4-(4,6-difluoro-1-(pyridazin-3-ylmethyl)-benzimidazol-2-yl)-1,2,5-oxadiazol-3-amine

MS ES⁺: 330.0

¹H NMR (400 MHz, DMSO-d₆) 9.17-9.08 (m, 1H), 7.75-7.65 (m, 3H), 7.37-7.28 (m, 1H), 6.90 (s, 2H), 6.22 (s, 2H).

### Example 143: 4-(7-fluoro-1-(pyridazin-3-ylmethyl)-benzimidazol-2-yl)-N-methyl-1,2,5-thiadiazol-3-amine

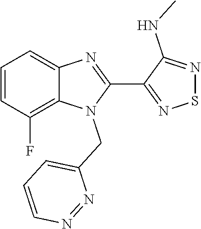 [SMILES: CNc1nsnc1-c1nc2cccc(F)c2n1Cc1cccnn1]

Step 1: A mixture of 4-(methylamino)-1,2,5-thiadiazole-3-carboxylic acid (20 mg, 0.126 mmol) and 6-fluoro-N1-(pyridazin-3-ylmethyl)benzene-1,2-diamine hydrochloride (38.4 mg, 0.151 mmol) in DMF (0.2 mL) was treated with HATU (72 mg, 0.188 mmol) and DIPEA (32.5 mg, 0.251 mmol) at 25° C., stirred at 25° C. for 1 hour, poured into H₂O (10 mL) and extracted with EtOAc (10 mL×3). The combined organic layers were concentrated to afford N-(3-fluoro-2-((pyridazin-3-ylmethyl)amino)phenyl)-4-(methylamino)-1,2,5-thiadiazole-3-carboxamide (40 mg, crude) as a brown oil, which was used for the next step directly without further purification.

MS ES⁺: 360.1

Step 2: A solution of N-(3-fluoro-2-((pyridazin-3-ylmethyl)amino)phenyl)-4-(methylamino)-1,2,5-thiadiazole-3-carboxamide (40 mg, 0.111 mmol) in AcOH (2 mL) was stirred at 110° C. for 1 hour. The mixture was concentrated and pH adjusted to around 8 with sat. aq. NaHCO₃ and extracted with EtOAc (10 mL×3). The combined organic layers were concentrated and the residue purified by prep. HPLC (column: Gemini NX C18 5 μm*10*150 mm, Mobile Phase A: 0.225% aq. HCOOH, Mobile Phase B: CH₃CN, 35% B to 65%). The pure fractions were collected and the volatiles removed in vacuo. The residue was partitioned between CH₃CN (2 mL) and H₂O (10 mL) and lyophilized to dryness to give the title compound (2 mg, 5%) as an off-white powder.

MS ES⁺: 342.3

¹H NMR (400 MHz, DMSO-d₆) 9.12 (t, J=3.2 Hz, 1H), 8.43 (d, J=5.1 Hz, 1H), 7.79-7.59 (m, 3H), 7.39-7.28 (m, 1H), 7.18 (dd, J=8.1, 12.1 Hz, 1H), 6.49 (s, 2H), 3.12 (d, J=4.8 Hz, 3H).

### Example 144: 4-(4-fluoro-1-(pyridazin-3-ylmethyl)-benzimidazol-2-yl)-N-methyl-1,2,5-thiadiazol-3-amine

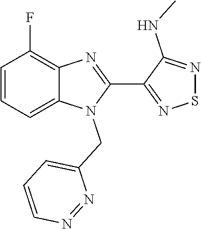 [SMILES: CNc1nsnc1-c1nc2c(F)cccc2n1Cc1cccnn1]

Step 1: A mixture of 3-fluorobenzene-1,2-diamine (30 mg, 0.238 mmol) and 4-(methylamino)-1,2,5-thiadiazole-3-carboxylic acid (38 mg, 0.238 mmol) in DMF (0.1 mL) was treated with HATU (181 mg, 0.476 mmol) and DIPEA (92 mg, 0.714 mmol) at 25° C., stirred for 1 hour, poured into H₂O (i mL) and extracted with EtOAc (10 mL×3). The combined organic layers were concentrated to afford N-(2-amino-3-fluorophenyl)-4-(methylamino)-1,2,5-thiadiazole-3-carboxamide (60 mg, crude) as a brown oil, which was used for the next step directly without further purification.

MS ES⁺: 268.0

Step 2: A mixture of N-(2-amino-3-fluorophenyl)-4-(methylamino)-1,2,5-thiadiazole-3-carboxamide (60 mg, crude) in AcOH (2 mL) was stirred at 110° C. for 1 hour. The mixture was pH adjusted to around 8 with sat. aq. NaHCO₃ and extracted with EtOAc (10 mL×3). The combined organic layers were concentrated and the residue purified by flash chromatography (ISCO®; 4 g SepaFlash®, 0-20% EtOAc in petroleum ether) to afford 4-(7-fluoro-benzimidazol-2-yl)-N-methyl-1,2,5-thiadiazol-3-amine (50 mg, 0.194 mmol, 86%) as a yellow solid.

MS ES⁺: 250.0

Step 3: A mixture of 4-(7-fluoro-benzimidazol-2-yl)-N-methyl-1,2,5-thiadiazol-3-amine (30 mg, 0.120 mmol), pyridazin-3-ylmethanol (15 mg, 0.132 mmol) and 2-(tributylphosphoranylidene)acetonitrile (58 mg, 0.241 mmol) in THF (1 mL) was stirred at 110° C. for 2 hours under microwave irradiation. The mixture was poured into H₂O (10 mL) and extracted with EtOAc (10 mL×3). The combined organic layers were concentrated to dryness to give a residue, which was purified by prep. TLC (petroleum ether:EtOAc=3:1) and further purified by prep. HPLC (column: Gemini NX C₁₈ μm*10*150 mm, Mobile Phase A: 0.225% aq. HCOOH, Mobile Phase B: CH₃CN, 40% to B 70%). The pure fractions were collected and the volatiles were removed in vacuo. The residue was partitioned between CH₃CN (2 mL) and H₂O (10 mL) and lyophilized to give the title compound (1.01 mg, 2.4%) as an off-white solid.

MS ES⁺: 342.3

¹H NMR (400 MHz, MeOD-d₄) 9.07 (d, J=3.5 Hz, 1H), 7.68-7.56 (m, 2H), 7.45-7.40 (m, 1H), 7.33 (dt, J=4.6, 8.1 Hz, 1H), 7.08 (dd, J=8.0, 10.6 Hz, 1H), 6.46 (s, 2H), 3.20 (s, 3H).

### Example 145: 4-(6,7-difluoro-1-(pyridin-4-ylmethyl)-benzimidazol-2-yl)-1,2,5-oxadiazol-3-amine

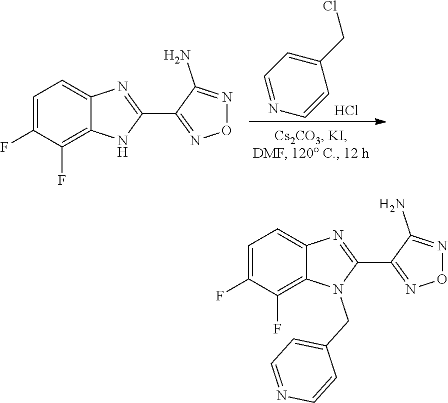

A mixture of 4-(6,7-difluoro-1H-benzimidazol-2-yl)-1,2,5-oxadiazol-3-amine (360 mg, 1.52 mmol), 4-(chloromethyl)pyridine hydrochloride (249 mg, 1.52 mmol), Cs₂CO₃ (1.48 g, 4.55 mmol) and KI (252 mg, 1.52 mmol) in DMF (3 mL) was stirred at 120° C. for 12 hours, diluted with H₂O (5 mL) and extracted with EtOAc (5 mL×3). The combined organic layers were dried over Na₂SO₄, filtered and concentrated under reduced pressure to give a residue, which was purified by flash chromatography (ISCO®; 4 g SepaFlash®, 0-50% EtOAc in petroleum ether) to afford the title compound (70.88 mg, 14%) as a white powder.

MS ES⁺: 329.3

¹H NMR (400 MHz, DMSO-d₆) 8.51 (d, J=5.88 Hz, 2H), 7.75 (dd, J=8.88, 3.50 Hz, 1H), 7.39-7.54 (m, 1H), 7.14 (d, J=5.63 Hz, 2H), 6.96 (s, 2H), 6.03 (s, 2H).

### Example 146: 3-(5,7-difluoro-1-(pyridazin-3-ylmethyl)-benzimidazol-2-yl)-4-methyl-1,2,5-oxadiazole

### Example 147: 3-(4,6-difluoro-1-(pyridazin-3-ylmethyl)-benzimidazol-2-yl)-4-methyl-1,2,5-oxadiazole

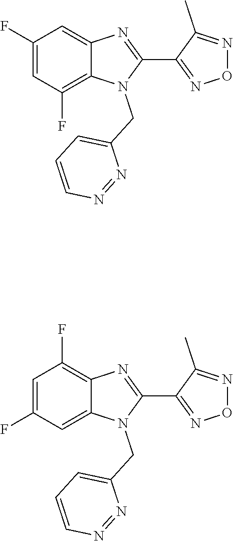 [SMILES: Cc1nonc1-c1nc2cc(F)cc(F)c2n1Cc1cccnn1 ; Cc1nonc1-c1nc2c(F)cc(F)cc2n1Cc1cccnn1]

Step 1: A mixture of 3,5-difluorobenzene-1,2-diamine (500 mg, 3.47 mmol), 4-methyl-1,2,5-oxadiazole-3-carboxylic acid (Intermediate 2) (444 mg, 3.47 mmol), triethylamine (1.05 g, 10.41 mmol) and TP (3.31 g, 5.20 mmol, 50% purity in EtOAc) in CH₂Cl₂ (5 mL) was stirred at 25° C. for 1 hour, diluted with H₂O (30 mL) and extracted with EtOAc (20×3 mL). The combined organic layers were dried over Na₂SO₄, filtered and concentrated under reduced pressure to give N-(2-amino-3,5-difluorophenyl)-4-methyl-1,2,5-oxadiazole-3-carboxamide (730 mg, crude) as a black oil, which was used for the next step.

Step 2: A mixture of N-(2-amino-3,5-difluorophenyl)-4-methyl-1,2,5-oxadiazole-3-carboxamide (1.70 g, 6.69 mmol) in AcOH (10 mL) was stirred at 110° C. for 1 hour, diluted with H₂O (30 mL) and extracted with EtOAc (20×3 mL). The combined organic layers were dried over Na₂SO₄, filtered and concentrated under reduced pressure to dryness. The residue was purified by flash chromatography (SiO₂, petroleum ether:EtOAc=1:0 to 5:1) to give 3-(5,7-difluoro-benzimidazol-2-yl)-4-methyl-1,2,5-oxadiazole (434 mg, 27.5%) as a white solid.

¹H NMR (400 MHz, DMSO-d₆) 7.32-7.07 (m, 2H), 2.76 (s, 3H).

Step 3: A solution of 3-(5,7-difluoro-benzimidazol-2-yl)-4-methyl-1,2,5-oxadiazole (200 mg, 0.85 mmol) and pyridazin-3-ylmethanol (93 mg, 0.85 mmol) in THF (2 mL) was treated with 2-(tributylphosphoranylidene)acetonitrile (409 mg, 1.69 mmol) under N₂ and stirred at 100° C. for 3 hours under microwave irradiation. The mixture was diluted with H₂O (5 mL) and extracted with EtOAc (5 mL×3). The combined organic layers were dried over Na₂SO₄, filtered and concentrated under reduced pressure and the residue purified by prep. HPLC (column: Phenomenex Luna 30*30 mm*10 μm+YMC AQ 100*30*10 μm, Mobile Phase A: 0.05% NH₃·H₂O in H₂O, Mobile Phase B: CH₃CN, 47% B to 80%) to give 3-(5,7-difluoro-1-(pyridazin-3-ylmethyl)-benzimidazol-2-yl)-4-methyl-1,2,5-oxadiazole (peak 1, 40 mg, 0.12 mmol, 14%) and 3-(4,6-difluoro-1-(pyridazin-3-ylmethyl)-benzimidazol-2-yl)-4-methyl-1,2,5-oxadiazole (peak 2, 78 mg, 0.24 mmol, 28%) as white powders.

### Example 146 (Peak 1): 3-(5,7-difluoro-1-(pyridazin-3-ylmethyl)-benzimidazol-2-yl)-4-methyl-1,2,5-oxadiazole

MS ES⁺: 329.3

¹H NMR (400 MHz, DMSO-d₆) 9.14 (d, J=2.38 Hz, 1H), 7.68-7.78 (m, 2H), 7.64 (d, J=9.01 Hz, 1H), 7.34 (t, J=10.69 Hz, 1H), 6.20 (s, 2H), 2.76 (s, 3H).

### Example 147 (Peak 2): 3-(4,6-difluoro-1-(pyridazin-3-ylmethyl)-benzimidazol-2-yl)-4-methyl-1,2,5-oxadiazole

MS ES⁺: 329.3

¹H NMR (400 MHz, DMSO-d₆) 9.13 (t, J=3.19 Hz, 1H), 7.69-7.74 (m, 2H), 7.65 (dd, J=8.88, 2.00 Hz, 1H), 7.31 (td, J=10.54, 2.06 Hz, 1H), 6.18 (s, 2H), 2.76 (s, 3H).

### Example 148: 4-(1-((6-chloropyridazin-3-yl)methyl)-7-fluoro-benzimidazol-2-yl)-1,2,5-oxadiazol-3-amine

### Example 149: 4-(1-((6-chloropyridazin-3-yl)methyl)-4-fluoro-benzimidazol-2-yl)-1,2,5-oxadiazol-3-amine

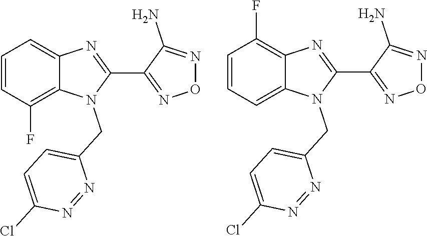

Step 1: A mixture of methyl 6-chloropyridazine-3-carboxylate (5.0 g, 29 mmol) in EtOH (50 mL) was treated with sodium tetrahydroborate (2.19 g, 58.0 mmol) in portions at ° C., stirred at 25° C. for 12 hours and quenched by addition of 1M HCl (aq.). The mixture was extracted with EtOAc (80 mL×3). The combined organic layers were washed with brine (50 mL×1), dried over Na₂SO₄, and concentrated under reduced pressure to give a residue, which was purified by flash chromatography (ISCO®; 20 g SepaFlash®, 0-60% EtOAc in petroleum ether) to give (6-chloropyridazin-3-yl)methanol (1.1 g, 7.61 mmol, 26.3%) as a yellow oil.

¹H NMR (400 MHz, DMSO-d₆) 7.91 (d, J=8.88 Hz, 1H), 7.80 (d, J=8.76 Hz, 1H), 5.75 (t, J=5.88 Hz, 1H), 4.75 (d, J=5.25 Hz, 2H).

Step 2: A mixture of (6-chloropyridazin-3-yl)methanol (500 mg, 3.46 mmol), 4-(7-fluoro-1H-benzimidazol-2-yl)-1,2,5-oxadiazol-3-amine (758 mg, 3.46 mmol) and 2-(tributylphosphoranylidene)acetonitrile (1.25 g, 5.19 mmol) in THF (2 mL) was heated at 100° C. for 3 hours under microwave irradiation and concentrated under reduced pressure to give a residue, which was purified by flash chromatography (ISCO®; 20 g SepaFlash®, 0-30% EtOAc in petroleum ether) and further purified by SFC (separation condition: DAICEL CHIRALCEL OD (250 mm*30 mm, 10 μm); Mobile Phase: A: Supercritical CO₂, B: 0.1% NH₃·H₂O in EtOH, A:B=35:65) to give 4-(1-((6-chloropyridazin-3-yl)methyl)-7-fluoro-benzimidazol-2-yl)-1,2,5-oxadiazol-3-amine (peak 1, 2.7 mg, 3%) as a grey solid and 4-(1-((6-chloropyridazin-3-yl)methyl)-4-fluoro-benzimidazol-2-yl)-1,2,5-oxadiazol-3-amine (peak 2, 71 mg, 74.6%) as an off-white solid.

### Example 148 (Peak 1): 4-(1-((6-chloropyridazin-3-yl)methyl)-7-fluoro-benzimidazol-2-yl)-1,2,5-oxadiazol-3-amine

MS ES⁺: 346.3

¹H NMR (400 MHz, DMSO-d₆) 7.89-8.01 (m, 2H), 7.73 (d, J=8.00 Hz, 1H), 7.32-7.40 (m, 1H), 7.20-7.26 (m, 1H), 6.99 (s, 2H), 6.30 (s, 2H).

### Example 149 (Peak 2): 4-(1-((6-chloropyridazin-3-yl)methyl)-4-fluoro-benzimidazol-2-yl)-1,2,5-oxadiazol-3-amine

MS ES⁺: 346.2

¹H NMR (400 MHz, DMSO-d₆) 7.91-7.97 (m, 1H), 7.84-7.90 (m, 1H), 7.63 (d, J=7.88 Hz, 1H), 7.37-7.47 (m, 1H), 7.19-7.29 (m, 1H), 6.91-6.99 (m, 2H), 6.27 (s, 2H).

### Example 150: 6-((2-(4-amino-1,2,5-oxadiazol-3-yl)-7-fluoro-benzimidazol-1-yl)methyl)pyridazin-3-ol

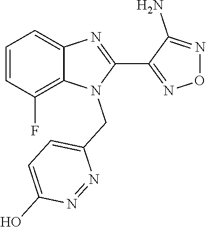 [SMILES: Nc1nonc1-c1nc2cccc(F)c2n1Cc1ccc(O)nn1]

A mixture of 4-(1-((6-chloropyridazin-3-yl)methyl)-7-fluoro-benzimidazol-2-yl)-1,2,5-oxadiazol-3-amine (Example 148) (10 mg, 0.029 mmol) in AcOH (0.2 mL) and H₂O (0.1 mL) was stirred at 120° C. for 30 min and concentrated to dryness to give a residue, which was purified by prep. HPLC (column: Xtimate C₁₈ 100*30 mm*10 μm, Mobile Phase A: 0.225% aq. HCOOH, Mobile Phase B: CH₃CN, 30% B to 60%). The pure fractions were collected and the volatiles were removed in vacuo. The residue was partitioned between CH₃CN (2 mL) and H₂O (i mL) and lyophilized to give the title compound (1 mg, 11%) as a white powder.

MS ES⁺: 328.1

¹H NMR (400 MHz, MeOD-d₄) 7.65 (d, J=8.1 Hz, 1H), 7.56 (d, J=9.8 Hz, 1H), 7.31 (dt, J=4.9, 8.1 Hz, 1H), 7.13 (dd, J=8.1, 11.8 Hz, 1H), 6.99 (d, J=9.8 Hz, 1H), 6.07 (s, 2H).

### Example 151: 4-(1-((6-deuteriopyridazin-3-yl)methyl)-7-fluoro-benzimidazol-2-yl)-1,2,5-oxadiazol-3-amine

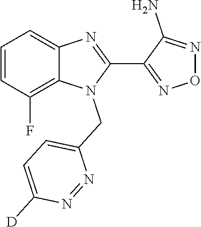 [SMILES: [2H]c1ccc(Cn2c(-c3nonc3N)nc3cccc(F)c32)nn1]

A mixture of 4-(1-((6-chloropyridazin-3-yl)methyl)-7-fluoro-benzimidazol-2-yl)-1,2,5-oxadiazol-3-amine (Example 148) (35 mg, 0.101 mmol) and palladium on activated charcoal (10 mg, 10%) in 2,2,3,3,4,4,5,5-octadeuteriotetrahydrofuran (2 mL) was degassed and purged with D2 three times and stirred at 25° C. for 1 hour under D2 (15 psi). The mixture was filtered and the filtrate concentrated under reduced pressure to give a residue, which was purified by prep. HPLC (column: Xtimate C₁₈ 100*30 mm*10 μm, Mobile Phase A: 0.225% aq. HCOOH, Mobile Phase B: CH₃CN, 35% B to 65%). The pure fractions were collected and the volatiles were removed in vacuo. The residue was partitioned between CH₃CN (2 mL) and H₂O (10 mL) and lyophilized to give the title compound (1 mg, 3%) as a white solid.

MS ES⁺: 313.2

¹H NMR (400 MHz, DMSO-d₆) 7.68-7.80 (m, 3H), 7.31-7.40 (m, 1H), 7.18-7.27 (m, 1H), 6.99 (s, 2H), 6.30 (s, 2H).

### Example 152: 4-(7-fluoro-1-((6-methoxypyridazin-3-yl)methyl)-benzimidazol-2-yl)-1,2,5-oxadiazol-3-amine

### Example 153: 4-(4-fluoro-1-((6-methoxypyridazin-3-yl)methyl)-benzimidazol-2-yl)-1,2,5-oxadiazol-3-amine

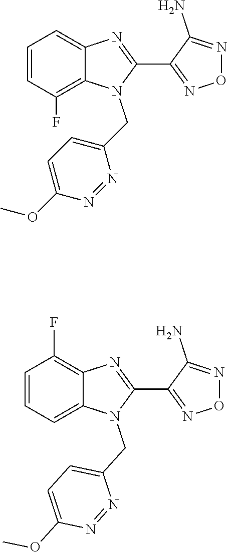 [SMILES: COc1ccc(Cn2c(-c3nonc3N)nc3cccc(F)c32)nn1 ; COc1ccc(Cn2c(-c3nonc3N)nc3c(F)cccc32)nn1]

A solution of (6-methoxypyridazin-3-yl)methanol (100 mg, 0.714 mmol), 4-(7-fluoro-1H-benzimidazol-2-yl)-1,2,5-oxadiazol-3-amine (156 mg, 0.714 mmol) and 2-(tributylphosphoranylidene)acetonitrile (258 mg, 1.07 mmol) in THF (1 mL) was stirred at 100° C. for 4 hours under microwave irradiation, poured into H₂O (30 mL) and extracted with EtOAc (30 mL×3). The combined organic layers were concentrated to give a residue, which was purified by flash chromatography (ISCO®; 4 g SepaFlash®, 0-20% EtOAc in petroleum ether) to give crude product, which was further separated by SFC (separation condition: DAICEL CHIRALCEL OJ (250 mm*30 mm, 10 μm); Mobile Phase: A: Supercritical CO₂, B: 0.1% NH₃·H₂O in EtOH, A:B=35:65) to give 4-(7-fluoro-1-((6-methoxypyridazin-3-yl)methyl)-benzimidazol-2-yl)-1,2,5-oxadiazol-3-amine (peak 1, 13 mg, 5%) and 4-(4-fluoro-1-((6-methoxypyridazin-3-yl)methyl)-benzimidazol-2-yl)-1,2,5-oxadiazol-3-amine (peak 2, 41 mg, 17%) as white solids.

### Example 152 (Peak 1): 4-(7-fluoro-1-((6-methoxypyridazin-3-yl)methyl)-benzimidazol-2-yl)-1,2,5-oxadiazol-3-amine

MS ES⁺: 341.1

¹H NMR (400 MHz, DMSO-d₆) 7.78-7.68 (m, 2H), 7.38-7.31 (m, 1H), 7.27-7.19 (m, 2H), 7.03-6.97 (m, 2H), 6.22-6.19 (m, 2H), 3.91 (s, 3H).

### Example 153 (Peak 2): 4-(4-fluoro-1-((6-methoxypyridazin-3-yl)methyl)-benzimidazol-2-yl)-1,2,5-oxadiazol-3-amine

MS ES⁺: 341.1

¹H NMR (400 MHz, DMSO-d₆) 7.73-7.68 (m, 1H), 7.60 (d, J=8.3 Hz, 1H), 7.44-7.36 (m, 1H), 7.27-7.17 (m, 2H), 7.00-6.91 (m, 2H), 6.20-6.12 (m, 2H), 3.92 (s, 3H).

### Example 154: 4-(4,7-difluoro-1-(pyridazin-3-ylmethyl)-benzimidazol-2-yl)-1,2,5-oxadiazol-3-amine

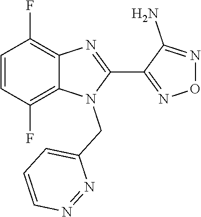 [SMILES: Nc1nonc1-c1nc2c(F)ccc(F)c2n1Cc1cccnn1]

A mixture of 4-(4,7-difluoro-benzimidazol-2-yl)-1,2,5-oxadiazol-3-amine (30 mg, 0.126 mmol), 3-(bromomethyl)pyridazine hydrobromide (32 mg, 0.13 mmol) and K₂CO₃ (52 mg, 0.38 mmol) in DMF (0.2 mL) was stirred at 90° C. for 1 hour. The mixture was concentrated to dryness and the residue purified by prep. HPLC (column: Phenomenex Luna C₁₈ 75*30 mm*3 μm, Mobile Phase A: 0.225% aq. FA, Mobile Phase B: CH₃CN, 17% B to 65%). The pure fractions were collected and the volatiles removed in vacuo. The residue was partitioned between CH₃CN (2 mL) and H₂O (i mL) and lyophilized to give the title compound (22 mg, 0.064 mmol, 51%) as a white solid.

MS ES⁺: 330.2

¹H NMR (400 MHz, DMSO-d₆) 9.14 (dd, J=1.6, 4.9 Hz, 1H), 7.83-7.69 (m, 2H), 7.31-7.14 (m, 2H), 6.93 (s, 2H), 6.29 (s, 2H).

### Example 155: 4-(4,7-difluoro-1-(pyridin-4-ylmethyl)-benzimidazol-2-yl)-1,2,5-oxadiazol-3-amine

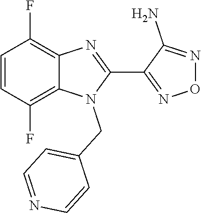 [SMILES: Nc1nonc1-c1nc2c(F)ccc(F)c2n1Cc1ccncc1]

A mixture of 4-(4,7-difluoro-benzimidazol-2-yl)-1,2,5-oxadiazol-3-amine (40 mg, 0.169 mmol), 4-(bromomethyl)pyridine hydrobromide (51 mg, 0.202 mmol) and K₂CO₃ (70 mg, 0.506 mmol) in DMF (0.2 mL) was stirred at 110° C. for 1 hour, concentrated and the residue was purified by prep. HPLC (column: Phenomenex Luna 30*30 mm*10 μm +YMC AQ 100*30*10 μm, Mobile Phase A: 0.225% aq. FA, Mobile Phase B: CH₃CN, 25% B to 45%). The pure fractions were collected and the volatiles removed in vacuo. The residue was partitioned between CH₃CN (2 mL) and H₂O (i mL) and lyophilized to give the title compound (26 mg, 47%) as an off-white solid.

MS ES⁺: 329.3

¹H NMR (400 MHz, DMSO-d₆) 8.54-8.47 (m, 2H), 7.30-7.19 (m, 2H), 7.13 (d, J=5.9 Hz, 2H), 6.92 (s, 2H), 6.02 (s, 2H).

### Example 156: 4-(7-fluoro-1-(pyridazin-3-ylmethyl)-benzimidazol-2-yl)-1,2,5-thiadiazol-3-amine

### Example 157: 4-(4-fluoro-1-(pyridazin-3-ylmethyl)-benzimidazol-2-yl)-1,2,5-thiadiazol-3-amine

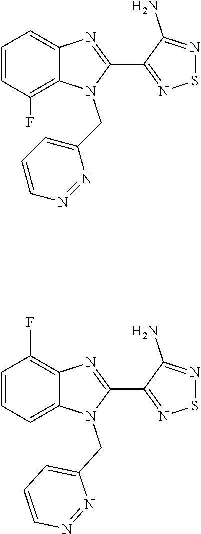 [SMILES: Nc1nsnc1-c1nc2cccc(F)c2n1Cc1cccnn1 ; Nc1nsnc1-c1nc2c(F)cccc2n1Cc1cccnn1]

Step 1: A mixture of 3-fluorobenzene-1,2-diamine (300 mg, 2.38 mmol) and 4-amino-1,2,5-thiadiazole-3-carboxylic acid (345 mg, 2.38 mmol) in CH₂Cl₂ (3 mL) was treated with TP (3.03 g, 4.76 mmol, 50% purity in EtOAc) and triethylamine (722 mg, 7.14 mmol) at 25° C., stirred for 2 hours, poured into H₂O (10 mL) and extracted with CH₂Cl₂ (10 mL×3). The combined organic layers were concentrated and the residue purified by flash chromatography (ISCO®; 4 g SepaFlash®, 0-30% EtOAc in petroleum ether) to afford 4-amino-N-(2-amino-3-fluorophenyl)-1,2,5-thiadiazole-3-carboxamide (300 mg, 42%) as a yellow solid.

MS ES⁺: 254.0

Step 2: A mixture of 4-amino-N-(2-amino-3-fluorophenyl)-1,2,5-thiadiazole-3-carboxamide (300 mg, 1.18 mmol) in AcOH (3 mL) was stirred at 90° C. for 5 hours. The mixture was poured into H₂O (10 mL), pH adjusted to 7 with sat. NaHCO₃ (aq.) and extracted with EtOAc (10 mL×3). The combined organic layers were concentrated and the residue purified by flash chromatography (ISCO®; 4 g SepaFlash®, 0-20% EtOAc in petroleum ether) to afford 4-(7-fluoro-benzimidazol-2-yl)-1,2,5-thiadiazol-3-amine (250 mg, 89%) as a yellow solid.

MS ES⁺: 235.9

¹H NMR (400 MHz, DMSO-d₆) 13.68 (br s, 1H), 7.64 (s, 2H), 7.44-7.38 (m, 1H), 7.31 (dt, J=4.9, 7.9 Hz, 1H), 7.17-7.05 (m, 1H).

Step 3: A mixture of 4-(7-fluoro-benzimidazol-2-yl)-1,2,5-thiadiazol-3-amine (200 mg, 0.85 mmol) and pyridazin-3-ylmethanol (94 mg, 0.85 mmol) in THF (2 mL) was treated with 2-(tributylphosphoranylidene)acetonitrile (410 mg, 1.70 mmol) in one portion at 25° C. under N₂. The mixture was stirred at 100° C. for 3 hours under microwave irradiation, poured into H₂O (10 mL) and extracted with EtOAc (10 mL×3). The combined organic layers were concentrated and the residue purified by flash chromatography (ISCO®; 4 g SepaFlash®, 0-50% EtOAc in petroleum ether) to afford 4-(7-fluoro-1-(pyridazin-3-ylmethyl)-benzimidazol-2-yl)-1,2,5-thiadiazol-3-amine (peak 1, 25 mg, 8%) as a yellow solid and a second product (peak 2), which was further purified by prep. HPLC (column: Xtimate C₁₈ 100*30 mm*10 μm, Mobile Phase A: 0.225% aq. FA, Mobile Phase B: CH₃CN, 30% B to 60%) to give 4-(4-fluoro-1-(pyridazin-3-ylmethyl)-benzimidazol-2-yl)-1,2,5-thiadiazol-3-amine (4.6 mg, 2%) as an off-white solid.

### Example 156 (Peak 1): 4-(7-fluoro-1-(pyridazin-3-ylmethyl)-benzimidazol-2-yl)-1,2,5-thiadiazol-3-amine

MS ES⁺: 328.3

¹H NMR (400 MHz, DMSO-d₆) 9.12 (t, J=3.2 Hz, 1H), 7.85 (s, 2H), 7.71 (d, J=8.2 Hz, 1H), 7.68 (d, J=3.2 Hz, 2H), 7.36-7.29 (m, 1H), 7.22-7.13 (m, 1H), 6.49 (s, 2H).

### Example 157 (Peak 2): 4-(4-fluoro-1-(pyridazin-3-ylmethyl)-benzimidazol-2-yl)-1,2,5-thiadiazol-3-amine

MS ES⁺: 328.3

¹H NMR (400 MHz, DMSO-d₆) 9.11 (t, J=3.2 Hz, 1H), 7.82 (s, 2H), 7.65 (d, J=3.6 Hz, 2H), 7.56 (d, J=8.1 Hz, 1H), 7.35 (dt, J=4.9, 8.1 Hz, 1H), 7.19 (dd, J=8.0, 10.9 Hz, 1H), 6.41 (s, 2H).

### Example 158: 4-(7-fluoro-1-(pyridazin-3-ylmethyl)-benzimidazol-2-yl)isoxazol-3-amine

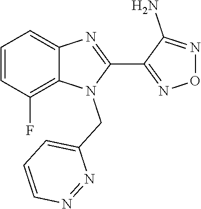 [SMILES: Nc1nonc1-c1nc2cccc(F)c2n1Cc1cccnn1]

Step 1: A mixture of pyridazin-3-ylmethanamine hydrochloride (3.58 g, 24.6 mmol), 1,2-difluoro-3-nitro-benzene (3.91 g, 24.6 mmol) and triethylamine (7.46 g, 73.8 mmol) in CH₃CN (35 mL) was stirred at 90° C. for 1 hour, diluted with H₂O (30 mL) and extracted with EtOAc (40 mL×3). The combined organic layers were dried over Na₂SO₄, filtered and concentrated under reduced pressure to give a residue, which was purified by flash chromatography (ISCO®; 20 g SepaFlash®, 0-100% EtOAc in petroleum ether) to give 2-fluoro-6-nitro-N-(pyridazin-3-ylmethyl)aniline (2.70 g, 44%) as a yellow solid.

MS ES⁺: 248.9

¹H NMR (400 MHz, DMSO-d₆) 9.21-9.09 (m, 1H), 8.72-8.62 (m, 1H), 7.98-7.85 (m, 1H), 7.75-7.61 (m, 2H), 7.53-7.36 (m, 1H), 6.80-6.70 (m, 1H), 5.10-4.97 (m, 2H).

Step 2: A mixture of 2-fluoro-6-nitro-N-(pyridazin-3-ylmethyl)aniline (1.00 g, 4.03 mmol) and palladium on activated charcoal (200 mg, 10% purity) in EtOAc (20 mL) was degassed and purged with H2 three times. The mixture was stirred at 25° C. for 2 hours under H2 (15 psi) and filtered. The filtrate was concentrated under reduced pressure to give crude 6-fluoro-N¹-(pyridazin-3-ylmethyl)benzene-1,2-diamine (860 mg, 97%) as a yellow solid, which was used for the next step without further purification.

MS ES⁺: 219.1

Step 3: A solution of 2-cyanoacetic acid (302 mg, 3.55 mmol), 6-fluoro-N¹-(pyridazin-3-ylmethyl)benzene-1,2-diamine (860 mg, 3.94 mmol) and DIPEA (1.53 g, 11.82 mmol) in CH₂Cl₂ (10 mL) was treated with HATU (2.25 g, 5.91 mmol) at ° C., stirred at 25° C. for 5 hours, poured into H₂O (10 mL), and extracted with EtOAc (20 mL×3). The combined organic layers were dried over Na₂SO₄, filtered and concentrated under reduced pressure to give a residue, which was purified by flash chromatography (ISCO®; 20 g SepaFlash®, 0-80% EtOAc in petroleum ether) to afford 2-cyano-N-(3-fluoro-2-((pyridazin-3-ylmethyl)amino)phenyl)acetamide (510 mg, 45%) as a pale yellow solid.

MS ES⁺: 286.1

Step 4: A mixture of 2-cyano-N-(3-fluoro-2-((pyridazin-3-ylmethyl)amino)phenyl)acetamide (510 mg, 1.79 mmol) in AcOH (6 mL) was stirred at 110° C. for 1 hour and concentrated under reduced pressure. The resulting residue was purified by flash chromatography (ISCO®; 12 g SepaFlash®, 0-95% EtOAc in petroleum ether) to afford 2-(7-fluoro-1-(pyridazin-3-ylmethyl)-benzimidazol-2-yl)acetonitrile (190 mg, 39%) as an orange solid.

MS ES⁺: 268.0

Step 5: A mixture of 2-(7-fluoro-1-(pyridazin-3-ylmethyl)-benzimidazol-2-yl)acetonitrile (190 mg, 0.711 mmol) in 1,1-dimethoxy-N,N-dimethylmethanamine (10 mL) was stirred at 6W° C. for 8 hours and concentrated under reduced pressure to give a residue, which was purified by prep. HPLC (column: Phenomenex Gemini-NX C₁₈ 75*30 mm*3 μm, Mobile Phase A: 10 mM aq. NH₄HCO₃, Mobile Phase B: CH₃CN, 13% B to 43%). The pure fractions were collected and the volatiles removed in vacuo. The residue was partitioned between CH₃CN (2 mL) and H₂O (10 mL) and lyophilized to give 3-(dimethylamino)-2-(7-fluoro-1-(pyridazin-3-ylmethyl)-benzimidazol-2-yl)acrylonitrile (90 mg, 390) as a yellow solid.

MS ES⁺: 323.1

¹H NMR (400 MHz, DMSO-d₆) 9.14 (dd, J=1.4, 4.9 Hz, 1H), 7.79 (s, 1H), 7.68 (dd, J=4.9, 8.5 Hz, 1H), 7.51-7.47 (m, 1H), 7.37 (d, J=8.0 Hz, 1H), 7.12 (d, J=5.0 Hz, 1H), 6.94-6.87 (m, 1H), 5.93 (s, 2H), 3.23 (br s, 6H).

Step 6: A solution of 3-(dimethylamino)-2-(7-fluoro-1-(pyridazin-3-ylmethyl)-benzimidazol-2-yl)acrylonitrile (40 mg, 0.124 mmol) in EtOH (1 mL) was treated with hydroxylamine hydrochloride (8.6 mg, 0.124 mmol) and stirred at 60° C. for 36 hours.

The mixture was concentrated under reduced pressure to give a residue, which was purified by prep. HPLC (column: Welch Xtimate C₁₈ 150*30 mm*5 μm, Mobile Phase A: 10 mM aq. NH₄HCO₃, Mobile Phase B: CH₃CN, 0% B to 35%). The pure fractions were collected and the volatiles were removed in vacuo. The residue was partitioned between CH₃CN (2 mL) and H₂O (10 mL) and lyophilized to give crude product (15 mg), which was further purified by prep. HPLC (column: Phenomenex Luna 30*30 mm*10 μm+YMC AQ 100*30*10 μm, Mobile Phase A: 0.05% NH₃·H₂O in H₂O, Mobile Phase B: CH₃CN, 0% B to 35%). The pure fractions were collected and the volatiles removed in vacuo. The residue was partitioned between CH₃CN (20 mL) and H₂O (100 mL) and lyophilized to give the title compound (1 mg, 2%) as a white solid.

MS ES⁺: 311.1

¹H NMR (400 MHz, DMSO-d₆) 13.59-12.67 (m, 1H), 9.17 (s, 1H), 7.75 (s, 2H), 7.48 (d, J=8.0 Hz, 1H), 7.25-7.11 (m, 1H), 7.06-6.93 (m, 1H), 6.83-6.37 (m, 2H), 5.99 (s, 2H).

### Example 159: 4-(1-((6-chloropyridin-3-yl)methyl)-6-fluoro-1H-imidazo[4,5-b]pyridin-2-yl)-1,2,5-oxadiazol-3-amine

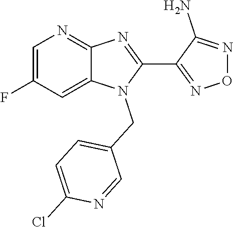 [SMILES: Nc1nonc1-c1nc2ncc(F)cc2n1Cc1ccc(Cl)nc1]

Step 1: A solution of 5-fluoro-3-nitro-pyridin-2-amine (2.00 g, 12.7 mmol) and DMAP (155 mg, 1.27 mmol) in THF (10 mL) was treated with Boc₂0 (5.56 g, 25.5 mmol), stirred at 90° C. for 1 hour, cooled to RT and poured into H₂O (30 mL). The mixture was extracted with EtOAc (30 mL×3). The combined organic layers were washed with brine (30 mL), dried over Na₂SO₄ and concentrated in vacuo to give a residue, which was purified by flash chromatography (ISCO®; 40 g SepaFlash®, 0-25% EtOAc in petroleum ether) to give tert-butyl (5-fluoro-3-nitropyridin-2-yl)carbamate (3.20 g, 12.4 mmol, 97%) as a yellow solid.

Step 2: A mixture of tert-butyl (5-fluoro-3-nitropyridin-2-yl)carbamate (3.20 g, 12.4 mmol), iron powder (3.47 g, 62.2 mmol) and NH₄Cl (3.33 g, 62.2 mmol) in EtOH (20 mL) and H₂O (20 mL) was stirred at 85° C. for 10 min, concentrated and extracted with EtOAc (30 mL×3). The combined organic layers were washed with brine (30 mL×2), dried over Na₂SO₄ and concentrated in vacuo to afford tert-butyl (3-amino-5-fluoropyridin-2-yl)carbamate (2.30 g, 10.1 mmol, 81%) as a yellow solid.

Step 3: A mixture of tert-butyl (3-amino-5-fluoropyridin-2-yl)carbamate (800 mg, 3.52 mmol) and 6-chloropyridine-3-carbaldehyde (498 mg, 3.52 mmol) in 1,2-dichloroethane (10 mL) was treated with AcOH (211 mg, 3.52 mmol) at 25° C., stirred at 25° C. for 2 hours, treated with sodium triacetoxyborohydride (2.24 g, 10.6 mmol) and stirred at 25° C. for 1 hour. The mixture was extracted with CH₂Cl₂ (40 mL×3). The combined organic layers were washed with brine (30 mL), dried over Na₂SO₄ and concentrated to give a residue, which was purified by flash chromatography (ISCO®; 20 g SepaFlash®, 0-25% EtOAc in petroleum ether) to give tert-butyl (3-(((6-chloropyridin-3-yl)methyl)amino)-5-fluoropyridin-2-yl)carbamate (421 mg, 19% yield, 55.3% purity) as a yellow solid.

MS ES⁺: 353.1

Step 4: A mixture of tert-butyl (3-(((6-chloropyridin-3-yl)methyl)amino)-5-fluoropyridin-2-yl)carbamate (421 mg, 0.660 mmol, 55.3% purity) in 4M HCl in dioxane (2 mL) was stirred at 25° C. for 15 min. The mixture was pH adjusted to around 8 with sat. NaHCO₃ (aq.) and extracted with EtOAc (20 mL×3). The combined organic layers were dried with anhydrous Na₂SO₄, filtered and concentrated in vacuo to afford N³-((6-chloropyridin-3-yl)methyl)-5-fluoropyridine-2,3-diamine (300 mg, crude purity) as a yellow solid.

MS ES⁺: 253.1

Step 5: A mixture of N³-((6-chloropyridin-3-yl)methyl)-5-fluoropyridine-2,3-diamine (150 mg, 0.594 mmol) and 4-amino-1,2,5-oxadiazole-3-carboxylic acid (76.6 mg, 0.594 mmol) in CH₂Cl₂ (1 mL) was treated with triethylamine (180 mg, 1.78 mmol) and TP (567 mg, 0.890 mmol, 50% purity in EtOAc) at 25° C. and stirred at 25° C. for 1 hour. The mixture was poured into H₂O (10 mL) and extracted with CH₂Cl₂ (10 mL×3). The combined organic layers were dried over Na₂SO₄ and concentrated in vacuo to give a residue, which was purified by flash chromatography (ISCO®; 20 g SepaFlash®, 0-50% EtOAc in petroleum ether) to give 4-amino-N-(3-(((6-chloropyridin-3-yl)methyl)amino)-5-fluoropyridin-2-yl)-1,2,5-oxadiazole-3-carboxamide (61 mg, 0.142 mmol, 24%) as a yellow solid.

MS ES⁺: 364.1

Step 6: A mixture of 4-amino-N-(3-(((6-chloropyridin-3-yl)methyl)amino)-5-fluoropyridin-2-yl)-1,2,5-oxadiazole-3-carboxamide (61 mg, 0.168 mmol) in AcOH (1 mL) was stirred at 110° C. for 30 min. The mixture was pH adjusted to around 9 with sat. NaHCO₃ (aq., 15 mL) and extracted with EtOAc (15 mL×3). The combined organic layers were dried over Na₂SO₄ and concentrated to afford crude product, which was purified by prep. HPLC (column: Phenomenex Gemini-NX C₁₈ 75*30 mm*3 μm, Mobile Phase A: water (0.05% NH₃·H₂O+10 mM NH₄HCO₃), Mobile Phase B: CH₃CN, 21% B to 61%) to give the title compound (6 mg, 10%) as a pink powder.

MS ES⁺: 346.2

¹H NMR (400 MHz, DMSO-d₆) 8.63 (s, 1H), 8.48-8.31 (m, 2H), 7.64 (dd, J=2.4, 8.3 Hz, 1H), 7.44 (d, J=8.4 Hz, 1H), 6.93 (s, 2H), 5.96 (s, 2H).

### Example 16o: 4-(6-fluoro-1-(pyrimidin-5-ylmethyl)-11H-imidazo[4,5-b]pyridin-2-yl)-1,2,5-oxadiazol-3-amine

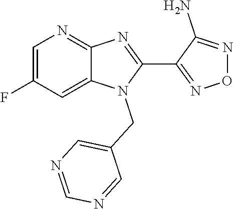 [SMILES: Nc1nonc1-c1nc2ncc(F)cc2n1Cc1cncnc1]

Step 1: A mixture of tert-butyl (3-amino-5-fluoropyridin-2-yl)carbamate (500 mg, 2.20 mmol), pyrimidine-5-carbaldehyde (238 mg, 2.20 mmol) and AcOH (132 mg, 2.20 mmol) in 1,2-dichloroethane (5 mL) was stirred at 25° C. for 2 hours, treated portionwise with sodium triacetoxyborohydride (1.40 g, 6.60 mmol) and stirred at RT for 3 hours. The mixture was poured into H₂O (30 mL) and extracted with CH₂Cl₂ (30 mL×3). The organic layers were washed with brine (30 mL), dried over Na₂SO₄ and concentrated in vacuo to give a residue, which was purified by flash chromatography (ISCO®; 4 g SepaFlash®, 0-5% MeOH in CH₂Cl₂) to give tert-butyl (5-fluoro-3-((pyrimidin-5-ylmethyl)amino)pyridin-2-yl)carbamate (258 mg, 0.667 mmol, 30% yield, 83% purity) as a yellow solid.

MS ES⁺: 320.1

Step 2: A mixture of tert-butyl (5-fluoro-3-((pyrimidin-5-ylmethyl)amino)pyridin-2-yl)carbamate (258 mg, 0.808 mmol) in 4M HCl in dioxane (4 mL) was stirred at 25° C. for 15 min. The mixture was pH adjusted to around 9 with sat. NaHCO₃ (aq., 15 mL) and extracted with EtOAc (10 mL×3). The combined organic layers were washed with brine (15 mL×2), dried over Na₂SO₄, filtered and concentrated in vacuo to afford 5-fluoro-N3-(pyrimidin-5-ylmethyl)pyridine-2,3-diamine (170 mg, 96%) as a yellow solid.

MS ES⁺: 220.1

Step 3: A mixture of 5-fluoro-N3-(pyrimidin-5-ylmethyl)pyridine-2,3-diamine (100 mg, 0.456 mmol) and 4-amino-1,2,5-oxadiazole-3-carboxylic acid (59 mg, 0.456 mmol) in DMF (1 mL) was treated with DIPEA (177 mg, 1.37 mmol) and HATU (260 mg, 0.684 mmol) at RT, stirred for 1 hour, poured into H₂O (10 mL) and extracted with EtOAc (10 mL×3). The combined organic layers were dried over Na₂SO₄ and concentrated in vacuo to afford 4-amino-N-(5-fluoro-3-((pyrimidin-5-ylmethyl)amino)pyridin-2-yl)-1,2,5-oxadiazole-3-carboxamide (140 mg, 0.424 mmol, 92.9%) as yellow gum which was used for the next step directly.

MS ES⁺: 331.2

Step 4: A mixture of 4-amino-N-(5-fluoro-3-((pyrimidin-5-ylmethyl)amino)pyridin-2-yl)-1,2,5-oxadiazole-3-carboxamide (140 mg, 0.424 mmol) in AcOH (1 mL) was stirred at 11° C. for 30 min. The mixture was pH adjusted to around 9 with sat. NaHCO₃ (aq., 10 mL) and extracted with EtOAc (10 mL×3). The combined organic layers were dried over Na₂SO₄ and concentrated to afford a residue, which was purified by prep. HPLC (column: Phenomenex Gemini-NX C₁₈ 75*30 mm*3 μm, Mobile Phase A: 10 mM aq. NH₄HCO₃, Mobile Phase B: CH₃CN, 0% B to 40%) to give the title compound (7.33 mg, 5%) as an off-white powder.

MS ES⁺: 313.1

¹H NMR (400 MHz, DMSO-d₆) 9.11 (s, 1H), 8.73 (s, 2H), 8.63 (dd, J=2.0, 2.4 Hz, 1H), 8.44 (dd, J=2.8, 8.8 Hz, 1H), 6.93 (s, 2H), 5.97 (s, 2H).

### Example 161: (S)-4-(7-fluoro-1-(1-(pyridin-3-yl)ethyl)-benzimidazol-2-yl)-1,2,5-oxadiazol-3-amine (0.5 eq. formate)

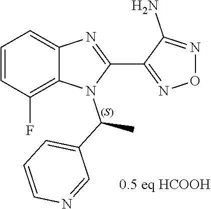

Step 1: A mixture of 1,2-difluoro-3-nitro-benzene (195 mg, 1.23 mmol), (1S)-1-(3-pyridyl)ethanamine (150 mg, 1.23 mmol) and triethylamine (124 mg, 1.23 mmol) in CH₃CN (1 mL) was stirred at 25° C. for 5 hours and concentrated to afford a residue, which was purified by flash chromatography (ISCO®; 4 g SepaFlash®, 0-25% EtOAc in petroleum ether) to give (S)-2-fluoro-6-nitro-N-(1-(pyridin-3-yl)ethyl)aniline (260 mg, 79%) as a yellow solid.

MS ES⁺: 261.9

SFC: 99% chiral purity, Rt 2.18 min.

¹H NMR (400 MHz, DMSO-d₆) 8.56 (d, J=2.0 Hz, 1H), 8.43 (dd, J=1.5, 4.8 Hz, 1H), 7.93-7.87 (m, 1H), 7.78-7.71 (m, 2H), 7.41 (ddd, J=1.0, 7.9, 13.9 Hz, 1H), 7.33 (dd, J=4.8, 7.9 Hz, 1H), 6.76 (dt, J=4.8, 8.3 Hz, 1H), 5.14-5.11 (m, 1H), 1.57 (d, J=6.8 Hz, 3H).

Step 2: A solution of Na₂S₂O₄ (173 mg, 0.995 mmol) in H₂O (1 mL) was added to (S)-2-fluoro-6-nitro-N-(1-(pyridin-3-yl)ethyl)aniline (260 mg, 0.995 mmol) in EtOH (1 mL). The mixture was stirred at 85° C. for 10 min, concentrated and extracted with EtOAc (10 mL×3). The combined organic layers were washed with brine (15 mL×2), dried over Na2SO₄, and evaporated to afford (S)-6-fluoro-N1-(1-(pyridin-3-yl)ethyl)benzene-1,2-diamine (164 mg, 71%) as yellow gum.

Step 3: A mixture of 4-amino-N-hydroxy-1,2,5-oxadiazole-3-carboximidoyl chloride hydrochloride (141 mg, 0.709 mmol) and (S)-6-fluoro-N1-(1-(pyridin-3-yl)ethyl)benzene-1,2-diamine (164 mg, 0.709 mmol) in EtOH (3 mL) was stirred at 85° C. for 10 hours and concentrated to afford a residue, which was purified by prep.

HPLC (column: Phenomenex Luna C₁₈ 75*30 mm*3 μm, Mobile Phase A: 0.225% aq.

FA, Mobile Phase B: CH₃CN, 10% B to 50%). The pure fractions were collected and the volatiles were removed in vacuo. The residue was partitioned between CH₃CN (20 mL) and H₂O (100 mL) and lyophilized to dryness to give the title compound (10 mg, 4%) as an off-white powder.

MS ES⁺: 325.0

SFC: 98.4% chiral purity, Rt 3.90 min.

¹H NMR (400 MHz, DMSO-d₆) 8.52-8.45 (m, 2H), 8.19 (s, 0.5H), 7.73 (d, J=8.0 Hz, 1H), 7.61 (d, J=8.0 Hz, 1H), 7.37-7.29 (m, 2H), 7.12 (dd, J=8.0, 12.0 Hz, 1H), 7.01-6.92 (m, 3H), 2.02 (dd, J=0.8, 6.8 Hz, 3H).

### Example 162: 4-(6,7-difluoro-1-(pyridazin-3-ylmethyl)-benzimidazol-2-yl)-1,2,5-oxadiazol-3-amine

### Example 163: 4-(4,5-difluoro-1-(pyridazin-3-ylmethyl)-benzimidazol-2-yl)-1,2,5-oxadiazol-3-amine

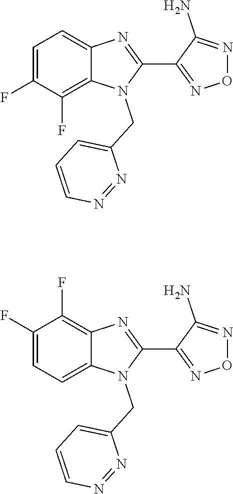 [SMILES: Nc1nonc1-c1nc2ccc(F)c(F)c2n1Cc1cccnn1 ; Nc1nonc1-c1nc2c(F)c(F)ccc2n1Cc1cccnn1]

Step 1: A mixture of 3,4-difluorobenzene-1,2-diamine (450 mg, 3.12 mmol) and 4-amino-N-hydroxy-1,2,5-oxadiazole-3-carboximidoyl chloride hydrochloride (621 mg, 3.12 mmol) in EtOH (8 mL) was stirred at 85° C. for 10 hours and concentrated to afford a residue, which was purified by flash chromatography (ISCO®; 20 g SepaFlash®, 0-30% EtOAc in petroleum ether) to give 4-(6,7-difluoro-1H-benzimidazol-2-yl)-1,2,5-oxadiazol-3-amine (650 mg, 84%) as yellow gum.

MS ES⁺: 238.0

¹H NMR (400 MHz, DMSO-d₆) 7.41 (s, 1H), 6.77 (s, 1H).

Step 2: A mixture of 4-(6,7-difluoro-1H-benzimidazol-2-yl)-1,2,5-oxadiazol-3-amine (300 mg, 1.26 mmol), pyridazin-3-ylmethanol (139 mg, 1.26 mmol) and 2-(tributylphosphoranylidene)acetonitrile (305 mg, 1.26 mmol) in THF (3 mL) was heated at 110° C. for 4 hours under microwave irradiation, cooled to RT and concentrated to afford a residue, which was purified by flash chromatography (ISCO®; 40 g SepaFlash®, 0-100% EtOAc in petroleum ether) to give 4-(6,7-difluoro-1-(pyridazin-3-ylmethyl)-benzimidazol-2-yl)-1,2,5-oxadiazol-3-amine (peak 1, 46 mg, 10%) as a grey solid and 4-(4,5-difluoro-1-(pyridazin-3-ylmethyl)-benzimidazol-2-yl)-1,2,5-oxadiazol-3-amine (peak 2, 179 mg, 43%) as a white powder.

### Example 162 (Peak 1): 4-(6,7-difluoro-1-(pyridazin-3-ylmethyl)-benzimidazol-2-yl)-1,2,5-oxadiazol-3-amine

MS ES⁺: 329.9

¹H NMR (400 MHz, DMSO-d₆) 9.15 (dd, J=1.6, 4.8 Hz, 1H), 7.83-7.70 (m, 3H), 7.54-7.39 (m, 1H), 6.96 (s, 2H), 6.29 (s, 2H).

### Example 163 (Peak 2): 4-(4,5-difluoro-1-(pyridazin-3-ylmethyl)-benzimidazol-2-yl)-1,2,5-oxadiazol-3-amine

MS ES⁺: 330.3

¹H NMR (400 MHz, DMSO-d₆) 9.13 (dd, J=2.0, 4.4 Hz, 1H), 7.80-7.63 (m, 3H), 7.59-7.47 (m, 1H), 6.93 (s, 2H), 6.26 (s, 2H).

### Example 164: N-methyl-5-((2-(4-methyl-1,2,5-oxadiazol-3-yl)-benzimidazol-1-yl)methyl)pyridine-2-sulfonamide

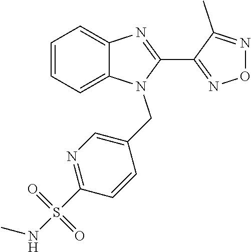 [SMILES: CNS(=O)(=O)c1ccc(Cn2c(-c3nonc3C)nc3ccccc32)cn1]

Step 1: A solution of 2-bromo-5-methyl-pyridine (5.00 g, 29.07 mmol) in CCl₄ (50 mL) was treated with NBS (5.69 g, 31.9 mmol) and benzoic peroxyanhydride (352 mg, 1.45 mmol) in portions, stirred at 90° C. for 16 hours, diluted with H₂O (30 mL) and extracted with CH₂Cl₂ (40 mL). The organic layer was dried over Na₂SO₄ and concentrated under reduced pressure to give a residue, which was purified by flash chromatography (ISCO®; 40 g SepaFlash®, 0-10% EtOAc in petroleum ether) to afford 2-bromo-5-(bromomethyl)pyridine (3.3 g, 36%) as a yellow solid.

MS ES⁺: 252.0

Step 2: A mixture of 3-(1H-benzimidazol-2-yl)-4-methyl-1,2,5-oxadiazole (1.28 g, 6.38 mmol), 2-bromo-5-(bromomethyl)pyridine (1.6 g, 6.38 mmol) and K₂CO₃ (2.64 g, 19.13 mmol) in DMF (40 mL) was stirred at 90° C. for 30 min, diluted with H₂O (40 mL) and extracted with EtOAc (40 mL×3). The combined organic layers were washed with LiCl (aq., 4%, 50 mL), dried over anhydrous Na₂SO₄, filtered and concentrated to dryness. The residue was triturated with petroleum ether/EtOAc (5/1, 20 mL) at RT for 1 hour. The solids were collected by filtration to afford 3-(1-((6-bromopyridin-3-yl)methyl)benzimidazol-2-yl)-4-methyl-1,2,5-oxadiazole (1.6 g, 3.63 mmol, 56.9% yield, 84.0% purity) as a white solid.

MS ES⁺: 372.0

¹H NMR (400 MHz, DMSO-d₆) 8.36 (d, J=2.25 Hz, 1H), 7.88 (d, J=7.63 Hz, 1H), 7.74 (d, J=7.75 Hz, 1H), 7.57 (d, J=1.00 Hz, 1H), 7.48 (dd, J=8.32, 2.56 Hz, 1H), 7.41 (dd, J=7.94, 1.19 Hz, 1H), 7.38 (dd, J=7.88, 1.13 Hz, 1H), 5.92 (s, 2H), 2.78 (s, 3H).

Step 3: A mixture of 3-(1-((6-bromopyridin-3-yl)methyl)benzimidazol-2-yl)-4-methyl-1,2,5-oxadiazole (1.00 g, 2.70 mmol), phenylmethanethiol (503 mg, 4.05 mmol) and K₂CO₃ (560 mg, 4.05 mmol) in DMSO (2 mL) was stirred at 145° C. for 3 hours and poured into H₂O (10 mL) with white precipitation forming. The precipitate was washed with H₂O (10 mL) and MeOH (10 mL), and dried to afford 3-(1-((6-(benzylthio)pyridin-3-yl)methyl)-benzimidazol-2-yl)-4-methyl-1,2,5-oxadiazole (1.2 g, 2.65 mmol, 98%) as a white solid.

MS ES⁺: 414.2

¹H NMR (400 MHz, DMSO-d₆) 8.42 (d, J=1.8 Hz, 1H), 7.87 (d, J=7.8 Hz, 1H), 7.75 (d, J=7.9 Hz, 1H), 7.46-7.33 (m, 5H), 7.31-7.18 (m, 4H), 5.89 (s, 2H), 4.36 (s, 2H), 2.78 (s, 3H).

Step 4: A solution of 3-(1-((6-(benzylthio)pyridin-3-yl)methyl)-benzimidazol-2-yl)-4-methyl-1,2,5-oxadiazole (100 mg, 0.242 mmol) in AcOH (1 mL) and H₂O (0.5 mL) was treated with N-chlorosuccinimide (129 mg, 0.967 mmol) at ° C., gradually warmed to RT and stirred for 5 hours. The mixture was poured into H₂O (10 mL) and extracted with EtOAc/THF (1/1, 10 mL×3). The combined organic layers were concentrated to afford 5-((2-(4-methyl-1,2,5-oxadiazol-3-yl)-benzimidazol-1-yl)methyl)pyridine-2-sulfonyl chloride (90 mg) as a white solid, which was used for the next step without further purification.

Step 5: To a mixture of 5-((2-(4-methyl-1,2,5-oxadiazol-3-yl)-benzimidazol-1-yl)methyl)pyridine-2-sulfonyl chloride (60 mg, 0.154 mmol) and methylamine hydrochloride (13 mg, 0.185 mmol) in THF (0.2 mL) was added triethylamine (31 mg, 0.308 mmol) and DMAP (2 mg, 0.002 mol) in one portion at RT. The mixture was stirred at 25° C. for 30 min and concentrated to give a residue, which was purified by prep. HPLC (column: Phenomenex Luna C₁₈ 75*30 mm*3 μm, Mobile Phase A: 0.225% aq. FA, Mobile Phase B: CH₃CN, 26% B to 70%). The pure fractions were collected and the volatiles removed in vacuo. The residue was partitioned between CH₃CN (2 mL) and H₂O (10 mL) and lyophilized. The residue was further purified by prep. HPLC (column: Phenomenex Luna C₁₈ 75*30 mm*3 μm, Mobile Phase A: 0.225% aq. FA, Mobile Phase B: CH₃CN, 16% B to 80%). The pure fractions were collected and the volatiles removed in vacuo. The residue was partitioned between CH₃CN (2 mL) and H₂O (10 mL) and lyophilized to give the title compound (2 mg, 3%) as a white solid.

MS ES⁺: 385.2

¹H NMR (400 MHz, DMSO-d₆) 8.70 (d, J=1.6 Hz, 1H), 7.93-7.89 (m, 1H), 7.84 (d, J=8.2 Hz, 1H), 7.80-7.64 (m, 3H), 7.46-7.37 (m, 2H), 6.05 (s, 2H), 3.31 (s, 3H), 2.79 (s, 3H).

### Example 165: 5-((2-(4-methyl-1,2,5-oxadiazol-3-yl)-3H-imidazo[4,5-b]pyridin-3-yl)methyl)pyrimidine-2-carbonitrile

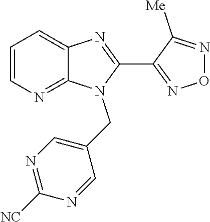 [SMILES: Cc1nonc1-c1nc2cccnc2n1Cc1cnc(C#N)nc1]

Step 1: A mixture of 5-methylpyrimidine-2-carbonitrile (1.5 g, 12.59 mmol), benzoic peroxyanhydride (153 mg, 0.63 mmol) and NBS (2.69 g, 15.1 mmol) in CCl₄ (20 mL) was stirred at 80° C. for 8 hours, cooled to RT and poured into H₂O (80 mL). The mixture was extracted with EtOAc (80 mL×3). The combined organic layers were dried with Na₂SO₄ and concentrated to give a residue, which was purified by flash chromatography (ISCO®; 40 g SepaFlash®, petroleum ether:EtOAc=8:1) to afford 5-(bromomethyl)pyrimidine-2-carbonitrile (1.4 g, 32%) as a yellow solid.

MS ES⁺: 198.1

Step 2: NaH (566 mg, 14.14 mmol, 60% purity) was added into tert-butyl N-tert-butoxycarbonylcarbamate (1.86 g, 8.55 mmol) in THF (15 mL). 5-(Bromomethyl)pyrimidine-2-carbonitrile (1.4 g, 7.07 mmol) was added to the mixture in one portion at ° C. under N₂. The mixture was stirred at RT for 2 hours, treated with sat. aq. NH₄C₁ (20 mL) and extracted with EtOAc (20 mL×3). The combined organic layers were concentrated to give a residue, which was purified by flash chromatography (ISCO®; 12 g SepaFlash®, 0-25% EtOAc in petroleum ether) to afford tert-butyl N-tert-butoxycarbonyl-N-[(2-cyanopyrimidin-5-yl)methyl]carbamate (2.2 g, 89%) as an off-white solid.

MS ES⁺: 335.2

Step 3: A mixture of tert-butyl N-tert-butoxycarbonyl-N-[(2-cyanopyrimidin-5-yl)methyl]carbamate (2.2 g, 6.58 mmol) in 4M HCl in dioxane (5 mL) was stirred at 25° C. for 1 hour and concentrated to afford 5-(aminomethyl)pyrimidine-2-carbonitrile dihydrochloride (800 mg, 58%) as a white solid.

Step 4: A mixture of 2-fluoro-3-nitropyridine (200 mg, 1.41 mmol), 5-(aminomethyl)pyrimidine-2-carbonitrile dihydrochloride (291 mg, 1.41 mmol) and triethylamine (427 mg, 4.22 mmol) in CH₃CN (2 mL) was stirred at 25° C. for 10 hours and concentrated to give a residue, which was purified by flash chromatography (ISCO®; 4 g SepaFlash®, 0-40% EtOAc in petroleum ether) to afford 5-(((3-nitropyridin-2-yl)amino)methyl)pyrimidine-2-carbonitrile (250 mg, 69%) as a yellow solid.

MS ES⁺: 256.8

¹H NMR (400 MHz, DMSO-d₆) 9.07 (t, J=5.8 Hz, 1H), 9.00 (s, 2H), 8.51-8.36 (m, 2H), 6.82 (dd, J=4.5, 8.3 Hz, 1H), 4.87 (d, J=6.0 Hz, 2H).

Step 5: A solution of Na₂S₂O₄ (510 mg, 2.93 mmol) in H₂O (1 mL) was added into 5-(((3-nitropyridin-2-yl)amino)methyl)pyrimidine-2-carbonitrile (150 mg, 0.585 mmol) in EtOH (3 mL) at 90° C. The mixture was stirred at 90° C. for 10 min, cooled and extracted with EtOAc (20 mL×3). The combined organic layers were concentrated to afford 5-(((3-aminopyridin-2-yl)amino)methyl)pyrimidine-2-carbonitrile (100 mg, 75%) as an off-white solid, which was used for the next step without further purification.

Step 6: A mixture of 5-(((3-aminopyridin-2-yl)amino)methyl)pyrimidine-2-carbonitrile (100 mg, 0.442 mmol), 4-methyl-1,2,5-oxadiazole-3-carboxylic acid (Intermediate 2) (56.6 mg, 0.442 mmol), HATU (252 mg, 0.663 mmol) and triethylamine (114 mg, 1.13 mmol) in DMF (2 mL) was stirred at 25° C. for 5 hours, poured into H₂O (10 mL) and extracted with EtOAc (10 mL×3). The combined organic layers were concentrated to afford N-(2-(((2-cyanopyrimidin-5-yl)methyl)amino)pyridin-3-yl)-4-methyl-1,2,5-oxadiazole-3-carboxamide (100 mg, 67%) as a brown oil, which was used for the next step without further purification.

MS ES⁺: 337.0

Step 7: A mixture of N-(2-(((2-cyanopyrimidin-5-yl)methyl)amino)pyridin-3-yl)-4-methyl-1,2,5-oxadiazole-3-carboxamide (100 mg, 0.297 mmol) in AcOH (2 mL) was stirred at 110° C. for 20 min, cooled to RT, pH adjusted to around 7 with sat. aq. NaHCO₃ and extracted with EtOAc (10 mL×3). The combined organic layers were concentrated to give a residue, which was purified by prep. HPLC (column: Phenomenex Luna C₁₈ 75*30 mm*3 μm, Mobile Phase A: 0.225% aq. FA, Mobile Phase B: CH₃CN, 25% B to 75%). The pure fractions were collected and the volatiles removed in vacuo. The residue was partitioned between CH₃CN (2 mL) and H₂O (10 mL) and lyophilized to dryness to give the title compound (2 mg, 2%) as a white powder.

MS ES⁺: 319.3

¹H NMR (400 MHz, MeOH-d₄) 9.00 (s, 2H), 8.57 (dd, J=1.3, 4.8 Hz, 1H), 8.28 (dd, J=1.2, 8.2 Hz, 1H), 7.48 (dd, J=4.8, 8.1 Hz, 1H), 6.07 (s, 2H), 2.82 (s, 3H).

### Example 166: 4-(7-fluoro-1-((6-(trifluoromethyl)pyridazin-3-yl)methyl)-benzimidazol-2-yl)-1,2,5-oxadiazol-3-amine

### Example 167: 4-(4-fluoro-1-((6-(trifluoromethyl)pyridazin-3-yl)methyl)-benzimidazol-2-yl)-1,2,5-oxadiazol-3-amine

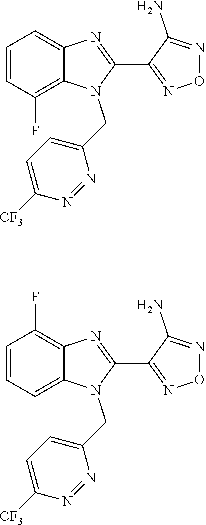 [SMILES: Nc1nonc1-c1nc2cccc(F)c2n1Cc1ccc(C(F)(F)F)nn1 ; Nc1nonc1-c1nc2c(F)cccc2n1Cc1ccc(C(F)(F)F)nn1]

Step 1: A solution of 3-chloro-6-(trifluoromethyl)pyridazine (1 g, 5.48 mmol) in dioxane (20 mL) was treated with Al(CH₃)₃(2M in toluene, 5.48 mL) and Pd(PPh₃)₄(633 mg, 0.548 mmol) under N₂. The mixture was degassed and purged with N₂ three times, stirred at 100° C. for 4 hours and quenched by adding MeOH (10 mL) at 0° C. The mixture was filtered and the filtrate concentrated. The residue was purified by flash chromatography (ISCO®; 40 g SepaFlash®, 0-30% EtOAc in petroleum ether) to afford 3-methyl-6-(trifluoromethyl)pyridazine (460 mg, 51%) as a pale yellow solid.

Step 2: A solution of 3-methyl-6-(trifluoromethyl)pyridazine (400 mg, 2.47 mmol) and NBS (483 mg, 2.71 mmol) in CCl₄ (8 mL) was treated with 2,2′-azobis(2-methylpropionitrile) (81 mg, 0.493 mmol), stirred at 80° C. for 16 hours and concentrated under reduced pressure. The residue was purified by flash chromatography (ISCO®; 12 g SepaFlash®, 0-18% EtOAc in petroleum ether) to afford 3-(bromomethyl)-6-(trifluoromethyl)pyridazine (168 mg, 28%) as a red oil.

Step 3: A solution of 3-(bromomethyl)-6-(trifluoromethyl)pyridazine (168 mg, 0.697 mmol) and 4-(7-fluoro-1H-benzimidazol-2-yl)-1,2,5-oxadiazol-3-amine (153 mg, 0.697 mmol) in DMF (2 mL) was treated with Cs₂CO₃ (454 mg, 1.39 mmol) and KI (23 mg, 0.139 mmol), stirred at 90° C. for 1 hour, diluted with H₂O (15 mL) and extracted with EtOAc (15 mL×3). The combined organic layers were washed with brine (30 mL), dried over Na₂SO₄ and concentrated to give a residue, which was purified by flash chromatography (ISCO®; 20 g SepaFlash®, 0-20% EtOAc in petroleum ether) followed by SFC (separation condition: DAICEL CHIRALPAKAD (250 mm*30 mm, 10 μm); Mobile Phase: A: Supercritical CO₂, B: 0.1% NH₃·H₂O in EtOH, A:B=75:25). The pure fractions were collected and the volatiles removed in vacuo. The residue was partitioned between CH₃CN (2 mL) and H₂O (i mL) and lyophilized to dryness to give 4-(7-fluoro-1-((6-(trifluoromethyl)pyridazin-3-yl)methyl)-benzimidazol-2-yl)-1,2,5-oxadiazol-3-amine (peak 1, 11.67 mg, 0.031 mmol, 8.6%) and 4-(4-fluoro-1-((6-(trifluoromethyl)pyridazin-3-yl)methyl)-benzimidazol-2-yl)-1,2,5-oxadiazol-3-amine (peak 2, 42.13 mg, 0.111 mmol, 31.2%) as white solids.

### Example 166 (Peak 1): 4-(7-fluoro-1-((6-(trifluoromethyl)pyridazin-3-yl)methyl)-benzimidazol-2-yl)-1,2,5-oxadiazol-3-amine

MS ES⁺: 380.3

¹H NMR (400 MHz, DMSO-d₆) 8.30 (d, J=8.8 Hz, 1H), 8.15 (d, J=8.8 Hz, 1H), 7.74 (d, J=8.2 Hz, 1H), 7.39-7.32 (m, 1H), 7.28-7.20 (m, 1H), 6.98 (s, 2H), 6.43 (s, 2H).

### Example 167 (Peak 2): 4-(4-fluoro-1-((6-(trifluoromethyl)pyridazin-3-yl)methyl)-benzimidazol-2-yl)-1,2,5-oxadiazol-3-amine

MS ES⁺: 380.3

¹H NMR (400 MHz, DMSO-d₆) 8.28 (d, J=8.8 Hz, 1H), 8.07 (d, J=8.8 Hz, 1H), 7.65 (d, J=8.2 Hz, 1H), 7.45-7.38 (m, 1H), 7.27-7.20 (m, 1H), 6.94 (s, 2H), 6.40 (s, 2H).

### Example 168: 4-(7-fluoro-1-((6-methylpyridazin-3-yl)methyl)-benzimidazol-2-yl)-1,2,5-oxadiazol-3-amine

### Example 169: 4-(4-fluoro-1-((6-methylpyridazin-3-yl)methyl)-benzimidazol-2-yl)-1,2,5-oxadiazol-3-amine

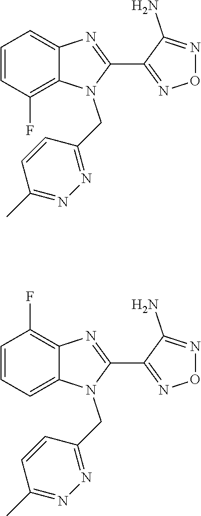 [SMILES: Cc1ccc(Cn2c(-c3nonc3N)nc3cccc(F)c32)nn1 ; Cc1ccc(Cn2c(-c3nonc3N)nc3c(F)cccc32)nn1]

Step 1: A mixture of methyl 6-methylpyridazine-3-carboxylate (200 mg, 1.31 mmol) in THF (2 mL) was treated with LiAlH₄ (100 mg, 2.63 mmol) in one portion at 0° C. under N₂ for 30 min, warmed to RT and stirred for 1.5 hours. The mixture was cooled to 0° C. and dropwise treated with NH₃·H₂O (28% purity in H₂O) until pH>8 was reached. The mixture was filtered and the filtrate extracted with CH₂Cl₂ (10 mL×3). The combined organic layers were dried over Na₂SO₄ and evaporated to give (6-methylpyridazin-3-yl)methanol (132 mg, 81%) as a yellow liquid.

¹H NMR (400 MHz, DMSO-d₆) 7.80-7.41 (m, 2H), 4.69-4.62 (m, 2H), 2.78-2.65 (m, 3H).

Step 2: (6-Methylpyridazin-3-yl)methanol (132 mg, 1.06 mmol), 4-(7-fluoro-1H-benzimidazol-2-yl)-1,2,5-oxadiazol-3-amine (233 mg, 1.06 mmol) and 2-(tributylphosphoranylidene)acetonitrile (257 mg, 1.06 mmol) in THF (2 mL) were heated at 100° C. for 3 hours under microwave irradiation, and then concentrated to give a residue, which was purified by prep. HPLC (column: Phenomenex Luna 30*30 mm*10 μm+YMC AQ 100*30*10 μm, Mobile Phase A: 0.05% NH₃·H₂O in H2O, Mobile Phase B: CH₃CN, 25% B to 85%). The pure fractions were collected and the volatiles removed in vacuo. The residue was partitioned between CH₃CN (20 mL) and H₂O (100 mL) and lyophilized to give 4-(7-fluoro-1-((6-methylpyridazin-3-yl)methyl)-benzimidazol-2-yl)-1,2,5-oxadiazol-3-amine (peak 1, 3 mg, 1%) and 4-(4-fluoro-1-((6-methylpyridazin-3-yl)methyl)-benzimidazol-2-yl)-1,2,5-oxadiazol-3-amine (peak 2, 5 mg, 1%) as white powders.

### Example 168 (Peak 1): 4-(7-fluoro-1-((6-methylpyridazin-3-yl)methyl)-benzimidazol-2-yl)-1,2,5-oxadiazol-3-amine

MS ES⁺: 326.1

¹H NMR (400 MHz, MeOH-d4) 7.68-7.56 (m, 1H), 7.55-7.41 (m, 2H), 7.34-7.23 (m, 1H), 7.04 (dd, J=8.0, 12.0 Hz, 1H), 6.31 (s, 2H), 2.56 (s, 3H).

### Example 169 (Peak 2): 4-(4-fluoro-1-((6-methylpyridazin-3-yl)methyl)-benzimidazol-2-yl)-1,2,5-oxadiazol-3-amine

MS ES⁺: 326.1

¹H NMR (400 MHz, MeOH-d₄) 7.49 (d, J=4.0 Hz, 2H), 7.40 (d, J=8.0 Hz, 1H), 7.30 (td, J=4.1, 8.1 Hz, 1H), 7.13-6.95 (m, 1H), 6.19 (s, 2H), 2.56 (s, 3H).

### Example 170: 3-(6,7-difluoro-1-(pyridazin-3-ylmethyl)-benzimidazol-2-yl)-4-methyl-1,2,5-oxadiazole

### Example 171: 3-(4,5-difluoro-1-(pyridazin-3-ylmethyl)-benzimidazol-2-yl)-4-methyl-1,2,5-oxadiazole

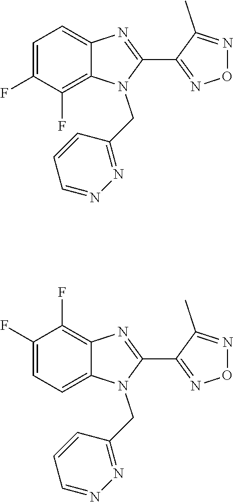 [SMILES: Cc1nonc1-c1nc2ccc(F)c(F)c2n1Cc1cccnn1 ; Cc1nonc1-c1nc2c(F)c(F)ccc2n1Cc1cccnn1]

Step 1: A mixture of 3,4-difluorobenzene-1,2-diamine (500 mg, 3.47 mmol) and 4-methyl-1,2,5-oxadiazole-3-carboxylic acid (Intermediate 2) (444 mg, 3.47 mmol) in CH₂Cl₂ (2 mL) was treated with triethylamine (702 mg, 6.94 mmol) and TP (3.31 g, 5.20 mmol, 50% purity in EtOAc) at 25° C., stirred at 25° C. for 1 hour, poured into H₂O (15 mL) and extracted with CH₂Cl₂ (15 mL×3). The combined organic layers were concentrated to afford N-(2-amino-3,4-difluorophenyl)-4-methyl-1,2,5-oxadiazole-3-carboxamide (800 mg, 90%) as a brown solid, which was used directly for the next step.

MS ES⁺: 255.0

Step 2: A mixture of N-(2-amino-3,4-difluorophenyl)-4-methyl-1,2,5-oxadiazole-3-carboxamide (800 mg, 3.15 mmol) in AcOH (5 mL) was stirred at 90° C. for 10 hours. The mixture was pH adjusted to 9 with sat. aq. NaHCO₃ (15 mL) and extracted with EtOAc (15 mL×3). The combined organic layers were dried over Na₂SO₄ and concentrated to give a residue, which was purified by flash chromatography (ISCO®; 12 g SepaFlash®, 0-25% EtOAc in petroleum ether) to afford 3-(6,7-difluoro-benzimidazol-2-yl)-4-methyl-1,2,5-oxadiazole (650 mg, 66%) as a brown solid.

MS ES⁺: 237.0

Step 3: A mixture of 3-(6,7-difluoro-benzimidazol-2-yl)-4-methyl-1,2,5-oxadiazole (300 mg, 1.27 mmol), pyridazin-3-ylmethanol (140 mg, 1.27 mmol) and 2-(tributylphosphoranylidene)acetonitrile (613 mg, 2.54 mmol) in THF (2 mL) was heated at 100° C. for 3 hours under microwave irradiation and concentrated to give a residue, which was purified by flash chromatography (ISCO®; 40 g SepaFlash®, 0-50% EtOAc in petroleum ether) to give 3-(6,7-difluoro-1-(pyridazin-3-ylmethyl)-benzimidazol-2-yl)-4-methyl-1,2,5-oxadiazole (peak 1, 95.4 mg, 22%) and 3-(4,5-difluoro-1-(pyridazin-3-ylmethyl)-benzimidazol-2-yl)-4-methyl-1,2,5-oxadiazole (peak 2, 56 mg, 13%) as white solids.

### Example 170 (Peak 1): 3-(6,7-difluoro-1-(pyridazin-3-ylmethyl)-benzimidazol-2-yl)-4-methyl-1,2,5-oxadiazole

MS ES⁺: 329.3

¹H NMR (400 MHz, DMSO-d₆) 9.15 (dd, J=1.2, 4.8 Hz, 1H), 7.85-7.64 (m, 3H), 7.53-7.37 (m, 1H), 6.22 (s, 2H), 2.76 (s, 3H).

### Example 171 (Peak 2): 3-(4,5-difluoro-1-(pyridazin-3-ylmethyl)-benzimidazol-2-yl)-4-methyl-1,2,5-oxadiazole

MS ES⁺: 329.3

¹H NMR (400 MHz, DMSO-d₆) 9.13 (t, J=3.2 Hz, 1H), 7.79-7.59 (m, 3H), 7.52 (ddd, J=7.2, 9.2, 11.2 Hz, 1H), 6.21 (s, 2H), 2.78 (s, 3H).

### Example 172: 4-(1-((6-(difluoromethyl)pyridazin-3-yl)methyl)-7-fluoro-benzimidazol-2-yl)-1,2,5-oxadiazol-3-amine

### Example 173: 4-(1-((6-(difluoromethyl)pyridazin-3-yl)methyl)-4-fluoro-benzimidazol-2-yl)-1,2,5-oxadiazol-3-amine

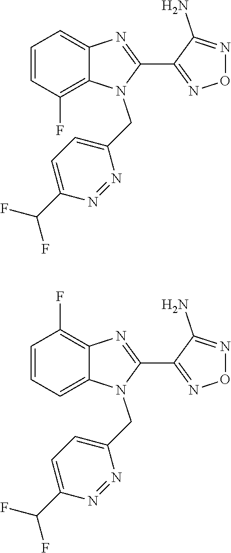 [SMILES: Nc1nonc1-c1nc2cccc(F)c2n1Cc1ccc(C(F)F)nn1 ; Nc1nonc1-c1nc2c(F)cccc2n1Cc1ccc(C(F)F)nn1]

Step 1: A mixture of 3-chloro-6-(difluoromethyl)pyridazine (500 mg, 3.04 mmol), sodium acetate (499 mg, 6.08 mmol) and PdCl₂(dppf).CH₂Cl₂ (248 mg, 0.304 mmol) in MeOH (5 mL) was degassed and purged with CO three times, stirred at 80° C. for 10 hours under CO (50 psi), and concentrated to give a residue, which was purified by flash chromatography (ISCO®; 12 g SepaFlash®, 0-20% EtOAc in petroleum ether) to afford methyl 6-(difluoromethyl)pyridazine-3-carboxylate (433 mg, 70%) as a white solid.

MS ES⁺: 189.0

Step 2: A mixture of methyl 6-(difluoromethyl)pyridazine-3-carboxylate (230 mg, 1.22 mmol) and LiOH—H₂O (103 mg, 2.45 mmol) in THF (1 mL) and H₂O (1 mL) was stirred at 25° C. for 1 hour. The mixture was pH adjusted to 4 with aq. HCl (1M in H₂O) and extracted with EtOAc (20 mL×3). The combined organic layers were dried over Na₂SO₄ and concentrated to afford 6-(difluoromethyl)pyridazine-3-carboxylic acid (210 mg, crude) as a yellow solid, which was used for the next step without further purification.

Step 3: A solution of 6-(difluoromethyl)pyridazine-3-carboxylic acid (100 mg, 0.574 mmol), ethyl carbonochloridate (312 mg, 2.87 mmol) and triethylamine (58 mg, 0.574 mmol) in THF (1 mL) was stirred at −10° C. for 30 min and filtered. The filtrate was added dropwise into a solution of NaBH₄ (65 mg, 1.72 mmol) in H₂O (1 mL) at RT and the mixture was stirred for 2 hours. The pH was adjusted to 4 with HCl (aq., 1M in H₂O). The mixture was extracted with CH₂Cl₂ (15 mL×3). The combined organic layers were concentrated to give a residue, which was purified by flash chromatography (ISCO®; 4 g SepaFlash®, 0-10% MeOH in CH₂Cl₂) to afford (6-(difluoromethyl)pyridazin-3-yl)methanol (40 mg, 43%) as a yellow solid.

MS ES⁺: 161

Step 4: A mixture of (6-(difluoromethyl)pyridazin-3-yl)methanol (40 mg, 0.250 mmol), 4-(7-fluoro-1H-benzimidazol-2-yl)-1,2,5-oxadiazol-3-amine (66 mg, 0.300 mmol) and 2-(tributylphosphoranylidene)acetonitrile (181 mg, 0.749 mmol) in THF (1 mL) was heated at 100° C. for 2 hours under microwave irradiation and concentrated to give a residue, which was purified by prep. HPLC (column: Phenomenex Luna 30*30 mm*10 μm+YMC AQ 100*30*10 μm, Mobile Phase A: 0.05% aq. NH₃·H₂O, Mobile Phase B: CH₃CN, 45% B to 95%). The pure fractions were collected and the volatiles removed in vacuo. The residue was partitioned between CH₃CN (20 mL) and H₂O (100 mL) and lyophilized to give crude product, which was further separated by SFC (separation condition: DAICEL CHIRALPAK AD (250 mm*30 mm, 10 μm); Mobile Phase: A: 0.1% NH₃·H₂O in EtOH, B: Supercritical CO₂, A:B=30%) to give 4-(1-((6-(difluoromethyl)pyridazin-3-yl)methyl)-7-fluoro-benzimidazol-2-yl)-1,2,5-oxadiazol-3-amine (peak 1, 1.02 mg, 1%) and 4-(1-((6-(difluoromethyl)pyridazin-3-yl)methyl)-4-fluoro-benzimidazol-2-yl)-1,2,5-oxadiazol-3-amine (peak 2, 4 mg, 4%) as white solids.

### Example 172 (Peak 1): 4-(1-((6-(difluoromethyl)pyridazin-3-yl)methyl)-7-fluoro-benzimidazol-2-yl)-1,2,5-oxadiazol-3-amine

MS ES⁺: 361.9

¹H NMR (400 MHz, MeOD-d₄) 7.98 (d, J=8.8 Hz, 1H), 7.84 (d, J=8.8 Hz, 1H), 7.69 (d, J=8.4 Hz, 1H), 7.33 (d, J=4.8 Hz, 1H), 7.15-7.08 (m, 1H), 7.00-6.79 (m, 1H), 6.48 (s, 2H).

### Example 173 (Peak 2): 4-(1-((6-(difluoromethyl)pyridazin-3-yl)methyl)-4-fluoro-benzimidazol-2-yl)-1,2,5-oxadiazol-3-amine

MS ES⁺: 362.3

¹H NMR (400 MHz, MeOD-d₄) 7.95 (d, J=8.8 Hz, 1H), 7.82 (d, J=8.8 Hz, 1H), 7.48 (d, J=8.4 Hz, 1H), 7.38 (d, J=4.8 Hz, 1H), 7.20-6.79 (m, 2H), 6.36 (s, 2H).

### Example 174: 6-((2-(4-amino-1,2,5-oxadiazol-3-yl)-7-fluoro-benzimidazol-1-yl)methyl)pyridazine-3-carbonitrile

### Example 175: 6-((2-(4-amino-1,2,5-oxadiazol-3-yl)-4-fluoro-benzimidazol-1-yl)methyl)pyridazine-3-carbonitrile

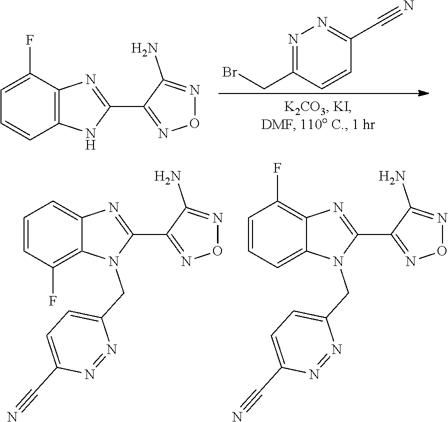

A mixture of 4-(4-fluoro-1H-benzimidazol-2-yl)-1,2,5-oxadiazol-3-amine (66.4 mg, 0.30 mmol), 6-(bromomethyl)pyridazine-3-carbonitrile (60 mg, 0.30 mmol), K₂CO₃ (83.8 mg, 0.61 mmol) and KI (10 mg, 0.06 mmol) in DMF (1 mL) was stirred at 110° C. for 1 hour and filtered. The filtrate was concentrated under reduced pressure to give a residue, which was purified by prep. HPLC (column: Phenomenex Luna C₁₈ 75*30 mm*3 μm, Mobile Phase A: 10 mM aq. NH₄HCO₃, Mobile Phase B: CH₃CN, 23% B to 68%) to give crude product, which was further separated by SFC (separation condition: DAICEL CHIRALCEL OD (250 mm*30 mm, 10 m); Mobile Phase: A: Supercritical CO₂, B: 0.1% NH₃·H₂O in EtOH, A:B=7:3) to give 6-((2-(4-amino-1,2,5-oxadiazol-3-yl)-7-fluoro-benzimidazol-1-yl)methyl)pyridazine-3-carbonitrile (peak 1, 3 mg, 3%) and 6-((2-(4-amino-1,2,5-oxadiazol-3-yl)-4-fluoro-benzimidazol-1-yl)methyl)pyridazine-3-carbonitrile (peak 2, 10 mg, 9%) as white solids.

### Example 174 (Peak 1): 6-((2-(4-amino-1,2,5-oxadiazol-3-yl)-7-fluoro-benzimidazol-1-yl)methyl)pyridazine-3-carbonitrile

MS ES⁺: 337.3

¹H NMR (400 MHz, DMSO-d₆) 8.40-8.34 (m, 1H), 8.14-8.09 (m, 1H), 7.77-7.72 (m, 1H), 7.40-7.33 (m, 1H), 7.27-7.21 (m, 1H), 6.98 (s, 2H), 6.41 (s, 2H).

### Example 175 (Peak 2): 6-((2-(4-amino-1,2,5-oxadiazol-3-yl)-4-fluoro-benzimidazol-1-yl)methyl)pyridazine-3-carbonitrile

MS ES⁺: 337.3

¹H NMR (400 MHz, DMSO-d₆) 8.37-8.33 (m, 1H), 8.06-8.01 (m, 1H), 7.66-7.61 (m, 1H), 7.45-7.38 (m, 1H), 7.27-7.20 (m, 1H), 6.94 (s, 2H), 6.38 (s, 2H).

### Example 176: 4-(7-fluoro-1-((6-(trifluoromethoxy)pyridin-3-yl)methyl)-benzimidazol-2-yl)-1,2,5-oxadiazol-3-amine

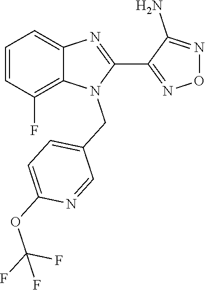 [SMILES: Nc1nonc1-c1nc2cccc(F)c2n1Cc1ccc(OC(F)(F)F)nc1]

Step 1: A solution of 6-(trifluoromethoxy)pyridine-3-carboxylic acid (400 mg, 1.93 mmol) in THF (8 mL) was treated dropwise with BH₃-THF (1M, 9.66 mL) at 0° C. under N₂, stirred at 0° C. for 30 min, and warmed to 25° C. and stirred for 10 hours. The mixture was quenched by adding MeOH (20 mL) at 0° C. and concentrated under reduced pressure to give (6-(trifluoromethoxy)pyridin-3-yl)methanol (375 mg) as a colourless oil, which was used for the next step without further purification.

MS ES⁺: 193.9

Step 2: A solution of (6-(trifluoromethoxy)pyridin-3-yl)methanol (100 mg, 0.52 mmol) in CH₂Cl₂ (1.5 mL) was treated dropwise with SOCl₂ (308 mg, 2.59 mmol) at ° C., stirred at 25° C. for 1 hour, and concentrated under reduced pressure to give 5-(chloromethyl)-2-(trifluoromethoxy)pyridine hydrochloride (130 mg, crude) as a white solid.

MS ES⁺: 212.2

Step 3: A mixture of 4-(7-fluoro-1H-benzimidazol-2-yl)-1,2,5-oxadiazol-3-amine (135 mg, 0.61 mmol), 5-(chloromethyl)-2-(trifluoromethoxy)pyridine hydrochloride (130 mg, 0.61 mmol), K₂CO₃ (170 mg, 1.23 mmol) and KI (20.4 mg, 0.12 mmol) in DMF (1.5 mL) was stirred at 110° C. for 12 hours and filtered. The filtrate was concentrated under reduced pressure to give a residue, which was purified by prep. HPLC (column: Phenomenex Luna C₁₈ 75*30 mm*3 μm, Mobile Phase A: 0.225% aq. FA, Mobile Phase B: CH₃CN, 38% B to 72%) to give the title compound (8 mg, 3%) as a white solid.

MS ES⁺: 395.2

¹H NMR (400 MHz, DMSO-d₆) 8.31-8.26 (m, 1H), 7.83-7.77 (m, 1H), 7.75-7.70 (m, 1H), 7.39-7.32 (m, 1H), 7.28-7.21 (m, 2H), 6.98 (s, 2H), 6.04 (s, 2H).

### Example 177: 6-((2-(4-amino-1,2,5-oxadiazol-3-yl)-3H-imidazo[4,5-b]pyridin-3-yl)methyl)pyridazine-3-carbonitrile

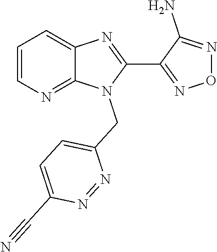 [SMILES: N#Cc1ccc(Cn2c(-c3nonc3N)nc3cccnc32)nn1]

Step 1: A mixture of 3-bromo-6-methyl-pyridazine (4 g, 23.12 mmol), Zn(CN)₂ (2.85 g, 24.27 mmol) and 1,1-bis(diphenylphosphino)ferrocene (1.28 g, 2.31 mmol) in DMF (40 mL) was treated with Pd(dba)₂ (665 mg, 1.16 mmol) under N₂, stirred at 110° C. for 12 hours under N₂, diluted with H₂O (200 mL) and extracted with EtOAc (300 mL×3). The combined organic layers were dried over Na2SO₄ and concentrated to give a residue, which was purified by flash chromatography (ISCO®; 20 g SepaFlash®, 0-50% EtOAc in petroleum ether) to give 6-methylpyridazine-3-carbonitrile (1.93 g, 63%) as a yellow solid.

MS ES⁺: 120.3

¹H NMR (400 MHz, CDCl₃-d) 7.75 (d, J=8.6 Hz, 1H), 7.52 (d, J=8.6 Hz, 1H), 2.86 (s, 3H).

Step 2: A solution of 6-methylpyridazine-3-carbonitrile (1.90 g, 15.9 mmol) and NBS (3.4 g, 19.1 mmol) in DMF (20 mL) was treated with 2,2′-(diazene-1,2-diyl)bis(2-methylpropanenitrile) (262 mg, 1.59 mmol), stirred at 80° C. for 45 min, diluted with H₂O (50 mL) and extracted with CH₂Cl₂ (50 mL×3). The combined organic layers were dried over Na2SO₄ and concentrated to give a residue, which was purified by flash chromatography (ISCO®; 20 g SepaFlash®, 0-25% EtOAc in petroleum ether) to give 6-(bromomethyl)pyridazine-3-carbonitrile (1.48 g, 45%) as a yellow solid.

¹H NMR (400 MHz, CDCl₃-d) 7.87 (s, 2H), 4.82 (s, 2H).

Step 3: A solution of tert-butyl N-tert-butoxycarbonylcarbamate (1.73 g, 7.98 mmol, 1.83 mL) in THF (20 mL) was treated with NaH (479 mg, 11.97 mmol, 60% purity) at 25° C. under N₂, stirred at 0° C. for 30 min, treated with 6-(bromomethyl)pyridazine-3-carbonitrile (1.58 g, 7.98 mmol) and stirred at 25° C. for 1 hour. The mixture was diluted with sat. aq. NH₄Cl (30 mL) and extracted with EtOAc (50 mL×3). The combined organic layers were dried over Na₂SO₄ and concentrated to give a residue, which was purified by flash chromatography (ISCO®; 20 g SepaFlash®, 0-200% EtOAc in petroleum ether) to give tert-butyl N-tert-butoxycarbonyl-N-[(6-cyanopyridazin-3-yl)methyl]carbamate (492 mg, 18%) as a yellow solid.

Step 4: A solution of tert-butyl N-tert-butoxycarbonyl-N-[(6-cyanopyridazin-3-yl)methyl]carbamate (290 mg, 0.87 mmol) in CH₂Cl₂ (2 mL) was treated with 4M HCl in dioxane (2.17 mL), stirred at 25° C. for 30 min, and concentrated to give 6-(aminomethyl)pyridazine-3-carbonitrile dihydrochloride (180 mg, crude) as a yellow solid.

Step 5: A solution of 6-(aminomethyl)pyridazine-3-carbonitrile dihydrochloride (180 mg, 0.87 mmol), 2-fluoro-3-nitro-pyridine (124 mg, 0.87 mmol) and triethylamine (440 mg, 4.35 mmol) in CH₃CN (3 mL) was stirred at 25° C. for 12 hours and diluted with H₂O (20 mL). The resulting precipitate was collected by filtration to give 6-(((3-nitropyridin-2-yl)amino)methyl)pyridazine-3-carbonitrile (183 mg, 73%) as a yellow solid.

MS ES⁺: 256.9

¹H NMR (400 MHz, DMSO-d₆) 9.23-9.14 (m, 1H), 8.49 (dd, J=1.8, 8.3 Hz, 1H), 8.37 (dd, J=1.8, 4.5 Hz, 1H), 8.25 (d, J=8.8 Hz, 1H), 7.90 (d, J=8.8 Hz, 1H), 6.83 (dd, J=4.5, 8.3 Hz, 1H), 5.16 (d, J=5.9 Hz, 2H).

Step 6: A solution of 6-(((3-nitropyridin-2-yl)amino)methyl)pyridazine-3-carbonitrile (90 mg, 0.35 mmol) in H₂O (0.8 mL) and THF (1.2 mL) was treated with Na₂S₂O₄ (306 mg, 1.76 mmol) at 80° C., stirred for 10 min, cooled and extracted with EtOAc (15 mL×3). The combined organic layers were dried over Na₂SO₄ and concentrated under reduced pressure to give 6-(((3-aminopyridin-2-yl)amino)methyl)pyridazine-3-carbonitrile (80 mg, 0.29 mmol, 83.6% yield, 83.0% purity) as a yellow solid.

MS ES⁺: 227.1

Step 7: A solution of 6-(((3-aminopyridin-2-yl)amino)methyl)pyridazine-3-carbonitrile (30 mg, 0.13 mmol), 4-amino-1,2,5-oxadiazole-3-carboxylic acid (17 mg, 0.13 mmol) and triethylamine (40 mg, 0.40 mmol) in DMF (1 mL) was treated with HATU (61 mg, 0.16 mmol), stirred at 25° C. for 12 hours, diluted with H₂O (10 mL) and extracted with EtOAc (15 mL×3). The combined organic layers were dried over Na₂SO₄ and concentrated under reduced pressure to give 4-amino-N-(2-(((6-cyanopyridazin-3-yl)methyl)amino)pyridin-3-yl)-1,2,5-oxadiazole-3-carboxamide (45 mg, crude) as a yellow solid.

MS ES⁺: 338.0

Step 8: A mixture of 4-amino-N-(2-(((6-cyanopyridazin-3-yl)methyl)amino)pyridin-3-yl)-1,2,5-oxadiazole-3-carboxamide (45 mg, 0.13 mmol) in AcOH (1 mL) was stirred at 110° C. for 30 min, filtered and concentrated to give a residue, which was purified by prep. HPLC (column: Phenomenex Luna 30*30 mm*10 m+YMC AQ 100*30*10 μm, Mobile Phase A: 0.05% aq. NH₃·H₂O, Mobile Phase B: CH₃CN, 20% B to 70%). The pure fractions were collected and the volatiles removed in vacuo. The residue was partitioned between CH₃CN (2 mL) and H₂O (10 mL) and lyophilized to give the title compound (2 mg, 4%) as an off-white solid.

MS ES⁺: 320.0

¹H NMR (400 MHz, DMSO-d₆) 8.54-8.48 (m, 1H), 8.40-8.31 (m, 2H), 8.14-8.07 (m, 1H), 7.53-7.47 (m, 1H), 6.99 (s, 2H), 6.34 (s, 2H).

### Example 178: 4-(7-fluoro-1-(pyridazin-3-ylmethyl)-benzimidazol-2-yl)-N-methyl-1,2,5-oxadiazol-3-amine

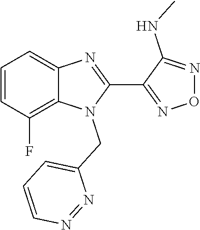 [SMILES: CNc1nonc1-c1nc2cccc(F)c2n1Cc1cccnn1]

Step 1: A solution of 4-(7-fluoro-1H-benzimidazol-2-yl)-1,2,5-oxadiazol-3-amine (1 g, 4.56 mmol) and pyridazin-3-ylmethanol (502 mg, 4.56 mmol) in THF (10 mL) was treated with 2-(tributylphosphoranylidene)acetonitrile (2.20 g, 9.13 mmol) under N₂, stirred at 100° C. for 4 hours under microwave irradiation, cooled, diluted with H₂O (50 mL) and extracted with EtOAc (50 mL×3). The combined organic layers were dried over Na₂SO₄ and concentrated to give a residue, which was purified by flash chromatography (SiO₂, petroleum ether:EtOAc=1:0 to 1:1) to afford 4-(7-fluoro-1-(pyridazin-3-ylmethyl)-benzimidazol-2-yl)-1,2,5-oxadiazol-3-amine (300 mg, 19%) as a yellow solid.

MS ES⁺: 311.9

Step 2: A solution of 4-(7-fluoro-1-(pyridazin-3-ylmethyl)-benzimidazol-2-yl)-1,2,5-oxadiazol-3-amine (200 mg, 0.64 mmol) in pyridine (2 mL) was treated with 2,2,2-trifluoroacetic anhydride (270 mg, 1.29 mmol) and DMAP (39 mg, 0.32 mmol), stirred at 25° C. for 10 min, diluted with H₂O (20 mL) and extracted with EtOAc (20 mL×3). The combined organic layers were dried over Na₂SO₄ and concentrated to give a residue, which was purified by flash chromatography (SiO₂, petroleum ether:EtOAc=1:0 to 0:1) to give 2,2,2-trifluoro-N-(4-(7-fluoro-1-(pyridazin-3-ylmethyl)-benzimidazol-2-yl)-1,2,5-oxadiazol-3-yl)acetamide (113 mg, 41%) as a yellow solid.

MS ES⁺: 407.9

¹H NMR (400 MHz, DMSO-d₆) 9.18-9.12 (m, 1H), 7.75-7.67 (m, 3H), 7.42-7.35 (m, 1H), 7.32-7.23 (m, 1H), 6.28 (s, 2H).

Step 3: A mixture of 2,2,2-trifluoro-N-(4-(7-fluoro-1-(pyridazin-3-ylmethyl)-benzimidazol-2-yl)-1,2,5-oxadiazol-3-yl)acetamide (90 mg, 0.22 mmol) in THF (1.5 mL) was treated with NaH (13.3 mg, 0.33 mmol, 60% purity) at 0° C., stirred at 0° C. for 30 min, treated with MeI (38 mg, 0.27 mmol) and stirred at 25° C. for 1 hour. The mixture was quenched with HCl (5 mL, 1M in H₂O), diluted with H₂O (10 mL) and extracted with EtOAc (20 mL×3). The combined organic layers were dried over Na₂SO₄ and concentrated to give a residue, which was purified by flash chromatography (SiO₂, petroleum ether:EtOAc=1:0 to 1:1) to give 2,2,2-trifluoro-N-(4-(7-fluoro-1-(pyridazin-3-ylmethyl)-benzimidazol-2-yl)-1,2,5-oxadiazol-3-yl)-N-methylacetamide (15 mg, 7%) as a yellow solid.

MS ES⁺: 422.2

Step 4: A solution of 2,2,2-trifluoro-N-(4-(7-fluoro-1-(pyridazin-3-ylmethyl)-benzimidazol-2-yl)-1,2,5-oxadiazol-3-yl)-N-methylacetamide (15 mg, 0.04 mmol) in MeOH (1 mL) and H₂O (0.2 mL) was treated with K₂CO₃ (9.8 mg, 0.07 mmol), stirred at 50° C. for 12 hours, filtered and concentrated under reduced pressure to give a residue, which was purified by prep. HPLC (column: Phenomenex Gemini-NX C₁₈ 75*30 mm*3 μm, Mobile Phase A: water (0.05% NH₃·H₂O+10 mM NH₄HCO₃), Mobile Phase B: CH₃CN, 22% B to 52%). The pure fractions were collected and the volatiles removed in vacuo. The residue was partitioned between CH₃CN (2 mL) and H₂O (10 mL) and lyophilized to give the title compound (3 mg, 23%) as white solid.

MS ES⁺: 326.0

¹H NMR (400 MHz, DMSO-d₆) 9.19-9.15 (m, 1H), 7.76-7.70 (m, 3H), 7.37-7.31 (m, 1H), 7.20-7.07 (m, 2H), 7.00 (s, 2H), 2.13-2.10 (m, 3H).

### Example 179: 4-(4-fluoro-1-(pyridazin-3-ylmethyl)-benzimidazol-2-yl)-1,2,5-oxadiazol-3-amine

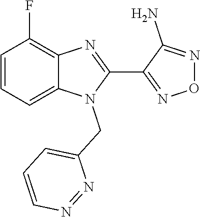 [SMILES: Nc1nonc1-c1nc2c(F)cccc2n1Cc1cccnn1]

Prepared as described for Example 6 using 4-(7-fluoro-1H-benzimidazol-2-yl)-1,2,5-oxadiazol-3-amine (500 mg, 2.28 mmol) and pyridazin-3-ylmethanol (251 mg, 2.28 mmol) to give the title compound (300 mg, 41%) as a yellow solid.

MS ES⁺: 312.0

¹H NMR (400 MHz, DMSO-d₆) 9.17-9.09 (m, 1H), 7.78-7.59 (m, 3H), 7.45-7.32 (m, 1H), 7.26-7.18 (m, 1H), 7.03-6.91 (m, 2H), 6.32-6.24 (m, 2H).

### Example 180: 3-(1-((6-(ethylsulfonyl)pyridin-3-yl)methyl)-benzimidazol-2-yl)-4-methyl-1,2,5-oxadiazole

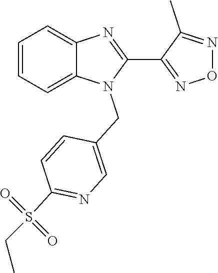 [SMILES: CCS(=O)(=O)c1ccc(Cn2c(-c3nonc3C)nc3ccccc32)cn1]

A mixture of 3-(1-((6-bromopyridin-3-yl)methyl)benzimidazol-2-yl)-4-methyl-1,2,5-oxadiazole (50 mg, 0.135 mmol), sodium ethanesulfinate (31 mg, 0.270 mmol), CuI (2.6 mg, 0.014 mmol), 2-(methylamino)acetic acid (2.4 mg, 0.027 mmol) and NaOH (5.4 mg, 0.135 mmol) in DMSO (0.5 mL) was heated at 100° C. for 3 hours under microwave irradiation, poured into ice H₂O (15 mL) and extracted with EtOAc (15 mL×3). The combined organic layers were dried over Na₂SO₄ and concentrated to afford crude product, which was purified by prep. HPLC (column: Phenomenex Gemini-NX C₁₈ 75*30 mm*3 μm, Mobile Phase A: water (0.05% NH₃·H₂O+10 mM NH₄HCO₃), Mobile Phase B: CH₃CN, 25% B to 65%). The pure fractions were collected and the volatiles removed in vacuo. The residue was partitioned between CH₃CN (2 mL) and H₂O (10 mL) and lyophilized to give the title compound (27 mg, 51%) as a white solid.

MS ES⁺: 383.9

¹H NMR (400 MHz, DMSO-d₆) 8.75 (d, J=1.6 Hz, 1H), 7.98 (d, J=8.0 Hz, 1H), 7.91 (d, J=7.2 Hz, 1H), 7.82-7.68 (m, 2H), 7.48-7.33 (m, 2H), 6.08 (s, 2H), 3.50-3.37 (m, 2H), 2.79 (s, 3H), 1.20-1.02 (m, 3H).

### Example 181: 3-methyl-4-(1-((6-(methylthio)pyridin-3-yl)methyl)-benzimidazol-2-yl)-1,2,5-oxadiazole

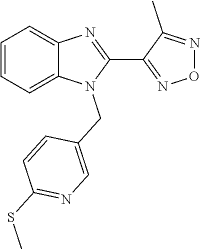 [SMILES: CSc1ccc(Cn2c(-c3nonc3C)nc3ccccc32)cn1]

A solution of 3-(1-((6-bromopyridin-3-yl)methyl)benzimidazol-2-yl)-4-methyl-1,2,5-oxadiazole (500 mg, 1.35 mmol) in DMSO (5 mL) was treated with CuI (257 mg, 1.35 mmol) and triethylamine (273 mg, 2.70 mmol) and stirred at 130° C. for 3 hours under microwave irradiation. The mixture was poured into H₂O (15 mL) and extracted with EtOAc (15 mL×3), dried over Na₂SO₄ and concentrated to afford a residue, which was purified by prep. HPLC (column: Xtimate C₁₈ 150*40 mm*10 μm, Mobile Phase A: 0.225% aq. FA, Mobile Phase B: CH₃CN, 45% B to 75%). The pure fractions were collected and the volatiles evaporated. The residue was partitioned between CH₃CN (2 mL) and H₂O (10 mL) and lyophilized to give the title compound (4 mg, 1%) as an off-white solid.

MS ES⁺: 337.9

¹H NMR (400 MHz, DMSO-d₆) 8.40 (d, J=1.6 Hz, 1H), 7.88 (d, J=7.6 Hz, 1H), 7.76 (d, J=7.6 Hz, 1H), 7.49-7.32 (m, 3H), 7.23 (d, J=8.4 Hz, 1H), 5.89 (s, 2H), 2.79 (s, 3H), 2.45 (s, 3H).

### Example 182: 3-(7-fluoro-1-(pyridazin-3-ylmethyl)-benzimidazol-2-yl)-4-methylisoxazole

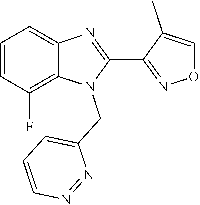 [SMILES: Cc1conc1-c1nc2cccc(F)c2n1Cc1cccnn1]

Step 1: Trifluoromethanesulfonic acid (42.54 g, 283.44 mmol) was added dropwise to a mixture of ethyl isoxazole-3-carboxylate (2 g, 14.17 mmol) and 2-bromoisoindoline-1,3-dione (7.57 g, 42.52 mmol) at ° C. for 30 min. The mixture was stirred at RT for 2 hours, poured into ice H₂O (200 mL), and pH adjusted to around 8 with sat. aq. NaHCO₃ (200 mL). The mixture was extracted with EtOAc (200 mL×3). The combined organic layers were washed with brine (200 mL), dried (Na₂SO₄) and evaporated to give a residue, which was purified by flash chromatography (ISCO®; 40 SepaFlash®, 0-30% EtOAc in petroleum ether) to afford ethyl 4-bromoisoxazole-3-carboxylate (1.2 g, 38%) as a white solid.

¹H NMR (400 MHz, DMSO-d₆) 9.49 (s, 1H), 4.39 (q, J=7.1 Hz, 2H), 1.33 (t, J=7.1 Hz, 3H).

Step 2: A solution of K₂CO₃ (1.13 g, 8.18 mmol) in H₂O (4 mL) was added to a solution of ethyl 4-bromoisoxazole-3-carboxylate (1.2 g, 5.45 mmol) in MeOH (8 mL) at 0° C. The mixture was stirred at RT for 30 min, pH adjusted to 4 with aq. 1M HCl, and extracted with EtOAc (15 mL×3). The combined organic layers were dried over Na₂SO₄ and concentrated to afford 4-bromoisoxazole-3-carboxylic acid (1.0 g, 96%) as a yellow solid.

¹H NMR (400 MHz, DMSO-d₆) 9.43 (s, 1H), 8.05 (s, 1H).

Step 3: A mixture of 4-bromoisoxazole-3-carboxylic acid (829 mg, 4.32 mmol) and 6-fluoro-N1-(pyridazin-3-ylmethyl)benzene-1,2-diamine hydrochloride (1.1 g, 4.32 mmol) in THF (1 mL) was treated with 1H-benzo[d][1,2,3]triazol-1-ol (584 mg, 4.32 mmol), DMAP (53 mg, 432 mmol) and N,N′-methanediylidenedicyclohexanamine (891 mg, 4.32 mmol) in one portion at RT. The mixture was stirred at 25° C. for 30 min, concentrated and extracted with EtOAc (60 mL×3). The combined organic layers were washed with brine (60 mL×2), dried over Na₂SO₄ and concentrated to afford 4-bromo-N-(3-fluoro-2-((pyridazin-3-ylmethyl)amino)phenyl)isoxazole-3-carboxamide (1.6 g, crude) as a brown solid.

MS ES⁺: 393.8

Step 4: A mixture of 4-bromo-N-(3-fluoro-2-((pyridazin-3-ylmethyl)amino)phenyl)isoxazole-3-carboxamide (1.6 g, 4.08 mmol) in AcOH (6 mL) was stirred at 110° C. for 30 min. The pH was adjusted to 9 with sat. aq. NaHCO₃ (50 mL) and the mixture was extracted with EtOAc (50 mL×3). The combined organic layers were dried with Na₂SO₄ and concentrated to give a residue, which was purified by flash chromatography (ISCO®; 20 g SepaFlash®, 0-50% EtOAc in petroleum ether) to afford 4-bromo-3-(7-fluoro-1-(pyridazin-3-ylmethyl)-benzimidazol-2-yl)isoxazole (1.2 g, 5⁸%) as a brown solid.

MS ES⁺: 375.9

¹H NMR (400 MHz, DMSO-d₆) 9.53 (s, 1H), 9.13 (dd, J=1.4, 4.8 Hz, 1H), 7.71-7.66 (m, 2H), 7.64-7.45 (m, 2H), 7.35-7.28 (m, 1H), 6.13 (s, 2H).

Step 5: A mixture of 4-bromo-3-(7-fluoro-1-(pyridazin-3-ylmethyl)-benzimidazol-2-yl)isoxazole (100 mg, 0.267 mmol) and 2,4,6-trimethyl-1,3,5,2,4,6-trioxatriborinane (0.267 mL, 2M in THF) in dioxane (1 mL) was treated with Cs₂CO₃ (261 mg, 0.802 mmol) and PdCl₂(dppf).CH₂Cl₂ (44 mg, 0.054 mmol) at 110° C. under N₂, stirred for 1 hour and filtered. The filtrate was concentrated to give a residue, which was purified by prep. HPLC (column: YMC Triart 30*150 mm*7 μm, Mobile Phase A: 10 mM aq. NH₄HCO₃, Mobile Phase B: CH₃CN, 0% B to 70%). The pure fractions were collected and the volatiles evaporated. The residue was partitioned between CH₃CN (20 mL) and H₂O (100 mL) and lyophilized to give the title compound (1.12 mg, 1%) as a brown powder.

MS ES⁺: 310.3

¹H NMR (400 MHz, MeOD-d₄) 9.07 (dd, J=1.1, 4.9 Hz, 1H), 8.64 (d, J=0.9 Hz, 1H), 7.70-7.63 (m, 2H), 7.59-7.54 (m, 1H), 7.31 (dt, J=4.9, 8.1 Hz, 1H), 7.08 (dd, J=8.1, 11.8 Hz, 1H), 6.28 (s, 2H), 2.36 (d, J=0.8 Hz, 3H).

### Example 183: 4-(7-fluoro-1-((2-methoxypyridin-4-yl)methyl)-benzimidazol-2-yl)-1,2,5-oxadiazol-3-amine

### Example 184: 4-(4-fluoro-1-((2-methoxypyridin-4-yl)methyl)-benzimidazol-2-yl)-1,2,5-oxadiazol-3-amine

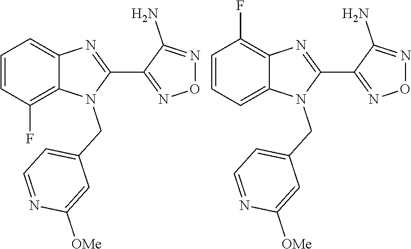

Step 1: A solution of (2-methoxy-4-pyridyl)methanol (200 mg, 1.44 mmol) in CH₂Cl₂ (2 mL) was treated dropwise with SOCl₂ (513 mg, 4.31 mmol) at ° C., stirred at RT for 8 hours and evaporated to give 4-(chloromethyl)-2-methoxy-pyridine (226 mg, crude) as a yellow solid, which was used for the next step without further purification.

MS ES⁺: 158.2

Step 2: A mixture of 4-(7-fluoro-1H-benzimidazol-2-yl)-1,2,5-oxadiazol-3-amine (250 mg, 1.14 mmol), 4-(chloromethyl)-2-methoxy-pyridine (216 mg, 1.37 mmol), K₂CO₃ (473 mg, 3.42 mmol) and KI (95 mg, 0.57 mmol) in DMF (4 mL) was stirred at 110° C. for 8 hours, cooled and filtered. The filtrate was concentrated and the residue purified by prep. HPLC (column: Phenomenex Luna 30*30 mm*10 μm+YMC AQ 100*30*10 um, Mobile Phase A: 0.225% aq. FA, Mobile Phase B: CH₃CN, 40% B to 80%) to give 4-(7-fluoro-1-((2-methoxypyridin-4-yl)methyl)-benzimidazol-2-yl)-1,2,5-oxadiazol-3-amine (peak 1, 5 mg, 1%) and 4-(4-fluoro-1-((2-methoxypyridin-4-yl)methyl)-benzimidazol-2-yl)-1,2,5-oxadiazol-3-amine (peak 2, 62 mg, 16%) as white solids.

### Example 183 (Peak 1): 4-(7-fluoro-1-((2-methoxypyridin-4-yl)methyl)-benzimidazol-2-yl)-1,2,5-oxadiazol-3-amine

MS ES⁺: 341.3

¹H NMR (400 MHz, DMSO-d₆) 8.09 (d, J=5.4 Hz, 1H), 7.74 (d, J=8.1 Hz, 1H), 7.37 (dt, J=5.0, 8.1 Hz, 1H), 7.25 (dd, J=7.9, 11.8 Hz, 1H), 6.97 (s, 2H), 6.71 (dd, J=1.1, 5.4 Hz, 1H), 6.41 (s, 1H), 5.98 (s, 2H), 3.78 (s, 3H).

### Example 184 (Peak 2): 4-(4-fluoro-1-((2-methoxypyridin-4-yl)methyl)-benzimidazol-2-yl)-1,2,5-oxadiazol-3-amine

MS ES⁺: 341.3

¹H NMR (400 MHz, DMSO-d₆) 8.07 (d, J=5.4 Hz, 1H), 7.54 (d, J=8.1 Hz, 1H), 7.41 (dt, J=4.8, 8.1 Hz, 1H), 7.23 (dd, J=7.9, 10.8 Hz, 1H), 6.94 (s, 2H), 6.71 (dd, J=1.2, 5.3 Hz, 1H), 6.46 (s, 1H), 5.96 (s, 2H), 3.83-3.74 (m, 3H).

### Example 185: 3-(4,7-difluoro-1-(pyridazin-3-ylmethyl)-benzimidazol-2-yl)-4-methyl-1,2,5-oxadiazole

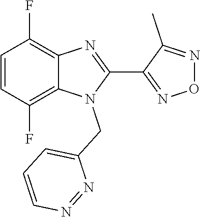 [SMILES: Cc1nonc1-c1nc2c(F)ccc(F)c2n1Cc1cccnn1]

A mixture of 3-(4,7-difluoro-1H-benzimidazol-2-yl)-4-methyl-1,2,5-oxadiazole (50 mg, 0.211 mmol) and pyridazin-3-ylmethanol (23 mg, 0.212 mmol) in THF (0.5 mL) was treated with 2-(tributylphosphoranylidene)acetonitrile (102 mg, 0.423 mmol) in one portion at RT under N₂, stirred at 100° C. for 5 hours under microwave irradiation, cooled to RT and concentrated. The crude product was purified by prep. HPLC (column: Phenomenex Luna C18 250*50 mm*10 μm, Mobile Phase A: 0.225% aq. FA, Mobile Phase B: CH₃CN, 20% B to 60%). The pure fractions were collected and the volatiles evaporated. The residue was partitioned between CH₃CN (2 mL) and H₂O (10 mL) and lyophilized to give the title compound (40 mg, 57%) as a brown powder.

MS ES⁺: 328.9

¹H NMR (400 MHz, DMSO-d₆) 9.20-9.10 (m, 1H), 7.79-7.67 (m, 2H), 7.31-7.14 (m, 2H), 6.22 (s, 2H), 2.77 (s, 3H).

### Example 186: 3-(7-fluoro-1-(pyridazin-3-ylmethyl)-benzimidazol-2-yl)isoxazol-4-amine

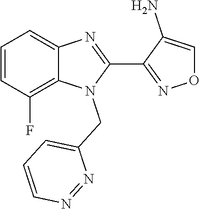 [SMILES: Nc1conc1-c1nc2cccc(F)c2n1Cc1cccnn1]

Step 1: A solution of ethyl 2-chloro-2-hydroxyimino-acetate (3 g, 19.80 mmol) in THF (30 mL) was treated with N,N-dimethyl-2-nitro-ethenamine (2.30 g, 19.80 mmol), stirred at 75° C. for 10 hours, concentrated and extracted with EtOAc (20 mL×3). The combined organic layers were washed with brine (20 mL×2), dried over Na₂SO₄ and concentrated to afford a residue, which was purified by flash chromatography (ISCO®; 12 g SepaFlash®, petroleum ether/EtOAc=3:1) to give ethyl 4-nitroisoxazole-3-carboxylate (2.8 g, 76%) as a colourless liquid.

¹H NMR (400 MHz, MeOD-d₄) 9.96 (s, 1H), 4.51 (q, J=7.1 Hz, 2H), 1.41 (t, J=7.1 Hz, 3H).

Step 2: A solution of ethyl 4-nitroisoxazole-3-carboxylate (1 g, 5.37 mmol) in MeOH (10 mL) was treated with Raney nickel (460 mg, 50% slurry in H₂O) under N₂, degassed and purged with H2 three times. The mixture was stirred under H2 (15 psi) at RT for 1 hour and filtered. The filtrate was concentrated to give ethyl 4-aminoisoxazole-3-carboxylate (800 mg, 95%) as a brown solid.

¹H NMR (400 MHz, DMSO-d₆) 8.47 (s, 1H), 4.72-4.58 (m, 2H), 4.36 (q, J=7.1 Hz, 2H), 1.32 (t, J=7.1 Hz, 3H).

Step 3: A solution of ethyl 4-aminoisoxazole-3-carboxylate (600 mg, 3.84 mmol) and DMAP (47 mg, 0.384 mmol) in THF (5 mL) was treated with di-tert-butyl dicarbonate (1.68 g, 7.69 mmol), stirred at 90° C. for 1 hour and filtered. The filtrate was concentrated under reduced pressure to give a residue, which was purified by flash chromatography (ISCO®; 4 g SepaFlash®, petroleum ether:EtOAc=3:1) to give ethyl 4-(tert-butoxycarbonylamino)isoxazole-3-carboxylate (350 mg, 35%) as a yellow oil.

¹H NMR (400 MHz, DMSO-d₆) 9.44-9.36 (m, 1H), 4.03 (d, J=7.1 Hz, 2H), 3.89 (s, 1H), 1.38 (s, 9H), 1.17 (t, J=7.1 Hz, 3H).

Step 4: A solution of K₂CO₃ (283 mg, 2.05 mmol) in H₂O (2 mL) was added to a solution of ethyl 4-(tert-butoxycarbonylamino)isoxazole-3-carboxylate (350 mg, 1.37 mmol) in MeOH (4 mL) at 0° C. The mixture was stirred at RT for 1 hour, concentrated and pH adjusted to 5 with 1M aq. HCl. The mixture was extracted with EtOAc (25 mL×3). The combined organic layers were concentrated to afford 4-(tert-butoxycarbonylamino)isoxazole-3-carboxylic acid (297 mg, 95%) as a white solid.

¹H NMR (400 MHz, DMSO-d₆) 1.99 (s, 1H), 1.91 (s, 1H), 1.43 (s, 9H). Step 5: A mixture of 6-fluoro-N1-(pyridazin-3-ylmethyl)benzene-1,2-diamine hydrochloride (150 mg, 0.589 mmol) and 1H-benzo[d][1,2,3]triazol-1-ol (80 mg, 0.589 mmol) in THF (0.5 mL) was treated with 4-(tert-butoxycarbonylamino)isoxazole-3-carboxylic acid (134 mg, 0.589 mmol) and N,N′-methanediylidenedicyclohexanamine (122 mg, 0.589 mmol) in one portion at RT, stirred for 1 hour, concentrated and extracted with EtOAc (10 mL×3). The combined organic layers were washed with brine (10 mL×2), dried over Na₂SO₄ and concentrated to afford a residue, which was purified by flash chromatography (ISCO®; 4 g SepaFlash®, 0-50% EtOAc in petroleum ether) to give tert-butyl (3-((3-fluoro-2-((pyridazin-3-ylmethyl)amino)phenyl)carbamoyl) isoxazol-4-yl)carbamate (79 mg, 21%) as a brown solid.

MS ES⁺: 429.1

Step 6: A mixture of tert-butyl (3-((3-fluoro-2-((pyridazin-3-ylmethyl)amino) phenyl)carbamoyl)isoxazol-4-yl)carbamate (20 mg, 0.047 mmol) in 4M HCl in MeOH (2 mL) was stirred at RT for 2 hours and concentrated to give a residue, which was purified by prep. HPLC (column: Phenomenex C₁₈ 75*30 mm*3 μm, Mobile Phase A: water (0.05% NH₃·H₂O+10 mM NH₄HCO₃), Mobile Phase B: CH₃CN, 15% B to 55%) to give the title compound (1 mg, 7%) as an off-white powder.

MS ES⁺: 311.0

¹H NMR (400 MHz, MeOD-d₄) 9.07 (d, J=4.8 Hz, 1H), 8.33 (s, 1H), 7.72-7.62 (m, 2H), 7.54 (d, J=8.8 Hz, 1H), 7.29 (dt, J=4.8, 8.0 Hz, 1H), 7.12-7.02 (m, 1H), 6.43 (s, 2H).

### Example 187: 4-(7-fluoro-1-(pyridazin-3-ylmethyl)-1H-imidazo[4,5-c]pyridin-2-yl)-1,2,5-oxadiazol-3-amine

### Example 188: 4-(7-fluoro-3-(pyridazin-3-ylmethyl)-3H-imidazo[4,5-c]pyridin-2-yl)-1,2,5-oxadiazol-3-amine

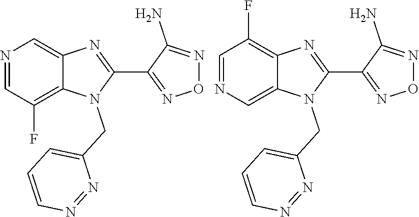

Step 1: Concentrated HNO₃ (5.06 g, 80.28 mmol) was added dropwise to a solution of 3-fluoropyridin-4-amine (3 g, 26.76 mmol) and con. H₂SO₄ (30 mL) at 0° C. The mixture was stirred at 25° C. for 2 hours, poured into ice H₂O (200 mL), pH adjusted to 8 with 2M aq. NaOH, and extracted with EtOAc (100 mL×3). The combined organic layers were washed with brine (100 mL). The filtrate was evaporated to give 3-fluoro-5-nitro-pyridin-4-amine (1.8 g, 43%) as a yellow solid.

MS ES⁺: 158.0

¹H NMR (400 MHz, DMSO-d₆) 8.86 (s, 1H), 8.34 (d, J=3.1 Hz, 1H), 8.06-7.92 (m, 2H).

Step 2: A solution of 3-fluoro-5-nitro-pyridin-4-amine (1 g, 6.37 mmol) and 1,1,2-trichloroethane (849 mg, 6.37 mmol) in EtOH (10 mL) was treated with palladium on activated charcoal (1 g, 10% purity) under N₂. The suspension was degassed and purged with H2 three times, stirred under H2 (15 psi) at RT for 1 hour, filtered and concentrated under reduced pressure to give 5-fluoropyridine-3,4-diamine hydrochloride (1.0 g, 96%) as a brown solid.

MS ES⁺: 128.3

Step 3: A mixture of 4-amino-1,2,5-oxadiazole-3-carboxylic acid (395 mg, 3.06 mmol) and 5-fluoropyridine-3,4-diamine hydrochloride (500 mg, 3.06 mmol) in CH₂Cl₂ (1 mL) was treated with DIPEA (790 mg, 6.11 mmol) and HATU (1.74 g, 4.58 mmol) in one portion at RT, stirred for 1 hour, concentrated and extracted with EtOAc (30 mL×3). The combined organic layers were washed with brine (30 mL×2), dried over Na₂SO₄ and concentrated to afford a residue, which was purified by prep. HPLC (column: Xtimate C₁₈ 150*40 mm*10 μm, Mobile Phase A: water (0.05% NH₃·H₂O+10 mM NH₄HCO₃), Mobile Phase B: CH₃CN, 0% B to 20%). The pure fractions were collected and the volatiles evaporated. The residue was partitioned between CH₃CN (20 mL) and H₂O (100 mL) and lyophilized to give 4-amino-N-(4-amino-5-fluoropyridin-3-yl)-1,2,5-oxadiazole-3-carboxamide (350 mg, 21%) as a white solid.

MS ES⁺: 239.1

¹H NMR (400 MHz, DMSO-d₆) 10.43 (s, 1H), 8.10 (d, J=2.6 Hz, 1H), 7.94 (s, 1H), 6.57-6.18 (m, 4H).

Step 4: A mixture of 4-amino-N-(4-amino-5-fluoropyridin-3-yl)-1,2,5-oxadiazole-3-carboxamide (350 mg, 1.47 mmol) in AcOH (5 mL) was stirred at 110° C. for 1 hour, concentrated and extracted with EtOAc (30 mL×3). The combined organic layers were washed with brine (30 mL×2), dried over Na₂SO₄ and concentrated to give a residue, which was purified by prep. HPLC (column: Xtimate C₁₈ 150*40 mm*10 μm, Mobile Phase A: 0.225% aq. FA, Mobile Phase B: CH₃CN, 0% B to 30%). The pure fractions were collected and the volatiles evaporated. The residue was partitioned between CH₃CN (20 mL) and H₂O (100 mL) and lyophilized to give 4-(7-fluoro-1H-imidazo[4,5-c]pyridin-2-yl)-1,2,5-oxadiazol-3-amine (87 mg, 26%) as a white solid.

MS ES⁺: 221.0

¹H NMR (400 MHz, DMSO-d₆) 8.93 (s, 1H), 8.41 (d, J=2.5 Hz, 1H), 6.82 (s, 2H).

Step 5: A mixture of pyridazin-3-ylmethanol (25 mg, 0.227 mmol), 4-(7-fluoro-1H-imidazo[4,5-c]pyridin-2-yl)-1,2,5-oxadiazol-3-amine (50 mg, 0.227 mmol) and 2-(tributylphosphoranylidene)acetonitrile (110 mg, 0.454 mmol) in THF (1 mL) was heated at 110° C. for 3 hours under microwave irradiation, concentrated and extracted with EtOAc (10 mL×3). The combined organic layers were washed with brine (10 mL×2), dried over Na₂SO₄ and concentrated to give a residue, which was purified by prep. HPLC (column: Xtimate C₁₈ 100*30 mm*10 μm, Mobile Phase A: 0.225% aq. FA, Mobile Phase B: CH₃CN, 10% B to 40%) to give 4-(7-fluoro-1-(pyridazin-3-ylmethyl)-1H-imidazo[4,5-c]pyridin-2-yl)-1,2,5-oxadiazol-3-amine (peak 1, 4 mg, 5%) and 4-(7-fluoro-3-(pyridazin-3-ylmethyl)-3H-imidazo[4,5-c]pyridin-2-yl)-1,2,5-oxadiazol-3-amine (peak 2, 5 mg, 6%) as off-white solids.

### Example 187 (Peak 1): 4-(7-fluoro-1-(pyridazin-3-ylmethyl)-1H-imidazo[4,5-c]pyridin-2-yl)-1,2,5-oxadiazol-3-amine

MS ES⁺: 312.9

¹H NMR (400 MHz, DMSO-d₆) 9.22-9.11 (m, 1H), 9.08 (d, J=2.0 Hz, 1H), 8.51-8.43 (m, 1H), 7.90-7.79 (m, 1H), 7.74 (dd, J=4.8, 8.4 Hz, 1H), 6.97 (s, 2H), 6.30 (s, 2H).

### Example 188 (Peak 2): 4-(7-fluoro-3-(pyridazin-3-ylmethyl)-3H-imidazo[4,5-c]pyridin-2-yl)-1,2,5-oxadiazol-3-amine

MS ES⁺: 312.9

¹H NMR (400 MHz, DMSO-d₆) 9.16-9.12 (m, 1H), 9.09 (d, J=1.6 Hz, 1H), 8.51 (d, J=2.0 Hz, 1H), 7.82 (dd, J=1.6, 8.8 Hz, 1H), 7.73 (dd, J=4.8, 8.4 Hz, 1H), 6.93 (s, 2H), 6.46-6.27 (m, 2H).

### 2. Synthetic Intermediates

### Intermediate 1: 3-(7-fluoro-benzimidazol-2-yl)-4-methyl-1,2,5-oxadiazole

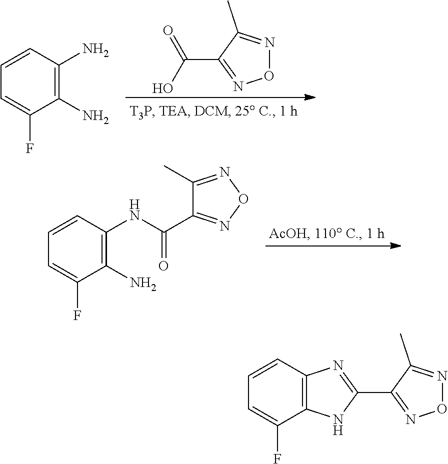

Step 1: T₃P (30.27 g, 47.57 mmol, 28.29 mL, 50% purity in ethyl acetate) was added dropwise to a solution of 3-fluorobenzene-1,2-diamine (4 g, 31.71 mmol), 4-methyl-1,2,5-oxadiazole-3-carboxylic acid (Intermediate 2) (4.06 g, 31.71 mmol) and TEA (9.63 g, 95.14 mmol) in DCM (100 mL) at 0° C. Then the mixture was stirred at 25° C. for 1 hour. The mixture was extracted with DCM (200 mL×3). The combined organic layers were dried over Na₂SO₄ and filtered. The filtrate was evaporated to dryness to give N-(2-amino-3-fluoro-phenyl)-4-methyl-1,2,5-oxadiazole-3-carboxamide (7.1 g, 30.06 mmol, 94.8% yield) as a black solid which was used for the next step directly.

MS ES⁺: 237.1

Step 2: A solution of N-(2-amino-3-fluoro-phenyl)-4-methyl-1,2,5-oxadiazole-3-carboxamide (7.1 g, 30.06 mmol) in AcOH (50 mL) was stirred at 110° C. for 1 hour. Then the mixture was evaporated to dryness. The residue was dissolved in ethyl acetate (200 mL) and the pH adjusted to 8-9 by sat. NaHCO₃ (aq.). The mixture was extracted with ethyl acetate (200 mL×3). The combined organic layers were washed with brine (200 mL), dried over Na₂SO₄ and filtered. The filtrate was evaporated to dryness. The residue was purified by silica gel chromatography (column height: 250 mm, diameter: 100 mm, 100-200 mesh silica gel, petroleum ether: ethyl acetate=5:1) to afford 3-(7-fluoro-benzimidazol-2-yl)-4-methyl-1,2,5-oxadiazole (1.2 g, 5.22 mmol, 17.4% yield, 95% purity) as a light yellow solid.

¹H NMR (400 MHz, DMSO-d₆) 14.25-13.77 (m, 1H), 7.51-7.40 (m, 1H), 7.39-7.24 (m, 1H), 7.24-7.03 (m, 1H), 2.80-2.77 (m, 3H).

### Intermediate 2: 4-methyl-1,2,5-oxadiazole-3-carboxylic acid

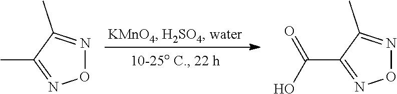

Finely ground KMnO₄ (40.27 g, 254.83 mmol) was added in small portions to a solution of 3,4-dimethyl-1,2,5-oxadiazole (5 g, 50.97 mmol) in a solution of H₂SO₄ (125 mL) and H₂O (125 mL) at 10-15° C. The mixture was maintained at 10° C. for 2 hours, then warmed to 25° C. for 20 hours. The mixture was filtered, and the filtrate extracted with ethyl acetate (300 mL×3). The combined organic layers were washed with sat. NaHSO₃ (aq.) (300 mL×3). The separated organic layer was dried with Na₂SO₄ and filtered. The filtrate was concentrated to afford 4-methyl-1,2,5-oxadiazole-3-carboxylic acid (3.9 g, 30.45 mmol, 59.7% yield) as a yellow solid which was used for the next step directly.

### Intermediate 3: 5-methyl-1,2,3-thiadiazole-4-carboxylic acid

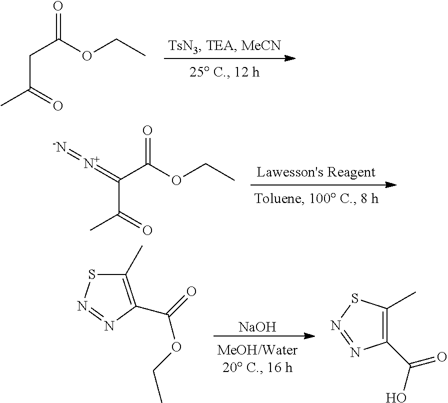

Step 1: To a solution of ethyl 3-oxobutanoate (50 g, 384.20 mmol) in MeCN (600 mL) was added TEA (77.75 g, 768.39 mmol) at 0° C. Then p-toluenesulfonyl azide (90.92 g, 461.04 mmol) was added slowly at ° C. The reaction was allowed to stir at 25° C. for 12 hours. The mixture was poured into water (1.5 L) and extracted with ethyl acetate (1 L×2). The combined organic layers were dried over Na₂SO₄, filtered and concentrated to give the crude product which was purified by column chromatography on silica gel (petroleum ether: ethyl acetate=100:0 to 90:10) to give ethyl 2-diazo-3-oxo-butanoate (41 g, 249.46 mmol, 64.9% yield, 95% purity) as a yellow oil.

Step 2: Lawesson's reagent (127.45 g, 315.10 mmol) was added to a solution of ethyl 2-diazo-3-oxo-butanoate (41 g, 262.59 mmol) in toluene (600 mL). Then the mixture was stirred at 100° C. under N₂ for 8 hours. The mixture was poured into water (1 L) and extracted with ethyl acetate (2 L×2). The combined organic layers were washed with water (1 L), dried over Na₂SO₄ and filtered. The filtrate was concentrated to give the crude product which was purified by column chromatography on silica gel (petroleum ether: ethyl acetate=100:0 to 80:20) to give ethyl 5-methylthiadiazole-4-carboxylate (40 g, 220.7 mmol, 84.0% yield, 95% purity) as a yellow solid.

¹H NMR (400 MHz, DMSO-d₆) 4.55-8.48 (m, 2H), 2.92-2.89 (m, 3H), 1.50-1.45 (m, 3H).

Step 3: A solution of NaOH (69.68 g, 1.74 mol) in water (60 mL) was added to a solution of ethyl 5-methylthiadiazole-4-carboxylate (30 g, 174.21 mmol) in MeOH (100 mL). Then the mixture was stirred at 20° C. for 16 hours. The mixture was concentrated in vacuum to remove the MeOH and the residue was adjusted to pH=4 with 1M HCl (aq.). Then the mixture was extracted with ethyl acetate (800 mL×3). The combined organic layers were dried over Na₂SO₄ and filtered. The filtrate was concentrated to give 5-methyl-1,2,3-thiadiazole-4-carboxylic acid (17.4 g, 114.67 mmol, 65.8% yield, 95% purity) as a white solid.

¹H NMR (400 MHz, DMSO-d₆) 13.74 (br s, 1H), 2.84 (s, 3H).

### Intermediate 4: 4-(4-fluoro-benzimidazol-2-yl)-1,2,5-oxadiazol-3-amine

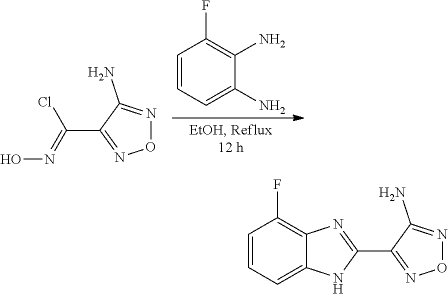

To a stirred solution of 3-fluorobenzene-1,2-diamine (0.4 g, 3.2 mmol) in ethanol (10 mL) was added (Z)-4-amino-N-hydroxy-1,2,5-oxadiazole-3-carbimidoyl chloride (0.4 g, 3.2 mmol). The reaction mixture was stirred for 12 hours at 80° C. After completion, the reaction mixture was concentrated under reduced pressure, diluted with water and extracted with ethyl acetate. The organic layer was dried over Na₂SO₄ and concentrated under reduced pressure to obtain the crude product which was purified by column chromatography on silica gel (230-400 mesh) to afford 4-(4-fluoro-benzimidazol-2-yl)-1,2,5-oxadiazol-3-amine (0.4 g, 58% yield).

MS ES⁺: 220.06

### Intermediate 5: 4-(benzimidazol-2-yl)-1,2,5-oxadiazol-3-amine

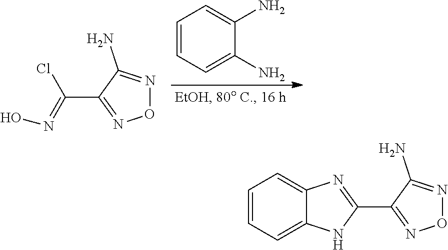

To a stirred solution of (Z)-4-amino-N-hydroxy-1,2,5-oxadiazole-3-carbimidoyl chloride (0.2 g, 1.2 mmol) in ethanol (6 mL) was added benzene-1,2-diamine (0.2 g, 1.6 mmol) and the reaction mixture was stirred for 16 hours at 80° C. Upon completion, the reaction mixture was concentrated. The residue was diluted with water and extracted with ethyl acetate. The organic layer was dried over Na₂SO₄ and concentrated under reduced pressure to obtain the crude product which was purified by column chromatography on silica gel (100-200 mesh) to afford 4-(benzimidazol-2-yl)-1,2,5-oxadiazol-3-amine (0.230 g, 95% yield) as a pale brown solid.

MS ES⁺: 202.00

### Intermediate 6: (6-bromopyridin-3-yl)methyl methanesulfonate

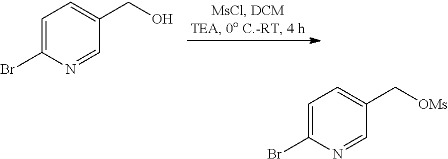 [SMILES: OCc1ccc(Br)nc1 ; CS(=O)(=O)OCc1ccc(Br)nc1]

To a stirred solution of (6-bromopyridin-3-yl)methanol (0.3 g, 1.59 mmol) in DCM at ° C. (10 mL) was added TEA (0.5 mL, 3.9 mmol) followed by methanesulfonyl chloride (0.18 g, 2.4 mmol). The resulting reaction mixture was then stirred at RT for 4 hours. After completion of the reaction, the reaction mixture was diluted with water and extracted with ethyl acetate. The organic layer was dried over Na₂SO₄ and concentrated under reduced pressure to afford (6-bromopyridin-3-yl)methyl methanesulfonate (0.3 g, 70.9% yield).

### Intermediate 7: 4-bromo-1,2,5-thiadiazole-3-carboxylic acid

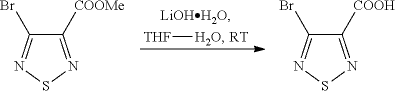

Methyl 4-bromo-1,2,5-thiadiazole-3-carboxylate (1.0 g, 4.5 mmol) was dissolved in THF (20 mL). Water (5 mL) was added, followed by the addition of LiOH·H₂O (0.3 g, 6.5 mmol). The reaction mixture was then stirred for 1 hour at RT. The reaction mixture was neutralised by adding conc. HCl and extracted with a solution of 10% MeOH in DCM, dried over Na₂SO₄ and concentrated under reduced pressure to obtain 4-bromo-1,2,5-thiadiazole-3-carboxylic acid (0.9 g, 96% yield) as off-white solid.

MS ES-: 206.95

### Intermediate 8: 4-(5-fluoro-benzimidazol-2-yl)-1,2,5-oxadiazol-3-amine

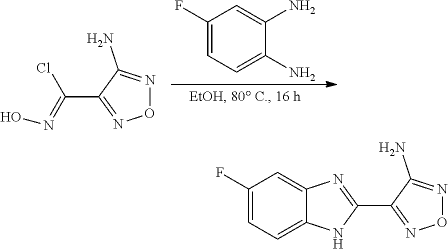 [SMILES: Nc1nonc1/C(Cl)=N/O ; Nc1ccc(F)cc1N ; Nc1nonc1-c1nc2cc(F)ccc2[nH]1]

To a stirred solution of (Z)-4-amino-N-hydroxy-1,2,5-oxadiazole-3-carbimidoyl chloride (0.2 g, 1.2 mmol) in ethanol (6 mL) was added 4-fluorobenzene-1,2-diamine (0.2 g, 1.6 mmol) and the reaction mixture was stirred for 16 hours at 80° C. After completion of the reaction, the solvent was evaporated from the reaction mixture, and the reaction mixture diluted with water and extracted with ethyl acetate. The organic layer was dried over Na₂SO₄, and concentrated under reduced pressure to obtain crude product which was purified by column chromatography on silica gel (100-200 mesh) to afford 4-(5-fluoro-benzimidazol-2-yl)-1,2,5-oxadiazol-3-amine (0.23 g, 82% yield) as an off-white solid.

MS ES⁺: 220.11

¹H NMR (400 MHz, DMSO-d₆) 13.8 (br s, 1H), 7.85-7.40 (m, 2H), 7.20 (s, 1H), 6.80 (s, 2H).

### Intermediate 9: 3-(4-fluoro-benzimidazol-2-yl)-4-methyl-1,2,5-oxadiazole

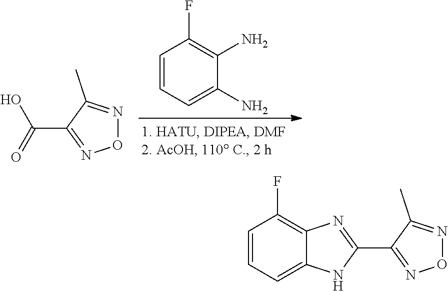

Following the procedure employed for Example 126 using 4-methyl-1,2,5-oxadiazole-3-carboxylic acid (Intermediate 2) (300 mg, 2.34 mmol) and 3-fluorobenzene-1,2-diamine (0.36 g, 2.81 mmol), gave 3-(4-fluoro-benzimidazol-2-yl)-4-methyl-1,2,5-oxadiazole (0.15 g, 30% yield) as a pale brown solid.

MS ES⁺: 219.15

### Intermediate 10: 3-(7-fluoro-benzimidazol-2-yl)-4-methyl-1,2,5-thiadiazole

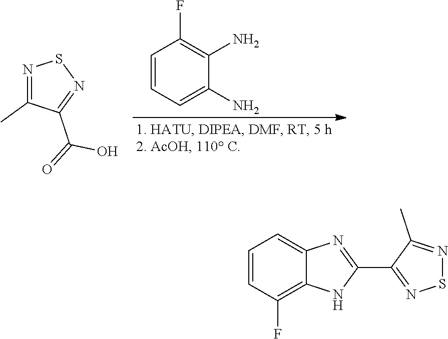

Following the procedure employed for Example 126 using 4-methyl-1,2,5-thiadiazole-3-carboxylic acid (200 mg, 1.38 mmol) and 3-fluorobenzene-1,2-diamine (190 mg, 1.53 mmol), gave 3-(7-fluoro-benzimidazol-2-yl)-4-methyl-1,2,5-thiadiazole (300 mg, 92% yield) as a pale yellow solid.

MS ES⁺: 235.16

### Intermediate 11: 4-(4-fluoro-benzimidazol-2-yl)-5-methyl-1,2,3-thiadiazole

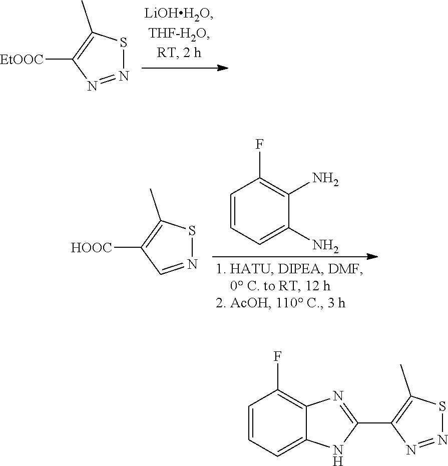

Following the procedures employed for Intermediate 7 (step 1) and Example 126 (step 2), using ethyl 5-methyl-1,2,3-thiadiazole-4-carboxylate (1 g, 5.8 mmol) and 3-fluorobenzene-1,2-diamine (485 mg, 3.85 mmol), gave 4-(4-fluoro-benzimidazol-2-yl)-5-methyl-1,2,3-thiadiazole (400 mg, 85% yield) as a pale yellow solid.

MS ES⁺: 235.16

¹H NMR (400 MHz, DMSO-d₆) 13.71 (s, 1H), 7.42 (s, 1H), 7.40-7.30 (m, 1H), 7.09-7.04 (m, 1H), 3.10 (s, 3H).

### Intermediate 12: 4-(5-methyl-benzimidazol-2-yl)-1,2,5-oxadiazol-3-amine

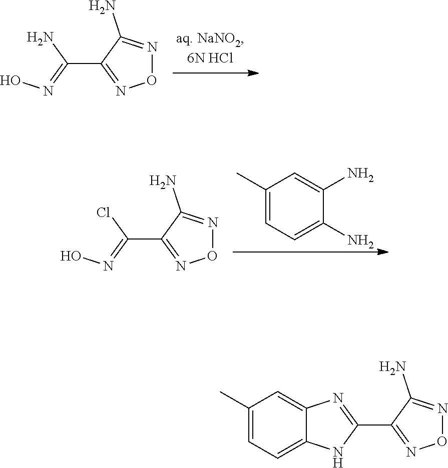

Step 1: 4-Amino-N′-hydroxy-1,2,5-oxadiazole-3-carboximidamide (10 g, 69.9 mmol) was added in portions to a mixture of water (50 ml) and 6N hydrochloric acid (35 mL) at 10° C. At this point, aqueous sodium nitrite (4.84 g, 70 mmol in 20 mL water) was added in portions while maintaining the temperature below 5° C. After complete addition, stirring was continued in the ice bath for 2 hours. Then the reaction mixture was allowed to warm to 15° C. The precipitate was collected by filtration, and washed well with water to obtain (Z)-4-amino-N-hydroxy-1,2,5-oxadiazole-3-carbimidoyl chloride (6.1 g, 54% yield) as an off-white solid.

¹H NMR (400 MHz, DMSO-d₆) 13.38 (s, 1H), 6.28 (br s, 2H).

Step 2: To stirred solution of 4-methylbenzene-1,2-diamine (3.66 g, 30 mmol) in EtOH (70 mL), (Z)-4-amino-N-hydroxy-1,2,5-oxadiazole-3-carbimidoyl chloride (5 g, 30.9 mmol) was added in portions and the resultant solution was refluxed at 80° C. for 30 min. Then the reaction mixture was allowed to come to ambient temperature. After 1 hour, the reaction mixture was diluted with water and neutralized with 0.1 M HCl and stirring was continued for an additional 1 hour. The obtained solid was filtered to obtain 4-(5-methyl-benzimidazol-2-yl)-1,2,5-oxadiazol-3-amine (3.87 g, 58% yield).

MS ES⁺: 216.23

### Intermediate 13: (2-methoxypyridin-4-yl)methyl methanesulfonate

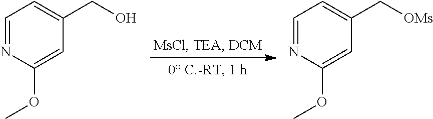

Triethylamine (181 mg, 1.8 mmol) was added to a stirred solution of (2-methoxypyridin-4-yl)methanol (100 mg, 0.72 mmol) in DCM (5 ml) at RT. The reaction mixture was cooled to 0° C. and methanesulfonyl chloride (99 mg, 0.86 mmol) was added dropwise under N₂ atmosphere. The reaction mixture was stirred at RT for 1 hour. After completion of the reaction, the mixture was diluted with DCM (30 mL) and washed with water (2×30 mL). The organic layer was dried over anhydrous Na₂SO₄, filtered and concentrated under reduced pressure to give (2-methoxypyridin-4-yl)methyl methanesulfonate (170 mg, quant.) as a pale yellow gummy material.

MS ES⁺: 218.20

¹H NMR (400 MHz, DMSO-d₆) 8.17 (d, J=5.2 Hz, 1H), 7.04-7.02 (m, 1H), 6.88 (s, 1H), 4.73 (s, 2H), 3.85 (s, 3H) (3H under solvent peak).

Intermediate 14: N¹-(pyridin-3-ylmethyl)benzene-1,2-diamine

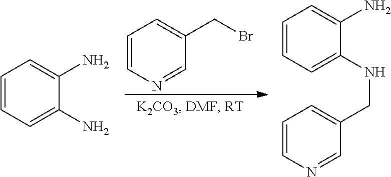

To a stirred solution of benzene-1,2-diamine (1.0 g, 9.2 mmol) and 3-(bromomethyl)pyridine (2.32 g, 9.2 mmol) in DMF (15 ml), was added K₂CO₃ (3.8 g, 27.6 mmol) and stirred for 2 hours at RT. After completion of the reaction, the mixture was diluted with water and extracted with ethyl acetate. The organic layer was dried over Na₂SO₄ and evaporated under reduced pressure to give crude product. Purification by column chromatography (gradient elution of 20-25% ethyl acetate in petroleum ether) afforded N¹-(pyridin-3-ylmethyl)benzene-1,2-diamine (0.50 g, 27% yield) as an off-white solid.

MS ES⁺: 200.37

¹H NMR (400 MHz, DMSO-d₆) 8.66 (s, 1H), 8.54-8.53 (m, 1H), 7.73-7.70 (m, 1H), 7.28 (s, 1H), 6.80-6.72 (m, 3H), 6.65-6.63 (m, 1H), 4.36-4.24 (m, 2H), 3.85 (br s, 1H), 3.36 (br s, 2H).

Intermediate 15: 4-formyl-3-methyl-1,2,5-oxadiazole 2-oxide

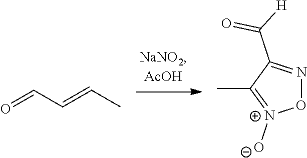

A stirred solution of (E)-but-2-enal (2 g, 0.028 mol) in acetic acid (10 mL) was cooled to 0° C. Then a saturated solution of sodium nitrite (4.9 g, in 6 ml water) was added to the reaction mass and stirred at RT for 2 hours. Then the reaction mixture was extracted with DCM (2×25 mL) and washed with water (30 mL). The organic layer was concentrated under vacuum and purified by chromatography using ethyl acetate: petroleum ether (30-50%) as an eluent to afford 4-formyl-3-methyl-1,2,5-oxadiazole 2-oxide (1.5 g, 41.1% yield) as a thick liquid.

¹H NMR (400 MHz, DMSO-d₆) 10.09 (s, 1H), 2.41 (s, 3H).

### Intermediate 16: N¹-(pyridin-4-ylmethyl)benzene-1,2-diamine

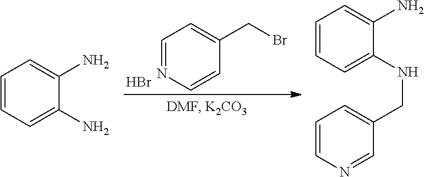

To a stirred solution of benzene-1,2-diamine (320 mg, 2.964 mmol) in DMF (8 mL), potassium carbonate (818 mg, 5.928 mmol) and 4-(bromomethyl)pyridine hydrobromide (500 mg, 1.976 mmol) were added and stirred at RT for 12 hours. Water was added to the reaction mixture and extracted with ethyl acetate (2×20 mL). The combined organic layer was concentrated under vacuum to afford N¹-(pyridin-4-ylmethyl)benzene-1,2-diamine (210 mg, 83.3% yield) as a pale yellow thick liquid.

¹H NMR (400 MHz, DMSO-d₆) 8.56 (d, J=5.6 Hz, 2H), 7.31 (d, J=5.6 Hz, 2H), 6.78-6.73 (m, 3H), 6.52-6.50 (m, 1H), 4.38 (s, 2H), 3.90 (br s, 1H), 3.37 (br s, 2H).

### Intermediate 17:4-(4-methyl-benzimidazol-2-yl)-1,2,5-oxadiazol-3-amine

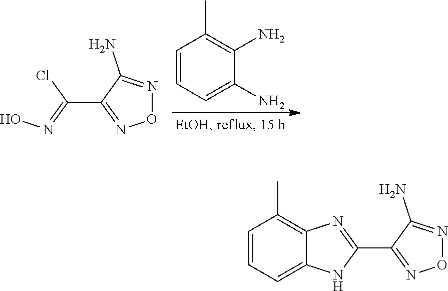

A stirred solution of (Z)-4-amino-N-hydroxy-1,2,5-oxadiazole-3-carbimidoyl chloride (0.35 g, 2.1084 mmol) and 3-methylbenzene-1,2-diamine (0.35 g, 3.1626 mmol) in EtOH (20 mL) was refluxed for 15 hours. The reaction mixture was then concentrated under reduced pressure, diluted with water (20 mL) and extracted with ethyl acetate (2×20 mL). The organic layer was dried and evaporated under reduced pressure to afford the crude product which was purified by flash column chromatography using Davisil as stationary phase and eluted with 25% EtOAc in petroleum ether to afford 4-(4-methyl-benzimidazol-2-yl)-1,2,5-oxadiazol-3-amine (0.3 g, 79% yield) as an off-white solid.

MS ES⁺: 216.07

¹H NMR (400 MHz, DMSO-d₆) 13.70-13.60 (m, 1H), 7.60-7.37 (m, 1H), 7.25-7.11 (m, 2H), 6.87 (s, 2H), 2.61 (s, 3H).

### Intermediate 18: pyrimidin-4-ylmethyl methanesulfonate

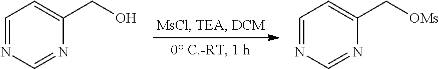 [SMILES: OCc1ccncn1 ; CS(=O)(=O)OCc1ccncn1]

Triethylamine (230 mg, 2.25 mmol) was added to a stirred solution of pyrimidin-4-ylmethanol (10 mg, 0.9 mmol) in DCM (5 ml) at RT. The reaction mixture was cooled to 0° C. and methanesulfonyl chloride (155 mg, 1.36 mmol) was added dropwise under N₂ atmosphere. The reaction mixture was stirred at RT for 1 hour. After complete consumption of starting material, the reaction mixture was diluted with DCM (30 mL) and washed with water (2×30 ml). The organic layer was dried over anhydrous Na₂SO₄, filtered and concentrated under reduced pressure to give pyrimidin-4-ylmethyl methanesulfonate (110 mg, 64% yield) as a pale yellow gummy material.

¹H NMR (400 MHz, DMSO-d₆) 9.33 (s, 1H), 8.91 (s, 1H), 7.69 (s, 1H), 4.80 (s, 2H), 3.32 (s, 3H).

Intermediate 19: pyridazin-4-ylmethyl 4-methylbenzenesulfonate

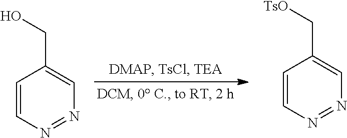 [SMILES: OCc1ccnnc1 ; Cc1ccc(S(=O)(=O)OCc2ccnnc2)cc1]

To a stirred solution of pyridazin-4-ylmethanol (300 mg, 2.7243 mmol) in DCM (5 mL) was added DMAP (33 mg, 0.2724 mmol) and triethylamine (0.8 mL, 5.4487 mmol) and the reaction was stirred for 15 min. At 0° C. 4-toluenesulfonyl chloride (623.2 mg, 3.2692 mmol) was added and the reaction mixture was stirred for 2 hours. Then the reaction mixture was evaporated under reduced pressure at low temperature to afford pyridazin-4-ylmethyl 4-methylbenzenesulfonate (400 mg, 80% yield) as a pale yellow thick liquid which was taken to the next step without further purification.

MS ES⁺: 265.09

### Intermediate 20: (2-(trifluoromethyl)pyridin-3-yl)methyl methanesulfonate

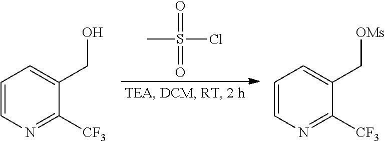

To a stirred solution of (2-(trifluoromethyl)pyridin-3-yl)methanol (0.2 g, 1.1 mmol) in DCM (5 mL) was added triethylamine (0.22 mL, 2.2 mmol) followed by methanesulfonyl chloride (0.18 mL, 1.65 mmol) dropwise. The resulting mixture was stirred at RT for 2 hours. Then the reaction mixture was evaporated under reduced pressure at 30° C. to afford (2-(trifluoromethyl)pyridin-3-yl)methyl methanesulfonate (0.21 g, 72% yield) as a pale yellow gum which was taken to the next step without further purification.

### Intermediate 21: N³-(pyridin-3-ylmethyl)pyridine-3,4-diamine

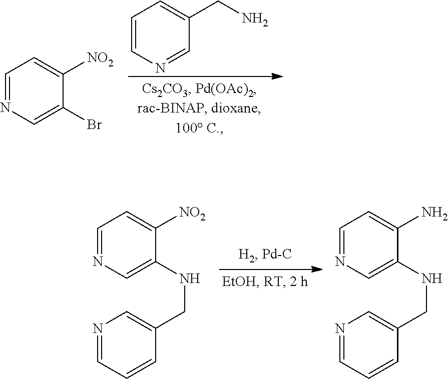

Step 1: A solution of 3-bromo-4-nitropyridine (200 mg, 0.98 mmol), pyridin-3-ylmethanamine (128 mg, 1.18 mmol) and Cs₂CO₃ (641 mg, 1.97 mmol) in dioxane (5 mL) in a sealed tube was purged with N₂ for 10 min. rac-BINAP (123 mg, 0.197 mmol) and Pd(OAc)₂ (22 mg. 0.098 mmol) were added under N₂ atmosphere and the reaction mixture was stirred at 100° C. for 16 hours. Then the reaction was diluted with EtOAc (100 mL) and filtered through a Celite® bed which was washed thoroughly with EtOAc (3×50 mL). The combined organics were washed with water (100 mL), dried over anhydrous Na₂SO₄, filtered and concentrated under reduced pressure to give crude product. Purification by flash chromatography to afforded 4-nitro-N-(pyridin-3-ylmethyl)pyridin-3-amine (100 mg, 44% yield) as a yellow solid.

MS ES⁺: 231.09

¹H NMR (400 MHz, DMSO-d₆) 8.65 (s, 1H), 8.56 (t, J=6.4 Hz, 1H), 8.47 (m, 2H), 7.90-7.80 (m, 2H), 7.80-7.75 (m, 1H), 7.38-7.35 (m, 1H), 4.78 (d, J=6.8 Hz, 2H) (1 NH not observed).

Step 2: To a stirred solution of 4-nitro-N-(pyridin-3-ylmethyl)pyridin-3-amine (100 mg, 1.55 mmol) in ethanol (6 mL) was added Pd/C (100 mg) and the reaction mixture was stirred under H₂ (balloon pressure) for 2 hours. Then the reaction mixture was diluted with methanol (50 mL) and filtered through a Celite® pad which was washed with methanol (2×50 mL). The filtrate was concentrated under reduced pressure to afford N³-(pyridin-3-ylmethyl)pyridine-3,4-diamine (100 mg, quant.) as a pale yellow gum.

MS ES⁺: 201.14

### Intermediate 22: 5-methyl-1,2,3-thiadiazole-4-carbaldehyde

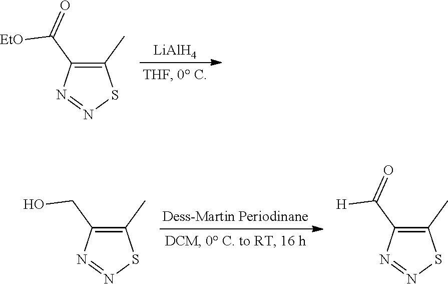

Step 1: Ethyl 5-methyl-1,2,3-thiadiazole-4-carboxylate (1.70 g, 9.87 mmol) was dissolved in dry THF, and LiAlH₄ (6.17 mL, 14.80 mmol, solution in THF) was added dropwise under N₂ atmosphere at 0° C. After 30 min, the reaction mixture was quenched with sodium sulfate solution, diluted with ethyl acetate and filtered through a Celite® pad which was washed with ethyl acetate (x2). The filtrate was dried over sodium sulfate and concentrated to afford (5-methyl-1,2,3-thiadiazol-4-yl)methanol (0.73 g, 57% yield) which was taken directly to the next step.

MS ES⁺: 131.03

Step 2: (5-Methyl-1,2,3-thiadiazol-4-yl)methanol (0.70 g, 5.60 mmol) was taken in dry DCM (10 mL), and Dess-Martin periodinane (2.61 g, 6.16 mmol) was added under N₂ atmosphere at ° C. The reaction mixture was stirred at RT for 16 hours. After completion of the reaction, the reaction mixture was filtered through a Celite® pad which was washed with ethyl acetate (x2). The filtrate was dried over sodium sulfate and concentrated in vacuum. The crude product was purified by normal phase column chromatography to afford 5-methyl-1,2,3-thiadiazole-4-carbaldehyde (0.2 g, 31% yield) as a pale brown thick gum.

MS ES⁺: 128.94

### Intermediate 23: 3-bromo-4-(7-fluoro-benzimidazol-2-yl)-1,2,5-thiadiazole

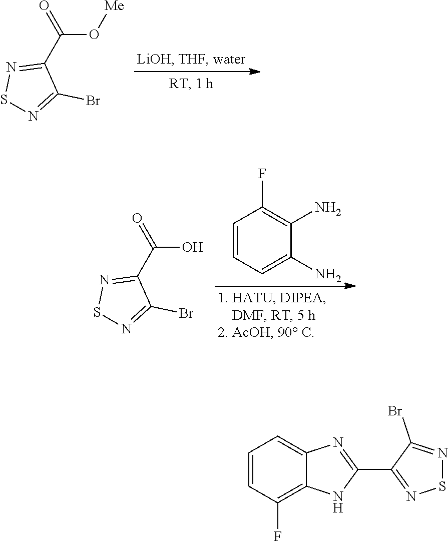 [SMILES: COC(=O)c1nsnc1Br ; O=C(O)c1nsnc1Br ; Nc1cccc(F)c1N ; Fc1cccc2nc(-c3nsnc3Br)[nH]c12]

Step 1: Methyl 4-bromo-1,2,5-thiadiazole-3-carboxylate (1.0 g, 4.3 mmol) was dissolved in THF (20 mL). Water (5 mL) was added, followed by the addition of LiOH·H₂O (0.3 g, 6.5 mmol). The reaction mixture was stirred for 1 hour at RT. The reaction was then neutralised by adding 1N HCl and extracted with 10% DCM in MeOH. The organic layer was dried over Na₂SO₄ and concentrated in vacuo to afford 4-bromo-1,2,5-thiadiazole-3-carboxylic acid (Intermediate 7) (0.9 g, 96% yield) as an off-white solid.

MS ES-: 206.95

Step 2: To a solution of 4-bromo-1,2,5-thiadiazole-3-carboxylic acid (Intermediate 7) (0.16 g, 0.8 mmol) in DMF (20.0 mL) at 0° C. was added HATU (0.46 g, 1.2 mmol) and DIPEA (0.28 mL, 1.6 mmol), followed by the addition of 3-fluorobenzene-1,2-diamine (0.1 g, 0.8 mmol). The resulting mixture was stirred at RT for 5 hours. The reaction mixture was then diluted with ice cooled water and extracted with EtOAc (20 mL). The organic layer was dried over Na₂SO₄ and concentrated in vacuo. The resulting brown gummy material was then dissolved in acetic acid (10 mL) and refluxed at 90° C. for 2 hours. The reaction mixture was diluted with water and extracted with EtOAc (15 mL). The organic layer was dried over Na₂SO₄ and concentrate in vacuo. The crude product was purified by reverse phase chromatography using 70% methanol in water to afford 3-bromo-4-(7-fluoro-benzimidazol-2-yl)-1,2,5-thiadiazole (016 g, 68% yield) as a pale yellow solid.

MS ES⁺: 298.96

### Intermediate 24: (2-(trifluoromethyl)pyridin-4-yl)methyl methanesulfonate

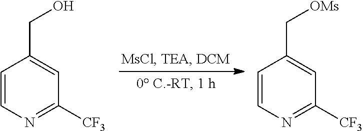 [SMILES: OCc1ccnc(C(F)(F)F)c1 ; CS(=O)(=O)OCc1ccnc(C(F)(F)F)c1]

To a stirred solution of (2-(trifluoromethyl)pyridin-4-yl)methanol (0.35 g, 2 mmol) in DCM (5 mL), triethylamine (0.42 g, 4 mmol) and methanesulfonyl chloride (0.35 g, 3 mmol) were added at ° C. Then the reaction mixture was diluted with water (10 mL) and extracted with DCM (20 mL). The organic layer was dried over Na₂SO₄ and concentrated under reduced pressure to afford (2-(trifluoromethyl)pyridin-4-yl)methyl methanesulfonate (0.45 g, 72% yield) as a pale brown residue.

MS ES⁺: 256.32

### 3. Biological Activity of Compounds of the Invention

KCNK13 antagonist activity was determined by measuring changes in intracellular Thallium (Tl⁺) concentrations using a Tl⁺ sensitive fluorescent dye. The changes in fluorescent signal were monitored by Fluorescent Imaging Plate Reader (FLIPR™) technology available from Molecular Devices, LLC, US. KCNK13 mediated increases in intracellular Tl+concentration were readily detected by addition of a thallium sulfate stimulus. 24 hours prior to the assay, human embryonic kidney 293 cells (HEK 293 cells) stably expressing human KCNK13 were seeded in cell culture medium in PDL coated black, clear-bottom 384-well plates (commercially available from Corning Inc., 356663) and grown overnight at 37° C., 5% CO₂. On the day of the assay, cell culture media was removed and cells were loaded with potassium dye (commercially sold by Molecular Devices, LLC, US, R8222) for 1 hour at room temperature in the dark. Test compounds (at 10 point half log concentration response curves from 10 PM) were added to cells for 15 minutes prior to the addition of thallium sulfate to all wells. The IC₅₀ values were determined from ten point concentration response curves. Curves were generated using the average of two wells for each data point. The results are summarised in table 2.

TABLE 2

| | hKCNK13 |
|---|---|
| Example | IC₅₀ (μM) |
| 1 | +++++ |
| 2 | ++++++ |
| 3 | ++++++ |
| 4 | ++++++ |
| 5 | +++++ |
| 6 | +++++ |
| 7 | +++++ |
| 8 | +++++ |
| 9 | +++++ |
| 10 | +++++ |
| 11 | ++++ |
| 12 | ++++ |
| 13 | ++++ |
| 14 | ++++ |
| 15 | ++++ |
| 16 | ++++ |
| 17 | +++ |
| 18 | +++ |
| 19 | +++ |
| 20 | ++ |
| 21 | ++++++ |
| 22 | ++++++ |
| 23 | ++++++ |
| 24 | +++++ |
| 25 | ++++++ |
| 26 | +++++ |
| 27 | ++++++ |
| 28 | ++++++ |
| 29 | ++++++ |
| 30 | ++++++ |
| 31 | ++++++ |
| 32 | ++++++ |
| 33 | ++++++ |
| 34 | ++ |
| 35 | ++++++ |
| 36 | +++++ |
| 37 | ++++++ |
| 38 | ++++++ |
| 39 | ++++ |
| 40 | +++++ |
| 41 | +++++ |
| 42 | ++ |
| 43 | ++ |
| 44 | ++++++ |
| 45 | +++++ |
| 46 | +++++ |
| 47 | ++++ |
| 48 | ++ |
| 49 | +++++ |
| 50 | +++++ |
| 51 | +++ |
| 52 | +++++ |
| 53 | +++++ |
| 54 | +++ |
| 55 | +++++ |
| 56 | +++++ |
| 57 | +++++ |
| 58 | +++++ |
| 59 | ++++ |
| 60 | +++++ |
| 61 | ++++ |
| 62 | ++++ |
| 63 | ++++ |
| 64 | ++++ |
| 65 | ++++++ |
| 66 | ++++ |
| 67 | ++++ |
| 68 | ++++ |
| 69 | ++++ |
| 70 | +++ |
| 71 | +++++ |
| 72 | +++ |
| 73 | +++ |
| 74 | +++ |
| 75 | +++ |
| 76 | +++ |
| 77 | ++ |
| 78 | +++ |
| 79 | +++ |
| 80 | ++ |
| 81 | ++ |
| 82 | ++ |
| 83 | ++++ |
| 84 | +++++ |
| 85 | + |
| 86 | ++++++ |
| 87 | ++++++ |
| 88 | ++++++ |
| 89 | ++++++ |
| 90 | ++++++ |
| 91 | ++++++ |
| 92 | ++++++ |
| 93 | ++++++ |
| 94 | +++++ |
| 95 | +++++ |
| 96 | +++++ |
| 97 | +++++ |
| 98 | +++++ |
| 99 | +++++ |
| 100 | ++++ |
| 101 | +++++ |
| 102 | ++++ |
| 103 | ++++ |
| 104 | ++++ |
| 105 | ++++ |
| 106 | ++++ |
| 107 | ++++ |
| 108 | +++ |
| 109 | +++ |
| 110 | +++ |
| 111 | +++ |
| 112 | +++ |
| 113 | +++ |
| 114 | ++ |
| 115 | ++ |
| 116 | ++ |
| 117 | ++ |
| 118 | ++++++ |
| 119 | ++++++ |
| 120 | ++++++ |
| 121 | +++++ |
| 122 | ++++++ |
| 123 | ++++++ |
| 124 | ++++++ |
| 125 | ++++++ |
| 126 | +++++ |
| 127 | +++++ |
| 128 | +++++ |
| 129 | +++++ |
| 130 | +++++ |
| 131 | +++++ |
| 132 | ++++++ |
| 133 | +++++ |
| 134 | ++++++ |
| 135 | ++++ |
| 136 | +++++ |
| 137 | +++ |
| 138 | ++ |
| 139 | +++++ |
| 140 | +++ |
| 141 | +++++ |
| 142 | ++++ |
| 143 | ++++ |
| 144 | ++ |
| 145 | ++++++ |
| 146 | ++++++ |
| 147 | ++++ |
| 148 | ++++++ |
| 149 | +++++ |
| 150 | ++++ |
| 151 | n.m. |
| 152 | ++++++ |
| 153 | ++++ |
| 154 | ++++++ |
| 155 | ++++++ |
| 156 | ++++++ |
| 157 | ++++ |
| 158 | + |
| 159 | ++++++ |
| 160 | +++ |
| 161 | ++ |
| 162 | ++++++ |
| 163 | ++++ |
| 164 | ++ |
| 165 | +++++ |
| 166 | +++++ |
| 167 | ++ |
| 168 | ++++++ |
| 169 | ++++ |
| 170 | +++++ |
| 171 | +++ |
| 172 | ++++ |
| 173 | +++ |
| 174 | +++++ |
| 175 | ++++ |
| 176 | + |
| 177 | ++ |
| 178 | + |
| 179 | ++++ |
| 180 | +++ |
| 181 | +++ |
| 182 | ++++ |
| 183 | +++++ |
| 184 | +++++ |
| 185 | ++++++ |
| 186 | +++ |
| 187 | +++ |
| 188 | + |

IC50 (≤5.0 μM = ‘+’; ≤1.0 μM = ‘++’; ≤0.50 μM = ‘+++’; ≤0.20 μM = ‘++++’; ≤0.10 μM = ‘+++++’; ≤0.05 μM = ‘++++++’; n.m. = not measured)

### 4. Inflammasome Assay in Primary Mouse Postnatal Microglia Using LPS/0 mM Extracellular K⁺

Newly shaken mouse microglia cells were added to a 96-well plate and left to adhere overnight. After this time, 100 ng/mL LPS was added to each well and incubated at 37° C. for 3.5 hours, at which point compound addition was undertaken and plates incubated at 37° C. for an additional 30 minutes. After this time, the medium in each well was removed and replaced with K⁺ free buffer and the plates then incubated for an additional 2 hours at 37° C. Measurement of IL-1βlevels in the sample wells was undertaken using MesoScale Discovery™ MESO QuickPlex SQ 120 and IL-1R antibodies from mouse IL-1β DuoSet ELISA kit (R&D System, DY401). The results are summarised in table 3.

TABLE 3

| | Mouse primary microglia |
|---|---|
| Compound | IL-1β, IC₅₀ (μM) |
| Example 21 | ++ |
| Example 32 | + |
| Example 49 | +++ |
| Example 57 | + |
| Example 71 | + |
| Example 86 | +++ |
| Example 120 | +++ |

IC50 (≤0.20 μM = ‘+’; ≤0.10 μM = ‘++’; ≤0.05 μM = ‘+++’)

It will be understood that the present invention has been described above by way of example only. The examples are not intended to limit the scope of the invention. Various modifications and embodiments can be made without departing from the scope and spirit of the invention, which is defined by the following claims only.

1-16. (canceled)

17. A compound of formula (I) 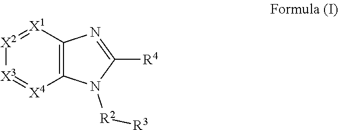
or a pharmaceutically acceptable salt, N-oxide, solvate or prodrug thereof, wherein:
each X¹, X², X³ and X⁴ is independently CH, CR¹ or N;
each —R¹ is independently halo, —CN, —R^{α}, —OH, —OR^{α}, —NH₂, —NHRα, —N(R^{α})₂, —SR^{α}, —SOR^{α}, —SO₂R^{α}, —SO(NH)R^{α}, —SO₂NHR^{α}, —SO₂N(R^{α})₂, —NH—SOR^{α}, —NH—SO₂R^{α}, —NH—SO₂NHR^{α}, —NH—SO₂N(R′)₂, —NR^{α}—SOR^{α}, —NR^{α}—SO₂R^{α}, —NR^{α}—SO₂NH₂, —NR^{α}—SO₂NHR^{α}, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²—is —C(R^{θ})₂-, —C(R^{θ})₂-C(R^{θ})₂-, —C(R^{θ})₂-O—, —C(R^{θ})₂-NR-, —C(R^{θ})₂-CO—, or —C(R^{θ})₂-CONR⁹—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{α}, —NH₂, —NHR^{α}, —N(R′)₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR⁶⁷—SOR^{δ}, —NR⁶⁷—SO₂R^{δ}, —NR⁶⁷—SO₂NH₂, —NR⁶⁷—SO₂NHR^{δ}, —NR⁶⁷—SO₂N(R^{δ})₂, —COR⁶⁷, —COOR⁶⁷, —OCOR⁶⁷, —NH—CHO, —NR^{δ}—CHO, —NH—COR^{δ}, —NR^{δ}—COR^{δ}, —NH—COOR^{δ}, —NR^{δ}—COOR^{δ}, —CONH₂, —CONHR^{α}, —CON(R^{δ})₂, —NH—CONHR^{δ}, —NR^{δ}—CONHR^{δ}, —NH—CON(R^{δ})₂, or —NR^{δ}—CON(R^{δ})₂;
R⁴ is a 5-membered heteroaryl group with one or more heteroatoms N, O or S in the ring structure, wherein the heteroaryl group is optionally substituted with one, two, three or four substituents independently selected from halo, —CN, —R^{ε}, —OH, —OR^{ε}, —NH₂, —NHR^{ε},-N(RE)₂, —SH, —SR^{ε}, —SOR, —SO₂R, —SO₂NH₂, —SO₂NHR^{ε}, —SO₂N(R^{ε})₂, —NH—SO₂R^{ε}, —NH—SO₂NHR^{ε}, —NH—SO₂N(R)₂, or —NR^{ε}—SO₂R^{ε};
each —R^{α} is independently C₁-C₃ alkyl, C₂-C₃ alkenyl, C₂-C₃ alkynyl or C₃-C₆ cycloalkyl, all optionally substituted with one or more halo, —OH,-NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more halo, —OH,-NH₂ or —SO₂CH₃;
each —R^{E} is independently C₁-C₃ alkyl, C₂-C₃ alkenyl, C₂-C₃ alkynyl or C₃-C₆ cycloalkyl, all optionally substituted with one or more halo, —OH,-NH₂ or —SO₂CH₃; and
each —R^{a} is independently hydrogen or methyl;
provided that the compound is not:
(i) 6-[[2-(4-amino-1,2,5-oxadiazol-3-yl)benzimidazol-1-yl]methyl]-1,3,5-triazine-2,4-diamine;
(ii) 6-[[2-(4-amino-1,2,5-oxadiazol-3-yl)benzimidazol-1-yl]methyl]-N²,N²-dimethyl-1,3,5-triazine-2,4-diamine;
(iii) 4-[1-(pyridin-2-ylmethyl)benzimidazol-2-yl]-1,2,5-oxadiazol-3-amine;
(iv) 4-[1-(pyridin-4-ylmethyl)benzimidazol-2-yl]-1,2,5-oxadiazol-3-amine;
(v) 2-(5-methylthiophen-2-yl)-1-(pyridin-2-ylmethyl)benzimidazole;
(vi) 2-(5-methylfuran-2-yl)-1-(pyridin-2-ylmethyl)benzimidazole;
(vii) 2-(5-methylfuran-2-yl)-1-(pyridin-3-ylmethyl)benzimidazole;
(viii) 2-(5-methylfuran-2-yl)-1-(pyridin-4-ylmethyl)benzimidazole;
(ix) 1-(pyridin-3-ylmethyl)-2-(pyrrol-2-yl)benzimidazole; or
(x) 1-(pyridin-3-ylmethyl)-2-(pyrrol-2-yl)-5-(trifluoromethyl)benzimidazole.

18. The compound, salt, N-oxide, solvate or prodrug as claimed in claim 17, wherein each X¹, X², X³ and X⁴ is independently CH or CR¹.

19. The compound, salt, N-oxide, solvate or prodrug as claimed in claim 17, wherein one of X¹, X², X³ and X⁴ is N, and the remaining of X¹, X², X³ and X⁴ are independently CH or CR¹.

20. The compound, salt, N-oxide, solvate or prodrug as claimed in claim 17, wherein each —R¹ is independently halo, —CN, —R^{α}, —OH, —OR^{α}, —NH₂, —NHR^{α}, —N(R^{α})₂, —SR^{α}, —SOR^{δ}, —SO₂R^{α}, —SO(NH)R^{δ}, —SO₂NHR^{α}, —SO₂N(R^{α})₂, —NH—SOR^{δ}, —NH—SO₂R^{α}, —NR^{α}—SOR^{δ}, —NR^{α}—SO₂R^{δ}, —COR^{α}, —COOR^{α}, —OCOR^{α}, —CONH₂, —CONHR^{α}, —CON(R^{α})₂, C₃-C₆ cycloalkyl, phenyl, a 3- to 6-membered heterocyclic group with one, two, three or four heteroatoms N, O or S in the ring structure, or a 5- or 6-membered heteroaryl group with one, two, three or four heteroatoms N, O or S in the ring structure, wherein the cycloalkyl, phenyl, heterocyclic or heteroaryl group is optionally substituted with one or two substituents independently selected from C₁-C₃ alkyl or —CO(C₁-C₃ alkyl); wherein each —R^{α} is independently C₁-C₃ alkyl, C₂-C₃ alkenyl, C₂-C₃ alkynyl or C₃-C₆ cycloalkyl, all optionally substituted with one, two, three, four or five substituents independently selected from halo, —OH, —NH₂ or —SO₂CH₃.

21. The compound, salt, N-oxide, solvate or prodrug as claimed in claim 17, wherein —R²— is —CH₂—, —CH(CH₃)—, —C(CH₃)₂—, —CH₂—CH₂—, —CH(CH₃)—CH₂—, —C(CH₃)₂—CH₂—, —CH(CH₃)—CH(CH₃)—, —CH₂—O—, —CH(CH₃)—O—, —C(CH₃)₂—O—, —CH₂—NH—, —CH(CH₃)—NH—, —C(CH₃)₂—NH—, CH₂—N(CH₃)—, —CH(CH₃)—N(CH₃)—, —CH₂—CO—, —CH(CH₃)—CO—, —C(CH₃)₂—CO—, —CH₂—CO—NH—, —CH(CH₃)—CO—NH—, —C(CH₃)₂—CO—NH—, —CH₂—CO—N(CH₃)—, or —CH(CH₃)—CO—N(CH₃)—.

22. The compound, salt, N-oxide, solvate or prodrug as claimed in claim 17, wherein —R³ is a 6-membered heteroaryl group with one, two, three or four nitrogen atoms in the ring structure (such as pyridinyl, pyridazinyl, pyrimidinyl, pyrazinyl, triazinyl or tetrazinyl), wherein the heteroaryl group is optionally substituted with one, two, three or four substituents independently selected from halo, —CN, —R^{δ}, —OH, —OR^{δ}, —NH₂, —NHR^{δ}, —N(R^{δ})₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NR⁶⁷—SOR^{δ}, —NR⁶⁷—SO₂R^{δ}, —COR⁶⁷, —COOR⁶⁷, —OCOR⁶⁷, —CONH₂, —CONHR⁶⁷, or —CON(R^{δ})₂; wherein each —R^{δ} is independently C₁-C₃ alkyl, C₂-C₃ alkenyl, C₂-C₃ alkynyl or C₃-C₆ cycloalkyl, all optionally substituted with one, two, three, four or five substituents independently selected from halo, —OH, —NH₂ or —SO₂CH₃.

23. The compound, salt, N-oxide, solvate or prodrug as claimed in claim 17, wherein —R⁴ is a 5-membered heteroaryl group with one, two, three or four heteroatoms N, O or S in the ring structure (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl or thiatriazolyl), wherein the heteroaryl group is optionally substituted with one, two, three or four substituents independently selected from halo, —CN, —R^{ε}, —OH, —OR^{ε}, —NH₂, —NHR^{ε}, —N(R^{ε})₂, —SH, —SR^{ε}, —SOR^{ε}, —SO₂R^{ε}, —SO₂NH₂, —SO₂NHR^{ε}, —SO₂N(RE)₂, —NH—SO₂R^{ε}, or —NR^{ε}—SO₂R^{ε}; wherein each —R^{ε} is independently C₁-C₃ alkyl, C₂-C₃ alkenyl, C₂-C₃ alkynyl or C₃-C₆ cycloalkyl, all optionally substituted with one, two, three, four or five substituents independently selected from halo, —OH, —NH₂ or —SO₂CH₃.

24. The compound, salt, N-oxide, solvate or prodrug as claimed in claim 17, wherein the compound is selected from:
5-[[6,7-difluoro-2-(4-methyl-1,2,5-oxadiazol-3-yl)benzimidazol-1-yl]methyl]pyrimidine-2-carbonitrile;
5-[[2-(4-amino-1,2,5-oxadiazol-3-yl)-6,7-difluoro-benzimidazol-1-yl]methyl]pyrimidine-2-carbonitrile;
4-[7-fluoro-1-(pyrimidin-5-ylmethyl)benzimidazol-2-yl]-1,2,5-oxadiazol-3-amine;
4-[6-fluoro-1-(pyrimidin-5-ylmethyl)benzimidazol-2-yl]-1,2,5-oxadiazol-3-amine;
4-[5-fluoro-1-(pyrimidin-5-ylmethyl)benzimidazol-2-yl]-1,2,5-oxadiazol-3-amine;
4-[7-fluoro-1-(pyridazin-3-ylmethyl)benzimidazol-2-yl]-1,2,5-oxadiazol-3-amine;
3-[6,7-difluoro-1-(pyrimidin-5-ylmethyl)benzimidazol-2-yl]-4-methyl-1,2,5-oxadiazole;
5-[[2-(4-amino-1,2,5-oxadiazol-3-yl)-4-fluoro-benzimidazol-1-yl]methyl]pyridine-2-carbonitrile;
3-[4-fluoro-1-(pyrimidin-5-ylmethyl)benzimidazol-2-yl]-4-methyl-1,2,5-oxadiazole;
4-[7-fluoro-1-[(6-methoxypyridin-3-yl)methyl]benzimidazol-2-yl]-1,2,5-oxadiazol-3-amine;
5-[[2-(4-amino-1,2,5-oxadiazol-3-yl)imidazo[4,5-b]pyridin-3-yl]methyl]pyrimidine-2-carbonitrile;
4-[6-fluoro-3-(pyrimidin-5-ylmethyl)imidazo[4,5-b]pyridin-2-yl]-1,2,5-oxadiazol-3-amine;
3-methyl-4-[3-(pyrimidin-5-ylmethyl)imidazo[4,5-b]pyridin-2-yl]-1,2,5-oxadiazole;
4-[3-[(6-methoxypyridin-3-yl)methyl]imidazo[4,5-b]pyridin-2-yl]-1,2,5-oxadiazol-3-amine;
4-[3-(pyrimidin-5-ylmethyl)imidazo[4,5-b]pyridin-2-yl]-1,2,5-oxadiazol-3-amine;
3-[3-[(6-methoxypyridin-3-yl)methyl]imidazo[4,5-b]pyridin-2-yl]-4-methyl-1,2,5-oxadiazole;
3-methyl-4-[3-[[6-(trifluoromethyl)pyridin-3-yl]methyl]imidazo[4,5-b]pyridin-2-yl]-1,2,5-oxadiazole;
6-[[2-(4-methyl-1,2,5-oxadiazol-3-yl)imidazo[4,5-b]pyridin-3-yl]methyl]pyridazine-3-carbonitrile;
4-[3-[[6-(trifluoromethyl)pyridin-3-yl]methyl]imidazo[4,5-b]pyridin-2-yl]-1,2,5-oxadiazol-3-amine;
3-methyl-4-[3-(pyridazin-3-ylmethyl)imidazo[4,5-b]pyridin-2-yl]-1,2,5-oxadiazole;
4-[1-(pyrimidin-5-ylmethyl)benzimidazol-2-yl]-1,2,5-oxadiazol-3-amine;
4-[4,7-difluoro-1-(pyridin-3-ylmethyl)benzimidazol-2-yl]-1,2,5-oxadiazol-3-amine;
4-[3-[(6-chloropyridin-3-yl)methyl]imidazo[4,5-b]pyridin-2-yl]-1,2,5-oxadiazol-3-amine;
3-[3-[(6-chloropyridin-3-yl)methyl]imidazo[4,5-b]pyridin-2-yl]-4-methyl-1,2,5-oxadiazole;
5-[[2-(4-amino-1,2,5-oxadiazol-3-yl)-7-fluoro-benzimidazol-1-yl]methyl]pyrimidine-2-carbonitrile;
5-[[2-(4-amino-1,2,5-oxadiazol-3-yl)-4-fluoro-benzimidazol-1-yl]methyl]pyrimidine-2-carbonitrile;
3-[5,7-difluoro-1-(pyridin-3-ylmethyl)benzimidazol-2-yl]-4-methyl-1,2,5-oxadiazole;
5-[[2-(4-amino-1,2,5-oxadiazol-3-yl)-4,7-difluoro-benzimidazol-1-yl]methyl]pyrimidine-2-carbonitrile;
4-[3-[(6-chloropyridin-3-yl)methyl]-6-fluoro-imidazo[4,5-b]pyridin-2-yl]-1,2,5-oxadiazol-3-amine;
3-[1-[dideuterio(pyridin-3-yl)methyl]-4-fluoro-benzimidazol-2-yl]-4-methyl-1,2,5-oxadiazole;
3-[1-[dideuterio(pyridin-3-yl)methyl]-7-fluoro-benzimidazol-2-yl]-4-methyl-1,2,5-oxadiazole;
4-[7-fluoro-1-(pyrazin-2-ylmethyl)benzimidazol-2-yl]-1,2,5-oxadiazol-3-amine;
4-[5-bromo-1-(pyridin-3-ylmethyl)benzimidazol-2-yl]-1,2,5-oxadiazol-3-amine;
4-[5-(dimethylamino)-1-(pyridin-3-ylmethyl)benzimidazol-2-yl]-1,2,5-oxadiazol-3-amine;
5-[[2-(4-amino-1,2,5-oxadiazol-3-yl)-7-fluoro-benzimidazol-1-yl]methyl]pyrazine-2-carbonitrile;
5-[[2-(4-amino-1,2,5-oxadiazol-3-yl)-4-fluoro-benzimidazol-1-yl]methyl]pyrazine-2-carbonitrile;
5-[[2-(4-methyl-1,2,5-oxadiazol-3-yl)benzimidazol-1-yl]methyl]pyridine-2-carbonitrile;
3-[1-[(6-chloropyridin-3-yl)methyl]-7-fluoro-benzimidazol-2-yl]-4-methyl-1,2,5-oxadiazole;
3-[1-[(6-chloropyridin-3-yl)methyl]-4-fluoro-benzimidazol-2-yl]-4-methyl-1,2,5-oxadiazole;
5-[[4-fluoro-2-(4-methyl-1,2,5-oxadiazol-3-yl)benzimidazol-1-yl]methyl]pyridine-2-carbonitrile;
5-[[7-fluoro-2-(4-methyl-1,2,5-oxadiazol-3-yl)benzimidazol-1-yl]methyl]pyrazine-2-carbonitrile;
5-[[4-fluoro-2-(4-methyl-1,2,5-oxadiazol-3-yl)benzimidazol-1-yl]methyl]pyrazine-2-carbonitrile;
3-[7-fluoro-1-[(6-methoxypyridin-3-yl)methyl]benzimidazol-2-yl]-4-methyl-1,2,5-oxadiazole;
5-[[7-fluoro-2-(4-methyl-1,2,5-oxadiazol-3-yl)benzimidazol-1-yl]methyl]pyrimidine-2-carbonitrile;
5-[[4-fluoro-2-(4-methyl-1,2,5-oxadiazol-3-yl)benzimidazol-1-yl]methyl]pyrimidine-2-carbonitrile;
6-[[7-fluoro-2-(4-methyl-1,2,5-oxadiazol-3-yl)benzimidazol-1-yl]methyl]pyridazine-3-carbonitrile;
6-[[4-fluoro-2-(4-methyl-1,2,5-oxadiazol-3-yl)benzimidazol-1-yl]methyl]pyridazine-3-carbonitrile;
3-[1-[(6-ethoxypyridin-3-yl)methyl]-4-fluoro-benzimidazol-2-yl]-4-methyl-1,2,5-oxadiazole;
3-[7-fluoro-1-(pyrimidin-5-ylmethyl)benzimidazol-2-yl]-4-methyl-1,2,5-oxadiazole;
5-[[2-(4-methyl-1,2,5-oxadiazol-3-yl)imidazo[4,5-b]pyridin-3-yl]methyl]pyridine-2-carbonitrile;
5-[[2-(4-amino-1,2,5-oxadiazol-3-yl)imidazo[4,5-b]pyridin-3-yl]methyl]pyridine-2-carbonitrile;
5-[[7-fluoro-2-(4-methyl-1,2,5-oxadiazol-3-yl)benzimidazol-1-yl]methyl]pyridine-2-carbonitrile;
3-[1-[(6-methoxypyridin-3-yl)methyl]benzimidazol-2-yl]-4-methyl-1,2,5-oxadiazole;
5-[[2-(4-methyl-1,2,5-oxadiazol-3-yl)benzimidazol-1-yl]methyl]pyridin-2-ol;
5-[[6-fluoro-2-(4-methyl-1,2,5-oxadiazol-3-yl)benzimidazol-1-yl]methyl]pyridine-2-carbonitrile;
3-[6,7-difluoro-1-(pyridin-3-ylmethyl)benzimidazol-2-yl]-4-methyl-1,2,5-oxadiazole;
3-methyl-4-[1-(pyrimidin-5-ylmethyl)benzimidazol-2-yl]-1,2,5-oxadiazole;
3-methyl-4-[1-[(6-(methylsulfonyl)pyridin-3-yl)methyl]benzimidazol-2-yl]-1,2,5-oxadiazole;
4-[3-(pyridin-3-ylmethyl)imidazo[4,5-b]pyridin-2-yl]-1,2,5-oxadiazol-3-amine;
3-[1-[(6-chloropyridin-3-yl)methyl]benzimidazol-2-yl]-4-methyl-1,2,5-oxadiazole;
5-[[4-chloro-2-(4-methyl-1,2,5-oxadiazol-3-yl)benzimidazol-1-yl]methyl]pyridine-2-carbonitrile;
5-[[7-chloro-2-(4-methyl-1,2,5-oxadiazol-3-yl)benzimidazol-1-yl]methyl]pyridine-2-carbonitrile;
3-methyl-4-[1-[(6-methylpyridin-3-yl)methyl]benzimidazol-2-yl]-1,2,5-oxadiazole;
3-methyl-4-[1-[(2-methylpyrimidin-5-yl)methyl]benzimidazol-2-yl]-1,2,5-oxadiazole;
3-[4,7-difluoro-1-(pyridin-3-ylmethyl)benzimidazol-2-yl]-4-methyl-1,2,5-oxadiazole;
3-[1-[(2-methoxypyridin-4-yl)methyl]benzimidazol-2-yl]-4-methyl-1,2,5-oxadiazole;
3-[[2-(4-methyl-1,2,5-oxadiazol-3-yl)benzimidazol-1-yl]methyl]pyridine-2-carbonitrile;
5-[7-fluoro-1-(pyridin-3-ylmethyl)benzimidazol-2-yl]-1,2,3-thiadiazole;
3-methyl-4-[1-[(3-methylpyridin-2-yl)methyl]benzimidazol-2-yl]-1,2,5-oxadiazole;
3-[7-ethoxy-1-(pyridin-4-ylmethyl)benzimidazol-2-yl]-4-methyl-1,2,5-oxadiazole;
2-(4-methyl-1,2,5-oxadiazol-3-yl)-3-(pyridin-3-ylmethyl)benzimidazol-4-amine;
N-methyl-5-[[2-(4-methyl-1,2,5-oxadiazol-3-yl)benzimidazol-1-yl]methyl]pyridin-2-amine;
3-methyl-4-[1-[(2-methylpyridin-4-yl)methyl]benzimidazol-2-yl]-1,2,5-oxadiazole;
3-[1-[(4,6-dimethylpyridin-2-yl)methyl]benzimidazol-2-yl]-4-methyl-1,2,5-oxadiazole;
3-methyl-4-[1-[(1-oxidopyridin-1-ium-3-yl)methyl]benzimidazol-2-yl]-1,2,5-oxadiazole;
3-methyl-4-[1-[(1-oxidopyridin-1-ium-4-yl)methyl]benzimidazol-2-yl]-1,2,5-oxadiazole;
3-methyl-4-[1-[(6-methylpyridin-2-yl)methyl]benzimidazol-2-yl]-1,2,5-oxadiazole;
3-methyl-4-[1-(pyridin-2-ylmethyl)benzimidazol-2-yl]-1,2,5-oxadiazole;
5-[[6-fluoro-2-(4-methyl-1,2,5-thiadiazol-3-yl)benzimidazol-1-yl]methyl]pyridine-2-carbonitrile;
N-methyl-2-(4-methyl-1,2,5-oxadiazol-3-yl)-3-(pyridin-3-ylmethyl)benzimidazol-4-amine;
3-[1-[(3-fluoropyridin-2-yl)methyl]benzimidazol-2-yl]-4-methyl-1,2,5-oxadiazole;
5-[[4,7-difluoro-2-(4-methyl-1,2,5-oxadiazol-3-yl)benzimidazol-1-yl]methyl]pyrimidine-2-carbonitrile;
3-[4,7-difluoro-1-(pyrimidin-5-ylmethyl)benzimidazol-2-yl]-4-methyl-1,2,5-oxadiazole;
rac-4-[1-[1-(pyridin-3-yl)ethyl]benzimidazol-2-yl]-1,2,5-oxadiazol-3-amine;
4-[7-fluoro-1-(pyridin-3-ylmethyl)benzimidazol-2-yl]-1,2,5-oxadiazol-3-amine;
4-[4-fluoro-1-(pyridin-3-ylmethyl)benzimidazol-2-yl]-1,2,5-oxadiazol-3-amine;
4-(1-((6-bromopyridin-3-yl)methyl)benzimidazol-2-yl)-1,2,5-oxadiazol-3-amine;
3-[1-(pyridin-3-ylmethyl)benzimidazol-2-yl]-4-vinyl-1,2,5-thiadiazole;
4-[1-(pyridin-3-ylmethyl)benzimidazol-2-yl]-1,2,5-oxadiazol-3-amine;
4-[6-fluoro-1-(pyridin-4-ylmethyl)benzimidazol-2-yl]-1,2,5-oxadiazol-3-amine;
4-[5-fluoro-1-(pyridin-4-ylmethyl)benzimidazol-2-yl]-1,2,5-oxadiazol-3-amine;
4-[1-[(2-methoxypyridin-4-yl)methyl]benzimidazol-2-yl]-1,2,5-oxadiazol-3-amine;
4-[1-[[2-(trifluoromethyl)pyridin-4-yl]methyl]benzimidazol-2-yl]-1,2,5-oxadiazol-3-amine;
4-[1-[[5-(trifluoromethyl)pyridin-3-yl]methyl]benzimidazol-2-yl]-1,2,5-oxadiazol-3-amine;
N-methyl-4-[1-(pyridin-3-ylmethyl)benzimidazol-2-yl]-1,2,5-thiadiazol-3-amine;
4-[1-[[6-(trifluoromethyl)pyridin-3-yl]methyl]benzimidazol-2-yl]-1,2,5-oxadiazol-3-amine;
3-methyl-4-[1-(pyridin-4-ylmethyl)benzimidazol-2-yl]-1,2,5-oxadiazole;
4-[1-(pyridin-3-ylmethyl)benzimidazol-2-yl]-1,2,5-thiadiazole-3-carbonitrile;
2-(3-methylthiophen-2-yl)-1-(pyridin-3-ylmethyl)benzimidazole;
3-methyl-4-[1-(pyridin-3-ylmethyl)benzimidazol-2-yl]-1,2,5-oxadiazole;
5-methyl-4-[1-(pyridin-3-ylmethyl)benzimidazol-2-yl]-1,2,3-thiadiazole;
5-methyl-4-[1-(pyridin-3-ylmethyl)benzimidazol-2-yl]oxazole;
4-methyl-3-[1-(pyridin-3-ylmethyl)benzimidazol-2-yl]isoxazole;
3-ethyl-4-[1-(pyridin-3-ylmethyl)benzimidazol-2-yl]-1,2,5-thiadiazole;
4-[1-(pyrimidin-4-ylmethyl)benzimidazol-2-yl]-1,2,5-oxadiazol-3-amine;
4-[1-(pyridazin-4-ylmethyl)benzimidazol-2-yl]-1,2,5-oxadiazol-3-amine;
3-fluoro-4-[1-(pyridin-3-ylmethyl)benzimidazol-2-yl]-1,2,5-thiadiazole;
4-[1-[[2-(trifluoromethyl)pyridin-3-yl]methyl]benzimidazol-2-yl]-1,2,5-oxadiazol-3-amine;
3-methyl-4-[3-(pyridin-3-ylmethyl)imidazo[4,5-c]pyridin-2-yl]-1,2,5-oxadiazole;
4,5-dimethyl-3-[1-(pyridin-3-ylmethyl)benzimidazol-2-yl]isoxazole;
3-methyl-4-[1-(pyridin-3-ylmethyl)benzimidazol-2-yl]isoxazole;
2-(1,4-dimethylpyrazol-3-yl)-1-(pyridin-3-ylmethyl)benzimidazole;
2-(1-methylpyrazol-5-yl)-1-(pyridin-3-ylmethyl)benzimidazole;
3-ethyl-4-[1-(pyridin-3-ylmethyl)benzimidazol-2-yl]-1,2,5-oxadiazole;
2-(furan-2-yl)-1-(pyridin-4-ylmethyl)benzimidazole;
4-[6-fluoro-1-(pyridin-3-ylmethyl)benzimidazol-2-yl]-1,2,5-oxadiazol-3-amine;
4-[5-fluoro-1-(pyridin-3-ylmethyl)benzimidazol-2-yl]-1,2,5-oxadiazol-3-amine;
4-[7-fluoro-1-(pyridin-4-ylmethyl)benzimidazol-2-yl]-1,2,5-oxadiazol-3-amine;
4-[4-fluoro-1-(pyridin-4-ylmethyl)benzimidazol-2-yl]-1,2,5-oxadiazol-3-amine;
3-[7-fluoro-1-(pyridin-3-ylmethyl)benzimidazol-2-yl]-4-methyl-1,2,5-oxadiazole;
3-[4-fluoro-1-(pyridin-3-ylmethyl)benzimidazol-2-yl]-4-methyl-1,2,5-oxadiazole;
3-[4-fluoro-1-(pyridin-3-ylmethyl)benzimidazol-2-yl]-4-methyl-1,2,5-thiadiazole;
3-[7-fluoro-1-(pyridin-3-ylmethyl)benzimidazol-2-yl]-4-methyl-1,2,5-thiadiazole;
3-bromo-4-(1-(pyridin-3-ylmethyl)benzimidazol-2-yl)-1,2,5-thiadiazole;
3-methyl-4-[1-(pyridin-3-ylmethyl)benzimidazol-2-yl]-1,2,5-thiadiazole;
4-[7-fluoro-1-(pyridin-3-ylmethyl)benzimidazol-2-yl]-5-methyl-1,2,3-thiadiazole;
4-[4-fluoro-1-(pyridin-3-ylmethyl)benzimidazol-2-yl]-5-methyl-1,2,3-thiadiazole;
4-methyl-5-[1-(pyridin-3-ylmethyl)benzimidazol-2-yl]isoxazole;
4-[4-fluoro-1-(pyrimidin-5-ylmethyl)benzimidazol-2-yl]-1,2,5-oxadiazol-3-amine;
4-(7-fluoro-1-(pyridazin-4-ylmethyl)-benzimidazol-2-yl)-1,2,5-oxadiazol-3-amine;
4-(4-fluoro-1-(pyridazin-4-ylmethyl)-benzimidazol-2-yl)-1,2,5-oxadiazol-3-amine;
4-(7-fluoro-1-(pyrimidin-4-ylmethyl)-benzimidazol-2-yl)-1,2,5-oxadiazol-3-amine;
4-(4-fluoro-1-(pyrimidin-4-ylmethyl)-benzimidazol-2-yl)-1,2,5-oxadiazol-3-amine;
4-(5,7-difluoro-1-(pyridin-4-ylmethyl)-benzimidazol-2-yl)-1,2,5-oxadiazol-3-amine;
3-(7-fluoro-1-((6-(methylsulfonyl)pyridin-3-yl)methyl)-benzimidazol-2-yl)-4-methyl-1,2,5-oxadiazole;
3-(4-fluoro-1-((6-(methylsulfonyl)pyridin-3-yl)methyl)-benzimidazol-2-yl)-4-methyl-1,2,5-oxadiazole;
3-(7-fluoro-1-(pyridazin-3-ylmethyl)-benzimidazol-2-yl)-4-methyl-1,2,5-thiadiazole;
3-(4-fluoro-1-(pyridazin-3-ylmethyl)-benzimidazol-2-yl)-4-methyl-1,2,5-thiadiazole;
4-(5,7-difluoro-1-(pyridazin-3-ylmethyl)-benzimidazol-2-yl)-1,2,5-oxadiazol-3-amine;
4-(4,6-difluoro-1-(pyridazin-3-ylmethyl)-benzimidazol-2-yl)-1,2,5-oxadiazol-3-amine;
4-(7-fluoro-1-(pyridazin-3-ylmethyl)-benzimidazol-2-yl)-N-methyl-1,2,5-thiadiazol-3-amine;
4-(4-fluoro-1-(pyridazin-3-ylmethyl)-benzimidazol-2-yl)-N-methyl-1,2,5-thiadiazol-3-amine;
4-(6,7-difluoro-1-(pyridin-4-ylmethyl)-benzimidazol-2-yl)-1,2,5-oxadiazol-3-amine;
3-(5,7-difluoro-1-(pyridazin-3-ylmethyl)-benzimidazol-2-yl)-4-methyl-1,2,5-oxadiazole;
3-(4,6-difluoro-1-(pyridazin-3-ylmethyl)-benzimidazol-2-yl)-4-methyl-1,2,5-oxadiazole;
4-(1-((6-chloropyridazin-3-yl)methyl)-7-fluoro-benzimidazol-2-yl)-1,2,5-oxadiazol-3-amine;
4-(1-((6-chloropyridazin-3-yl)methyl)-4-fluoro-benzimidazol-2-yl)-1,2,5-oxadiazol-3-amine;
6-((2-(4-amino-1,2,5-oxadiazol-3-yl)-7-fluoro-benzimidazol-1-yl)methyl)pyridazin-3-ol;
4-(1-((6-deuteriopyridazin-3-yl)methyl)-7-fluoro-benzimidazol-2-yl)-1,2,5-oxadiazol-3-amine;
4-(7-fluoro-1-((6-methoxypyridazin-3-yl)methyl)-benzimidazol-2-yl)-1,2,5-oxadiazol-3-amine;
4-(4-fluoro-1-((6-methoxypyridazin-3-yl)methyl)-benzimidazol-2-yl)-1,2,5-oxadiazol-3-amine;
4-(4,7-difluoro-1-(pyridazin-3-ylmethyl)-benzimidazol-2-yl)-1,2,5-oxadiazol-3-amine;
4-(4,7-difluoro-1-(pyridin-4-ylmethyl)-benzimidazol-2-yl)-1,2,5-oxadiazol-3-amine;
4-(7-fluoro-1-(pyridazin-3-ylmethyl)-benzimidazol-2-yl)-1,2,5-thiadiazol-3-amine;
4-(4-fluoro-1-(pyridazin-3-ylmethyl)-benzimidazol-2-yl)-1,2,5-thiadiazol-3-amine;
4-(7-fluoro-1-(pyridazin-3-ylmethyl)-benzimidazol-2-yl)isoxazol-3-amine;
4-(1-((6-chloropyridin-3-yl)methyl)-6-fluoro-1H-imidazo[4,5-b]pyridin-2-yl)-1,2,5-oxadiazol-3-amine;
4-(6-fluoro-1-(pyrimidin-5-ylmethyl)-1H-imidazo[4,5-b]pyridin-2-yl)-1,2,5-oxadiazol-3-amine;
(S)-4-(7-fluoro-1-(1-(pyridin-3-yl)ethyl)-benzimidazol-2-yl)-1,2,5-oxadiazol-3-amine;
4-(6,7-difluoro-1-(pyridazin-3-ylmethyl)-benzimidazol-2-yl)-1,2,5-oxadiazol-3-amine;
4-(4,5-difluoro-1-(pyridazin-3-ylmethyl)-benzimidazol-2-yl)-1,2,5-oxadiazol-3-amine;
N-methyl-5-((2-(4-methyl-1,2,5-oxadiazol-3-yl)-benzimidazol-1-yl)methyl)pyridine-2-sulfonamide;
5-((2-(4-methyl-1,2,5-oxadiazol-3-yl)-3H-imidazo[4,5-b]pyridin-3-yl)methyl)pyrimidine-2-carbonitrile;
4-(7-fluoro-1-((6-(trifluoromethyl)pyridazin-3-yl)methyl)-benzimidazol-2-yl)-1,2,5-oxadiazol-3-amine;
4-(4-fluoro-1-((6-(trifluoromethyl)pyridazin-3-yl)methyl)-benzimidazol-2-yl)-1,2,5-oxadiazol-3-amine;
4-(7-fluoro-1-((6-methylpyridazin-3-yl)methyl)-benzimidazol-2-yl)-1,2,5-oxadiazol-3-amine;
4-(4-fluoro-1-((6-methylpyridazin-3-yl)methyl)-benzimidazol-2-yl)-1,2,5-oxadiazol-3-amine;
3-(6,7-difluoro-1-(pyridazin-3-ylmethyl)-benzimidazol-2-yl)-4-methyl-1,2,5-oxadiazole;
3-(4,5-difluoro-1-(pyridazin-3-ylmethyl)-benzimidazol-2-yl)-4-methyl-1,2,5-oxadiazole;
4-(1-((6-(difluoromethyl)pyridazin-3-yl)methyl)-7-fluoro-benzimidazol-2-yl)-1,2,5-oxadiazol-3-amine;
4-(1-((6-(difluoromethyl)pyridazin-3-yl)methyl)-4-fluoro-benzimidazol-2-yl)-1,2,5-oxadiazol-3-amine;
6-((2-(4-amino-1,2,5-oxadiazol-3-yl)-7-fluoro-benzimidazol-1-yl)methyl)pyridazine-3-carbonitrile;
6-((2-(4-amino-1,2,5-oxadiazol-3-yl)-4-fluoro-benzimidazol-1-yl)methyl)pyridazine-3-carbonitrile;
4-(7-fluoro-1-((6-(trifluoromethoxy)pyridin-3-yl)methyl)-benzimidazol-2-yl)-1,2,5-oxadiazol-3-amine;
6-((2-(4-amino-1,2,5-oxadiazol-3-yl)-3H-imidazo[4,5-b]pyridin-3-yl)methyl)pyridazine-3-carbonitrile;
4-(7-fluoro-1-(pyridazin-3-ylmethyl)-benzimidazol-2-yl)-N-methyl-1,2,5-oxadiazol-3-amine;
4-(4-fluoro-1-(pyridazin-3-ylmethyl)-benzimidazol-2-yl)-1,2,5-oxadiazol-3-amine;
3-(1-((6-(ethylsulfonyl)pyridin-3-yl)methyl)-benzimidazol-2-yl)-4-methyl-1,2,5-oxadiazole;
3-methyl-4-(1-((6-(methylthio)pyridin-3-yl)methyl)-benzimidazol-2-yl)-1,2,5-oxadiazole;
3-(7-fluoro-1-(pyridazin-3-ylmethyl)-benzimidazol-2-yl)-4-methylisoxazole;
4-(7-fluoro-1-((2-methoxypyridin-4-yl)methyl)-benzimidazol-2-yl)-1,2,5-oxadiazol-3-amine;
4-(4-fluoro-1-((2-methoxypyridin-4-yl)methyl)-benzimidazol-2-yl)-1,2,5-oxadiazol-3-amine;
3-(4,7-difluoro-1-(pyridazin-3-ylmethyl)-benzimidazol-2-yl)-4-methyl-1,2,5-oxadiazole;
3-(7-fluoro-1-(pyridazin-3-ylmethyl)-benzimidazol-2-yl)isoxazol-4-amine;
4-(7-fluoro-1-(pyridazin-3-ylmethyl)-1H-imidazo[4,5-c]pyridin-2-yl)-1,2,5-oxadiazol-3-amine;
4-(7-fluoro-3-(pyridazin-3-ylmethyl)-3H-imidazo[4,5-c]pyridin-2-yl)-1,2,5-oxadiazol-3-amine;
or an enantiomer of any of the foregoing;
or a pharmaceutically acceptable salt, solvate or prodrug of any of the foregoing.

25. A process for the preparation of the compound of formula (I) or a pharmaceutically acceptable salt, N-oxide, solvate or prodrug thereof, as claimed in claim 17, wherein the process comprises:
(A) reacting a compound of formula (V) 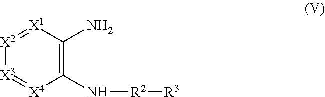
or a salt thereof, with a compound of formula (VI), R⁴—CO₂H (VI), or a salt thereof, or a compound of formula (VIII), R⁴—CHO (VIII), or a salt thereof, or a compound of formula (IX), Cl—C(NOH)—R⁴ (IX), or a salt thereof, wherein R², R³, R⁴, X¹, X², X³ and X⁴ are as defined in claim 17; or
(B) reacting a compound of formula (XII) 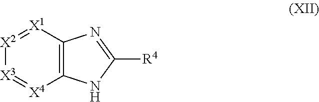
or a salt thereof, with a compound of formula (XIII), Z—R²—R³ (XIII), or a salt thereof, wherein R², R³, R⁴, X¹, X², X³ and X⁴ are as defined in claim 17, and Z is a leaving group;
and optionally thereafter carrying out one or more of the following procedures:
converting a compound of formula (I) into another compound of formula (I);
removing any protecting groups;
forming a pharmaceutically acceptable salt or N-oxide.

26. A pharmaceutical composition comprising the compound of formula (I) or a pharmaceutically acceptable salt, N-oxide, solvate or prodrug thereof, as claimed in claim 17, in association with a pharmaceutically acceptable adjuvant, diluent or carrier, and optionally one or more other therapeutic agents.

27. A method of treating or preventing a disease, disorder or condition associated with KCNK13 activity, the method comprising administering to a patient in need thereof a therapeutically or prophylactically effective amount of the compound of formula (I) or a pharmaceutically acceptable salt, N-oxide, solvate or prodrug thereof, as claimed in claim 17.

28. The method as claimed in claim 27, wherein the disease, disorder or condition associated with KCNK13 activity is a neurodegenerative disease, a psychiatric disease, a genetic disease, hearing loss, an ocular or retinal disease, a cardiovascular disease, an inflammatory disease, an autoimmune disease, or a metabolic disease.

29. The method as claimed in claim 27, wherein the disease, disorder or condition associated with KCNK13 activity is Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes.

30. A method of treating or preventing a disease, disorder or condition associated with KCNK13 activity, the method comprising administering to a patient in need thereof a therapeutically or prophylactically effective amount of a compound of formula (I) 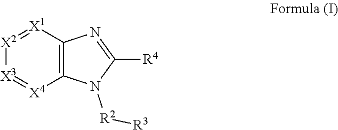
or a pharmaceutically acceptable salt, N-oxide, solvate or prodrug thereof, wherein:
each X¹, X², X³ and X⁴ is independently CH, CR¹ or N;
each —R¹ is independently halo, —CN, —R^{α}, —OH, —OR^{α}, —NH₂, —NHRα, —N(R^{α})₂, —SR^{α}, —SOR^{α}, —SO₂R^{α}, —SO(NH)R^{α}, —SO₂NHR^{α}, —SO₂N(R^{α})₂, —NH—SOR^{α}, —NH—SO₂R^{α}, —NH—SO₂NHR^{α}, —NH—SO₂N(R′)₂, —NR^{α}—SOR^{α}, —NR^{α}—SO₂R^{α}, —NR^{α}—SO₂NH₂, —NR^{α}—SO₂NHR^{α}, —NR^{α}—SO₂N(R^{α})₂, —COR^{α}, —COOR^{α}, —OCOR^{α}, —NH—CHO, —NR^{α}—CHO, —NH—COR^{α}, —NR^{α}—COR^{α}, —NH—COOR^{α}, —NR^{α}—COOR^{α}, —CONH₂, —CONHR^{α}, —CON(R^{α})₂, —NH—CON(R^{α})₂, —NR^{α}—CON(R^{α})₂, or a C₃-C₆ cycloalkyl, phenyl, 3- to 6-membered heterocyclic, or 5- or 6-membered heteroaryl group, wherein the cycloalkyl, phenyl, heterocyclic or heteroaryl group is optionally substituted with one or two substituents independently selected from C₁-C₃ alkyl or —CO(C₁-C₃ alkyl);
R²— is —C(R^{θ})₂-, —C(R^{θ})₂-C(R^{θ})₂-, —C(R^{θ})₂-O—, —C(R^{θ})₂-NR—, —C(R^{θ})₂-CO—, or —C(R^{θ})₂-CONR⁹—;
R³ is a 6-membered heteroaryl group with one or more nitrogen atoms in the ring structure, wherein the heteroaryl group is optionally substituted with one, two, three or four substituents independently selected from halo, —CN, —R^{δ}, —OH, —OR^{α}, —NH₂, —NHR^{α}, —N(R^{δ})₂, —SH, —SR^{δ}, —SOR^{δ}, —SO₂R^{δ}, —SO(NH)R^{δ}, —SO(NR^{δ})R^{δ}, —SO₂NH₂, —SO₂NHR^{δ}, —SO₂N(R^{δ})₂, —NH—SOR^{δ}, —NH—SO₂R^{δ}, —NH—SO₂NHR^{δ}, —NH—SO₂N(R^{δ})₂, —NR⁶⁷—SOR^{δ}, —NR⁶⁷—SO₂R^{δ}, —NR⁶⁷—SO₂NH₂, —NR⁶⁷—SO₂NHR^{δ}, —NR⁶⁷—SO₂N(R^{δ})₂, —COR⁶⁷, —COOR⁶⁷, —OCOR⁶⁷, —NH—CHO, —NR⁶⁷—CHO, —NH—COR⁶⁷, —NR⁶⁷—COR⁶⁷, —NH—COOR⁶⁷, —NR⁶⁷—COOR⁶⁷, —CONH₂, —CONHR⁶⁷, —CON(R^{δ})₂, —NH—CONHR⁶⁷, —NR⁶⁷—CONHR⁶⁷, —NH—CON(R^{δ})₂, or —NR⁶⁷—CON(R^{δ})₂;
R⁴ is a 5-membered heteroaryl group with one or more heteroatoms N, O or S in the ring structure, wherein the heteroaryl group is optionally substituted with one, two, three or four substituents independently selected from halo, —CN, —R^{ε}, —OH,-ORE, —NH₂, —NHR^{ε}, —N(R^{ε})₂, —SH, —SR^{ε}, —SOR^{ε}, —SO₂RE, —SO₂NH₂, —SO₂NHR^{ε}, —SO₂N(RE)₂, —NH—SO₂RE, —NH—SO₂NHR^{ε}, —NH—SO₂N(RE)₂, or —NR^{ε}SO₂R^{ε};
each —R^{α} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{δ} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃;
each —R^{E} is independently C₁-C₃ alkyl, C₂-C₃ alkenyl, C₂-C₃ alkynyl or C₃-C₆ cycloalkyl, all optionally substituted with one or more substituents independently selected from halo, —OH, —NH₂ or —SO₂CH₃; and
each —R^{a} is independently hydrogen or methyl.

31. The method as claimed in claim 30, wherein the disease, disorder or condition associated with KCNK13 activity is a neurodegenerative disease, a psychiatric disease, a genetic disease, hearing loss, an ocular or retinal disease, a cardiovascular disease, an inflammatory disease, an autoimmune disease, or a metabolic disease.

32. The method as claimed in claim 30, wherein the disease, disorder or condition associated with KCNK13 activity is Alzheimer's disease, Parkinson's disease, frontal temporal dementia, progressive supranuclear palsy (PSP) and related tauopathies, amyotrophic lateral sclerosis (ALS)/motor neuron disease (MND), traumatic brain injury, multiple sclerosis, stroke, ischemic insult, depression, stress, anxiety related disorder (including social and generalised anxiety), post-traumatic stress disorder (PTSD), schizophrenia, bipolar disorder, cryopyrin-associated periodic syndrome (CAPS) (including Muckle-Wells syndrome (MWS), familial cold autoinflammatory syndrome (FCAS), chronic infantile neurological cutaneous and articular (CINCA) syndrome, and neonatal onset multisystem inflammatory disease (NOMID)), age related hearing loss, genetic related hearing loss (including NLRP3 mutation related hearing loss), autoimmune related hearing loss, macular degeneration, age related macular degeneration, diabetic retinopathy, atherosclerosis, myocardial infarction, ischemia, rheumatoid arthritis, gout, Lupus, asthma, respiratory inflammation, inflammatory or autoimmune skin disease, psoriasis, inflammatory bowel disease, colitis, metabolic syndrome, non-alcoholic fatty liver disease (NAFLD), non-alcoholic steatohepatitis (NASH), fibrosis, or diabetes.
